SEQUENCE LISTING (85 sequences)

SEQ ID NO: 1 - protein, 1273 aa, Artificial Sequence
  spike protein published under GenBank Accession YP_009724390
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1259 aa, Artificial Sequence
  spike protein
MVNLTTRTQLPPAYTNSFTRGVYYPDKVFRSSVLHSTQDLFLPFFSNVTWFHAIHVSGTNGTKRFDNPVLPFNDGVYFASTEKSKIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASLSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1282 aa, Artificial Sequence
  spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 4 - protein, 1280 aa, Artificial Sequence
  spike protein
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 5 - protein, 1282 aa, Artificial Sequence
  spike protein
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 6 - protein, 1281 aa, Artificial Sequence
  spike protein
MFVFLVLLPLVSIQCVNLTTRTQLPPAYTNSFTRGVYYPDKVFRSSVLHSTQDLFLPFFSNVTWFHAIVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 7 - protein, 1282 aa, Artificial Sequence
  spike protein
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 8 - protein, 1281 aa, Artificial Sequence
  spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYRYRLFRKSNLKPFERDISTEIYQAGSTPCNGVQGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 9 - protein, 1252 aa, Artificial Sequence
  spike proteins where the furin cleavage site is absent
MVNLTTRTQLPPAYTNSFTRGVYYPDKVFRSSVLHSTQDLFLPFFSNVTWFHAIHVSGTNGTKRFDNPVLPFNDGVYFASTEKSKIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASLSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1199 aa, Artificial Sequence
  spike proteins where the furin cleavage site is absent
MVNLTTRTQLPPAYTNSFTRGVYYPDKVFRSSVLHSTQDLFLPFFSNVTWFHAIHVSGTNGTKRFDNPVLPFNDGVYFASTEKSKIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASLSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 11 - protein, 1199 aa, Artificial Sequence
  spike protein extracellular domain
MVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 12 - protein, 1208 aa, Artificial Sequence
  spike protein extracellular domain
MVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 13 - protein, 1206 aa, Artificial Sequence
  spike protein extracellular domain
MVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 14 - protein, 1208 aa, Artificial Sequence
  spike protein extracellular domain
MVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 15 - protein, 1207 aa, Artificial Sequence
  spike protein extracellular domain
MVNLTTRTQLPPAYTNSFTRGVYYPDKVFRSSVLHSTQDLFLPFFSNVTWFHAIVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 16 - protein, 1208 aa, Artificial Sequence
  spike protein extracellular domain
MVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPSRLEEELRRRLTE

SEQ ID NO: 17 - protein, 1207 aa, Artificial Sequence
  spike protein extracellular domain
MVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYRYRLFRKSNLKPFERDISTEIYQAGSTPCNGVQGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 18 - DNA, 3855 bp, Artificial Sequence
  nucleic acid encoding a spike protein
gccaccatgttcgtatttctcgtcctcctcccacttgtttctagtcagtgtgtgaacctcactacgcgaactcaactgccgcctgcttacactaatagttttacacgaggagtctactacccggacaaggtgttccgctcaagtgtcttgcacagcacgcaagacctgttcttgccatttttcagcaatgtaacgtggtttcatgccatccacgtgtcaggtactaacggaacgaagagatttgataacccggtcctcccgttcaacgacggggtttattttgccagtacagaaaagtccaatattataaggggatggattttcggcacgactctcgactctaagacacaaagcttgctcatcgttaataatgctacaaacgtggttataaaagtatgcgagttccaattctgtaacgacccctttctgggggtttactaccacaaaaataataagagttggatggagagcgaattcagagtttactcttccgcgaataattgcaccttcgagtacgtatcccagccttttcttatggatcttgaggggaaacaaggtaatttcaaaaaccttagagaattcgtttttaagaatatagatggatacttcaagatttattctaaacataccccaatcaatctggtgcgcgatcttcctcagggtttctctgcgctggagcctctcgttgatctgccgatcggaattaatataaccagattccagacccttttggcattgcaccgctcctatctgacacctggggatagtagttccggctggacagcgggcgccgcggcatactacgttggatatctccaaccccgcacttttcttctgaaatataatgagaatggaacgattacagacgcagtagattgcgcgcttgaccccctttcagaaacaaagtgcacgctgaaatctttcacggtcgagaagggcatttaccaaacgtccaatttcagagttcaacccactgagtctatagtcaggtttccgaacatcaccaatctctgcccatttggagaggttttcaacgcaacacggttcgcctctgtatacgcatggaatagaaagagaatatccaattgtgtcgcagactatagtgtcttgtacaactctgcgtcatttagcacttttaaatgttatggggtgagtccaactaaattgaacgatctctgctttacaaatgtttacgcagatagttttgtgatacgaggcgacgaggtacgacaaattgctcctggccagaccgggaaaatagcggactacaattataaactgccagacgatttcaccggctgtgtgatcgcgtggaactcaaacaacttggatagtaaggtaggtggtaactataactacttgtaccggctcttccgcaaaagtaatcttaaaccgttcgagcgggacatctctaccgagatctatcaagcaggtagcactccctgcaatggagtcgagggttttaattgctattttcccctccaatcatacgggtttcagcccacaaatggtgttgggtatcagccctatcgagttgtggtgctttcatttgaattgcttcatgcaccggcgacagtttgtggtcctaaaaagagtaccaatttggttaaaaacaaatgtgtcaactttaactttaacggtctgactggtacgggggtgcttacggaatctaataaaaagtttcttccgttccagcaattcggacgggacatagcggacacgactgacgcggttagagacccacaaacactcgagattcttgacataaccccctgttctttcggcggagtttctgtaataacgcctgggacaaatacgtcaaaccaggttgcagtattgtaccaggatgtcaactgcactgaagtgccagtcgcgatccacgcagatcagttgactccaacatggagggtctactctaccggttctaacgtgtttcagaccagagccgggtgcctcattggcgcggaacatgtgaacaattcttacgaatgcgacatacccatcggcgccggcatttgtgcgtcataccaaactcaaactaattcccggagtgtggcctcccaatctattattgcttatactatgagtcttggcgcggaaaacagtgtagcgtactcaaataactctattgcgatccccacgaatttcactataagcgtaactactgaaattttgccggtaagcatgacgaaaacttcagtggactgcacaatgtacatttgtggtgacagcactgagtgctcaaatctcctccttcagtatggttctttctgcacacaacttaacagggcacttaccggaatcgcagtagaacaagacaaaaacacgcaagaagtattcgcacaggtcaaacaaatttataaaaccccgccgatcaaggattttggagggtttaacttcagtcagatccttccagacccatccaaacccagcaaacggagctttattgaagacctgctgtttaataaagttacccttgcagatgccggtttcatcaagcaatacggggattgtctgggcgacattgccgcccgcgacttgatctgtgcccagaagttcaacggtctcacagtcttgccgccacttctcactgatgaaatgatagcgcagtatacatccgcgctgctggctgggacgataacatctggttggactttcggggccggggcggctttgcaaattcctttcgccatgcagatggcctataggtttaacggcataggagtgactcaaaatgttctttatgagaatcagaagcttatcgccaaccaatttaactccgcgattgggaagatacaggactcattgagctctactgctagcgcccttggcaagcttcaagacgtagtcaaccaaaatgctcaagcgctcaatactctcgtaaagcagctctcaagcaactttggcgcgatatcttcagttttgaacgacatcttgagcagactcgacaaggtggaggctgaagttcaaattgatcgactgattacgggccggctccagtcactccaaacgtacgtgacccaacaactcattagagcggcagagattagagcgagcgctaatctggcggccacaaagatgtcagaatgtgtgcttggtcaatccaaaagggtggatttctgcggaaagggctatcacttgatgagtttcccgcagtccgcgcctcatggagttgttttcctccacgtaacctacgttcccgctcaggaaaagaatttcacgacagctccggcaatatgtcatgacggtaaagcacatttccccagggaaggcgtttttgtcagtaatggaacccactggttcgtcacccagcgaaacttttacgagccccaaataataactactgacaataccttcgtcagcggcaattgcgacgtcgtcataggcatagtaaataatacggtttacgaccctcttcaacccgagttggacagcttcaaagaagaacttgataagtattttaaaaaccacactagcccagatgtagaccttggcgacatcagcggtatcaatgcttccgttgtgaacattcaaaaggaaattgaccgactcaatgaagtagcgaagaacttgaatgagtctctgatcgatcttcaggagctcgggaaatatgagcaatacatcaaatggccgtggtacatctggctgggttttatagctggcctgatcgcaattgtaatggtaactataatgttgtgttgcatgacctcttgctgcagctgtttgaagggatgctgttcttgcgggagttgctgtaaatttgatgaggatgacagcgagccggtgttgaaaggagtgaagcttcattatacttcacgactggaggaagaactgcgccgacgcctgactgaatga

SEQ ID NO: 19 - DNA, 3849 bp, Artificial Sequence
  sequence encoding a spike protein
gccaccatgttcgtctttctcgtgctgttgcctcttgtatcttctcagtgcgttaacctgacaacacgcacccagttgccaccggcctatacgaactctttcacgcgcggagtgtattatcccgataaagtctttcggagttctgtgctgcactctacacaagatctttttctgccctttttctccaacgttacgtggttccatgcaataagtggtacgaatggtactaaaaggttcgataatcccgtacttcccttcaatgacggcgtgtatttcgcgagcacagaaaagagcaacataataagaggttggatttttgggactaccctggactctaaaacacaaagcctcctcattgttaataacgcaactaatgtcgtaataaaggtatgcgaatttcagttttgtaacgacccttttttgggagtctatcataagaacaataagtcctggatggaaagtgaatttagggtgtatagttcagcgaacaactgtacatttgagtatgtgtcacagccgttccttatggatcttgaagggaaacaggggaacttcaagaacctgcgggaattcgtgtttaaaaatatagacggttattttaaaatctatagcaaacacaccccaataaacctggttcgggatcttcctcagggattttcagctttggaaccgttggttgacctccccatcgggataaatatcacgcgatttcaaacactgctggcactgcaccggagttatctgactcctggagatagcagctccggctggacggctggcgccgcggcttattatgtcgggtaccttcaaccgcgaacgtttcttttgaagtataacgaaaatggcactataaccgatgcagtagattgtgcgttggaccctctttccgagactaagtgcaccctgaagtcatttacagtagagaaggggatataccagacaagcaacttccgcgtccaaccaacggaaagcatcgtgcgctttcccaatattactaacctctgtccgttcggcgaggtttttaatgctaccagatttgcctcagtgtatgcctggaaccgcaagaggattagcaactgcgttgctgactatagcgtgctttacaattccgccagttttagtacgttcaagtgttatggggtaagccctaccaagcttaacgatctttgcttcacgaatgtttacgcagactcattcgtaatacgaggtgatgaagtgcgacagatagccccgggtcagacggggaaaatagccgactacaattacaagctcccggacgacttcacgggctgtgttattgcgtggaacagcaataacctggacagtaaagttggagggaattataactacttgtacaggctgttccgcaaatctaatttgaaaccgtttgaaagggacatttccaccgagatttaccaagccggcagtaccccatgtaacggagttgagggatttaattgctactttccgctccaatcatatggattccagcctacatacggcgtcggatatcaaccttatagggtggtcgttctttcttttgagctgcttcacgccccagcaaccgtttgcggcccgaaaaaatcaacgaacttggtcaaaaacaagtgcgtcaacttcaatttcaacggactgacaggcaccggagtgctcaccgagtcaaataaaaaattcctcccgtttcaacaattcggtagagatatagacgacaccacagatgcggtacgcgaccctcagactcttgaaattttggacattacaccatgttccttcggtggcgtctcagtgattacgcctggcactaacactagcaaccaagtcgcagtactttatcagggcgttaactgtacagaagtaccagttgcgatacacgcggaccagctcaccccaacctggcgcgtatattctaccggatctaatgtgtttcaaacgcgggcggggtgcctcataggtgcagagcatgtcaataactcctacgaatgtgacattcctataggagcaggaatatgtgcgagctaccagactcaaaccaattcacacaggagcgtagctagtcagtccataattgcctacactatgtcacttggtgctgaaaacagtgtggcttattcaaataattccatagctattccaatcaatttcactattagcgtaacaactgagatacttccagtcagcatgactaagacttcagttgattgtacgatgtatatttgtggcgattctacagaatgtagtaaccttctgttgcaatacggctccttttgcacacagctcaatcgcgcattgacgggtatagctgtagagcaagataaaaacacccaagaagtcttcgcgcaggtgaagcaaatatataagacaccaccgattaaagatttcggtggattcaattttagccaaattctgcctgatccaagcaaaccgtcaaaaagatccttcatagaggacctgcttttcaataaggtaacgctggcggacgcagggtttattaaacaatacggcgattgccttggtgacattgctgcacgagacctcatttgcgcccagaagttcaacggactcacagtactgcctccactgctcacagacgagatgatcgcccagtacacatctgccttgttggctggtacgataacttctggttggacctttggagcgggagcagcacttcagattccctttgccatgcagatggcgtacagattcaacggcattggtgtcacgcagaacgtactgtatgagaaccagaagttgatagctaatcaattcaactccgcgataggtaagatccaagacagtctctcctcaacagcctccgcactcgggaaactccaggatgtggtaaatcagaatgcacaggcattgaatacgcttgttaaacaattgtcctcaaattttggcgccatatcatcagtgctcaacgacatccttgccagactcgataaagtggaggcagaagtgcagatagatcgcttgattactgggcgcctccaatctctccaaacgtacgtgacacaacagcttattcgggcagcggaaatccgcgcctctgcgaaccttgcagcgaccaagatgagcgaatgtgttctcgggcagtcaaaacgagtagatttctgcggtaaaggatatcatcttatgtcattcccccaaagcgctccacatggtgttgtatttctgcacgtgacttacgtgccggcacaggagaagaatttcacaacggctcccgccatctgtcacgacggaaaggcacacttccctagggagggggtgtttgtaagcaatggtacccactggtttgtcacacaacgaaacttctatgaacctcagataattacaacgcataatacttttgtaagtgggaactgcgacgtcgttatcggaatagttaacaatactgtatatgatccgttgcagccggagttggactcatttaaagaagagctggacaagtacttcaaaaaccatacaagcccagatgtagatcttggggacataagtgggataaacgccagcgttgtgaacatacagaaggaaatagacaggctgaatgaggtagcaaagaatcttaacgaaagccttatagatcttcaggaacttgggaagtacgaacagtacataaagtggccttggtatatttggctcggtttcatcgctggactcatagcgatagtaatggtgactatcatgctgtgttgtatgaccagttgctgctcatgcctgaagggttgctgctcttgcggctcctgctgtaaattcgacgaagatgactccgagccagtgctgaagggggtgaagctgcattatacttcacgactggaggaagaactgcgccgacgcctgactgaatga

SEQ ID NO: 20 - DNA, 3855 bp, Artificial Sequence
  sequence encoding a spike protein
gccaccatgttcgtattccttgttcttttgccgcttgtgtcttctcagtgtgtcaatttcactacacgcacacagctgcccccggcgtacactaacagttttacgaggggagtctactacccggacaaagtgttcaggtcttccgtactccatagcacacaagacctcttcttgccttttttcagcaatgttacgtggttccatgcaatccatgtgagcggtacaaacgggactaaacgctttgcaaatccagtgttgcccttcaacgacggtgtatacttcgcgagtacagagaagagcaacataatacgcgggtggatatttggaacgacgcttgattctaagacacagagcttgctcatcgtaaacaacgcaacgaatgttgtaatcaaggtctgcgaattccaattctgcaatgacccatttcttggagtttattaccataagaacaataagtcttggatggaatccgagttccgcgtatactctagcgcgaacaactgcaccttcgagtatgtgtcccaaccgtttctgatggatcttgagggaaagcagggtaattttaaaaatctccgggagttcgttttcaaaaatattgacggatatttcaaaatctacagcaagcataccccaatcaatttggtgcggggtctgccacaaggattttccgctcttgaacctcttgttgaccttcccataggaatcaatataactcggttccaaactcttcttgcgctccatatatcctacttgacgcccggagattcatcaagtggctggacggctggggctgccgcatactacgtcggctatctgcaaccaaggacgtttctcctcaaatacaatgaaaatggtacgatcaccgacgctgtggactgcgctctcgacccgttgtcagagacgaagtgcaccttgaagtccttcaccgtggaaaagggaatttaccagactagtaacttccgggtgcagcccacggaatcaattgtgcgatttcctaatatcactaatctctgtcccttcggggaagtgttcaatgcgactcgattcgcttcagtatacgcttggaatagaaagagaatctcaaattgcgtggctgactattccgtactgtataatagtgcgtcttttagcacgtttaagtgttatggcgtatccccgacaaagctcaacgatctctgcttcacgaatgtctatgccgactccttcgtgatacgcggtgacgaggtacgacaaattgcacccggccaaactggaaatattgccgactacaattataagttgccggacgattttaccggttgcgtaatagcgtggaactctaataacctcgactctaaggtaggaggcaactacaattacttgtaccgactgttcaggaaatccaacctgaagcccttcgaaagagacatcagtacagagatttatcaagccgggtccaccccttgcaatggtgtcaaaggcttcaactgctattttccgttgcagtcttacggtttccagccgacgtacggtgtgggttatcaaccctaccgcgttgtggtgctgagctttgagcttctccacgctccagccactgtctgcgggcctaagaaatccacaaacctcgttaagaataaatgcgtaaatttcaacttcaacgggcttactggcacgggcgtgcttacggagtccaataagaaattcctgcctttccagcagttcggccgcgacatcgcggacacaacagatgctgtgcgagacccacaaaccctggagattctggacatcacgccttgctcttttggcggtgtcagcgtaataacgccaggcaccaacaccagtaatcaggtcgccgtgctttatcagggtgtgaactgtacggaggtaccggttgccatacacgcagaccaactcacacctacgtggagagtttacagcaccgggtcaaacgtatttcagacccgcgcaggctgtctgatcggcgcagagcatgtcaataactcatacgagtgtgatataccaatcggagccggcatctgtgcgagctatcaaacccaaactaattcacgaagcgtggccagccagtccatcattgcctacacgatgtctttgggagtagagaatagcgtggcatactctaacaacagtatcgctattcctaccaacttcacaatatcagttactacagaaatcctcccggtgagcatgactaagacatccgtagactgtacaatgtacatttgtggggacagcacggagtgttcaaacctgctgttgcaatacggaagcttctgcacccagctcaaccgagccctgaccggcattgccgtagaacaagataagaatacgcaggaagtgttcgcccaggttaagcagatttacaagactccgcctatcaaggacttcgggggtttcaactttagtcagattttgcctgacccgtcaaagcctagcaaacgaagttttatcgaagatttgttgtttaacaaggtgactttggctgatgcgggattcataaaacaatacggcgattgccttggcgatattgcggcccgagatctcatttgcgcgcaaaagttcaacggtcttaccgtccttccgcccctgcttacggacgagatgatcgcacaatacacttcagcactgctggctggtacaataaccagtgggtggacattcggggctggtgcagcgctccagatcccctttgcgatgcagatggcgtatagattcaatggcatcggggtcactcagaacgtgctttatgagaaccaaaagcttattgcaaatcaatttaactctgcgattggaaagattcaagacagcctctcatccacagctagtgcgcttggcaagcttcaggacgtcgtaaaccaaaacgcccaagctctcaacacactcgttaaacagctttcctcaaactttggggctattagctctgtgctgaatgatattcttagcagactcgacaaagtcgaggctgaagtacaaatagatagactgataacaggtagacttcagtctctccaaacctacgtgacacagcagcttataagagctgctgagatcagagcttctgcgaaccttgcggcgactaaaatgtccgagtgtgtactcgggcagtctaaacgcgtcgacttctgtggcaagggttaccatctcatgagttttccccaatcagcgccccatggggtagtttttctgcacgtaacatacgtcccagcccaagaaaagaattttaccaccgcccctgcgatttgtcacgacggaaaagcacactttccaagagaaggcgtatttgttagcaatggcacccattggttcgtcacccagagaaacttttacgaaccgcagattatcacgacagataatacatttgtatctggtaattgtgacgtcgtgatcggcatcgtaaacaacactgtttacgaccccctccagccagagcttgactcatttaaagaggagctcgacaaatattttaagaaccacacaagtccagacgtcgacttgggtgacatttccggcatcaatgcaagtgtggtcaacatccaaaaggaaattgacagattgaacgaggttgctaagaaccttaatgagtcactcattgacctccaggaactgggaaaatacgaacagtatattaagtggccctggtacatctggctgggatttatcgcgggacttatcgcgattgttatggtgacgataatgctttgctgcatgaccagctgttgtagttgcctcaagggttgctgctcatgcgggagctgctgtaaattcgacgaggacgattctgagcccgttttgaagggagtcaagcttcactacacgtcacgactggaggaagaactgcgccgacgcctgactgaatga

SEQ ID NO: 21 - DNA, 3852 bp, Artificial Sequence
  sequence encoding a spike protein
gccaccatgttcgtgttcctggtccttctccccttggtgtcaatccagtgtgtaaatttgacaacgcgcacccaattgcccccagcatatacgaactctttcacgaggggcgtatattatccggataaggtatttcggtcatctgttctgcacagcacccaggacctcttccttccattcttttcaaatgtaacttggttccatgcgatagtatcaggaacgaatgggacaaaaaggttcgataatccggtcttgccgttcaacgatggggtgtacttcgccagtaccgaaaagtctaacattatacgcggttggatttttggcactacgcttgactcaaagacacagtcactcctcattgtaaataatgctactaatgtcgtgatcaaagtttgtgagttccaattctgcaacgacccgttcctgggagtgtattatcacaaaaataataagtcctgcatggagtcagagttcagggtttactcaagtgcgaacaattgcacatttgagtacgtttctcaaccatttctcatggatttggaggggaagcagggaaactttaaaaacctgagagaatttgtttttaagaacattgatgggtatttcaagatttatagtaaacacacccctatcaacttggttagggatctccctcaaggtttctctgctctcgagccccttgtagatctgccaataggcatcaatatcacacgctttcagacactcctcgcacttcataggagctacctgacgccaggtgactcttcctcaggttggacagccggcgcagccgcatactacgttggctacctccaaccaaggacatttctgttgaaatacaacgaaaatgggaccatcaccgacgcagtcgattgtgctctcgaccctctttccgagactaaatgtaccctcaaaagctttactgttgagaagggtatctatcagacatctaactttcgggtgcaacccactgagtcaattgtgcgattcccaaatattacgaacctctgtccttttggcgaggtttttaacgccactaggttcgccagtgtatatgcttggaaccgaaaacggataagcaattgtgttgctgactactccgtcctctacaatagcgctagtttctcaacatttaagtgttacggtgtgagccctacgaaacttaacgatttgtgcttcactaacgtctatgccgacagtttcgtaatccgaggcgatgaggtcaggcaaattgccccgggccaaacggggaaaatcgctgattacaattataagttgccagatgattttacgggatgtgtcattgcatggaacagtaataacctcgattcaaaggttggcggaaattataattacagatatcggctttttagaaaatctaaccttaaaccatttgagcgggacataagcacggagatttaccaggctggtagcactccgtgcaacggtgtagaaggattcaattgctattttccattgcagtcttatggattccaacccaccaatggggtagggtaccaaccatacagggtggtagtccttagctttgaacttttgcatgcgccagctaccgtctgcggtcccaaaaagagtacgaacttggtaaaaaataaatgcgtcaactttaattttaacggtctgacgggaacgggggttctcaccgagtctaacaaaaagtttttgccatttcagcagttcggacgagatattgccgacactaccgacgccgtgcgcgatccacaaaccttggagatactcgacatcacaccttgcagcttcggtggtgttagcgttattacgccaggaacgaacacttcaaatcaggtggccgtcttgtatcaagatgttaactgtacagaggtgcccgtcgcaatacacgcagatcagctcacccctacctggagagtttactctacaggctctaatgtgttccaaactagggcaggctgcttgattggagctgaacacgtaaacaactcatacgagtgcgatatcccgattggtgcgggtatttgcgccagctatcagacgcagactaactcacgatcagtggcgtcacagtcaataattgcgtatacaatgagtctcggtgcagagaattccgtcgcatactcaaacaacagcatagcgattccaactaattttaccattagcgttacaacggaaatcctccccgttagcatgaccaagacctccgtggattgcacaatgtacatctgtggagacagcaccgagtgctccaatcttcttcttcaatatgggagcttttgcacacagctgaaccgcgcgctcactggcattgcggtagaacaagacaaaaacacccaagaagtgtttgcgcaggtcaaacagatttacaagacaccaccaatcaaagacttcggggggttcaatttctcccaaattcttcctgatccaagcaaaccatctaaaaggtccttcatagaagacctcctgttcaataaggtgacactcgcggacgcagggtttattaagcaatatggagattgtctcggggatattgcagcgagagatcttatttgtgctcaaaagtttaatggcttgactgtcctcccgcccttgctcacagacgagatgatagcacaatataccagtgcgctgcttgcgggcacgattacttccggatggactttcggggcaggagcggcgctccagattcctttcgcgatgcaaatggcctaccgatttaacgggattggcgtcacacaaaacgttctttacgaaaaccagaaacttattgccaaccaattcaatagtgcaattggtaaaattcaggattcactctcctccacggcgtcagctctcggcaagctccaagacgtggttaaccaaaatgcgcaggcgcttaacacgcttgtaaaacaactctcttctaattttggcgcgattagtagcgttctcaacgatatcttgtcacgcttggacaaagtagaagcagaagtccaaatagaccggctgatcactggtcgattgcaatctctgcagacatacgtgacgcagcagctcatccgggcagccgagatccgggcgtccgctaatctggccgcaaccaaaatgagcgagtgtgtccttgggcaaagcaaacgcgtggatttttgcggaaagggctaccacctcatgagtttcccccaatctgccccgcacggtgtcgtctttttgcatgtaacgtatgtcccggcgcaggagaaaaatttcactacagctcccgccatctgtcacgacggcaaggcacacttccctcgagaaggggtgtttgtaagcaatggaacacactggttcgtcacccagcggaatttttatgaacctcagataatcacaacagacaacactttcgtttccggaaactgcgatgtcgtaattgggatagtgaataataccgtatatgacccattgcagccggagttggactcctttaaagaggaattggacaaatactttaaaaatcacacttcacccgatgtggacctgggagatatatccgggattaacgcaagtgtggttaacatacaaaaggaaattgaccgccttaacgaggttgctaagaacttgaatgagtctctcatagatctccaggaactggggaaatacgagcaatatatcaaatggccgtggtacatatggctcggatttatagccggcctcatagcaatagttatggtcactataatgttgtgctgcatgacgtcctgttgtagttgcctcaaggggtgctgttcctgcggtagttgctgcaaatttgatgaagatgactccgaaccggttctcaagggagtcaagcttcattatacctcacgactggaggaagaactgcgccgacgcctgactgaatga

SEQ ID NO: 22 - DNA, 3855 bp, Artificial Sequence
  sequence encoding a spike protein
gccaccatgtttgtgttcctggtccttctcccgcttgtaagctcccaatgcgttaactttactaatcggacccaactcccgagtgcgtacactaactcttttacccggggcgtctattatcctgacaaagtgtttcgatcatctgttttgcactccacccaagatctctttcttccatttttcagcaacgttacgtggttccacgcgatacatgttagtggtactaatggcacaaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaggagctatctgaccccgggcgactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaacattgctgattataactacaagttgcccgatgacttcacggggtgtgttattgcatggaattcaaataatcttgatagtaaggtagggggtaactataattacctttatcgcttgttccgcaaatccaatctgaaacccttcgaaagggatatcagtaccgagatttatcaagccggctccacaccttgtaacggagtaaaaggttttaactgttatttcccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagcagaagcgttgcgtcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgacggttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctattaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttggagacatctctgggataaacgctagcttcgtaaacatacaaaaagaaatcgataggcttaacgaggtcgcaaaaaatcttaatgaaagtcttatcgatttgcaagagcttggaaagtatgagcagtatatcaaatggccttggtatatatggctgggatttattgctggtctcatagccatagttatggtgacaataatgctgtgctgtatgacgtcatgttgttcctgcctgaaagggtgctgtagttgtgggagttgttgcaagtttgacgaggacgactcagagcccgttttgaaaggtgtgaaacttcattatacatcacgactggaggaagaactgcgccgacgcctgactgaatga

SEQ ID NO: 23 - DNA, 3851 bp, Artificial Sequence
  sequence encoding a spike protein
ccaccatgttcgtatttctcgtcctcctcccacttgtttctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatctggctgggttttatagctggcctgatcgcaattgtaatggtaactataatgttgtgttgcatgacctcttgctgcagctgtttgaagggatgctgttcttgcgggagttgctgtaaatttgatgaggatgacagcgagccggtgttgaaaggagtgaagcttcattatacttcacgactggaggaagaactgcgccgacgcctgactgaataa

SEQ ID NO: 24 - DNA, 3789 bp, Artificial Sequence
  sequence encoding a spike protein
gccgccaccatggtgaacctgaccacccggacccagctacctcctgcctacacaaacagcttcacccgcggcgtgtactaccctgataaggtgttccggagctctgtgctccacagcacacaggacctgttcctgcctttcttcagcaacgtgacgtggttccacgccatccacgtgtccggcaccaatggtacaaaaaggtttgacaaccccgtgctccccttcaacgacggcgtgtattttgccagcactgaaaagtccaatatcatccgaggatggatcttcggcacaacgctggactctaagacccagagcctgctgatcgtgaacaacgccaccaacgtggtgatcaaggtgtgcgagtttcagttctgcaacgatccttttctgggcgtttactaccacaagaacaacaagagctggatggaaagcgagttcagagtgtattctagcgccaacaactgtaccttcgaatacgtcagccagcctttcctgatggacctggaaggcaagcagggcaacttcaagaacctgagagagttcgtgttcaagaacatcgacggctacttcaagatctacagcaagcacacacctatcaacctggtgcgggaccttccgcagggattctccgcgctggaacccctggtggacctgcctattggcatcaacatcaccagatttcagaccctgctggccctgcacagaagctacctgacacctggcgactcttctagcggctggaccgccggcgccgccgcttattacgtggggtacctgcagcccagaaccttcctgctgaaatacaatgagaacggaaccatcaccgatgccgtggactgcgccctggaccccctgtctgaaaccaagtgcaccctgaaatcttttacagtggaaaagggaatttaccagacctccaactttagagtccagcctaccgaaagcatcgtgcggttccctaacatcaccaacctgtgtcctttcggcgaggttttcaacgccacacggttcgccagcgtatacgcctggaaccggaagcggatctctaactgtgtggcagactactctgtgctatacaactcggccagctttagcaccttcaagtgctacggcgtgtccccaaccaagctgaacgacctgtgcttcaccaacgtgtacgccgatagcttcgtgatcagaggcgatgaggtgcgccagatcgctcctggacagaccggaaagatcgctgactacaattacaagctgcccgacgacttcaccggctgcgtgatcgcctggaacagcaacaacctggattccaaagtgggcggcaactacaactatctgtaccggctcttcagaaagtctaacctgaagccctttgagagagacatctccacagaaatctaccaggccggaagcacaccttgcaacggcgtggaaggatttaattgctattttcctctgcagtcctacggcttccaacctaccaacggagtgggctaccagccttacagagtggtcgtgctgagctttgagctgctgcatgcccctgctaccgtgtgtggccctaagaaaagcacgaacctggtcaagaacaaatgcgtgaacttcaacttcaatggactgacaggcaccggcgtgctcacagagagcaacaagaagttcctgccatttcagcagttcggcagagacatagctgacaccaccgacgccgtgcgggacccccagaccctggaaatcctggatatcacaccctgcagctttggaggcgtatctgtgatcacacctggcaccaacacaagcaatcaggtcgccgtgctgtaccaagacgtgaactgcacagaggtgccggtggccatccacgccgatcagctgacccccacctggcgggtgtactccaccggcagcaacgtgttccaaacccgggccggctgcctgatcggcgccgaacatgtgaataacagctacgaatgtgatatccctattggagccggaatttgcgccagctaccaaacccaaaccaactctccaagaagagccagaagcgtggctagccagtctatcatcgcctacaccatgagcctgggcgctgaaaacagcgtggcttacagcaacaacagcatcgccattcctactaactttaccatcagcgtgactacagagatcctgcccgtgagcatgaccaagaccagcgtggactgcaccatgtacatctgtggcgattctaccgagtgcagcaacttgctactgcagtatggtagcttttgcacccaactgaacagagccctgactggcatcgccgttgagcaggacaagaacacccaggaggtgttcgcccaggtcaagcagatctataagaccccaccaatcaaggacttcggcggcttcaacttcagccagatcctgcccgacccgagcaaacctagcaagagaagcttcatcgaggatcttctgttcaacaaggtgacgctggccgatgccggcttcatcaaacagtacggcgattgtctgggcgacatcgccgccagagatctgatctgcgcccagaagttcaatggcctgaccgtgctgccgcctctgctcacagacgagatgatcgcccagtacacctctgccctgttagcaggcacaatcacgagcggctggacatttggcgcgggcgccgctctgcagatccctttcgctatgcagatggcctatagatttaatggcatcggcgtgacccagaatgtgctgtacgagaaccaaaagctgatcgctaatcagttcaatagcgccatcggaaagatccaggactctctgagcagcaccgcctccgctctgggcaaactgcaggacgtggtcaatcaaaacgctcaggctctaaacaccctggtgaagcagctgtcaagcaacttcggcgccatctcctcagttcttaacgacatcttgtctagactggacaaggtggaggctgaggtgcagatcgatagactgatcacaggcagattacagtctctgcagacatacgtgacccaacagctgatccgggccgccgagatcagagcctccgccaacctggctgccaccaagatgagcgagtgtgtgcttggccagtccaagagagtggacttctgcggcaaaggctaccacctgatgagcttcccacagagcgctcctcacggagttgtgttcctgcacgtgacctacgtgcctgcccaggagaagaatttcacaaccgcccctgccatctgccacgacggtaaggcccacttccccagagagggcgtgttcgtgtctaatgggacccactggttcgtgacacagaggaacttctacgagccccagatcatcacaacagataataccttcgtgtctggcaactgcgatgtggtgatcggcatcgtgaacaataccgtgtacgaccctctgcagcccgagctggacagcttcaaagaggaactggacaagtacttcaagaatcacacctcccctgacgtggacctgggagatatctccggcatcaacgcctccgttgtgaacattcaaaaagaaatcgacagactgaacgaggtggccaagaacctaaacgagtctctgatcgacctgcaagaactgggcaagtacgagcagtacatcaagtggccttggtacatctggctgggctttatcgccggcctgatcgccattgtgatggtgaccatcatgctgtgctgcatgacaagttgctgcagctgcctgaagggctgttgttcttgtggcagctgctgtaaattcgacgaggacgatagcgaaccagtgctgaaaggcgtcaagctgcattacacctga

SEQ ID NO: 25 - DNA, 3768 bp, Artificial Sequence
  sequence encoding a spike protein without furin cleavage site
gccgccaccatggtgaacctgaccacccggacccagctaccaccagcctacacaaacagcttcacccgcggcgtgtactacccagacaaggtgttccggagctcagtgctccacagcacacaggacctgttcctgccattcttcagcaacgtgacgtggttccacgccatccacgtgtccggcaccaacggaacaaaaaggttcgacaaccccgtgctccccttcaacgacggcgtgtacttcgccagcacagaaaagtccaaaatcatccgaggatggatcttcggcacaacgctggactcaaagacccagagcctgctgatcgtgaacaacgccaccaacgtggtgatcaaggtgtgcgagttccagttctgcaacgatccattcctgggcgtatactaccacaagaacaacaagagctggatggaaagcgagttcagagtgtactcaagcgccaacaactgcaccttcgaatacgtcagccagccattcctgatggacctggaaggcaagcagggcaacttcaagaacctgagagagttcgtgttcaagaacatcgacggctacttcaagatctacagcaagcacacacccatcaacctggtgcgggacctaccgcagggattctccgcgctggaacccctggtggacctgccaataggcatcaacatcaccagattccagaccctgctggccctgcacagaagctacctgacaccaggcgacagcagcagcggctggaccgccggcgccgccgcatactacgtggggtacctgcagcccagaaccttcctgctgaaatacaacgagaacggaaccatcaccgacgccgtggactgcgccctggaccccctgagcgaaaccaagtgcaccctgaaaagcttcacagtggaaaagggaatataccagacctccaacttcagagtccagccaaccgaaagcatcgtgcggttcccaaacatcaccaacctgtgcccattcggcgaggtattcaacgccacacggttcgccagcgtatacgcctggaaccggaagcggatcagcaactgcgtggcagactacagcgtgctatacaacagcgccagcttaagcaccttcaagtgctacggcgtgtccccaaccaagctgaacgacctgtgcttcaccaacgtgtacgccgacagcttcgtgatcagaggcgacgaggtgcgccagatcgcaccaggacagaccggaaagatcgcagactacaactacaagctgcccgacgacttcaccggctgcgtgatcgcctggaacagcaacaacctggactccaaagtgggcggcaactacaactacctgtaccggctcttcagaaagagcaacctgaagcccttcgagagagacatcagcacagaaatctaccaggccggaagcacaccatgcaacggcgtggaaggattcaactgctatttcccactgcagtcctacggcttccaaccaaccaacggagtgggctaccagccatacagagtggtcgtgctgagcttcgagctgctgcacgccccagcaaccgtgtgcggcccaaagaaaagcacgaacctggtcaagaacaaatgcgtgaacttcaacttcaacggactgacaggcaccggcgtgctcacagagagcaacaagaagttcctgccattccagcagttcggcagagacatagcagacaccaccgacgccgtgcgggacccccagaccctggaaatcctggacatcacaccctgcagcttcggaggcgtaagcgtgatcacaccaggcaccaacacaagcaaccaggtcgccgtgctgtaccaagacgtgaactgcacagaggtgccggtggccatccacgccgaccagctgacccccacctggcgggtgtactccaccggcagcaacgtgttccaaacccgggccggctgcctgatcggcgccgaacacgtgaacaacagctacgaatgcgacatcccaatcggagccggaatctgcgccagctaccaaacccaaaccaacagcgcaagccagagcatcatcgcctacaccatgagcctgggcgcagaaaacagcgtggcatacagcaacaacagcatcgccatcccaaccaacttcaccatcagcgtgaccacagagatcctgcccgtgagcatgaccaagaccagcgtggactgcaccatgtacatctgcggcgacagcaccgagtgcagcaacttgctactgcagtacggaagcttctgcacccaactgaacagagccctgaccggcatcgccgtcgagcaggacaagaacacccaggaggtgttcgcccaggtcaagcagatctacaagaccccaccaatcaaggacttcggcggcttcaacttcagccagatcctgcccgacccgagcaaaccaagcaagagaagcttcatcgaggacctactgttcaacaaggtgacgctggccgacgccggcttcatcaaacagtacggcgactgcctgggcgacatcgccgccagagacctgatctgcgcccagaagttcaatggcctgaccgtgctgccgccactgctcacagacgagatgatcgcccagtacaccagcgccctgttagcaggcacaatcacgagcggctggacattcggcgcgggcgccgcactgcagatcccattcgcaatgcagatggcctacagattcaatggcatcggcgtgacccagaacgtgctgtacgagaaccaaaagctgatcgcaaaccagttcaacagcgccatcggaaagatccaggacagcctgagcagcaccgcctccgcactgggcaaactgcaggacgtggtcaaccaaaacgcacaggcactaaacaccctggtgaagcagctgtcaagcaacttcggcgccatcagctcagtcctaaacgacatcttgagcagactggacaaggtggaggcagaggtgcagatcgacagactgatcacaggcagattacagagcctgcagacatacgtgacccaacagctgatccgggccgccgagatcagagcctccgccaacctggcagccaccaagatgagcgagtgcgtgctaggccagagcaagagagtggacttctgcggcaaaggctaccacctgatgagcttcccacagagcgcaccacacggagtagtgttcctgcacgtgacctacgtgccagcccaggagaagaacttcacaaccgccccagccatctgccacgacggaaaggcccacttccccagagagggcgtgttcgtgagcaacgggacccactggttcgtgacacagaggaacttctacgagccccagatcatcacaacagacaacaccttcgtgagcggcaactgcgacgtggtgatcggcatcgtgaacaacaccgtgtacgacccactgcagcccgagctggacagcttcaaagaggaactggacaagtacttcaagaaccacacctccccagacgtggacctgggagacatcagcggcatcaacgcctccgtagtgaacatccaaaaagaaatcgacagactgaacgaggtggccaagaacctaaacgagagcctgatcgacctgcaagaactgggcaagtacgagcagtacatcaagtggccatggtacatctggctgggcttcatcgccggcctgatcgccatcgtgatggtgaccatcatgctgtgctgcatgacaagctgctgcagctgcctgaagggctgctgcagctgcggcagctgctgcaaattcgacgaggacgacagcgaaccagtgctgaaaggcgtcaagctgcactacacctga

SEQ ID NO: 26 - DNA, 3633 bp, Artificial Sequence
  sequence encoding a spike protein extracellular domain
gccaccatggtgaacctcactacgcgaactcaactgccgcctgcttacactaatagttttacacgaggagtctactacccggacaaggtgttccgctcaagtgtcttgcacagcacgcaagacctgttcttgccatttttcagcaatgtaacgtggtttcatgccatccacgtgtcaggtactaacggaacgaagagatttgataacccggtcctcccgttcaacgacggggtttattttgccagtacagaaaagtccaatattataaggggatggattttcggcacgactctcgactctaagacacaaagcttgctcatcgttaataatgctacaaacgtggttataaaagtatgcgagttccaattctgtaacgacccctttctgggggtttactaccacaaaaataataagagttggatggagagcgaattcagagtttactcttccgcgaataattgcaccttcgagtacgtatcccagccttttcttatggatcttgaggggaaacaaggtaatttcaaaaaccttagagaattcgtttttaagaatatagatggatacttcaagatttattctaaacataccccaatcaatctggtgcgcgatcttcctcagggtttctctgcgctggagcctctcgttgatctgccgatcggaattaatataaccagattccagacccttttggcattgcaccgctcctatctgacacctggggatagtagttccggctggacagcgggcgccgcggcatactacgttggatatctccaaccccgcacttttcttctgaaatataatgagaatggaacgattacagacgcagtagattgcgcgcttgaccccctttcagaaacaaagtgcacgctgaaatctttcacggtcgagaagggcatttaccaaacgtccaatttcagagttcaacccactgagtctatagtcaggtttccgaacatcaccaatctctgcccatttggagaggttttcaacgcaacacggttcgcctctgtatacgcatggaatagaaagagaatatccaattgtgtcgcagactatagtgtcttgtacaactctgcgtcatttagcacttttaaatgttatggggtgagtccaactaaattgaacgatctctgctttacaaatgtttacgcagatagttttgtgatacgaggcgacgaggtacgacaaattgctcctggccagaccgggaaaatagcggactacaattataaactgccagacgatttcaccggctgtgtgatcgcgtggaactcaaacaacttggatagtaaggtaggtggtaactataactacttgtaccggctcttccgcaaaagtaatcttaaaccgttcgagcgggacatctctaccgagatctatcaagcaggtagcactccctgcaatggagtcgagggttttaattgctattttcccctccaatcatacgggtttcagcccacaaatggtgttgggtatcagccctatcgagttgtggtgctttcatttgaattgcttcatgcaccggcgacagtttgtggtcctaaaaagagtaccaatttggttaaaaacaaatgtgtcaactttaactttaacggtctgactggtacgggggtgcttacggaatctaataaaaagtttcttccgttccagcaattcggacgggacatagcggacacgactgacgcggttagagacccacaaacactcgagattcttgacataaccccctgttctttcggcggagtttctgtaataacgcctgggacaaatacgtcaaaccaggttgcagtattgtaccaggatgtcaactgcactgaagtgccagtcgcgatccacgcagatcagttgactccaacatggagggtctactctaccggttctaacgtgtttcagaccagagccgggtgcctcattggcgcggaacatgtgaacaattcttacgaatgcgacatacccatcggcgccggcatttgtgcgtcataccaaactcaaactaattcccggagtgtggcctcccaatctattattgcttatactatgagtcttggcgcggaaaacagtgtagcgtactcaaataactctattgcgatccccacgaatttcactataagcgtaactactgaaattttgccggtaagcatgacgaaaacttcagtggactgcacaatgtacatttgtggtgacagcactgagtgctcaaatctcctccttcagtatggttctttctgcacacaacttaacagggcacttaccggaatcgcagtagaacaagacaaaaacacgcaagaagtattcgcacaggtcaaacaaatttataaaaccccgccgatcaaggattttggagggtttaacttcagtcagatccttccagacccatccaaacccagcaaacggagctttattgaagacctgctgtttaataaagttacccttgcagatgccggtttcatcaagcaatacggggattgtctgggcgacattgccgcccgcgacttgatctgtgcccagaagttcaacggtctcacagtcttgccgccacttctcactgatgaaatgatagcgcagtatacatccgcgctgctggctgggacgataacatctggttggactttcggggccggggcggctttgcaaattcctttcgccatgcagatggcctataggtttaacggcataggagtgactcaaaatgttctttatgagaatcagaagcttatcgccaaccaatttaactccgcgattgggaagatacaggactcattgagctctactgctagcgcccttggcaagcttcaagacgtagtcaaccaaaatgctcaagcgctcaatactctcgtaaagcagctctcaagcaactttggcgcgatatcttcagttttgaacgacatcttgagcagactcgacaaggtggaggctgaagttcaaattgatcgactgattacgggccggctccagtcactccaaacgtacgtgacccaacaactcattagagcggcagagattagagcgagcgctaatctggcggccacaaagatgtcagaatgtgtgcttggtcaatccaaaagggtggatttctgcggaaagggctatcacttgatgagtttcccgcagtccgcgcctcatggagttgttttcctccacgtaacctacgttcccgctcaggaaaagaatttcacgacagctccggcaatatgtcatgacggtaaagcacatttccccagggaaggcgtttttgtcagtaatggaacccactggttcgtcacccagcgaaacttttacgagccccaaataataactactgacaataccttcgtcagcggcaattgcgacgtcgtcataggcatagtaaataatacggtttacgaccctcttcaacccgagttggacagcttcaaagaagaacttgataagtattttaaaaaccacactagcccagatgtagaccttggcgacatcagcggtatcaatgcttccgttgtgaacattcaaaaggaaattgaccgactcaatgaagtagcgaagaacttgaatgagtctctgatcgatcttcaggagctcgggaaatatgagcaatacatcaaatggccgtcacgactggaggaagaactgcgccgacgcctgactgaatga

SEQ ID NO: 27 - DNA, 3627 bp, Artificial Sequence
  sequence encoding a spike protein extracellular domain
gccaccatggttaacctgacaacacgcacccagttgccaccggcctatacgaactctttcacgcgcggagtgtattatcccgataaagtctttcggagttctgtgctgcactctacacaagatctttttctgccctttttctccaacgttacgtggttccatgcaataagtggtacgaatggtactaaaaggttcgataatcccgtacttcccttcaatgacggcgtgtatttcgcgagcacagaaaagagcaacataataagaggttggatttttgggactaccctggactctaaaacacaaagcctcctcattgttaataacgcaactaatgtcgtaataaaggtatgcgaatttcagttttgtaacgacccttttttgggagtctatcataagaacaataagtcctggatggaaagtgaatttagggtgtatagttcagcgaacaactgtacatttgagtatgtgtcacagccgttccttatggatcttgaagggaaacaggggaacttcaagaacctgcgggaattcgtgtttaaaaatatagacggttattttaaaatctatagcaaacacaccccaataaacctggttcgggatcttcctcagggattttcagctttggaaccgttggttgacctccccatcgggataaatatcacgcgatttcaaacactgctggcactgcaccggagttatctgactcctggagatagcagctccggctggacggctggcgccgcggcttattatgtcgggtaccttcaaccgcgaacgtttcttttgaagtataacgaaaatggcactataaccgatgcagtagattgtgcgttggaccctctttccgagactaagtgcaccctgaagtcatttacagtagagaaggggatataccagacaagcaacttccgcgtccaaccaacggaaagcatcgtgcgctttcccaatattactaacctctgtccgttcggcgaggtttttaatgctaccagatttgcctcagtgtatgcctggaaccgcaagaggattagcaactgcgttgctgactatagcgtgctttacaattccgccagttttagtacgttcaagtgttatggggtaagccctaccaagcttaacgatctttgcttcacgaatgtttacgcagactcattcgtaatacgaggtgatgaagtgcgacagatagccccgggtcagacggggaaaatagccgactacaattacaagctcccggacgacttcacgggctgtgttattgcgtggaacagcaataacctggacagtaaagttggagggaattataactacttgtacaggctgttccgcaaatctaatttgaaaccgtttgaaagggacatttccaccgagatttaccaagccggcagtaccccatgtaacggagttgagggatttaattgctactttccgctccaatcatatggattccagcctacatacggcgtcggatatcaaccttatagggtggtcgttctttcttttgagctgcttcacgccccagcaaccgtttgcggcccgaaaaaatcaacgaacttggtcaaaaacaagtgcgtcaacttcaatttcaacggactgacaggcaccggagtgctcaccgagtcaaataaaaaattcctcccgtttcaacaattcggtagagatatagacgacaccacagatgcggtacgcgaccctcagactcttgaaattttggacattacaccatgttccttcggtggcgtctcagtgattacgcctggcactaacactagcaaccaagtcgcagtactttatcagggcgttaactgtacagaagtaccagttgcgatacacgcggaccagctcaccccaacctggcgcgtatattctaccggatctaatgtgtttcaaacgcgggcggggtgcctcataggtgcagagcatgtcaataactcctacgaatgtgacattcctataggagcaggaatatgtgcgagctaccagactcaaaccaattcacacaggagcgtagctagtcagtccataattgcctacactatgtcacttggtgctgaaaacagtgtggcttattcaaataattccatagctattccaatcaatttcactattagcgtaacaactgagatacttccagtcagcatgactaagacttcagttgattgtacgatgtatatttgtggcgattctacagaatgtagtaaccttctgttgcaatacggctccttttgcacacagctcaatcgcgcattgacgggtatagctgtagagcaagataaaaacacccaagaagtcttcgcgcaggtgaagcaaatatataagacaccaccgattaaagatttcggtggattcaattttagccaaattctgcctgatccaagcaaaccgtcaaaaagatccttcatagaggacctgcttttcaataaggtaacgctggcggacgcagggtttattaaacaatacggcgattgccttggtgacattgctgcacgagacctcatttgcgcccagaagttcaacggactcacagtactgcctccactgctcacagacgagatgatcgcccagtacacatctgccttgttggctggtacgataacttctggttggacctttggagcgggagcagcacttcagattccctttgccatgcagatggcgtacagattcaacggcattggtgtcacgcagaacgtactgtatgagaaccagaagttgatagctaatcaattcaactccgcgataggtaagatccaagacagtctctcctcaacagcctccgcactcgggaaactccaggatgtggtaaatcagaatgcacaggcattgaatacgcttgttaaacaattgtcctcaaattttggcgccatatcatcagtgctcaacgacatccttgccagactcgataaagtggaggcagaagtgcagatagatcgcttgattactgggcgcctccaatctctccaaacgtacgtgacacaacagcttattcgggcagcggaaatccgcgcctctgcgaaccttgcagcgaccaagatgagcgaatgtgttctcgggcagtcaaaacgagtagatttctgcggtaaaggatatcatcttatgtcattcccccaaagcgctccacatggtgttgtatttctgcacgtgacttacgtgccggcacaggagaagaatttcacaacggctcccgccatctgtcacgacggaaaggcacacttccctagggagggggtgtttgtaagcaatggtacccactggtttgtcacacaacgaaacttctatgaacctcagataattacaacgcataatacttttgtaagtgggaactgcgacgtcgttatcggaatagttaacaatactgtatatgatccgttgcagccggagttggactcatttaaagaagagctggacaagtacttcaaaaaccatacaagcccagatgtagatcttggggacataagtgggataaacgccagcgttgtgaacatacagaaggaaatagacaggctgaatgaggtagcaaagaatcttaacgaaagccttatagatcttcaggaacttgggaagtacgaacagtacataaagtggccttcacgactggaggaagaactgcgccgacgcctgactgaatga

SEQ ID NO: 28 - DNA, 3633 bp, Artificial Sequence
  sequence encoding a spike protein extracellular domain
gccaccatggtcaatttcactacacgcacacagctgcccccggcgtacactaacagttttacgaggggagtctactacccggacaaagtgttcaggtcttccgtactccatagcacacaagacctcttcttgccttttttcagcaatgttacgtggttccatgcaatccatgtgagcggtacaaacgggactaaacgctttgcaaatccagtgttgcccttcaacgacggtgtatacttcgcgagtacagagaagagcaacataatacgcgggtggatatttggaacgacgcttgattctaagacacagagcttgctcatcgtaaacaacgcaacgaatgttgtaatcaaggtctgcgaattccaattctgcaatgacccatttcttggagtttattaccataagaacaataagtcttggatggaatccgagttccgcgtatactctagcgcgaacaactgcaccttcgagtatgtgtcccaaccgtttctgatggatcttgagggaaagcagggtaattttaaaaatctccgggagttcgttttcaaaaatattgacggatatttcaaaatctacagcaagcataccccaatcaatttggtgcggggtctgccacaaggattttccgctcttgaacctcttgttgaccttcccataggaatcaatataactcggttccaaactcttcttgcgctccatatatcctacttgacgcccggagattcatcaagtggctggacggctggggctgccgcatactacgtcggctatctgcaaccaaggacgtttctcctcaaatacaatgaaaatggtacgatcaccgacgctgtggactgcgctctcgacccgttgtcagagacgaagtgcaccttgaagtccttcaccgtggaaaagggaatttaccagactagtaacttccgggtgcagcccacggaatcaattgtgcgatttcctaatatcactaatctctgtcccttcggggaagtgttcaatgcgactcgattcgcttcagtatacgcttggaatagaaagagaatctcaaattgcgtggctgactattccgtactgtataatagtgcgtcttttagcacgtttaagtgttatggcgtatccccgacaaagctcaacgatctctgcttcacgaatgtctatgccgactccttcgtgatacgcggtgacgaggtacgacaaattgcacccggccaaactggaaatattgccgactacaattataagttgccggacgattttaccggttgcgtaatagcgtggaactctaataacctcgactctaaggtaggaggcaactacaattacttgtaccgactgttcaggaaatccaacctgaagcccttcgaaagagacatcagtacagagatttatcaagccgggtccaccccttgcaatggtgtcaaaggcttcaactgctattttccgttgcagtcttacggtttccagccgacgtacggtgtgggttatcaaccctaccgcgttgtggtgctgagctttgagcttctccacgctccagccactgtctgcgggcctaagaaatccacaaacctcgttaagaataaatgcgtaaatttcaacttcaacgggcttactggcacgggcgtgcttacggagtccaataagaaattcctgcctttccagcagttcggccgcgacatcgcggacacaacagatgctgtgcgagacccacaaaccctggagattctggacatcacgccttgctcttttggcggtgtcagcgtaataacgccaggcaccaacaccagtaatcaggtcgccgtgctttatcagggtgtgaactgtacggaggtaccggttgccatacacgcagaccaactcacacctacgtggagagtttacagcaccgggtcaaacgtatttcagacccgcgcaggctgtctgatcggcgcagagcatgtcaataactcatacgagtgtgatataccaatcggagccggcatctgtgcgagctatcaaacccaaactaattcacgaagcgtggccagccagtccatcattgcctacacgatgtctttgggagtagagaatagcgtggcatactctaacaacagtatcgctattcctaccaacttcacaatatcagttactacagaaatcctcccggtgagcatgactaagacatccgtagactgtacaatgtacatttgtggggacagcacggagtgttcaaacctgctgttgcaatacggaagcttctgcacccagctcaaccgagccctgaccggcattgccgtagaacaagataagaatacgcaggaagtgttcgcccaggttaagcagatttacaagactccgcctatcaaggacttcgggggtttcaactttagtcagattttgcctgacccgtcaaagcctagcaaacgaagttttatcgaagatttgttgtttaacaaggtgactttggctgatgcgggattcataaaacaatacggcgattgccttggcgatattgcggcccgagatctcatttgcgcgcaaaagttcaacggtcttaccgtccttccgcccctgcttacggacgagatgatcgcacaatacacttcagcactgctggctggtacaataaccagtgggtggacattcggggctggtgcagcgctccagatcccctttgcgatgcagatggcgtatagattcaatggcatcggggtcactcagaacgtgctttatgagaaccaaaagcttattgcaaatcaatttaactctgcgattggaaagattcaagacagcctctcatccacagctagtgcgcttggcaagcttcaggacgtcgtaaaccaaaacgcccaagctctcaacacactcgttaaacagctttcctcaaactttggggctattagctctgtgctgaatgatattcttagcagactcgacaaagtcgaggctgaagtacaaatagatagactgataacaggtagacttcagtctctccaaacctacgtgacacagcagcttataagagctgctgagatcagagcttctgcgaaccttgcggcgactaaaatgtccgagtgtgtactcgggcagtctaaacgcgtcgacttctgtggcaagggttaccatctcatgagttttccccaatcagcgccccatggggtagtttttctgcacgtaacatacgtcccagcccaagaaaagaattttaccaccgcccctgcgatttgtcacgacggaaaagcacactttccaagagaaggcgtatttgttagcaatggcacccattggttcgtcacccagagaaacttttacgaaccgcagattatcacgacagataatacatttgtatctggtaattgtgacgtcgtgatcggcatcgtaaacaacactgtttacgaccccctccagccagagcttgactcatttaaagaggagctcgacaaatattttaagaaccacacaagtccagacgtcgacttgggtgacatttccggcatcaatgcaagtgtggtcaacatccaaaaggaaattgacagattgaacgaggttgctaagaaccttaatgagtcactcattgacctccaggaactgggaaaatacgaacagtatattaagtggccctcacgactggaggaagaactgcgccgacgcctgactgaatga

SEQ ID NO: 29 - DNA, 3630 bp, Artificial Sequence
  sequence encoding a spike protein extracellular domain
gccaccatggtaaatttgacaacgcgcacccaattgcccccagcatatacgaactctttcacgaggggcgtatattatccggataaggtatttcggtcatctgttctgcacagcacccaggacctcttccttccattcttttcaaatgtaacttggttccatgcgatagtatcaggaacgaatgggacaaaaaggttcgataatccggtcttgccgttcaacgatggggtgtacttcgccagtaccgaaaagtctaacattatacgcggttggatttttggcactacgcttgactcaaagacacagtcactcctcattgtaaataatgctactaatgtcgtgatcaaagtttgtgagttccaattctgcaacgacccgttcctgggagtgtattatcacaaaaataataagtcctgcatggagtcagagttcagggtttactcaagtgcgaacaattgcacatttgagtacgtttctcaaccatttctcatggatttggaggggaagcagggaaactttaaaaacctgagagaatttgtttttaagaacattgatgggtatttcaagatttatagtaaacacacccctatcaacttggttagggatctccctcaaggtttctctgctctcgagccccttgtagatctgccaataggcatcaatatcacacgctttcagacactcctcgcacttcataggagctacctgacgccaggtgactcttcctcaggttggacagccggcgcagccgcatactacgttggctacctccaaccaaggacatttctgttgaaatacaacgaaaatgggaccatcaccgacgcagtcgattgtgctctcgaccctctttccgagactaaatgtaccctcaaaagctttactgttgagaagggtatctatcagacatctaactttcgggtgcaacccactgagtcaattgtgcgattcccaaatattacgaacctctgtccttttggcgaggtttttaacgccactaggttcgccagtgtatatgcttggaaccgaaaacggataagcaattgtgttgctgactactccgtcctctacaatagcgctagtttctcaacatttaagtgttacggtgtgagccctacgaaacttaacgatttgtgcttcactaacgtctatgccgacagtttcgtaatccgaggcgatgaggtcaggcaaattgccccgggccaaacggggaaaatcgctgattacaattataagttgccagatgattttacgggatgtgtcattgcatggaacagtaataacctcgattcaaaggttggcggaaattataattacagatatcggctttttagaaaatctaaccttaaaccatttgagcgggacataagcacggagatttaccaggctggtagcactccgtgcaacggtgtagaaggattcaattgctattttccattgcagtcttatggattccaacccaccaatggggtagggtaccaaccatacagggtggtagtccttagctttgaacttttgcatgcgccagctaccgtctgcggtcccaaaaagagtacgaacttggtaaaaaataaatgcgtcaactttaattttaacggtctgacgggaacgggggttctcaccgagtctaacaaaaagtttttgccatttcagcagttcggacgagatattgccgacactaccgacgccgtgcgcgatccacaaaccttggagatactcgacatcacaccttgcagcttcggtggtgttagcgttattacgccaggaacgaacacttcaaatcaggtggccgtcttgtatcaagatgttaactgtacagaggtgcccgtcgcaatacacgcagatcagctcacccctacctggagagtttactctacaggctctaatgtgttccaaactagggcaggctgcttgattggagctgaacacgtaaacaactcatacgagtgcgatatcccgattggtgcgggtatttgcgccagctatcagacgcagactaactcacgatcagtggcgtcacagtcaataattgcgtatacaatgagtctcggtgcagagaattccgtcgcatactcaaacaacagcatagcgattccaactaattttaccattagcgttacaacggaaatcctccccgttagcatgaccaagacctccgtggattgcacaatgtacatctgtggagacagcaccgagtgctccaatcttcttcttcaatatgggagcttttgcacacagctgaaccgcgcgctcactggcattgcggtagaacaagacaaaaacacccaagaagtgtttgcgcaggtcaaacagatttacaagacaccaccaatcaaagacttcggggggttcaatttctcccaaattcttcctgatccaagcaaaccatctaaaaggtccttcatagaagacctcctgttcaataaggtgacactcgcggacgcagggtttattaagcaatatggagattgtctcggggatattgcagcgagagatcttatttgtgctcaaaagtttaatggcttgactgtcctcccgcccttgctcacagacgagatgatagcacaatataccagtgcgctgcttgcgggcacgattacttccggatggactttcggggcaggagcggcgctccagattcctttcgcgatgcaaatggcctaccgatttaacgggattggcgtcacacaaaacgttctttacgaaaaccagaaacttattgccaaccaattcaatagtgcaattggtaaaattcaggattcactctcctccacggcgtcagctctcggcaagctccaagacgtggttaaccaaaatgcgcaggcgcttaacacgcttgtaaaacaactctcttctaattttggcgcgattagtagcgttctcaacgatatcttgtcacgcttggacaaagtagaagcagaagtccaaatagaccggctgatcactggtcgattgcaatctctgcagacatacgtgacgcagcagctcatccgggcagccgagatccgggcgtccgctaatctggccgcaaccaaaatgagcgagtgtgtccttgggcaaagcaaacgcgtggatttttgcggaaagggctaccacctcatgagtttcccccaatctgccccgcacggtgtcgtctttttgcatgtaacgtatgtcccggcgcaggagaaaaatttcactacagctcccgccatctgtcacgacggcaaggcacacttccctcgagaaggggtgtttgtaagcaatggaacacactggttcgtcacccagcggaatttttatgaacctcagataatcacaacagacaacactttcgtttccggaaactgcgatgtcgtaattgggatagtgaataataccgtatatgacccattgcagccggagttggactcctttaaagaggaattggacaaatactttaaaaatcacacttcacccgatgtggacctgggagatatatccgggattaacgcaagtgtggttaacatacaaaaggaaattgaccgccttaacgaggttgctaagaacttgaatgagtctctcatagatctccaggaactggggaaatacgagcaatatatcaaatggccgtcacgactggaggaagaactgcgccgacgcctgactgaatga

SEQ ID NO: 30 - DNA, 3633 bp, Artificial Sequence
  sequence encoding a spike protein extracellular domain
gccaccatggttaactttactaatcggacccaactcccgagtgcgtacactaactcttttacccggggcgtctattatcctgacaaagtgtttcgatcatctgttttgcactccacccaagatctctttcttccatttttcagcaacgttacgtggttccacgcgatacatgttagtggtactaatggcacaaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaggagctatctgaccccgggcgactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaacattgctgattataactacaagttgcccgatgacttcacggggtgtgttattgcatggaattcaaataatcttgatagtaaggtagggggtaactataattacctttatcgcttgttccgcaaatccaatctgaaacccttcgaaagggatatcagtaccgagatttatcaagccggctccacaccttgtaacggagtaaaaggttttaactgttatttcccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagcagaagcgttgcgtcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgacggttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctattaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttggagacatctctgggataaacgctagcttcgtaaacatacaaaaagaaatcgataggcttaacgaggtcgcaaaaaatcttaatgaaagtcttatcgatttgcaagagcttggaaagtatgagcagtatatcaaatggccttcacgactggaggaagaactgcgccgacgcctgactgaatga

SEQ ID NO: 31 - DNA, 3630 bp, Artificial Sequence
  sequence encoding a spike protein extracellular domain
gccaccatggttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatcacgactggaggaagaactgcgccgacgcctgactgaataa

SEQ ID NO: 32 - DNA, 3609 bp, Artificial Sequence
  sequence encoding the spike protein extracellular domain
gccgccaccatggtgaacctgaccacccggacccagctacctcctgcctacacaaacagcttcacccgcggcgtgtactaccctgataaggtgttccggagctctgtgctccacagcacacaggacctgttcctgcctttcttcagcaacgtgacgtggttccacgccatccacgtgtccggcaccaatggtacaaaaaggtttgacaaccccgtgctccccttcaacgacggcgtgtattttgccagcactgaaaagtccaatatcatccgaggatggatcttcggcacaacgctggactctaagacccagagcctgctgatcgtgaacaacgccaccaacgtggtgatcaaggtgtgcgagtttcagttctgcaacgatccttttctgggcgtttactaccacaagaacaacaagagctggatggaaagcgagttcagagtgtattctagcgccaacaactgtaccttcgaatacgtcagccagcctttcctgatggacctggaaggcaagcagggcaacttcaagaacctgagagagttcgtgttcaagaacatcgacggctacttcaagatctacagcaagcacacacctatcaacctggtgcgggaccttccgcagggattctccgcgctggaacccctggtggacctgcctattggcatcaacatcaccagatttcagaccctgctggccctgcacagaagctacctgacacctggcgactcttctagcggctggaccgccggcgccgccgcttattacgtggggtacctgcagcccagaaccttcctgctgaaatacaatgagaacggaaccatcaccgatgccgtggactgcgccctggaccccctgtctgaaaccaagtgcaccctgaaatcttttacagtggaaaagggaatttaccagacctccaactttagagtccagcctaccgaaagcatcgtgcggttccctaacatcaccaacctgtgtcctttcggcgaggttttcaacgccacacggttcgccagcgtatacgcctggaaccggaagcggatctctaactgtgtggcagactactctgtgctatacaactcggccagctttagcaccttcaagtgctacggcgtgtccccaaccaagctgaacgacctgtgcttcaccaacgtgtacgccgatagcttcgtgatcagaggcgatgaggtgcgccagatcgctcctggacagaccggaaagatcgctgactacaattacaagctgcccgacgacttcaccggctgcgtgatcgcctggaacagcaacaacctggattccaaagtgggcggcaactacaactatctgtaccggctcttcagaaagtctaacctgaagccctttgagagagacatctccacagaaatctaccaggccggaagcacaccttgcaacggcgtggaaggatttaattgctattttcctctgcagtcctacggcttccaacctaccaacggagtgggctaccagccttacagagtggtcgtgctgagctttgagctgctgcatgcccctgctaccgtgtgtggccctaagaaaagcacgaacctggtcaagaacaaatgcgtgaacttcaacttcaatggactgacaggcaccggcgtgctcacagagagcaacaagaagttcctgccatttcagcagttcggcagagacatagctgacaccaccgacgccgtgcgggacccccagaccctggaaatcctggatatcacaccctgcagctttggaggcgtatctgtgatcacacctggcaccaacacaagcaatcaggtcgccgtgctgtaccaagacgtgaactgcacagaggtgccggtggccatccacgccgatcagctgacccccacctggcgggtgtactccaccggcagcaacgtgttccaaacccgggccggctgcctgatcggcgccgaacatgtgaataacagctacgaatgtgatatccctattggagccggaatttgcgccagctaccaaacccaaaccaactctccaagaagagccagaagcgtggctagccagtctatcatcgcctacaccatgagcctgggcgctgaaaacagcgtggcttacagcaacaacagcatcgccattcctactaactttaccatcagcgtgactacagagatcctgcccgtgagcatgaccaagaccagcgtggactgcaccatgtacatctgtggcgattctaccgagtgcagcaacttgctactgcagtatggtagcttttgcacccaactgaacagagccctgactggcatcgccgttgagcaggacaagaacacccaggaggtgttcgcccaggtcaagcagatctataagaccccaccaatcaaggacttcggcggcttcaacttcagccagatcctgcccgacccgagcaaacctagcaagagaagcttcatcgaggatcttctgttcaacaaggtgacgctggccgatgccggcttcatcaaacagtacggcgattgtctgggcgacatcgccgccagagatctgatctgcgcccagaagttcaatggcctgaccgtgctgccgcctctgctcacagacgagatgatcgcccagtacacctctgccctgttagcaggcacaatcacgagcggctggacatttggcgcgggcgccgctctgcagatccctttcgctatgcagatggcctatagatttaatggcatcggcgtgacccagaatgtgctgtacgagaaccaaaagctgatcgctaatcagttcaatagcgccatcggaaagatccaggactctctgagcagcaccgcctccgctctgggcaaactgcaggacgtggtcaatcaaaacgctcaggctctaaacaccctggtgaagcagctgtcaagcaacttcggcgccatctcctcagttcttaacgacatcttgtctagactggacaaggtggaggctgaggtgcagatcgatagactgatcacaggcagattacagtctctgcagacatacgtgacccaacagctgatccgggccgccgagatcagagcctccgccaacctggctgccaccaagatgagcgagtgtgtgcttggccagtccaagagagtggacttctgcggcaaaggctaccacctgatgagcttcccacagagcgctcctcacggagttgtgttcctgcacgtgacctacgtgcctgcccaggagaagaatttcacaaccgcccctgccatctgccacgacggtaaggcccacttccccagagagggcgtgttcgtgtctaatgggacccactggttcgtgacacagaggaacttctacgagccccagatcatcacaacagataataccttcgtgtctggcaactgcgatgtggtgatcggcatcgtgaacaataccgtgtacgaccctctgcagcccgagctggacagcttcaaagaggaactggacaagtacttcaagaatcacacctcccctgacgtggacctgggagatatctccggcatcaacgcctccgttgtgaacattcaaaaagaaatcgacagactgaacgaggtggccaagaacctaaacgagtctctgatcgacctgcaagaactgggcaagtacgagcagtacatcaagtggccataa

SEQ ID NO: 33 - DNA, 3609 bp, Artificial Sequence
  sequence encoding a spike protein extracellular domain without the furin sequence
gccgccaccatggtgaacctgaccacccggacccagctaccaccagcctacacaaacagcttcacccgcggcgtgtactacccagacaaggtgttccggagctcagtgctccacagcacacaggacctgttcctgccattcttcagcaacgtgacgtggttccacgccatccacgtgtccggcaccaacggaacaaaaaggttcgacaaccccgtgctccccttcaacgacggcgtgtacttcgccagcacagaaaagtccaaaatcatccgaggatggatcttcggcacaacgctggactcaaagacccagagcctgctgatcgtgaacaacgccaccaacgtggtgatcaaggtgtgcgagttccagttctgcaacgatccattcctgggcgtatactaccacaagaacaacaagagctggatggaaagcgagttcagagtgtactcaagcgccaacaactgcaccttcgaatacgtcagccagccattcctgatggacctggaaggcaagcagggcaacttcaagaacctgagagagttcgtgttcaagaacatcgacggctacttcaagatctacagcaagcacacacccatcaacctggtgcgggacctaccgcagggattctccgcgctggaacccctggtggacctgccaataggcatcaacatcaccagattccagaccctgctggccctgcacagaagctacctgacaccaggcgacagcagcagcggctggaccgccggcgccgccgcatactacgtggggtacctgcagcccagaaccttcctgctgaaatacaacgagaacggaaccatcaccgacgccgtggactgcgccctggaccccctgagcgaaaccaagtgcaccctgaaaagcttcacagtggaaaagggaatataccagacctccaacttcagagtccagccaaccgaaagcatcgtgcggttcccaaacatcaccaacctgtgcccattcggcgaggtattcaacgccacacggttcgccagcgtatacgcctggaaccggaagcggatcagcaactgcgtggcagactacagcgtgctatacaacagcgccagcttaagcaccttcaagtgctacggcgtgtccccaaccaagctgaacgacctgtgcttcaccaacgtgtacgccgacagcttcgtgatcagaggcgacgaggtgcgccagatcgcaccaggacagaccggaaagatcgcagactacaactacaagctgcccgacgacttcaccggctgcgtgatcgcctggaacagcaacaacctggactccaaagtgggcggcaactacaactacctgtaccggctcttcagaaagagcaacctgaagcccttcgagagagacatcagcacagaaatctaccaggccggaagcacaccatgcaacggcgtggaaggattcaactgctatttcccactgcagtcctacggcttccaaccaaccaacggagtgggctaccagccatacagagtggtcgtgctgagcttcgagctgctgcacgccccagcaaccgtgtgcggcccaaagaaaagcacgaacctggtcaagaacaaatgcgtgaacttcaacttcaacggactgacaggcaccggcgtgctcacagagagcaacaagaagttcctgccattccagcagttcggcagagacatagcagacaccaccgacgccgtgcgggacccccagaccctggaaatcctggacatcacaccctgcagcttcggaggcgtaagcgtgatcacaccaggcaccaacacaagcaaccaggtcgccgtgctgtaccaagacgtgaactgcacagaggtgccggtggccatccacgccgaccagctgacccccacctggcgggtgtactccaccggcagcaacgtgttccaaacccgggccggctgcctgatcggcgccgaacacgtgaacaacagctacgaatgcgacatcccaatcggagccggaatctgcgccagctaccaaacccaaaccaacagcccaagaagagccagaagcgtggcaagccagagcatcatcgcctacaccatgagcctgggcgcagaaaacagcgtggcatacagcaacaacagcatcgccatcccaaccaacttcaccatcagcgtgaccacagagatcctgcccgtgagcatgaccaagaccagcgtggactgcaccatgtacatctgcggcgacagcaccgagtgcagcaacttgctactgcagtacggaagcttctgcacccaactgaacagagccctgaccggcatcgccgtcgagcaggacaagaacacccaggaggtgttcgcccaggtcaagcagatctacaagaccccaccaatcaaggacttcggcggcttcaacttcagccagatcctgcccgacccgagcaaaccaagcaagagaagcttcatcgaggacctactgttcaacaaggtgacgctggccgacgccggcttcatcaaacagtacggcgactgcctgggcgacatcgccgccagagacctgatctgcgcccagaagttcaatggcctgaccgtgctgccgccactgctcacagacgagatgatcgcccagtacaccagcgccctgttagcaggcacaatcacgagcggctggacattcggcgcgggcgccgcactgcagatcccattcgcaatgcagatggcctacagattcaatggcatcggcgtgacccagaacgtgctgtacgagaaccaaaagctgatcgcaaaccagttcaacagcgccatcggaaagatccaggacagcctgagcagcaccgcctccgcactgggcaaactgcaggacgtggtcaaccaaaacgcacaggcactaaacaccctggtgaagcagctgtcaagcaacttcggcgccatcagctcagtcctaaacgacatcttgagcagactggacaaggtggaggcagaggtgcagatcgacagactgatcacaggcagattacagagcctgcagacatacgtgacccaacagctgatccgggccgccgagatcagagcctccgccaacctggcagccaccaagatgagcgagtgcgtgctaggccagagcaagagagtggacttctgcggcaaaggctaccacctgatgagcttcccacagagcgcaccacacggagtagtgttcctgcacgtgacctacgtgccagcccaggagaagaacttcacaaccgccccagccatctgccacgacggaaaggcccacttccccagagagggcgtgttcgtgagcaacgggacccactggttcgtgacacagaggaacttctacgagccccagatcatcacaacagacaacaccttcgtgagcggcaactgcgacgtggtgatcggcatcgtgaacaacaccgtgtacgacccactgcagcccgagctggacagcttcaaagaggaactggacaagtacttcaagaaccacacctccccagacgtggacctgggagacatcagcggcatcaacgcctccgtagtgaacatccaaaaagaaatcgacagactgaacgaggtggccaagaacctaaacgagagcctgatcgacctgcaagaactgggcaagtacgagcagtacatcaagtggccataa

SEQ ID NO: 34 - protein, 71 aa, Artificial Sequence
  CD74 transmembrane and cytoplasmic domain
HRRRSRSCREDQKPVMDDQRDLISNNEQLPMLGRRPGAPESKCSRGALYTGFSILVTLLLAGQATTAYFLY

SEQ ID NO: 35 - protein, 58 aa, Artificial Sequence
  HLA fragment
MVKCATLSVDSGQASDSSAAQTYSGGKRDSSKRRWMVAAVVAGTIVAGLLVLGAIIGV

SEQ ID NO: 36 - protein, 69 aa, Artificial Sequence
  targeting moiety comprising CD74 cytoplasmic domain fused to HLA transmembrane domain
HRRRSRSCREDQKPVMDDQRDLISNNEQLPMLGRRPGAPESKCSRWMVAAVVAGTIVAGLLVLGAIIGV

SEQ ID NO: 37 - protein, 1330 aa, Artificial Sequence
  fusion protein comprising CD74 cytoplasmic domain and transmembrane domain and spike protein
MHRRRSRSCREDQKPVMDDQRDLISNNEQLPMLGRRPGAPESKCSRGALYTGFSILVTLLLAGQATTAYFLYVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 1275 aa, Artificial Sequence
  fusion protein comprising CD74 cytoplasmic domain and extracellular domain with spike extracellular domain
MAATMAHRRRSRSCREDQKPVMDDQRDLISNNEQLPMLGRRPGAPESKCSRGALYTGFSILVTLLLAGQATTAYFLYVNLTTRTQLPPAYTNSFTRGVYYPDKVFRSSVLHSTQDLFLPFFSNVTWFHAIHVSGTNGTKRFDNPVLPFNDGVYFASTEKSKIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASLSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 39 - protein, 1323 aa, Artificial Sequence
  fusion protein comprising CD74 cytoplasmic domain and transmembrane domain with spike lacking furin cleavage site
MHRRRSRSCREDQKPVMDDQRDLISNNEQLPMLGRRPGAPESKCSRGALYTGFSILVTLLLAGQATTAYFLYVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - RNA, 4002 nt, Artificial Sequence
  sequence encoding the CD74 Cytoplasmic domain and transmembrane domain with full length spike
gccgccaccaugcacaggaggagaagcaggagcugucgggaagaucagaagccagucauggaugaccagcgcgaccuuaucuccaacaaugagcaacugcccaugcugggccggcgcccuggggccccggagagcaagugcagccgcggagcccuguacacaggcuuuuccauccuggugacucugcuccucgcuggccaggccaccaccgccuacuuccuguacgugaaccugaccacccggacccagcuaccuccugccuacacaaacagcuucacccgcggcguguacuacccugauaagguguuccggagcucugugcuccacagcacacaggaccuguuccugccuuucuucagcaacgugacgugguuccacgccauccacguguccggcaccaaugguacaaaaagguuugacaaccccgugcuccccuucaacgacggcguguauuuugccagcacugaaaaguccaauaucauccgaggauggaucuucggcacaacgcuggacucuaagacccagagccugcugaucgugaacaacgccaccaacguggugaucaaggugugcgaguuucaguucugcaacgauccuuuucugggcguuuacuaccacaagaacaacaagagcuggauggaaagcgaguucagaguguauucuagcgccaacaacuguaccuucgaauacgucagccagccuuuccugauggaccuggaaggcaagcagggcaacuucaagaaccugagagaguucguguucaagaacaucgacggcuacuucaagaucuacagcaagcacacaccuaucaaccuggugcgggaccuuccgcagggauucuccgcgcuggaaccccugguggaccugccuauuggcaucaacaucaccagauuucagacccugcuggcccugcacagaagcuaccugacaccuggcgacucuucuagcggcuggaccgccggcgccgccgcuuauuacgugggguaccugcagcccagaaccuuccugcugaaauacaaugagaacggaaccaucaccgaugccguggacugcgcccuggacccccugucugaaaccaagugcacccugaaaucuuuuacaguggaaaagggaauuuaccagaccuccaacuuuagaguccagccuaccgaaagcaucgugcgguucccuaacaucaccaaccuguguccuuucggcgagguuuucaacgccacacgguucgccagcguauacgccuggaaccggaagcggaucucuaacuguguggcagacuacucugugcuauacaacucggccagcuuuagcaccuucaagugcuacggcguguccccaaccaagcugaacgaccugugcuucaccaacguguacgccgauagcuucgugaucagaggcgaugaggugcgccagaucgcuccuggacagaccggaaagaucgcugacuacaauuacaagcugcccgacgacuucaccggcugcgugaucgccuggaacagcaacaaccuggauuccaaagugggcggcaacuacaacuaucuguaccggcucuucagaaagucuaaccugaagcccuuugagagagacaucuccacagaaaucuaccaggccggaagcacaccuugcaacggcguggaaggauuuaauugcuauuuuccucugcaguccuacggcuuccaaccuaccaacggagugggcuaccagccuuacagaguggucgugcugagcuuugagcugcugcaugccccugcuaccguguguggcccuaagaaaagcacgaaccuggucaagaacaaaugcgugaacuucaacuucaauggacugacaggcaccggcgugcucacagagagcaacaagaaguuccugccauuucagcaguucggcagagacauagcugacaccaccgacgccgugcgggacccccagacccuggaaauccuggauaucacacccugcagcuuuggaggcguaucugugaucacaccuggcaccaacacaagcaaucaggucgccgugcuguaccaagacgugaacugcacagaggugccgguggccauccacgccgaucagcugacccccaccuggcggguguacuccaccggcagcaacguguuccaaacccgggccggcugccugaucggcgccgaacaugugaauaacagcuacgaaugugauaucccuauuggagccggaauuugcgccagcuaccaaacccaaaccaacucuccaagaagagccagaagcguggcuagccagucuaucaucgccuacaccaugagccugggcgcugaaaacagcguggcuuacagcaacaacagcaucgccauuccuacuaacuuuaccaucagcgugacuacagagauccugcccgugagcaugaccaagaccagcguggacugcaccauguacaucuguggcgauucuaccgagugcagcaacuugcuacugcaguaugguagcuuuugcacccaacugaacagagcccugacuggcaucgccguugagcaggacaagaacacccaggagguguucgcccaggucaagcagaucuauaagaccccaccaaucaaggacuucggcggcuucaacuucagccagauccugcccgacccgagcaaaccuagcaagagaagcuucaucgaggaucuucuguucaacaaggugacgcuggccgaugccggcuucaucaaacaguacggcgauugucugggcgacaucgccgccagagaucugaucugcgcccagaaguucaauggccugaccgugcugccgccucugcucacagacgagaugaucgcccaguacaccucugcccuguuagcaggcacaaucacgagcggcuggacauuuggcgcgggcgccgcucugcagaucccuuucgcuaugcagauggccuauagauuuaauggcaucggcgugacccagaaugugcuguacgagaaccaaaagcugaucgcuaaucaguucaauagcgccaucggaaagauccaggacucucugagcagcaccgccuccgcucugggcaaacugcaggacguggucaaucaaaacgcucaggcucuaaacacccuggugaagcagcugucaagcaacuucggcgccaucuccucaguucuuaacgacaucuugucuagacuggacaagguggaggcugaggugcagaucgauagacugaucacaggcagauuacagucucugcagacauacgugacccaacagcugauccgggccgccgagaucagagccuccgccaaccuggcugccaccaagaugagcgagugugugcuuggccaguccaagagaguggacuucugcggcaaaggcuaccaccugaugagcuucccacagagcgcuccucacggaguuguguuccugcacgugaccuacgugccugcccaggagaagaauuucacaaccgccccugccaucugccacgacgguaaggcccacuuccccagagagggcguguucgugucuaaugggacccacugguucgugacacagaggaacuucuacgagccccagaucaucacaacagauaauaccuucgugucuggcaacugcgauguggugaucggcaucgugaacaauaccguguacgacccucugcagcccgagcuggacagcuucaaagaggaacuggacaaguacuucaagaaucacaccuccccugacguggaccugggagauaucuccggcaucaacgccuccguugugaacauucaaaaagaaaucgacagacugaacgagguggccaagaaccuaaacgagucucugaucgaccugcaagaacugggcaaguacgagcaguacaucaaguggccuugguacaucuggcugggcuuuaucgccggccugaucgccauugugauggugaccaucaugcugugcugcaugacaaguugcugcagcugccugaagggcuguuguucuuguggcagcugcuguaaauucgacgaggacgauagcgaaccagugcugaaaggcgucaagcugcauuacaccuga

SEQ ID NO: 41 - DNA, 3825 bp, Artificial Sequence
  sequence encoding CD74 vytoplasmic domain and transmembrane domain with the extracellular domain of spike
gccgccaccatggcacacaggaggagaagcaggagctgccgggaagaccagaagccagtcatggacgaccagcgcgacctcatctccaacaacgagcaactgcccatgctgggccggcgcccaggggccccggagagcaagtgcagccgcggagccctgtacacaggcttctccatcctggtgaccctgctcctcgccggccaggccaccaccgcctacttcctgtacgtgaacctgaccacccggacccagctaccaccagcctacacaaacagcttcacccgcggcgtgtactacccagacaaggtgttccggagctcagtgctccacagcacacaggacctgttcctgccattcttcagcaacgtgacgtggttccacgccatccacgtgtccggcaccaacggaacaaaaaggttcgacaaccccgtgctccccttcaacgacggcgtgtacttcgccagcacagaaaagtccaaaatcatccgaggatggatcttcggcacaacgctggactcaaagacccagagcctgctgatcgtgaacaacgccaccaacgtggtgatcaaggtgtgcgagttccagttctgcaacgatccattcctgggcgtatactaccacaagaacaacaagagctggatggaaagcgagttcagagtgtactcaagcgccaacaactgcaccttcgaatacgtcagccagccattcctgatggacctggaaggcaagcagggcaacttcaagaacctgagagagttcgtgttcaagaacatcgacggctacttcaagatctacagcaagcacacacccatcaacctggtgcgggacctaccgcagggattctccgcgctggaacccctggtggacctgccaataggcatcaacatcaccagattccagaccctgctggccctgcacagaagctacctgacaccaggcgacagcagcagcggctggaccgccggcgccgccgcatactacgtggggtacctgcagcccagaaccttcctgctgaaatacaacgagaacggaaccatcaccgacgccgtggactgcgccctggaccccctgagcgaaaccaagtgcaccctgaaaagcttcacagtggaaaagggaatataccagacctccaacttcagagtccagccaaccgaaagcatcgtgcggttcccaaacatcaccaacctgtgcccattcggcgaggtattcaacgccacacggttcgccagcgtatacgcctggaaccggaagcggatcagcaactgcgtggcagactacagcgtgctatacaacagcgccagcttaagcaccttcaagtgctacggcgtgtccccaaccaagctgaacgacctgtgcttcaccaacgtgtacgccgacagcttcgtgatcagaggcgacgaggtgcgccagatcgcaccaggacagaccggaaagatcgcagactacaactacaagctgcccgacgacttcaccggctgcgtgatcgcctggaacagcaacaacctggactccaaagtgggcggcaactacaactacctgtaccggctcttcagaaagagcaacctgaagcccttcgagagagacatcagcacagaaatctaccaggccggaagcacaccatgcaacggcgtggaaggattcaactgctatttcccactgcagtcctacggcttccaaccaaccaacggagtgggctaccagccatacagagtggtcgtgctgagcttcgagctgctgcacgccccagcaaccgtgtgcggcccaaagaaaagcacgaacctggtcaagaacaaatgcgtgaacttcaacttcaacggactgacaggcaccggcgtgctcacagagagcaacaagaagttcctgccattccagcagttcggcagagacatagcagacaccaccgacgccgtgcgggacccccagaccctggaaatcctggacatcacaccctgcagcttcggaggcgtaagcgtgatcacaccaggcaccaacacaagcaaccaggtcgccgtgctgtaccaagacgtgaactgcacagaggtgccggtggccatccacgccgaccagctgacccccacctggcgggtgtactccaccggcagcaacgtgttccaaacccgggccggctgcctgatcggcgccgaacacgtgaacaacagctacgaatgcgacatcccaatcggagccggaatctgcgccagctaccaaacccaaaccaacagcccaagaagagccagaagcgtggcaagccagagcatcatcgcctacaccatgagcctgggcgcagaaaacagcgtggcatacagcaacaacagcatcgccatcccaaccaacttcaccatcagcgtgaccacagagatcctgcccgtgagcatgaccaagaccagcgtggactgcaccatgtacatctgcggcgacagcaccgagtgcagcaacttgctactgcagtacggaagcttctgcacccaactgaacagagccctgaccggcatcgccgtcgagcaggacaagaacacccaggaggtgttcgcccaggtcaagcagatctacaagaccccaccaatcaaggacttcggcggcttcaacttcagccagatcctgcccgacccgagcaaaccaagcaagagaagcttcatcgaggacctactgttcaacaaggtgacgctggccgacgccggcttcatcaaacagtacggcgactgcctgggcgacatcgccgccagagacctgatctgcgcccagaagttcaatggcctgaccgtgctgccgccactgctcacagacgagatgatcgcccagtacaccagcgccctgttagcaggcacaatcacgagcggctggacattcggcgcgggcgccgcactgcagatcccattcgcaatgcagatggcctacagattcaatggcatcggcgtgacccagaacgtgctgtacgagaaccaaaagctgatcgcaaaccagttcaacagcgccatcggaaagatccaggacagcctgagcagcaccgcctccgcactgggcaaactgcaggacgtggtcaaccaaaacgcacaggcactaaacaccctggtgaagcagctgtcaagcaacttcggcgccatcagctcagtcctaaacgacatcttgagcagactggacaaggtggaggcagaggtgcagatcgacagactgatcacaggcagattacagagcctgcagacatacgtgacccaacagctgatccgggccgccgagatcagagcctccgccaacctggcagccaccaagatgagcgagtgcgtgctaggccagagcaagagagtggacttctgcggcaaaggctaccacctgatgagcttcccacagagcgcaccacacggagtagtgttcctgcacgtgacctacgtgccagcccaggagaagaacttcacaaccgccccagccatctgccacgacggaaaggcccacttccccagagagggcgtgttcgtgagcaacgggacccactggttcgtgacacagaggaacttctacgagccccagatcatcacaacagacaacaccttcgtgagcggcaactgcgacgtggtgatcggcatcgtgaacaacaccgtgtacgacccactgcagcccgagctggacagcttcaaagaggaactggacaagtacttcaagaaccacacctccccagacgtggacctgggagacatcagcggcatcaacgcctccgtagtgaacatccaaaaagaaatcgacagactgaacgaggtggccaagaacctaaacgagagcctgatcgacctgcaagaactgggcaagtacgagcagtacatcaagtggccataa

SEQ ID NO: 42 - RNA, 3981 nt, Artificial Sequence
  sequence encoding CD74 cytoplasmic domain and transmembrane domain with full length spike (without furin cleavage site)
gccgccaccaugcacaggaggagaagcaggagcugucgggaagaucagaagccagucauggaugaccagcgcgaccuuaucuccaacaaugagcaacugcccaugcugggccggcgcccuggggccccggagagcaagugcagccgcggagcccuguacacaggcuuuuccauccuggugacucugcuccucgcuggccaggccaccaccgccuacuuccuguacgugaaccugaccacccggacccagcuaccuccugccuacacaaacagcuucacccgcggcguguacuacccugauaagguguuccggagcucugugcuccacagcacacaggaccuguuccugccuuucuucagcaacgugacgugguuccacgccauccacguguccggcaccaaugguacaaaaagguuugacaaccccgugcuccccuucaacgacggcguguauuuugccagcacugaaaaguccaauaucauccgaggauggaucuucggcacaacgcuggacucuaagacccagagccugcugaucgugaacaacgccaccaacguggugaucaaggugugcgaguuucaguucugcaacgauccuuuucugggcguuuacuaccacaagaacaacaagagcuggauggaaagcgaguucagaguguauucuagcgccaacaacuguaccuucgaauacgucagccagccuuuccugauggaccuggaaggcaagcagggcaacuucaagaaccugagagaguucguguucaagaacaucgacggcuacuucaagaucuacagcaagcacacaccuaucaaccuggugcgggaccuuccgcagggauucuccgcgcuggaaccccugguggaccugccuauuggcaucaacaucaccagauuucagacccugcuggcccugcacagaagcuaccugacaccuggcgacucuucuagcggcuggaccgccggcgccgccgcuuauuacgugggguaccugcagcccagaaccuuccugcugaaauacaaugagaacggaaccaucaccgaugccguggacugcgcccuggacccccugucugaaaccaagugcacccugaaaucuuuuacaguggaaaagggaauuuaccagaccuccaacuuuagaguccagccuaccgaaagcaucgugcgguucccuaacaucaccaaccuguguccuuucggcgagguuuucaacgccacacgguucgccagcguauacgccuggaaccggaagcggaucucuaacuguguggcagacuacucugugcuauacaacucggccagcuuuagcaccuucaagugcuacggcguguccccaaccaagcugaacgaccugugcuucaccaacguguacgccgauagcuucgugaucagaggcgaugaggugcgccagaucgcuccuggacagaccggaaagaucgcugacuacaauuacaagcugcccgacgacuucaccggcugcgugaucgccuggaacagcaacaaccuggauuccaaagugggcggcaacuacaacuaucuguaccggcucuucagaaagucuaaccugaagcccuuugagagagacaucuccacagaaaucuaccaggccggaagcacaccuugcaacggcguggaaggauuuaauugcuauuuuccucugcaguccuacggcuuccaaccuaccaacggagugggcuaccagccuuacagaguggucgugcugagcuuugagcugcugcaugccccugcuaccguguguggcccuaagaaaagcacgaaccuggucaagaacaaaugcgugaacuucaacuucaauggacugacaggcaccggcgugcucacagagagcaacaagaaguuccugccauuucagcaguucggcagagacauagcugacaccaccgacgccgugcgggacccccagacccuggaaauccuggauaucacacccugcagcuuuggaggcguaucugugaucacaccuggcaccaacacaagcaaucaggucgccgugcuguaccaagacgugaacugcacagaggugccgguggccauccacgccgaucagcugacccccaccuggcggguguacuccaccggcagcaacguguuccaaacccgggccggcugccugaucggcgccgaacaugugaauaacagcuacgaaugugauaucccuauuggagccggaauuugcgccagcuaccaaacccaaaccaacucugcuagccagucuaucaucgccuacaccaugagccugggcgcugaaaacagcguggcuuacagcaacaacagcaucgccauuccuacuaacuuuaccaucagcgugacuacagagauccugcccgugagcaugaccaagaccagcguggacugcaccauguacaucuguggcgauucuaccgagugcagcaacuugcuacugcaguaugguagcuuuugcacccaacugaacagagcccugacuggcaucgccguugagcaggacaagaacacccaggagguguucgcccaggucaagcagaucuauaagaccccaccaaucaaggacuucggcggcuucaacuucagccagauccugcccgacccgagcaaaccuagcaagagaagcuucaucgaggaucuucuguucaacaaggugacgcuggccgaugccggcuucaucaaacaguacggcgauugucugggcgacaucgccgccagagaucugaucugcgcccagaaguucaauggccugaccgugcugccgccucugcucacagacgagaugaucgcccaguacaccucugcccuguuagcaggcacaaucacgagcggcuggacauuuggcgcgggcgccgcucugcagaucccuuucgcuaugcagauggccuauagauuuaauggcaucggcgugacccagaaugugcuguacgagaaccaaaagcugaucgcuaaucaguucaauagcgccaucggaaagauccaggacucucugagcagcaccgccuccgcucugggcaaacugcaggacguggucaaucaaaacgcucaggcucuaaacacccuggugaagcagcugucaagcaacuucggcgccaucuccucaguucuuaacgacaucuugucuagacuggacaagguggaggcugaggugcagaucgauagacugaucacaggcagauuacagucucugcagacauacgugacccaacagcugauccgggccgccgagaucagagccuccgccaaccuggcugccaccaagaugagcgagugugugcuuggccaguccaagagaguggacuucugcggcaaaggcuaccaccugaugagcuucccacagagcgcuccucacggaguuguguuccugcacgugaccuacgugccugcccaggagaagaauuucacaaccgccccugccaucugccacgacgguaaggcccacuuccccagagagggcguguucgugucuaaugggacccacugguucgugacacagaggaacuucuacgagccccagaucaucacaacagauaauaccuucgugucuggcaacugcgauguggugaucggcaucgugaacaauaccguguacgacccucugcagcccgagcuggacagcuucaaagaggaacuggacaaguacuucaagaaucacaccuccccugacguggaccugggagauaucuccggcaucaacgccuccguugugaacauucaaaaagaaaucgacagacugaacgagguggccaagaaccuaaacgagucucugaucgaccugcaagaacugggcaaguacgagcaguacaucaaguggccuugguacaucuggcugggcuuuaucgccggccugaucgccauugugauggugaccaucaugcugugcugcaugacaaguugcugcagcugccugaagggcuguuguucuuguggcagcugcuguaaauucgacgaggacgauagcgaaccagugcugaaaggcgucaagcugcauuacaccuga

SEQ ID NO: 43 - DNA, 3840 bp, Artificial Sequence
  sequence encoding a Spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagtgaacctcactacgcgaactcaactgccgcctgcttacactaatagttttacacgaggagtctactacccggacaaggtgttccgctcaagtgtcttgcacagcacgcaagacctgttcttgccatttttcagcaatgtaacgtggtttcatgccatccacgtgtcaggtactaacggaacgaagagatttgataacccggtcctcccgttcaacgacggggtttattttgccagtacagaaaagtccaatattataaggggatggattttcggcacgactctcgactctaagacacaaagcttgctcatcgttaataatgctacaaacgtggttataaaagtatgcgagttccaattctgtaacgacccctttctgggggtttactaccacaaaaataataagagttggatggagagcgaattcagagtttactcttccgcgaataattgcaccttcgagtacgtatcccagccttttcttatggatcttgaggggaaacaaggtaatttcaaaaaccttagagaattcgtttttaagaatatagatggatacttcaagatttattctaaacataccccaatcaatctggtgcgcgatcttcctcagggtttctctgcgctggagcctctcgttgatctgccgatcggaattaatataaccagattccagacccttttggcattgcaccgctcctatctgacacctggggatagtagttccggctggacagcgggcgccgcggcatactacgttggatatctccaaccccgcacttttcttctgaaatataatgagaatggaacgattacagacgcagtagattgcgcgcttgaccccctttcagaaacaaagtgcacgctgaaatctttcacggtcgagaagggcatttaccaaacgtccaatttcagagttcaacccactgagtctatagtcaggtttccgaacatcaccaatctctgcccatttggagaggttttcaacgcaacacggttcgcctctgtatacgcatggaatagaaagagaatatccaattgtgtcgcagactatagtgtcttgtacaactctgcgtcatttagcacttttaaatgttatggggtgagtccaactaaattgaacgatctctgctttacaaatgtttacgcagatagttttgtgatacgaggcgacgaggtacgacaaattgctcctggccagaccgggaaaatagcggactacaattataaactgccagacgatttcaccggctgtgtgatcgcgtggaactcaaacaacttggatagtaaggtaggtggtaactataactacttgtaccggctcttccgcaaaagtaatcttaaaccgttcgagcgggacatctctaccgagatctatcaagcaggtagcactccctgcaatggagtcgagggttttaattgctattttcccctccaatcatacgggtttcagcccacaaatggtgttgggtatcagccctatcgagttgtggtgctttcatttgaattgcttcatgcaccggcgacagtttgtggtcctaaaaagagtaccaatttggttaaaaacaaatgtgtcaactttaactttaacggtctgactggtacgggggtgcttacggaatctaataaaaagtttcttccgttccagcaattcggacgggacatagcggacacgactgacgcggttagagacccacaaacactcgagattcttgacataaccccctgttctttcggcggagtttctgtaataacgcctgggacaaatacgtcaaaccaggttgcagtattgtaccaggatgtcaactgcactgaagtgccagtcgcgatccacgcagatcagttgactccaacatggagggtctactctaccggttctaacgtgtttcagaccagagccgggtgcctcattggcgcggaacatgtgaacaattcttacgaatgcgacatacccatcggcgccggcatttgtgcgtcataccaaactcaaactaattcccggagtgtggcctcccaatctattattgcttatactatgagtcttggcgcggaaaacagtgtagcgtactcaaataactctattgcgatccccacgaatttcactataagcgtaactactgaaattttgccggtaagcatgacgaaaacttcagtggactgcacaatgtacatttgtggtgacagcactgagtgctcaaatctcctccttcagtatggttctttctgcacacaacttaacagggcacttaccggaatcgcagtagaacaagacaaaaacacgcaagaagtattcgcacaggtcaaacaaatttataaaaccccgccgatcaaggattttggagggtttaacttcagtcagatccttccagacccatccaaacccagcaaacggagctttattgaagacctgctgtttaataaagttacccttgcagatgccggtttcatcaagcaatacggggattgtctgggcgacattgccgcccgcgacttgatctgtgcccagaagttcaacggtctcacagtcttgccgccacttctcactgatgaaatgatagcgcagtatacatccgcgctgctggctgggacgataacatctggttggactttcggggccggggcggctttgcaaattcctttcgccatgcagatggcctataggtttaacggcataggagtgactcaaaatgttctttatgagaatcagaagcttatcgccaaccaatttaactccgcgattgggaagatacaggactcattgagctctactgctagcgcccttggcaagcttcaagacgtagtcaaccaaaatgctcaagcgctcaatactctcgtaaagcagctctcaagcaactttggcgcgatatcttcagttttgaacgacatcttgagcagactcgacaaggtggaggctgaagttcaaattgatcgactgattacgggccggctccagtcactccaaacgtacgtgacccaacaactcattagagcggcagagattagagcgagcgctaatctggcggccacaaagatgtcagaatgtgtgcttggtcaatccaaaagggtggatttctgcggaaagggctatcacttgatgagtttcccgcagtccgcgcctcatggagttgttttcctccacgtaacctacgttcccgctcaggaaaagaatttcacgacagctccggcaatatgtcatgacggtaaagcacatttccccagggaaggcgtttttgtcagtaatggaacccactggttcgtcacccagcgaaacttttacgagccccaaataataactactgacaataccttcgtcagcggcaattgcgacgtcgtcataggcatagtaaataatacggtttacgaccctcttcaacccgagttggacagcttcaaagaagaacttgataagtattttaaaaaccacactagcccagatgtagaccttggcgacatcagcggtatcaatgcttccgttgtgaacattcaaaaggaaattgaccgactcaatgaagtagcgaagaacttgaatgagtctctgatcgatcttcaggagctcgggaaatatgagcaatacatcaaatggccgtcacgactggaggaagaactgcgccgacgcctgactgaatga

SEQ ID NO: 44 - protein, 1277 aa, Artificial Sequence
  Spike S1+S2 ECD_CD74+HLA
MHRRRSRSCREDQNPVMDDQRDLISNNEQLPMLGRRPGAPESKCSRWMVAAVVAGTIVAGLLVLGAIIGVVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 45 - DNA, 3834 bp, Artificial Sequence
  sequence encoding Kent spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagttaacctgacaacacgcacccagttgccaccggcctatacgaactctttcacgcgcggagtgtattatcccgataaagtctttcggagttctgtgctgcactctacacaagatctttttctgccctttttctccaacgttacgtggttccatgcaataagtggtacgaatggtactaaaaggttcgataatcccgtacttcccttcaatgacggcgtgtatttcgcgagcacagaaaagagcaacataataagaggttggatttttgggactaccctggactctaaaacacaaagcctcctcattgttaataacgcaactaatgtcgtaataaaggtatgcgaatttcagttttgtaacgacccttttttgggagtctatcataagaacaataagtcctggatggaaagtgaatttagggtgtatagttcagcgaacaactgtacatttgagtatgtgtcacagccgttccttatggatcttgaagggaaacaggggaacttcaagaacctgcgggaattcgtgtttaaaaatatagacggttattttaaaatctatagcaaacacaccccaataaacctggttcgggatcttcctcagggattttcagctttggaaccgttggttgacctccccatcgggataaatatcacgcgatttcaaacactgctggcactgcaccggagttatctgactcctggagatagcagctccggctggacggctggcgccgcggcttattatgtcgggtaccttcaaccgcgaacgtttcttttgaagtataacgaaaatggcactataaccgatgcagtagattgtgcgttggaccctctttccgagactaagtgcaccctgaagtcatttacagtagagaaggggatataccagacaagcaacttccgcgtccaaccaacggaaagcatcgtgcgctttcccaatattactaacctctgtccgttcggcgaggtttttaatgctaccagatttgcctcagtgtatgcctggaaccgcaagaggattagcaactgcgttgctgactatagcgtgctttacaattccgccagttttagtacgttcaagtgttatggggtaagccctaccaagcttaacgatctttgcttcacgaatgtttacgcagactcattcgtaatacgaggtgatgaagtgcgacagatagccccgggtcagacggggaaaatagccgactacaattacaagctcccggacgacttcacgggctgtgttattgcgtggaacagcaataacctggacagtaaagttggagggaattataactacttgtacaggctgttccgcaaatctaatttgaaaccgtttgaaagggacatttccaccgagatttaccaagccggcagtaccccatgtaacggagttgagggatttaattgctactttccgctccaatcatatggattccagcctacatacggcgtcggatatcaaccttatagggtggtcgttctttcttttgagctgcttcacgccccagcaaccgtttgcggcccgaaaaaatcaacgaacttggtcaaaaacaagtgcgtcaacttcaatttcaacggactgacaggcaccggagtgctcaccgagtcaaataaaaaattcctcccgtttcaacaattcggtagagatatagacgacaccacagatgcggtacgcgaccctcagactcttgaaattttggacattacaccatgttccttcggtggcgtctcagtgattacgcctggcactaacactagcaaccaagtcgcagtactttatcagggcgttaactgtacagaagtaccagttgcgatacacgcggaccagctcaccccaacctggcgcgtatattctaccggatctaatgtgtttcaaacgcgggcggggtgcctcataggtgcagagcatgtcaataactcctacgaatgtgacattcctataggagcaggaatatgtgcgagctaccagactcaaaccaattcacacaggagcgtagctagtcagtccataattgcctacactatgtcacttggtgctgaaaacagtgtggcttattcaaataattccatagctattccaatcaatttcactattagcgtaacaactgagatacttccagtcagcatgactaagacttcagttgattgtacgatgtatatttgtggcgattctacagaatgtagtaaccttctgttgcaatacggctccttttgcacacagctcaatcgcgcattgacgggtatagctgtagagcaagataaaaacacccaagaagtcttcgcgcaggtgaagcaaatatataagacaccaccgattaaagatttcggtggattcaattttagccaaattctgcctgatccaagcaaaccgtcaaaaagatccttcatagaggacctgcttttcaataaggtaacgctggcggacgcagggtttattaaacaatacggcgattgccttggtgacattgctgcacgagacctcatttgcgcccagaagttcaacggactcacagtactgcctccactgctcacagacgagatgatcgcccagtacacatctgccttgttggctggtacgataacttctggttggacctttggagcgggagcagcacttcagattccctttgccatgcagatggcgtacagattcaacggcattggtgtcacgcagaacgtactgtatgagaaccagaagttgatagctaatcaattcaactccgcgataggtaagatccaagacagtctctcctcaacagcctccgcactcgggaaactccaggatgtggtaaatcagaatgcacaggcattgaatacgcttgttaaacaattgtcctcaaattttggcgccatatcatcagtgctcaacgacatccttgccagactcgataaagtggaggcagaagtgcagatagatcgcttgattactgggcgcctccaatctctccaaacgtacgtgacacaacagcttattcgggcagcggaaatccgcgcctctgcgaaccttgcagcgaccaagatgagcgaatgtgttctcgggcagtcaaaacgagtagatttctgcggtaaaggatatcatcttatgtcattcccccaaagcgctccacatggtgttgtatttctgcacgtgacttacgtgccggcacaggagaagaatttcacaacggctcccgccatctgtcacgacggaaaggcacacttccctagggagggggtgtttgtaagcaatggtacccactggtttgtcacacaacgaaacttctatgaacctcagataattacaacgcataatacttttgtaagtgggaactgcgacgtcgttatcggaatagttaacaatactgtatatgatccgttgcagccggagttggactcatttaaagaagagctggacaagtacttcaaaaaccatacaagcccagatgtagatcttggggacataagtgggataaacgccagcgttgtgaacatacagaaggaaatagacaggctgaatgaggtagcaaagaatcttaacgaaagccttatagatcttcaggaacttgggaagtacgaacagtacataaagtggccttcacgactggaggaagaactgcgccgacgcctgactgaatga

SEQ ID NO: 46 - protein, 1275 aa, Artificial Sequence
  Kent spike S1+S2 ECD_CD74+HLA
MHRRRSRSCREDQNPVMDDQRDLISNNEQLPMLGRRPGAPESKCSRWMVAAVVAGTIVAGLLVLGAIIGVVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 47 - DNA, 3840 bp, Artificial Sequence
  sequence encoding South Africa Spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagtcaatttcactacacgcacacagctgcccccggcgtacactaacagttttacgaggggagtctactacccggacaaagtgttcaggtcttccgtactccatagcacacaagacctcttcttgccttttttcagcaatgttacgtggttccatgcaatccatgtgagcggtacaaacgggactaaacgctttgcaaatccagtgttgcccttcaacgacggtgtatacttcgcgagtacagagaagagcaacataatacgcgggtggatatttggaacgacgcttgattctaagacacagagcttgctcatcgtaaacaacgcaacgaatgttgtaatcaaggtctgcgaattccaattctgcaatgacccatttcttggagtttattaccataagaacaataagtcttggatggaatccgagttccgcgtatactctagcgcgaacaactgcaccttcgagtatgtgtcccaaccgtttctgatggatcttgagggaaagcagggtaattttaaaaatctccgggagttcgttttcaaaaatattgacggatatttcaaaatctacagcaagcataccccaatcaatttggtgcggggtctgccacaaggattttccgctcttgaacctcttgttgaccttcccataggaatcaatataactcggttccaaactcttcttgcgctccatatatcctacttgacgcccggagattcatcaagtggctggacggctggggctgccgcatactacgtcggctatctgcaaccaaggacgtttctcctcaaatacaatgaaaatggtacgatcaccgacgctgtggactgcgctctcgacccgttgtcagagacgaagtgcaccttgaagtccttcaccgtggaaaagggaatttaccagactagtaacttccgggtgcagcccacggaatcaattgtgcgatttcctaatatcactaatctctgtcccttcggggaagtgttcaatgcgactcgattcgcttcagtatacgcttggaatagaaagagaatctcaaattgcgtggctgactattccgtactgtataatagtgcgtcttttagcacgtttaagtgttatggcgtatccccgacaaagctcaacgatctctgcttcacgaatgtctatgccgactccttcgtgatacgcggtgacgaggtacgacaaattgcacccggccaaactggaaatattgccgactacaattataagttgccggacgattttaccggttgcgtaatagcgtggaactctaataacctcgactctaaggtaggaggcaactacaattacttgtaccgactgttcaggaaatccaacctgaagcccttcgaaagagacatcagtacagagatttatcaagccgggtccaccccttgcaatggtgtcaaaggcttcaactgctattttccgttgcagtcttacggtttccagccgacgtacggtgtgggttatcaaccctaccgcgttgtggtgctgagctttgagcttctccacgctccagccactgtctgcgggcctaagaaatccacaaacctcgttaagaataaatgcgtaaatttcaacttcaacgggcttactggcacgggcgtgcttacggagtccaataagaaattcctgcctttccagcagttcggccgcgacatcgcggacacaacagatgctgtgcgagacccacaaaccctggagattctggacatcacgccttgctcttttggcggtgtcagcgtaataacgccaggcaccaacaccagtaatcaggtcgccgtgctttatcagggtgtgaactgtacggaggtaccggttgccatacacgcagaccaactcacacctacgtggagagtttacagcaccgggtcaaacgtatttcagacccgcgcaggctgtctgatcggcgcagagcatgtcaataactcatacgagtgtgatataccaatcggagccggcatctgtgcgagctatcaaacccaaactaattcacgaagcgtggccagccagtccatcattgcctacacgatgtctttgggagtagagaatagcgtggcatactctaacaacagtatcgctattcctaccaacttcacaatatcagttactacagaaatcctcccggtgagcatgactaagacatccgtagactgtacaatgtacatttgtggggacagcacggagtgttcaaacctgctgttgcaatacggaagcttctgcacccagctcaaccgagccctgaccggcattgccgtagaacaagataagaatacgcaggaagtgttcgcccaggttaagcagatttacaagactccgcctatcaaggacttcgggggtttcaactttagtcagattttgcctgacccgtcaaagcctagcaaacgaagttttatcgaagatttgttgtttaacaaggtgactttggctgatgcgggattcataaaacaatacggcgattgccttggcgatattgcggcccgagatctcatttgcgcgcaaaagttcaacggtcttaccgtccttccgcccctgcttacggacgagatgatcgcacaatacacttcagcactgctggctggtacaataaccagtgggtggacattcggggctggtgcagcgctccagatcccctttgcgatgcagatggcgtatagattcaatggcatcggggtcactcagaacgtgctttatgagaaccaaaagcttattgcaaatcaatttaactctgcgattggaaagattcaagacagcctctcatccacagctagtgcgcttggcaagcttcaggacgtcgtaaaccaaaacgcccaagctctcaacacactcgttaaacagctttcctcaaactttggggctattagctctgtgctgaatgatattcttagcagactcgacaaagtcgaggctgaagtacaaatagatagactgataacaggtagacttcagtctctccaaacctacgtgacacagcagcttataagagctgctgagatcagagcttctgcgaaccttgcggcgactaaaatgtccgagtgtgtactcgggcagtctaaacgcgtcgacttctgtggcaagggttaccatctcatgagttttccccaatcagcgccccatggggtagtttttctgcacgtaacatacgtcccagcccaagaaaagaattttaccaccgcccctgcgatttgtcacgacggaaaagcacactttccaagagaaggcgtatttgttagcaatggcacccattggttcgtcacccagagaaacttttacgaaccgcagattatcacgacagataatacatttgtatctggtaattgtgacgtcgtgatcggcatcgtaaacaacactgtttacgaccccctccagccagagcttgactcatttaaagaggagctcgacaaatattttaagaaccacacaagtccagacgtcgacttgggtgacatttccggcatcaatgcaagtgtggtcaacatccaaaaggaaattgacagattgaacgaggttgctaagaaccttaatgagtcactcattgacctccaggaactgggaaaatacgaacagtatattaagtggccctcacgactggaggaagaactgcgccgacgcctgactgaatga

SEQ ID NO: 48 - protein, 1277 aa, Artificial Sequence
  South Africa Spike S1+S2 ECD_CD74+HLA
MHRRRSRSCREDQNPVMDDQRDLISNNEQLPMLGRRPGAPESKCSRWMVAAVVAGTIVAGLLVLGAIIGVVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 49 - DNA, 3837 bp, Artificial Sequence
  California Spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagtaaatttgacaacgcgcacccaattgcccccagcatatacgaactctttcacgaggggcgtatattatccggataaggtatttcggtcatctgttctgcacagcacccaggacctcttccttccattcttttcaaatgtaacttggttccatgcgatagtatcaggaacgaatgggacaaaaaggttcgataatccggtcttgccgttcaacgatggggtgtacttcgccagtaccgaaaagtctaacattatacgcggttggatttttggcactacgcttgactcaaagacacagtcactcctcattgtaaataatgctactaatgtcgtgatcaaagtttgtgagttccaattctgcaacgacccgttcctgggagtgtattatcacaaaaataataagtcctgcatggagtcagagttcagggtttactcaagtgcgaacaattgcacatttgagtacgtttctcaaccatttctcatggatttggaggggaagcagggaaactttaaaaacctgagagaatttgtttttaagaacattgatgggtatttcaagatttatagtaaacacacccctatcaacttggttagggatctccctcaaggtttctctgctctcgagccccttgtagatctgccaataggcatcaatatcacacgctttcagacactcctcgcacttcataggagctacctgacgccaggtgactcttcctcaggttggacagccggcgcagccgcatactacgttggctacctccaaccaaggacatttctgttgaaatacaacgaaaatgggaccatcaccgacgcagtcgattgtgctctcgaccctctttccgagactaaatgtaccctcaaaagctttactgttgagaagggtatctatcagacatctaactttcgggtgcaacccactgagtcaattgtgcgattcccaaatattacgaacctctgtccttttggcgaggtttttaacgccactaggttcgccagtgtatatgcttggaaccgaaaacggataagcaattgtgttgctgactactccgtcctctacaatagcgctagtttctcaacatttaagtgttacggtgtgagccctacgaaacttaacgatttgtgcttcactaacgtctatgccgacagtttcgtaatccgaggcgatgaggtcaggcaaattgccccgggccaaacggggaaaatcgctgattacaattataagttgccagatgattttacgggatgtgtcattgcatggaacagtaataacctcgattcaaaggttggcggaaattataattacagatatcggctttttagaaaatctaaccttaaaccatttgagcgggacataagcacggagatttaccaggctggtagcactccgtgcaacggtgtagaaggattcaattgctattttccattgcagtcttatggattccaacccaccaatggggtagggtaccaaccatacagggtggtagtccttagctttgaacttttgcatgcgccagctaccgtctgcggtcccaaaaagagtacgaacttggtaaaaaataaatgcgtcaactttaattttaacggtctgacgggaacgggggttctcaccgagtctaacaaaaagtttttgccatttcagcagttcggacgagatattgccgacactaccgacgccgtgcgcgatccacaaaccttggagatactcgacatcacaccttgcagcttcggtggtgttagcgttattacgccaggaacgaacacttcaaatcaggtggccgtcttgtatcaagatgttaactgtacagaggtgcccgtcgcaatacacgcagatcagctcacccctacctggagagtttactctacaggctctaatgtgttccaaactagggcaggctgcttgattggagctgaacacgtaaacaactcatacgagtgcgatatcccgattggtgcgggtatttgcgccagctatcagacgcagactaactcacgatcagtggcgtcacagtcaataattgcgtatacaatgagtctcggtgcagagaattccgtcgcatactcaaacaacagcatagcgattccaactaattttaccattagcgttacaacggaaatcctccccgttagcatgaccaagacctccgtggattgcacaatgtacatctgtggagacagcaccgagtgctccaatcttcttcttcaatatgggagcttttgcacacagctgaaccgcgcgctcactggcattgcggtagaacaagacaaaaacacccaagaagtgtttgcgcaggtcaaacagatttacaagacaccaccaatcaaagacttcggggggttcaatttctcccaaattcttcctgatccaagcaaaccatctaaaaggtccttcatagaagacctcctgttcaataaggtgacactcgcggacgcagggtttattaagcaatatggagattgtctcggggatattgcagcgagagatcttatttgtgctcaaaagtttaatggcttgactgtcctcccgcccttgctcacagacgagatgatagcacaatataccagtgcgctgcttgcgggcacgattacttccggatggactttcggggcaggagcggcgctccagattcctttcgcgatgcaaatggcctaccgatttaacgggattggcgtcacacaaaacgttctttacgaaaaccagaaacttattgccaaccaattcaatagtgcaattggtaaaattcaggattcactctcctccacggcgtcagctctcggcaagctccaagacgtggttaaccaaaatgcgcaggcgcttaacacgcttgtaaaacaactctcttctaattttggcgcgattagtagcgttctcaacgatatcttgtcacgcttggacaaagtagaagcagaagtccaaatagaccggctgatcactggtcgattgcaatctctgcagacatacgtgacgcagcagctcatccgggcagccgagatccgggcgtccgctaatctggccgcaaccaaaatgagcgagtgtgtccttgggcaaagcaaacgcgtggatttttgcggaaagggctaccacctcatgagtttcccccaatctgccccgcacggtgtcgtctttttgcatgtaacgtatgtcccggcgcaggagaaaaatttcactacagctcccgccatctgtcacgacggcaaggcacacttccctcgagaaggggtgtttgtaagcaatggaacacactggttcgtcacccagcggaatttttatgaacctcagataatcacaacagacaacactttcgtttccggaaactgcgatgtcgtaattgggatagtgaataataccgtatatgacccattgcagccggagttggactcctttaaagaggaattggacaaatactttaaaaatcacacttcacccgatgtggacctgggagatatatccgggattaacgcaagtgtggttaacatacaaaaggaaattgaccgccttaacgaggttgctaagaacttgaatgagtctctcatagatctccaggaactggggaaatacgagcaatatatcaaatggccgtcacgactggaggaagaactgcgccgacgcctgactgaatga

SEQ ID NO: 50 - protein, 1276 aa, Artificial Sequence
  California Spike S1+S2 ECD_CD74+HLA
MHRRRSRSCREDQNPVMDDQRDLISNNEQLPMLGRRPGAPESKCSRWMVAAVVAGTIVAGLLVLGAIIGVVNLTTRTQLPPAYTNSFTRGVYYPDKVFRSSVLHSTQDLFLPFFSNVTWFHAIVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 51 - DNA, 3840 bp, Artificial Sequence
  sequence encoding Brazil Spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagttaactttactaatcggacccaactcccgagtgcgtacactaactcttttacccggggcgtctattatcctgacaaagtgtttcgatcatctgttttgcactccacccaagatctctttcttccatttttcagcaacgttacgtggttccacgcgatacatgttagtggtactaatggcacaaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaggagctatctgaccccgggcgactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaacattgctgattataactacaagttgcccgatgacttcacggggtgtgttattgcatggaattcaaataatcttgatagtaaggtagggggtaactataattacctttatcgcttgttccgcaaatccaatctgaaacccttcgaaagggatatcagtaccgagatttatcaagccggctccacaccttgtaacggagtaaaaggttttaactgttatttcccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagcagaagcgttgcgtcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgacggttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctattaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttggagacatctctgggataaacgctagcttcgtaaacatacaaaaagaaatcgataggcttaacgaggtcgcaaaaaatcttaatgaaagtcttatcgatttgcaagagcttggaaagtatgagcagtatatcaaatggccttcacgactggaggaagaactgcgccgacgcctgactgaatga

SEQ ID NO: 52 - protein, 1277 aa, Artificial Sequence
  Brazil Spike S1+S2 ECD_CD74+HLA
MHRRRSRSCREDQNPVMDDQRDLISNNEQLPMLGRRPGAPESKCSRWMVAAVVAGTIVAGLLVLGAIIGVVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPSRLEEELRRRLTE

SEQ ID NO: 53 - DNA, 3837 bp, Artificial Sequence
  sequence encoding Delta Spike S1+S2 ECD_CD74+HLA
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatcacgactggaggaagaactgcgccgacgcctgactgaataa

SEQ ID NO: 54 - protein, 1276 aa, Artificial Sequence
  Delta Spike S1+S2 ECD_CD74+HLA
MHRRRSRSCREDQNPVMDDQRDLISNNEQLPMLGRRPGAPESKCSRWMVAAVVAGTIVAGLLVLGAIIGVVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYRYRLFRKSNLKPFERDISTEIYQAGSTPCNGVQGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPSRLEEELRRRLTE

SEQ ID NO: 55 - DNA, 3873 bp, Artificial Sequence
  sequence encoding Wuhan Spike S1+S2 ECD_HLA
gccaccatggcggtcatggcaccacgcacgctccttcttttgttgtctggtgcccttgcactcacccaaacgtgggcagtgaacctcactacgcgaactcaactgccgcctgcttacactaatagttttacacgaggagtctactacccggacaaggtgttccgctcaagtgtcttgcacagcacgcaagacctgttcttgccatttttcagcaatgtaacgtggtttcatgccatccacgtgtcaggtactaacggaacgaagagatttgataacccggtcctcccgttcaacgacggggtttattttgccagtacagaaaagtccaatattataaggggatggattttcggcacgactctcgactctaagacacaaagcttgctcatcgttaataatgctacaaacgtggttataaaagtatgcgagttccaattctgtaacgacccctttctgggggtttactaccacaaaaataataagagttggatggagagcgaattcagagtttactcttccgcgaataattgcaccttcgagtacgtatcccagccttttcttatggatcttgaggggaaacaaggtaatttcaaaaaccttagagaattcgtttttaagaatatagatggatacttcaagatttattctaaacataccccaatcaatctggtgcgcgatcttcctcagggtttctctgcgctggagcctctcgttgatctgccgatcggaattaatataaccagattccagacccttttggcattgcaccgctcctatctgacacctggggatagtagttccggctggacagcgggcgccgcggcatactacgttggatatctccaaccccgcacttttcttctgaaatataatgagaatggaacgattacagacgcagtagattgcgcgcttgaccccctttcagaaacaaagtgcacgctgaaatctttcacggtcgagaagggcatttaccaaacgtccaatttcagagttcaacccactgagtctatagtcaggtttccgaacatcaccaatctctgcccatttggagaggttttcaacgcaacacggttcgcctctgtatacgcatggaatagaaagagaatatccaattgtgtcgcagactatagtgtcttgtacaactctgcgtcatttagcacttttaaatgttatggggtgagtccaactaaattgaacgatctctgctttacaaatgtttacgcagatagttttgtgatacgaggcgacgaggtacgacaaattgctcctggccagaccgggaaaatagcggactacaattataaactgccagacgatttcaccggctgtgtgatcgcgtggaactcaaacaacttggatagtaaggtaggtggtaactataactacttgtaccggctcttccgcaaaagtaatcttaaaccgttcgagcgggacatctctaccgagatctatcaagcaggtagcactccctgcaatggagtcgagggttttaattgctattttcccctccaatcatacgggtttcagcccacaaatggtgttgggtatcagccctatcgagttgtggtgctttcatttgaattgcttcatgcaccggcgacagtttgtggtcctaaaaagagtaccaatttggttaaaaacaaatgtgtcaactttaactttaacggtctgactggtacgggggtgcttacggaatctaataaaaagtttcttccgttccagcaattcggacgggacatagcggacacgactgacgcggttagagacccacaaacactcgagattcttgacataaccccctgttctttcggcggagtttctgtaataacgcctgggacaaatacgtcaaaccaggttgcagtattgtaccaggatgtcaactgcactgaagtgccagtcgcgatccacgcagatcagttgactccaacatggagggtctactctaccggttctaacgtgtttcagaccagagccgggtgcctcattggcgcggaacatgtgaacaattcttacgaatgcgacatacccatcggcgccggcatttgtgcgtcataccaaactcaaactaattcccggagtgtggcctcccaatctattattgcttatactatgagtcttggcgcggaaaacagtgtagcgtactcaaataactctattgcgatccccacgaatttcactataagcgtaactactgaaattttgccggtaagcatgacgaaaacttcagtggactgcacaatgtacatttgtggtgacagcactgagtgctcaaatctcctccttcagtatggttctttctgcacacaacttaacagggcacttaccggaatcgcagtagaacaagacaaaaacacgcaagaagtattcgcacaggtcaaacaaatttataaaaccccgccgatcaaggattttggagggtttaacttcagtcagatccttccagacccatccaaacccagcaaacggagctttattgaagacctgctgtttaataaagttacccttgcagatgccggtttcatcaagcaatacggggattgtctgggcgacattgccgcccgcgacttgatctgtgcccagaagttcaacggtctcacagtcttgccgccacttctcactgatgaaatgatagcgcagtatacatccgcgctgctggctgggacgataacatctggttggactttcggggccggggcggctttgcaaattcctttcgccatgcagatggcctataggtttaacggcataggagtgactcaaaatgttctttatgagaatcagaagcttatcgccaaccaatttaactccgcgattgggaagatacaggactcattgagctctactgctagcgcccttggcaagcttcaagacgtagtcaaccaaaatgctcaagcgctcaatactctcgtaaagcagctctcaagcaactttggcgcgatatcttcagttttgaacgacatcttgagcagactcgacaaggtggaggctgaagttcaaattgatcgactgattacgggccggctccagtcactccaaacgtacgtgacccaacaactcattagagcggcagagattagagcgagcgctaatctggcggccacaaagatgtcagaatgtgtgcttggtcaatccaaaagggtggatttctgcggaaagggctatcacttgatgagtttcccgcagtccgcgcctcatggagttgttttcctccacgtaacctacgttcccgctcaggaaaagaatttcacgacagctccggcaatatgtcatgacggtaaagcacatttccccagggaaggcgtttttgtcagtaatggaacccactggttcgtcacccagcgaaacttttacgagccccaaataataactactgacaataccttcgtcagcggcaattgcgacgtcgtcataggcatagtaaataatacggtttacgaccctcttcaacccgagttggacagcttcaaagaagaacttgataagtattttaaaaaccacactagcccagatgtagaccttggcgacatcagcggtatcaatgcttccgttgtgaacattcaaaaggaaattgaccgactcaatgaagtagcgaagaacttgaatgagtctctgatcgatcttcaggagctcgggaaatatgagcaatacatcaaatggccggtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 56 - protein, 1288 aa, Artificial Sequence
  Wuhan Spike S1+S2 ECD_HLA
MAVMAPRTLLLLLSGALALTQTWAVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 57 - DNA, 3867 bp, Artificial Sequence
  sequence encoding Kent Spike S1+S2 ECD_HLA
gccaccatggcggtcatggcaccacgcacgctccttcttttgttgtctggtgcccttgcactcacccaaacgtgggcagttaacctgacaacacgcacccagttgccaccggcctatacgaactctttcacgcgcggagtgtattatcccgataaagtctttcggagttctgtgctgcactctacacaagatctttttctgccctttttctccaacgttacgtggttccatgcaataagtggtacgaatggtactaaaaggttcgataatcccgtacttcccttcaatgacggcgtgtatttcgcgagcacagaaaagagcaacataataagaggttggatttttgggactaccctggactctaaaacacaaagcctcctcattgttaataacgcaactaatgtcgtaataaaggtatgcgaatttcagttttgtaacgacccttttttgggagtctatcataagaacaataagtcctggatggaaagtgaatttagggtgtatagttcagcgaacaactgtacatttgagtatgtgtcacagccgttccttatggatcttgaagggaaacaggggaacttcaagaacctgcgggaattcgtgtttaaaaatatagacggttattttaaaatctatagcaaacacaccccaataaacctggttcgggatcttcctcagggattttcagctttggaaccgttggttgacctccccatcgggataaatatcacgcgatttcaaacactgctggcactgcaccggagttatctgactcctggagatagcagctccggctggacggctggcgccgcggcttattatgtcgggtaccttcaaccgcgaacgtttcttttgaagtataacgaaaatggcactataaccgatgcagtagattgtgcgttggaccctctttccgagactaagtgcaccctgaagtcatttacagtagagaaggggatataccagacaagcaacttccgcgtccaaccaacggaaagcatcgtgcgctttcccaatattactaacctctgtccgttcggcgaggtttttaatgctaccagatttgcctcagtgtatgcctggaaccgcaagaggattagcaactgcgttgctgactatagcgtgctttacaattccgccagttttagtacgttcaagtgttatggggtaagccctaccaagcttaacgatctttgcttcacgaatgtttacgcagactcattcgtaatacgaggtgatgaagtgcgacagatagccccgggtcagacggggaaaatagccgactacaattacaagctcccggacgacttcacgggctgtgttattgcgtggaacagcaataacctggacagtaaagttggagggaattataactacttgtacaggctgttccgcaaatctaatttgaaaccgtttgaaagggacatttccaccgagatttaccaagccggcagtaccccatgtaacggagttgagggatttaattgctactttccgctccaatcatatggattccagcctacatacggcgtcggatatcaaccttatagggtggtcgttctttcttttgagctgcttcacgccccagcaaccgtttgcggcccgaaaaaatcaacgaacttggtcaaaaacaagtgcgtcaacttcaatttcaacggactgacaggcaccggagtgctcaccgagtcaaataaaaaattcctcccgtttcaacaattcggtagagatatagacgacaccacagatgcggtacgcgaccctcagactcttgaaattttggacattacaccatgttccttcggtggcgtctcagtgattacgcctggcactaacactagcaaccaagtcgcagtactttatcagggcgttaactgtacagaagtaccagttgcgatacacgcggaccagctcaccccaacctggcgcgtatattctaccggatctaatgtgtttcaaacgcgggcggggtgcctcataggtgcagagcatgtcaataactcctacgaatgtgacattcctataggagcaggaatatgtgcgagctaccagactcaaaccaattcacacaggagcgtagctagtcagtccataattgcctacactatgtcacttggtgctgaaaacagtgtggcttattcaaataattccatagctattccaatcaatttcactattagcgtaacaactgagatacttccagtcagcatgactaagacttcagttgattgtacgatgtatatttgtggcgattctacagaatgtagtaaccttctgttgcaatacggctccttttgcacacagctcaatcgcgcattgacgggtatagctgtagagcaagataaaaacacccaagaagtcttcgcgcaggtgaagcaaatatataagacaccaccgattaaagatttcggtggattcaattttagccaaattctgcctgatccaagcaaaccgtcaaaaagatccttcatagaggacctgcttttcaataaggtaacgctggcggacgcagggtttattaaacaatacggcgattgccttggtgacattgctgcacgagacctcatttgcgcccagaagttcaacggactcacagtactgcctccactgctcacagacgagatgatcgcccagtacacatctgccttgttggctggtacgataacttctggttggacctttggagcgggagcagcacttcagattccctttgccatgcagatggcgtacagattcaacggcattggtgtcacgcagaacgtactgtatgagaaccagaagttgatagctaatcaattcaactccgcgataggtaagatccaagacagtctctcctcaacagcctccgcactcgggaaactccaggatgtggtaaatcagaatgcacaggcattgaatacgcttgttaaacaattgtcctcaaattttggcgccatatcatcagtgctcaacgacatccttgccagactcgataaagtggaggcagaagtgcagatagatcgcttgattactgggcgcctccaatctctccaaacgtacgtgacacaacagcttattcgggcagcggaaatccgcgcctctgcgaaccttgcagcgaccaagatgagcgaatgtgttctcgggcagtcaaaacgagtagatttctgcggtaaaggatatcatcttatgtcattcccccaaagcgctccacatggtgttgtatttctgcacgtgacttacgtgccggcacaggagaagaatttcacaacggctcccgccatctgtcacgacggaaaggcacacttccctagggagggggtgtttgtaagcaatggtacccactggtttgtcacacaacgaaacttctatgaacctcagataattacaacgcataatacttttgtaagtgggaactgcgacgtcgttatcggaatagttaacaatactgtatatgatccgttgcagccggagttggactcatttaaagaagagctggacaagtacttcaaaaaccatacaagcccagatgtagatcttggggacataagtgggataaacgccagcgttgtgaacatacagaaggaaatagacaggctgaatgaggtagcaaagaatcttaacgaaagccttatagatcttcaggaacttgggaagtacgaacagtacataaagtggcctgtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 58 - protein, 1286 aa, Artificial Sequence
  Kent Spike S1+S2 ECD_HLA
MAVMAPRTLLLLLSGALALTQTWAVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 59 - DNA, 3873 bp, Artificial Sequence
  sequence encoding South Africa Spike S1+S2 ECD_HLA
gccaccatggcggtcatggcaccacgcacgctccttcttttgttgtctggtgcccttgcactcacccaaacgtgggcagtcaatttcactacacgcacacagctgcccccggcgtacactaacagttttacgaggggagtctactacccggacaaagtgttcaggtcttccgtactccatagcacacaagacctcttcttgccttttttcagcaatgttacgtggttccatgcaatccatgtgagcggtacaaacgggactaaacgctttgcaaatccagtgttgcccttcaacgacggtgtatacttcgcgagtacagagaagagcaacataatacgcgggtggatatttggaacgacgcttgattctaagacacagagcttgctcatcgtaaacaacgcaacgaatgttgtaatcaaggtctgcgaattccaattctgcaatgacccatttcttggagtttattaccataagaacaataagtcttggatggaatccgagttccgcgtatactctagcgcgaacaactgcaccttcgagtatgtgtcccaaccgtttctgatggatcttgagggaaagcagggtaattttaaaaatctccgggagttcgttttcaaaaatattgacggatatttcaaaatctacagcaagcataccccaatcaatttggtgcggggtctgccacaaggattttccgctcttgaacctcttgttgaccttcccataggaatcaatataactcggttccaaactcttcttgcgctccatatatcctacttgacgcccggagattcatcaagtggctggacggctggggctgccgcatactacgtcggctatctgcaaccaaggacgtttctcctcaaatacaatgaaaatggtacgatcaccgacgctgtggactgcgctctcgacccgttgtcagagacgaagtgcaccttgaagtccttcaccgtggaaaagggaatttaccagactagtaacttccgggtgcagcccacggaatcaattgtgcgatttcctaatatcactaatctctgtcccttcggggaagtgttcaatgcgactcgattcgcttcagtatacgcttggaatagaaagagaatctcaaattgcgtggctgactattccgtactgtataatagtgcgtcttttagcacgtttaagtgttatggcgtatccccgacaaagctcaacgatctctgcttcacgaatgtctatgccgactccttcgtgatacgcggtgacgaggtacgacaaattgcacccggccaaactggaaatattgccgactacaattataagttgccggacgattttaccggttgcgtaatagcgtggaactctaataacctcgactctaaggtaggaggcaactacaattacttgtaccgactgttcaggaaatccaacctgaagcccttcgaaagagacatcagtacagagatttatcaagccgggtccaccccttgcaatggtgtcaaaggcttcaactgctattttccgttgcagtcttacggtttccagccgacgtacggtgtgggttatcaaccctaccgcgttgtggtgctgagctttgagcttctccacgctccagccactgtctgcgggcctaagaaatccacaaacctcgttaagaataaatgcgtaaatttcaacttcaacgggcttactggcacgggcgtgcttacggagtccaataagaaattcctgcctttccagcagttcggccgcgacatcgcggacacaacagatgctgtgcgagacccacaaaccctggagattctggacatcacgccttgctcttttggcggtgtcagcgtaataacgccaggcaccaacaccagtaatcaggtcgccgtgctttatcagggtgtgaactgtacggaggtaccggttgccatacacgcagaccaactcacacctacgtggagagtttacagcaccgggtcaaacgtatttcagacccgcgcaggctgtctgatcggcgcagagcatgtcaataactcatacgagtgtgatataccaatcggagccggcatctgtgcgagctatcaaacccaaactaattcacgaagcgtggccagccagtccatcattgcctacacgatgtctttgggagtagagaatagcgtggcatactctaacaacagtatcgctattcctaccaacttcacaatatcagttactacagaaatcctcccggtgagcatgactaagacatccgtagactgtacaatgtacatttgtggggacagcacggagtgttcaaacctgctgttgcaatacggaagcttctgcacccagctcaaccgagccctgaccggcattgccgtagaacaagataagaatacgcaggaagtgttcgcccaggttaagcagatttacaagactccgcctatcaaggacttcgggggtttcaactttagtcagattttgcctgacccgtcaaagcctagcaaacgaagttttatcgaagatttgttgtttaacaaggtgactttggctgatgcgggattcataaaacaatacggcgattgccttggcgatattgcggcccgagatctcatttgcgcgcaaaagttcaacggtcttaccgtccttccgcccctgcttacggacgagatgatcgcacaatacacttcagcactgctggctggtacaataaccagtgggtggacattcggggctggtgcagcgctccagatcccctttgcgatgcagatggcgtatagattcaatggcatcggggtcactcagaacgtgctttatgagaaccaaaagcttattgcaaatcaatttaactctgcgattggaaagattcaagacagcctctcatccacagctagtgcgcttggcaagcttcaggacgtcgtaaaccaaaacgcccaagctctcaacacactcgttaaacagctttcctcaaactttggggctattagctctgtgctgaatgatattcttagcagactcgacaaagtcgaggctgaagtacaaatagatagactgataacaggtagacttcagtctctccaaacctacgtgacacagcagcttataagagctgctgagatcagagcttctgcgaaccttgcggcgactaaaatgtccgagtgtgtactcgggcagtctaaacgcgtcgacttctgtggcaagggttaccatctcatgagttttccccaatcagcgccccatggggtagtttttctgcacgtaacatacgtcccagcccaagaaaagaattttaccaccgcccctgcgatttgtcacgacggaaaagcacactttccaagagaaggcgtatttgttagcaatggcacccattggttcgtcacccagagaaacttttacgaaccgcagattatcacgacagataatacatttgtatctggtaattgtgacgtcgtgatcggcatcgtaaacaacactgtttacgaccccctccagccagagcttgactcatttaaagaggagctcgacaaatattttaagaaccacacaagtccagacgtcgacttgggtgacatttccggcatcaatgcaagtgtggtcaacatccaaaaggaaattgacagattgaacgaggttgctaagaaccttaatgagtcactcattgacctccaggaactgggaaaatacgaacagtatattaagtggcccgtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 60 - protein, 1288 aa, Artificial Sequence
  South Africa Spike S1+S2 ECD_HLA
MAVMAPRTLLLLLSGALALTQTWAVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 61 - DNA, 3870 bp, Artificial Sequence
  sequence encoding California Spike S1+S2 ECD_HLA
gccaccatggcggtcatggcaccacgcacgctccttcttttgttgtctggtgcccttgcactcacccaaacgtgggcagtaaatttgacaacgcgcacccaattgcccccagcatatacgaactctttcacgaggggcgtatattatccggataaggtatttcggtcatctgttctgcacagcacccaggacctcttccttccattcttttcaaatgtaacttggttccatgcgatagtatcaggaacgaatgggacaaaaaggttcgataatccggtcttgccgttcaacgatggggtgtacttcgccagtaccgaaaagtctaacattatacgcggttggatttttggcactacgcttgactcaaagacacagtcactcctcattgtaaataatgctactaatgtcgtgatcaaagtttgtgagttccaattctgcaacgacccgttcctgggagtgtattatcacaaaaataataagtcctgcatggagtcagagttcagggtttactcaagtgcgaacaattgcacatttgagtacgtttctcaaccatttctcatggatttggaggggaagcagggaaactttaaaaacctgagagaatttgtttttaagaacattgatgggtatttcaagatttatagtaaacacacccctatcaacttggttagggatctccctcaaggtttctctgctctcgagccccttgtagatctgccaataggcatcaatatcacacgctttcagacactcctcgcacttcataggagctacctgacgccaggtgactcttcctcaggttggacagccggcgcagccgcatactacgttggctacctccaaccaaggacatttctgttgaaatacaacgaaaatgggaccatcaccgacgcagtcgattgtgctctcgaccctctttccgagactaaatgtaccctcaaaagctttactgttgagaagggtatctatcagacatctaactttcgggtgcaacccactgagtcaattgtgcgattcccaaatattacgaacctctgtccttttggcgaggtttttaacgccactaggttcgccagtgtatatgcttggaaccgaaaacggataagcaattgtgttgctgactactccgtcctctacaatagcgctagtttctcaacatttaagtgttacggtgtgagccctacgaaacttaacgatttgtgcttcactaacgtctatgccgacagtttcgtaatccgaggcgatgaggtcaggcaaattgccccgggccaaacggggaaaatcgctgattacaattataagttgccagatgattttacgggatgtgtcattgcatggaacagtaataacctcgattcaaaggttggcggaaattataattacagatatcggctttttagaaaatctaaccttaaaccatttgagcgggacataagcacggagatttaccaggctggtagcactccgtgcaacggtgtagaaggattcaattgctattttccattgcagtcttatggattccaacccaccaatggggtagggtaccaaccatacagggtggtagtccttagctttgaacttttgcatgcgccagctaccgtctgcggtcccaaaaagagtacgaacttggtaaaaaataaatgcgtcaactttaattttaacggtctgacgggaacgggggttctcaccgagtctaacaaaaagtttttgccatttcagcagttcggacgagatattgccgacactaccgacgccgtgcgcgatccacaaaccttggagatactcgacatcacaccttgcagcttcggtggtgttagcgttattacgccaggaacgaacacttcaaatcaggtggccgtcttgtatcaagatgttaactgtacagaggtgcccgtcgcaatacacgcagatcagctcacccctacctggagagtttactctacaggctctaatgtgttccaaactagggcaggctgcttgattggagctgaacacgtaaacaactcatacgagtgcgatatcccgattggtgcgggtatttgcgccagctatcagacgcagactaactcacgatcagtggcgtcacagtcaataattgcgtatacaatgagtctcggtgcagagaattccgtcgcatactcaaacaacagcatagcgattccaactaattttaccattagcgttacaacggaaatcctccccgttagcatgaccaagacctccgtggattgcacaatgtacatctgtggagacagcaccgagtgctccaatcttcttcttcaatatgggagcttttgcacacagctgaaccgcgcgctcactggcattgcggtagaacaagacaaaaacacccaagaagtgtttgcgcaggtcaaacagatttacaagacaccaccaatcaaagacttcggggggttcaatttctcccaaattcttcctgatccaagcaaaccatctaaaaggtccttcatagaagacctcctgttcaataaggtgacactcgcggacgcagggtttattaagcaatatggagattgtctcggggatattgcagcgagagatcttatttgtgctcaaaagtttaatggcttgactgtcctcccgcccttgctcacagacgagatgatagcacaatataccagtgcgctgcttgcgggcacgattacttccggatggactttcggggcaggagcggcgctccagattcctttcgcgatgcaaatggcctaccgatttaacgggattggcgtcacacaaaacgttctttacgaaaaccagaaacttattgccaaccaattcaatagtgcaattggtaaaattcaggattcactctcctccacggcgtcagctctcggcaagctccaagacgtggttaaccaaaatgcgcaggcgcttaacacgcttgtaaaacaactctcttctaattttggcgcgattagtagcgttctcaacgatatcttgtcacgcttggacaaagtagaagcagaagtccaaatagaccggctgatcactggtcgattgcaatctctgcagacatacgtgacgcagcagctcatccgggcagccgagatccgggcgtccgctaatctggccgcaaccaaaatgagcgagtgtgtccttgggcaaagcaaacgcgtggatttttgcggaaagggctaccacctcatgagtttcccccaatctgccccgcacggtgtcgtctttttgcatgtaacgtatgtcccggcgcaggagaaaaatttcactacagctcccgccatctgtcacgacggcaaggcacacttccctcgagaaggggtgtttgtaagcaatggaacacactggttcgtcacccagcggaatttttatgaacctcagataatcacaacagacaacactttcgtttccggaaactgcgatgtcgtaattgggatagtgaataataccgtatatgacccattgcagccggagttggactcctttaaagaggaattggacaaatactttaaaaatcacacttcacccgatgtggacctgggagatatatccgggattaacgcaagtgtggttaacatacaaaaggaaattgaccgccttaacgaggttgctaagaacttgaatgagtctctcatagatctccaggaactggggaaatacgagcaatatatcaaatggccggtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 62 - protein, 1287 aa, Artificial Sequence
  California Spike S1+S2 ECD_HLA
MAVMAPRTLLLLLSGALALTQTWAVNLTTRTQLPPAYTNSFTRGVYYPDKVFRSSVLHSTQDLFLPFFSNVTWFHAIVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 63 - DNA, 3873 bp, Artificial Sequence
  sequence encoding Brazil Spike S1+S2 ECD_HLA
gccaccatggcggtcatggcaccacgcacgctccttcttttgttgtctggtgcccttgcactcacccaaacgtgggcagttaactttactaatcggacccaactcccgagtgcgtacactaactcttttacccggggcgtctattatcctgacaaagtgtttcgatcatctgttttgcactccacccaagatctctttcttccatttttcagcaacgttacgtggttccacgcgatacatgttagtggtactaatggcacaaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaggagctatctgaccccgggcgactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaacattgctgattataactacaagttgcccgatgacttcacggggtgtgttattgcatggaattcaaataatcttgatagtaaggtagggggtaactataattacctttatcgcttgttccgcaaatccaatctgaaacccttcgaaagggatatcagtaccgagatttatcaagccggctccacaccttgtaacggagtaaaaggttttaactgttatttcccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagcagaagcgttgcgtcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgacggttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctattaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttggagacatctctgggataaacgctagcttcgtaaacatacaaaaagaaatcgataggcttaacgaggtcgcaaaaaatcttaatgaaagtcttatcgatttgcaagagcttggaaagtatgagcagtatatcaaatggcctgtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 64 - protein, 1288 aa, Artificial Sequence
  Brazil Spike S1+S2 ECD_HLA
MAVMAPRTLLLLLSGALALTQTWAVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 65 - DNA, 3870 bp, Artificial Sequence
  sequence encoding Delta Spike S1+S2 ECD_HLA
gccaccatggcggtcatggcaccacgcacgctccttcttttgttgtctggtgcccttgcactcacccaaacgtgggcagttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccagtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaataa

SEQ ID NO: 66 - protein, 1287 aa, Artificial Sequence
  Delta Spike S1+S2 ECD_HLA
MAVMAPRTLLLLLSGALALTQTWAVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYRYRLFRKSNLKPFERDISTEIYQAGSTPCNGVQGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

SEQ ID NO: 67 - protein, 46 aa, Artificial Sequence
  CD74 cytoplasmic domain
MHRRRSRSCREDQNPVMDDQRDLISNNEQLPMLGRRPGAPESKCSR

SEQ ID NO: 68 - protein, 24 aa, Artificial Sequence
  HLA signal sequence
MAVMAPRTLLLLLSGALALTQTWA

SEQ ID NO: 69 - DNA, 72 bp, Artificial Sequence
  sequence encoding HLA signal sequence
atggcggtcatggcaccacgcacgctccttcttttgttgtctggtgcccttgcactcacccaaacgtgggca

SEQ ID NO: 70 - protein, 24 aa, Artificial Sequence
  HLA transmembrane domain
WMVAAVVAGTIVAGLLVLGAIIGV

SEQ ID NO: 71 - DNA, 72 bp, Artificial Sequence
  sequence encoding HLA transmembrane domain
tggatggtggctgcggtcgtggcaggtacgattgtggctggcctcctcgttcttggggctattatcggtgta

SEQ ID NO: 72 - protein, 57 aa, Artificial Sequence
  HLA cytoplasmic domain
VGIIAGLVLLGAVITGAVVAAVMWRRKSSDRKGGSYTQAASSDSAQGSDVSLTACKV

SEQ ID NO: 73 - DNA, 171 bp, Artificial Sequence
  sequence encoding HLA cytoplasmic domain
gtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtt

SEQ ID NO: 74 - protein, 70 aa, Artificial Sequence
  targeting moiety
MHRRRSRSCREDQNPVMDDQRDLISNNEQLPMLGRRPGAPESKCSRWMVAAVVAGTIVAGLLVLGAIIGV

SEQ ID NO: 75 - DNA, 135 bp, Artificial Sequence
  CD74 cytoplasmic domain
caccggaggagatccagaagttgcagagaagaccaaaatccagtaatggatgaccaaagggatcttatttctaataacgagcagctccctatgctgggacgccgaccgggggcacccgaatcaaagtgcagtcgc

SEQ ID NO: 76 - protein, 1271 aa, Artificial Sequence
  spike protein
MFVFLVLLPLVSSQCVNFTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVKGFNCYSPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLADISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTSRLEEELRRRLTE

SEQ ID NO: 77 - protein, 1197 aa, Artificial Sequence
  spike protein extracellular domain
MVNFTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVKGFNCYSPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLADISGINASVVNIQKEIDRLNEVAKNLNESLIDLQELGKYEQYIKWPSRLEEELRRRLTE

SEQ ID NO: 78 - DNA, 3822 bp, Artificial Sequence
  sequence encoding a spike protein
gccaccatgtttgtgttcctggtccttctcccgcttgtaagctcccaatgcgttaactttactactcggacccaactcccgccggcgtacactaactcttttacccggggcgtctattatcctgacaaagtgtttcgatcatctgttttgcactccacccaagatctctttcttccatttttcagcaacgttacgtggttccacgcgatacatgttagtggtactaatgttataaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaaaattgctgattataactacaagttgcccgatgacttcacggggtgtgttattgcatggaattcaaataatcttgatagtaaggtagggggtaactataattaccaatatcgcttgttccgcaaatccaatctgaaacccttcgaaagggatatcagtaccgagatttatcaagccggctccacaccttgtaacggagtaaaaggttttaactgttattccccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagctcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgaatgttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctacaaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttgctgacatctctgggataaacgctagcgtagtaaacatacaaaaagaaatcgataggcttaacgaggtcgcaaaaaatcttaatgaaagtcttatcgatttgcaagagcttggaaagtatgagcagtatatcaaatggccttggtatatatggctgggatttattgctggtctcatagccatagttatggtgacaataatgctgtgctgtatgacgtcatgttgttcctgcctgaaagggtgctgtagttgtgggagttgttgcaagtttgacgaggacgactcagagcccgttttgaaaggtgtgaaacttcattatacatcacgactggaggaagaactgcgccgacgcctgactgaatga

SEQ ID NO: 79 - DNA, 3600 bp, Artificial Sequence
  sequence encoding a spike protein extracellular domain
gccaccatggttaactttactactcggacccaactcccgccggcgtacactaactcttttacccggggcgtctattatcctgacaaagtgtttcgatcatctgttttgcactccacccaagatctctttcttccatttttcagcaacgttacgtggttccacgcgatacatgttagtggtactaatgttataaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaaaattgctgattataactacaagttgcccgatgacttcacggggtgtgttattgcatggaattcaaataatcttgatagtaaggtagggggtaactataattaccaatatcgcttgttccgcaaatccaatctgaaacccttcgaaagggatatcagtaccgagatttatcaagccggctccacaccttgtaacggagtaaaaggttttaactgttattccccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagctcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgaatgttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctacaaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttgctgacatctctgggataaacgctagcgtagtaaacatacaaaaagaaatcgataggcttaacgaggtcgcaaaaaatcttaatgaaagtcttatcgatttgcaagagcttggaaagtatgagcagtatatcaaatggccttcacgactggaggaagaactgcgccgacgcctgactgaatga

SEQ ID NO: 80 - protein, 24 aa, Artificial Sequence
  HLA cytoplasmic domain
RRKSSDRKGGSYTQAASSDSAQGS

SEQ ID NO: 81 - protein, 17 aa, Artificial Sequence
  fragment of HLA cytoplasmic domain
RRKSSDRKGGSYTQAAV

SEQ ID NO: 82 - DNA, 3807 bp, Artificial Sequence
  sequence encoding a Lambda Spike S1+S2ECD_CD74CD+HLATM
gccaccatgcaccggaggagatccagaagttgcagagaagaccaaaatccagtaatggatgaccaaagggatcttatttctaataacgagcagctccctatgctgggacgccgaccgggggcacccgaatcaaagtgcagtcgctggatggtggctgcggtcgtggcaggtacgattgtggctggcctcctcgttcttggggctattatcggtgtagttaactttactactcggacccaactcccgccggcgtacactaactcttttacccggggcgtctattatcctgacaaagtgtttcgatcatctgttttgcactccacccaagatctctttcttccatttttcagcaacgttacgtggttccacgcgatacatgttagtggtactaatgttataaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaaaattgctgattataactacaagttgcccgatgacttcacggggtgtgttattgcatggaattcaaataatcttgatagtaaggtagggggtaactataattaccaatatcgcttgttccgcaaatccaatctgaaacccttcgaaagggatatcagtaccgagatttatcaagccggctccacaccttgtaacggagtaaaaggttttaactgttattccccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagctcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgaatgttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctacaaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttgctgacatctctgggataaacgctagcgtagtaaacatacaaaaagaaatcgataggcttaacgaggtcgcaaaaaatcttaatgaaagtcttatcgatttgcaagagcttggaaagtatgagcagtatatcaaatggccttcacgactggaggaagaactgcgccgacgcctgactgaatga

SEQ ID NO: 83 - protein, 1266 aa, Artificial Sequence
  encodes Lambda Spike S1+S2ECD_CD74CD+HLATM
MHRRRSRSCREDQNPVMDDQRDLISNNEQLPMLGRRPGAPESKCSRWMVAAVVAGTIVAGLLVLGAIIGVVNFTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVKGFNCYSPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLADISGINASVVNIQKEIDRLNEVAKNLNESLIDLQELGKYEQYIKWPSRLEEELRRRLTE

SEQ ID NO: 84 - DNA, 3840 bp, Artificial Sequence
  sequence encoding a Lambda Spike S1+S2ECD_HLA SS/TM/CD
gccaccatggcggtcatggcaccacgcacgctccttcttttgttgtctggtgcccttgcactcacccaaacgtgggcagttaactttactactcggacccaactcccgccggcgtacactaactcttttacccggggcgtctattatcctgacaaagtgtttcgatcatctgttttgcactccacccaagatctctttcttccatttttcagcaacgttacgtggttccacgcgatacatgttagtggtactaatgttataaagagatttgacaaccctgtgctcccgtttaacgatggggtctatttcgcttccactgagaagtccaacattattcgcggttggatcttcggcaccacactcgattcaaaaacccaaagcttgctcatagttaacaacgccacaaacgtagtcattaaagtgtgcgaatttcagttttgtaactatccctttttgggcgtctactatcacaagaacaacaagtcctggatggaatctgagtttcgggtctacagttcagccaacaactgtacgtttgagtacgttagccagccctttctgatggatcttgaagggaagcaaggaaatttcaagaatctttccgagtttgtgtttaaaaatatagacggatatttcaagatatacagtaagcacacaccgatcaatttggtacgggatttgccgcaaggattttccgcccttgagccccttgtagatctccccatcggaattaacataaccaggtttcaaacactgctggcgctccacaactcatccagtgggtggaccgccggagcagccgcatactacgtggggtatctccagcccagaacgtttctcctcaagtataatgaaaacgggaccatcaccgacgcggttgattgtgcactcgatcccctcagcgagacaaagtgcacacttaaatcatttaccgtcgagaaggggatctaccaaacatccaactttcgggtgcagcctaccgagtcaatagtacggtttcctaacatcaccaatttgtgcccttttggggaagtatttaatgcaacccggtttgcgagcgtatacgcttggaaccgaaaacggataagtaattgtgttgcagattattctgtgctctataactcagcttccttctccacctttaagtgttacggagtatctccgacaaaattgaatgatctttgttttacgaatgtttacgctgactcatttgttatccgaggtgacgaagtgcgccagatagcccccggtcaaacaggcaaaattgctgattataactacaagttgcccgatgacttcacggggtgtgttattgcatggaattcaaataatcttgatagtaaggtagggggtaactataattaccaatatcgcttgttccgcaaatccaatctgaaacccttcgaaagggatatcagtaccgagatttatcaagccggctccacaccttgtaacggagtaaaaggttttaactgttattccccactgcagagttacggctttcaacccacatacggggtgggatatcagccctatcgcgttgtggtcttgtcctttgaacttctccacgcccctgcaacagtttgtggtcctaaaaagagtacaaatctggtaaaaaataagtgcgtcaactttaacttcaacggactgactggtaccggggtcctcactgagagcaacaagaaatttttgcccttccagcaattcgggagggatattgccgatactaccgatgctgtacgggacccccagacactcgaaatattggacataaccccgtgctcttttggaggggtctctgtgattaccccgggtacgaacacgagtaatcaggtggcagtgctttatcagggagtgaattgcacagaagtaccagtcgcgatccatgccgaccagctcacgccgacctggagggtttatagtacgggtagtaacgttttccaaacacgggccggttgtctcatcggggcagagtacgttaataacagctatgaatgtgatatacccataggtgccgggatttgtgcttcttatcaaacccaaaccaatagctcacagtctataatcgcctatacgatgagcctcggagcggaaaactcagttgcctactccaacaatagcattgctatcccaacgaacttcactattagcgtaaccacggagatccttccagttagcatgacaaaaactagcgtcgattgcactatgtatatttgcggcgacagtacagagtgtagtaatttgctcctgcagtacgggtctttctgcacccaattgaaccgagcactgactggcatcgccgtggaacaagacaaaaacacgcaagaagtatttgcgcaagtcaagcagatttacaagactcccccgatcaaagactttggcggatttaattttagccagatactccccgatccatctaaaccatcaaaacggtcctttatagaagatctcttgttcaataaagtgacactcgcggacgcgggctttatcaaacagtatggggactgccttggagatatagctgcgagggacttgatttgcgcccagaaattcaacgggctgaatgttctgccgccgctcttgacagacgaaatgattgcacagtatacgagtgccttgctggcagggactatcacttccggttggaccttcggcgcaggagcagcgctccagatcccattcgctatgcaaatggcttaccgattcaacggaattggcgtcacacagaacgtgctttacgagaatcaaaagctgattgccaatcaattcaactcagcgattggcaaaatacaagatagtctctcttcaactgcatccgcgctcggtaagttgcaggacgtagtcaatcagaacgcacaggccctgaacacgcttgttaaacaactgtcttcaaacttcggtgcaataagttccgtacttaatgatatcttgtcaaggttggataaggtcgaagctgaggtacaaatcgaccgccttataacaggcagactccaatccctgcagacatacgtgacccagcaactcattagggcagccgagatcagagcaagtgccaacctcgctgctacaaaaatgtctgaatgcgttttggggcaatctaagcgggttgatttctgcggtaagggataccacttgatgtcctttcctcagtccgctcctcatggggtagtatttcttcatgttacatatgtaccagcacaagagaaaaattttactactgccccggccatatgccatgatggcaaagcgcacttccctcgggagggggtttttgtgtctaatggaacccactggttcgtgactcagcggaatttctatgaaccccagattataactactgacaacactttcgtttctggaaactgcgatgtagtaataggcatagtaaataatacagtatatgatcccttgcaaccagaattggactcttttaaagaagagctcgacaaatattttaaaaaccatacatctccagacgttgatcttgctgacatctctgggataaacgctagcgtagtaaacatacaaaaagaaatcgataggcttaacgaggtcgcaaaaaatcttaatgaaagtcttatcgatttgcaagagcttggaaagtatgagcagtatatcaaatggcctgtaggtattatagccgggctcgtgttgttgggtgcagtaatcacaggagccgtcgtggcagcggttatgtggagacggaaatcttcagaccgcaaaggtggtagttacactcaagcggcttccagtgattctgcccagggctccgacgtatccctcactgcgtgtaaggtttcacgactggaggaagaactgcgccgacgcctgactgaatga

SEQ ID NO: 85 - protein, 1277 aa, Artificial Sequence
  Lambda Spike S1+S2ECD_HLA SS/TM/CD
MAVMAPRTLLLLLSGALALTQTWAVNFTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVKGFNCYSPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLADISGINASVVNIQKEIDRLNEVAKNLNESLIDLQELGKYEQYIKWPVGIIAGLVLLGAVITGAVVAAVMWRRKSSDRKGGSYTQAASSDSAQGSDVSLTACKVSRLEEELRRRLTE

(19) United States
(12) Patent Application Publication
(10) Pub. No.: US 2023/0310585 A1
(43) Pub. Date: Oct. 5, 2023

(54) VACCINE FOR VIRAL PATHOGENS

(71) Applicant: THE UNIVERSITY OF BRITISH COLUMBIA, Vancouver, Bristish Columbia (CA)

(72) Inventor: Wilfred JEFFERIES, Vancouver (CA)

(73) Assignee: THE UNIVERSITY OF BRITISH COLUMBIA, Vancouver, Bristish Columbia (CA)

(21) Appl. No.: 18/024,868

(22) PCT Filed: Sep. 13, 2021
(86) PCT No.: PCT/CA2021/051265

§ 371 Date: Mar. 6, 2023

Related U.S. Application Data
(60) Provisional application No. 63/077,107, filed on Sep. 11, 2020.

(51) Int. Cl. A61K 39/215; A61K 38/17; A61P 31/14

(52) U.S. Cl. CPC A61K 39/215; A61K 38/1774; A61P 31/14; A61K 2039/53

(57) ABSTRACT

The present invention provides vaccines against respiratory viruses including coronavirus, such as SARS-CoV-2, and influenza viruses. In particular, the present invention provides vaccines against SARS-CoV-2 which encode a targeting domain and a SARS-CoV-2 spike protein or fragment thereof.

### FIELD OF THE INVENTION

This invention pertains generally to vaccines and, more particularly vaccines for viral pathogens including influenza and coronavirus, including but not limited to SARS-CoV-2, the causative agent of COVID-19.

### BACKGROUND OF THE INVENTION

SARS-CoV-2, has spread across the world and become a global pandemic with more than 225 million confirmed cases and 4.6 million deaths worldwide.

One of the key antigen targets of vaccines which have been developed so far is the spike (S) protein (Le et al. Nature Reviews Drug Discovery 19, 305-306 (2020)). Enhancement of antigen presentation has been found to enhance vaccine performance. In particular, it has been found that specific CD4 and CD8 T-cell memory responses (a parameter of vaccine performance) by 50-fold when Influenza M1 protein is linked to CD74 transmembrane and cytoplasmic domain that delivers the recombinant protein to the endolysosome for antigen processing and loading on MHC I and II molecules. The data shows enhanced resistance to lethal Influenza A viral challenge using constructs containing the CD74 targeting signal.

A variety of platforms, including but not limited to nucleic acid, viral vectors, attenuated viruses and recombinant protein, are being examined for the development of the SARS-COV-2 vaccine. The use of nucleic acid-based vaccines allow for vaccines to be obtained in a short timeframe Furthermore, nucleic acid-based vaccine manufacturing is safe and time-saving, and bypasses the need to grow highly pathogenic organisms at a large scale, resulting in a lower risk of contamination with live infectious reagents and release of dangerous pathogens.

The Self-Amplifying mRNA (SAM) vaccine platform is composed of a non-viral, engineered replicon that drive high levels of expression of encoding antigens. Very low doses are required (mgs) as tens of thousands of copies are made by transfected cells. They may be delivered via intramuscular (i.m.), in the same manner as earlier mRNA vaccines, and can be encapsulated within a lipid nanoparticle to further boost performance. This manufacturing process makes GMP grade SAM a promising vaccine approach for filling the gap between emerging infectious disease and the desperate need for effective vaccines. SAMs are an innovative platform for vaccine development. Within an alphavirus backbone, the mRNA replicates through a double stranded RNA intermediate, and the antigen of interest replaces structural proteins, so no infectious virus is made. They may be delivered via intramuscular (i.m.), in the same manner as earlier mRNA vaccines, and may be delivered as naked RNA or encapsulated within a lipid nanoparticle. Comparatively, mRNA vaccines confer several advantages over vaccines introduced by virus vectors and DNA vaccines: the production procedure to generate mRNA vaccines is cell-free, simple and rapid if compared to production of whole microbe, or live attenuated or subunit vaccines.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a vaccine for viral pathogens. In one aspect of the present invention, there is provided a vaccine comprising or encoding a first fusion protein comprising a first targeting domain and a coronavirus spike protein or fragment thereof. In certain embodiments, coronavirus is a SARs-CoV virus, optionally a SARs-CoV2 virus. In certain embodiments, the first targeting domain is a lysosomal targeting domain. In certain embodiments, the first targeting domain comprises a HLA signal sequence and a HLA transmembrane domain. In certain embodiments, the first targeting domain comprises a HLA signal sequence and a HLA transmembrane domain and a HLA Cytoplasmic domain. In certain embodiments, the first targeting domain comprises CD74 Cytoplasmic domain and a HLA transmembrane domain. In certain embodiments, the first targeting domain comprises a HLA signal sequence. In certain embodiments, the vaccine further comprises or encodes a second fusion protein comprising a second targeting domain and an influenza immunogen. In certain embodiments, the first targeting domain and the second targeting domain are the same or are different.

In another aspect of the present invention, there is provided a vaccine encoding one or more polypeptides comprising the sequence as set forth in any one of SEQ ID NOs: 44, 46, 48, 50, 52, 54, 56, 58, 60, 62, 64 and 66 or comprising one or more sequences as set forth in any one of SEQ ID NOs: 43; 45, 47, 49, 51, 53, 55, 57, 59, 61, 63 and 65. In certain embodiments, the vaccine is a viral expression vector-based vaccine, such as an adenoviral vector, a vesicular stomatitis virus vector or a vaccinia vector. In certain embodiments, the vaccine is a nucleic acid-based vaccine. In certain embodiments, the vaccine is a SAM RNA-based vaccine; optionally the SAM RNA-based vaccine is encapsulated in a lipid nanoparticle (LNP). Optionally, the LNP comprises a cationic lipid; optionally the LNP comprises phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA).

In certain embodiments the vaccine further comprises an adjuvant.

In another aspect of the present invention, there is provided a method of treating, protecting against, and/or preventing COVID-19 in a subject in need thereof, said method comprising administering the vaccine of the invention to the subject.

In another aspect of the present invention, there is provided a method of generating an immune response against one or more strains of SARS-CoV-2, said method comprising administering one or more of the vaccines of the invention to the subject. In certain embodiments, the vaccine is administered more than once. The subject may be a mammal including a human, from non-human primates, cats, dogs, equines, sheep, goats; bovine, pangolins and marsupials; reptile, amphibian or bird.

In another aspect of the present invention, there is provided a method of treating, protecting against, and/or preventing COVID-19 and influenza in a subject in need thereof, said method comprising administering one or more of vaccines of targeting SARS-CoV2 and influenza to the subject.

In another aspect of the present invention, there is provided a method of generating an immune response against SARS-CoV-2 and influenza virus, said method comprising administering one or more of vaccines of targeting SARS-CoV2 and influenza to the subject.

In another aspect of the present invention, there is provided a vaccine comprising or encoding a targeting domain and an influenza immunogen. Optionally, the targeting domain comprises CD74 Cytoplasmic domain and a HLA transmembrane domain. Optionally the influenza immunogen is selected from the group consisting of one or more of M, N, HA, fragments thereof, variants thereof and combinations thereof.

In another aspect of the present invention, there is provided a method of treating, protecting against, and/or preventing influenza in a subject in need thereof, said method comprising administering the vaccine targeting influenza to the subject.

In another aspect of the present invention, there is provided a method of generating an immune response against influenza said method comprising administering the vaccine of the invention to the subject.

### BRIEF DESCRIPTION OF THE DRAWINGS

These and other features of the invention will become more apparent in the following detailed description in which reference is made to the appended drawings.

FIG. 1 illustrates that no differences in body weight change were seen in mice inoculated with different vaccine formulations. Weights on Days 0 to 7 were compared to the initial weight taken on Day −1 (pre-treatment). There did not appear to be any significant weight loss due to vaccine toxicity. However, there was some weight loss seen due to the stress of the handling and manipulation of the mice (averaged <5% in most cases) and the mice recovered over time. Mice were weighed every day immediately prior to immunization and blood collection, and on the days immediately following as part of the health monitoring; euthanasia end point was at 20% weight loss. Mice were inoculated intramuscularly with 25 ul of the vaccine on Day 0. Blood (50 μl) from the saphenous leg vein were taken on Day −1 (pre-treatment) and Day 7 (post-treatment). Four mice were used per vaccine group (n=4). Note: mouse #710604 was removed from the study due to weight loss that was not related to vaccine-induced toxicity.

FIG. 2 illustrates no differences in body weight change were seen according to sex in mice inoculated with different vaccine Formulations. Mice were inoculated intramuscularly (IM) with 25 μl of the vaccine on Day 0; saphenous bleeds (50 μl) were taken on Day −1 and Day 7. Note: mouse #710604 was removed from the study due to weight loss that was not related to vaccine-induced toxicity.

FIG. 3 illustrates no differences in body weight change were seen according to age in mice inoculated with different vaccine formulations. Mice were inoculated intramuscularly (IM) with 25 μl of the vaccine on Day 0; saphenous bleeds (50 ul) were taken on Day −1 and Day 7. Note: mouse #710604 was removed from the study due to weight loss that was not related to vaccine-induced toxicity.

FIG. 4 illustrates immune biomarkers 7 days post vaccination.

FIG. 5 illustrates that a vaccine containing CD74 peptide promotes augmentation of immune response to a sample viral antigen.

### DETAILED DESCRIPTION OF THE INVENTION

The present invention provides vaccines. In particular, vaccines against one or more viral pathogens including respiratory viruses are provided. Such respiratory viruses include but are not limited to influenza viruses and coronaviruses. In certain embodiments, the present invention provides vaccines against coronaviruses, including but not limited to SARS-associated coronaviruses (SARS-CoV). In specific embodiments, the invention provides vaccines against SARS-CoV-2. Various strains of SARS-CoV-2 have now been identified. Accordingly, in certain embodiments there is provided vaccines against one or more coronaviruses, including one or more strains of SARS-CoV-2.

In certain embodiments, the present invention provides vaccines against influenza viruses, including but not limited to influenza A virus, influenza B virus, influenza C virus and influenza D virus. In certain embodiments, there is provided vaccines against one or more influenza viruses.

In certain embodiments, the present invention provides a vaccine against coronaviruses, including but not limited to SARS-CoV and influenza. In specific embodiments, the present invention provides a vaccine against SARS-CoV-2 and influenza.

Also provided are pharmaceutical compositions comprising the vaccines and methods of generating a protective immune response against the one or more viral pathogens.

The vaccines of the present invention comprise one or more viral immunogens alone or in combination with one or more targeting molecules and/or one/or more immune stimulating molecules or nucleic acids comprising sequences that encode one or more viral immunogens alone or in combination with one or more targeting molecules and/or one or more immune stimulating molecules.

Vaccine Platforms

A variety of platforms may be used to generate the vaccines of the present invention. Exemplary vaccine platforms which may be used include but are not limited to protein-based platforms, virus-like particle-based vaccines, viral vector-based platforms and nucleic acid-based vaccine platforms.

In certain embodiments, the vaccine platform is a viral vector-based platform. The viral vectors may be attenuated viruses, may be replicating or non-replicating. Exemplary viral vectors include not are not limited to adenovirus, vaccinia or adeno associated virus, lentivirus or vesicular stomatitis virus (VSV). Accordingly, in certain embodiments the vaccine platform is an adenovirus, vaccinia or adeno associated virus, lentivirus or Vesicular stomatitis virus based vaccine.

In specific embodiments, the viral vector platform is an adenovirus vector platform. Various serotypes of adenoviruses have been used in vaccine development including Ad5, Ad26 and Ad35. In certain embodiments, the adenovirus vector is based on a simian adenovirus. Use of simian adenovirus vaccine vectors circumvent pre-existing human adenovirus immunity. Exemplary, simian adenovirus serotypes used in vaccine development include simian adenovirus type 23.

In certain embodiments, the vaccine platform is a nucleic acid-based platform. Nucleic acid-based vaccine platforms may be DNA or RNA-based. Optionally, the nucleic acids include one or more modified nucleosides.

In certain embodiments, the nucleic acid-based vaccine platform is a DNA-based vaccine platform. Appropriate DNA expression vectors for use as a DNA-based vaccine platform are known in the art. A worker skilled in the art would readily appreciate that such expression vectors include the necessary elements to allow for expression of the one or more immunogens. Such elements include a promoter, such as the CMV promoter which directs transcription of the mRNA encoded by the transgene, a polyadenylation signal which mediates mRNA cleavage and polyadenylation, and Kozak sequence which directs efficient transgene translation. In specific embodiments, the DNA-based vaccine is a plasmid-based vaccine.

In certain embodiments, the nucleic acid-based vaccine is a RNA-based vaccine platform. In specific embodiments, a mRNA platform. The mRNA-based vaccine platform may be non-replicating or self-amplifying. In certain embodiments, the nucleic acid-base vaccine platform is a self-amplifying (SAM) RNA platform. A variety of RNA based expression systems are known in the art and commercially available, including but not limited to expression systems based on either positive-sense and negative-sense RNA viruses. Positive-strand RNA viruses used in the development expression system include but are not limited to alphaviruses and flaviviruses. Exemplary alphaviruses used for expression systems include but are not limited to Semliki Forest virus, Venezuelan equine encephalitis virus and Sindbis virus and poliovirus. Alphavirus replicon particle-based vaccine vectors derived from Sindbis virus (SIN), Semliki Forest virus (SFV), and Venezuelan equine encephalitis virus (VEE) have been shown to induce robust antigen-specific cellular, humoral, and mucosal immune responses in many animal models of infectious disease and cancer (Perri et al.; Journal of Virology September 2003, 77 (19) 10394-10403; DOI: 10.1128/JVI.77.19.10394-10403.2003; Karl Ljungberg & Peter Liljeström (2015) Self-replicating alphavirus RNA vaccines, Expert Review of Vaccines, 14:2, 177-194, DOI: 10.1586/14760584.2015.965690). Exemplary flavivirus used for expression systems include Kunjin flavivirus. Negative sense RNA virus systems include measles and rhabdoviruses.

In specific embodiments, the SAM RNA vaccine platform is derived from an alphavirus. In such embodiments, the mRNA replicates through a double stranded RNA intermediate, and the antigen of interest replaces structural proteins, so no infectious virus is made.

Immunogens:

The vaccines of the present invention comprise or encode one or more viral immunogens. The viral immunogens may be wild-type viral proteins, fragments and variants thereof. Non-limiting examples of protein fragments include but are not limited to fragments comprising an extracellular domain (ectodomain) only, fragments comprising a cytoplasmic domain and extracellular domain, or fragments comprising a transmembrane domain and cytoplasmic domain and an extracellular domain. The variants may comprise one or more substitutions, insertions and/or deletions of one or more amino acid residues as compared to the wild-type proteins. The variants may comprise a sequence having at least 50%, at least 60%, at least 70%, at least 75%, at least 80%, at least 85%, at least 90%, at least 95%, at least 98%, at least 99% sequence identity as compared to a reference protein sequence. The reference sequence may be any of the viral immunogen sequences disclosed herein or known in the art. In certain embodiments, the variants are functionally inactive.

In certain embodiments, two or more immunogens are in the form of a polyprotein.

In certain embodiments, the one or more viral immunogens are coronavirus immunogens, such as SARS-CoV immunogens, including but not limited to SARS-CoV2 immunogens.

In certain embodiments, the one or more viral immunogens are influenza virus immunogens. In specific embodiments, the one or more influenza virus immunogens comprise one or more of influenza nucleoprotein (NP), polymerase basic 1 (PB1), and matrix 1 (M1), fragments and variants thereof.

In certain embodiments, the vaccines comprise or encode viral immunogens from two or more viruses. In certain embodiments, the vaccines comprise or encode viral immunogens from two or more strains of the same virus. In specific embodiments, the vaccines comprise or encode viral immunogens from two or more strains of SARs-CoV-2. In certain embodiments, the vaccines comprise or encode viral immunogens from one or more influenza viruses. In certain embodiments, the vaccines comprise or encode viral immunogens from one or more coronaviruses and one or more influenza viruses. In specific embodiments, the vaccines comprise or encode viral immunogens from one or more strains of SARs-CoV-2 and one or more influenza viruses.

The complete genome of SARs-CoV-2 is known in the art. The complete genome of the isolate Wuhan-Hu-1 of SARs-CoV-2 is published under GenBank Accession NC_045512.2 (Nature 579 (7798), 265-269 (2020)). Variants of this SARs-CoV-2 and their mutations have also been identified (Bull World Health Organ. 2020 Jul. 1; 98(7):495-504. doi: 10.2471/BLT.20.253591).

Accordingly, the one or more SARS-CoV2 immunogens may be from one or more strains of SARs-CoV2 or may be derived from one or more strains of SARs-CoV2.

In certain embodiments, the one or more SARS-CoV immunogens comprise one or more of SARS-CoV Spike proteins, fragments, derivatives and variants thereof.

In specific embodiments, the one or more SARS-CoV2 immunogens comprise one or more of SARS-CoV2 Spike proteins, fragments, derivatives and variants thereof.

In certain embodiments, the one or more viral immunogens are non-functional. In certain embodiments, the one or more SARS-CoV2 immunogens comprise wild type spike protein or immunogenic fragment thereof. In certain embodiments, the spike protein is full length.

In certain embodiments, the spike protein comprises the spike signal peptide, extracellular, transmembrane and cytoplasmic domains. The sequence of the SARS-COV2 spike protein from the Wuhan-Hu-1 isolate of SARs-CoV2 is known in the art and is published under GenBank Accession: YP_009724390 and is set forth below as SEQ ID NO:1:

MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT

In certain embodiments, the one or more SARS-CoV2 immunogens comprise a variant spike protein or immunogenic fragment thereof. Naturally occurring SARS-CoV2 variants having a substitutions and/or deletions in the spike protein are known in the art and include but are not limited to:

| Variant | Spike Protein Substitutions or Deletions |
|---|---|
| Alpha | T19R, (V70F*), T95I, G142D, E156-, F157-, |
| | R158G, (A222V*), (W258L*), (K417N*), |
| | L452R, T478K, D614G, P681R, D950N |
| Beta | D80A, D215G, 241del, 242del, 243del, |
| | K417N, E484K, N501Y, D614G, A701V |
| Delta | T19R, (V70F*), T95I, G142D, E156-, F157-, |
| | R158G, (A222V*), (W258L*), (K417N*), |
| | L452R, T478K, D614G, P681R, D950N |
| Gamma | L18F, T20N, P26S, D138Y, R190S, K417T, |
| | E484K, N501Y, D614G, H655Y, T1027I |
| Eta | A67V, 69del, 70del, 144del, E484K, D614G, |
| | Q677H, F888L |
| Iota | L5F, (D80G*), T95I, (Y144-*), (F157S*), |
| | D253G, (L452R*), (S477N*), E484K, D614G, |
| | A701V, (T859N*), (D950H*), (Q957R*) |
| Kappa | (T95I), G142D, E154K, L452R, E484Q, |
| | D614G, P681R, Q1071H |
| Lambda | G75V, T76I, Δ246-252, L452Q, F490S, |
| | D614G and T859N |
| B.1.617.3 | T19R, G142D, L452R, E484Q, D614G, |
| | P681R, D950N |
| Mu | T95I, Y144S, Y145N, R346K, E484K, N501Y, |
| | D614G, P681H, and D109N |

Accordingly, in certain embodiments, the one or more SARS-CoV2 immunogens comprise one or more spike proteins, fragments or derivatives thereof from one or more SARs-CoV2 strains or derivatives thereof. In certain embodiments, the one or more spike protein, or fragments thereof comprises one or more substitutions and/or deletions in comparison to a reference sequence. In certain embodiments, the one or more spike proteins comprises one or more substitutions and/or deletions as compared to the sequence of the spike protein of the Wuhan-Hu-1 isolate of SARs-CoV2. Exemplary substitutions and deletions are detailed in the above table. In certain embodiments, the one or more SARS-CoV2 immunogens comprise one or more proteins comprising or encoded by the sequence as set forth below, fragments, variants or derivatives thereof.

In certain embodiments, the spike protein comprises the sequence published under GenBank Accession: YP_009724390 (SEQ ID NO:1), fragments, variants or derivatives thereof.

In certain embodiments, the spike protein comprises the sequence as set forth below (SEQ ID NO:2), fragments, variants or derivatives thereof:

MVNLTTRTQLPPAYTNSFTRGVYYPDKVFRSSVLHSTQDL
FLPFFSNVTWFHAIHVSGTNGTKRFDNPVLPFNDGVYFAS
TEKSKIIRGWIFGTTLDSKTQSLLIVNNATNVVIKVCEFQ
FCNDPFLGVYYHKNNKSWMESEFRVYSSANNCTFEYVSQP
FLMDLEGKQGNFKNLREFVFKNIDGYFKIYSKHTPINLVR
DLPQGFSALEPLVDLPIGINITRFQTLLALHRSYLTPGDS
SSGWTAGAAAYYVGYLQPRTFLLKYNENGTITDAVDCALD
PLSETKCTLKSFTVEKGIYQTSNFRVQPTESIVRFPNITN
LCPFGEVFNATRFASVYAWNRKRISNCVADYSVLYNSASL
STFKCYGVSPTKLNDLCFTNVYADSFVIRGDEVRQIAPGQ
TGKIADYNYKLPDDFTGCVIAWNSNNLDSKVGGNYNYLYR
LFRKSNLKPFERDISTEIYQAGSTPCNGVEGFNCYFPLQS
YGFQPTNGVGYQPYRVVVLSFELLHAPATVCGPKKSTNLV
KNKCVNFNFNGLTGTGVLTESNKKFLPFQQFGRDIADTTD
AVRDPQTLEILDITPCSFGGVSVITPGTNTSNQVAVLYQD
VNCTEVPVAIHADQLTPTWRVYSTGSNVFQTRAGCLIGAE
HVNNSYECDIPIGAGICASYQTQTNSPRRARSVASQSIIA
YTMSLGAENSVAYSNNSIAIPTNFTISVTTEILPVSMTKT
SVDCTMYICGDSTECSNLLLQYGSFCTQLNRALTGIAVEQ
DKNTQEVFAQVKQIYKTPPIKDFGGFNFSQILPDPSKPSK
RSFIEDLLFNKVTLADAGFIKQYGDCLGDIAARDLICAQK
FNGLTVLPPLLTDEMIAQYTSALLAGTITSGWTFGAGAAL
QIPFAMQMAYRFNGIGVTQNVLYENQKLIANQFNSAIGKI
QDSLSSTASALGKLQDVVNQNAQALNTLVKQLSSNFGAIS
SVLNDILSRLDKVEAEVQIDRLITGRLQSLQTYVTQQLIR
AAEIRASANLAATKMSECVLGQSKRVDFCGKGYHLMSFPQ
SAPHGVVFLHVTYVPAQEKNFTTAPAICHDGKAHFPREGV
FVSNGTHWFVTQRNFYEPQIITTDNTFVSGNCDVVIGIVN
NTVYDPLQPELDSFKEELDKYFKNHTSPDVDLGDISGINA
SVVNIQKEIDRLNEVAKNLNESLIDLQELGKYEQYIKWPW
YIWLGFIAGLIAIVMVTIMLCCMTSCCSCLKGCCSCGSCC
KFDEDDSEPVLKGVKLHYT

In certain embodiments, the spike protein comprises the sequence as set forth below (SEQ ID NO:3), fragments, variants or derivatives thereof:

MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
RSVASQSIIAYTMSLGAENSVAYSNNSIAIPTNFTISVTT
EILPVSMTKTSVDCTMYICGDSTECSNLLLQYGSFCTQLN
RALTGIAVEQDKNTQEVFAQVKQIYKTPPIKDFGGFNFSQ
ILPDPSKPSKRSFIEDLLFNKVTLADAGFIKQYGDCLGDI
AARDLICAQKFNGLTVLPPLLTDEMIAQYTSALLAGTITS
GWTFGAGAALQIPFAMQMAYRFNGIGVTQNVLYENQKLIA
NQFNSAIGKIQDSLSSTASALGKLQDVVNQNAQALNTLVK
QLSSNFGAISSVLNDILSRLDKVEAEVQIDRLITGRLQSL
QTYVTQQLIRAAEIRASANLAATKMSECVLGQSKRVDFCG
KGYHLMSFPQSAPHGVVFLHVTYVPAQEKNFTTAPAICHD
GKAHFPREGVFVSNGTHWFVTQRNFYEPQIITTDNTFVSG
NCDVVIGIVNNTVYDPLQPELDSFKEELDKYFKNHTSPDV
DLGDISGINASVVNIQKEIDRLNEVAKNLNESLIDLQELG
KYEQYIKWPWYIWLGFIAGLIAIVMVTIMLCCMTSCCSCL
KGCCSCGSCCKFDEDDSEPVLKGVKLHYTSRLEEELRRRL
TE*

In certain embodiments, the spike protein comprises the sequence as set forth below (SEQ ID NO:4), fragments, variants or derivatives thereof:

MFVFLVLLPLVSSQCVNLTTRTQLPPAYTNSFTRGVYYPD
KVFRSSVLHSTQDLFLPFFSNVTWFHAISGTNGTKRFDNP
VLPFNDGVYFASTEKSNIIRGWIFGTTLDSKTQSLLIVNN
ATNVVIKVCEFQFCNDPFLGVYHKNNKSWMESEFRVYSSA
NNCTFEYVSQPFLMDLEGKQGNFKNLREFVFKNIDGYFKI
YSKHTPINLVRDLPQGFSALEPLVDLPIGINITRFQTLLA
LHRSYLTPGDSSSGWTAGAAAYYVGYLQPRTFLLKYNENG
TITDAVDCALDPLSETKCTLKSFTVEKGIYQTSNFRVQPT
ESIVRFPNITNLCPFGEVFNATRFASVYAWNRKRISNCVA
DYSVLYNSASFSTFKCYGVSPTKLNDLCFTNVYADSFVIR
GDEVRQIAPGQTGKIADYNYKLPDDFTGCVIAWNSNNLDS
KVGGNYNYLYRLFRKSNLKPFERDISTEIYQAGSTPCNGV
EGFNCYFPLQSYGFQPTYGVGYQPYRVVVLSFELLHAPAT
VCGPKKSTNLVKNKCVNFNFNGLTGTGVLTESNKKFLPFQ
QFGRDIDDTTDAVRDPQTLEILDITPCSFGGVSVITPGTN
TSNQVAVLYQGVNCTEVPVAIHADQLTPTWRVYSTGSNVF
QTRAGCLIGAEHVNNSYECDIPIGAGICASYQTQTNSHRS
VASQSIIAYTMSLGAENSVAYSNNSIAIPINFTISVTTEI
LPVSMTKTSVDCTMYICGDSTECSNLLLQYGSFCTQLNRA
LTGIAVEQDKNTQEVFAQVKQIYKTPPIKDFGGFNFSQIL
PDPSKPSKRSFIEDLLFNKVTLADAGFIKQYGDCLGDIAA
RDLICAQKFNGLTVLPPLLTDEMIAQYTSALLAGTITSGW
TFGAGAALQIPFAMQMAYRFNGIGVTQNVLYENQKLIANQ
FNSAIGKIQDSLSSTASALGKLQDVVNQNAQALNTLVKQL
SSNFGAISSVLNDILARLDKVEAEVQIDRLITGRLQSLQT
YVTQQLIRAAEIRASANLAATKMSECVLGQSKRVDFCGKG
YHLMSFPQSAPHGVVFLHVTYVPAQEKNFTTAPAICHDGK
AHFPREGVFVSNGTHWFVTQRNFYEPQIITTHNTFVSGNC
DVVIGIVNNTVYDPLQPELDSFKEELDKYFKNHTSPDVDL
GDISGINASVVNIQKEIDRLNEVAKNLNESLIDLQELGKY
EQYIKWPWYIWLGFIAGLIAIVMVTIMLCCMTSCCSCLKG
CCSCGSCCKFDEDDSEPVLKGVKLHYTSRLEEELRRRLTE
*

In certain embodiments, the spike protein comprises the sequence as set forth below (SEQ ID NO:5), fragments, variants or derivatives thereof:

MFVFLVLLPLVSSQCVNFTTRTQLPPAYTNSFTRGVYYPD
KVFRSSVLHSTQDLFLPFFSNVTWFHAIHVSGTNGTKRFA
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRGLPQGFSALEPLVDLPIGINITRFQT
LLALHISYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGNIADYNYKLPDDFTGCVIAWNSNN
LDSKVGGNYNYLYRLFRKSNLKPFERDISTEIYQAGSTPC
NGVKGFNCYFPLQSYGFQPTYGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQGVNCTEVPVAIHADQLTPTWRVYSTGS
NVFQTRAGCLIGAEHVNNSYECDIPIGAGICASYQTQTNS
RSVASQSIIAYTMSLGVENSVAYSNNSIAIPTNFTISVTT
EILPVSMTKTSVDCTMYICGDSTECSNLLLQYGSFCTQLN
RALTGIAVEQDKNTQEVFAQVKQIYKTPPIKDFGGFNFSQ
ILPDPSKPSKRSFIEDLLFNKVTLADAGFIKQYGDCLGDI
AARDLICAQKFNGLTVLPPLLTDEMIAQYTSALLAGTITS
GWTFGAGAALQIPFAMQMAYRFNGIGVTQNVLYENQKLIA
NQFNSAIGKIQDSLSSTASALGKLQDVVNQNAQALNTLVK
QLSSNFGAISSVLNDILSRLDKVEAEVQIDRLITGRLQSL
QTYVTQQLIRAAEIRASANLAATKMSECVLGQSKRVDFCG
KGYHLMSFPQSAPHGVVFLHVTYVPAQEKNFTTAPAICHD
GKAHFPREGVFVSNGTHWFVTQRNFYEPQIITTDNTFVSG
NCDVVIGIVNNTVYDPLQPELDSFKEELDKYFKNHTSPDV
DLGDISGINASVVNIQKEIDRLNEVAKNLNESLIDLQELG
KYEQYIKWPWYIWLGFIAGLIAIVMVTIMLCCMTSCCSCL
KGCCSCGSCCKFDEDDSEPVLKGVKLHYTSRLEEELRRRL
TE*

In certain embodiments, the spike protein comprises the sequence as set forth below (SEQ ID NO:6), fragments, variants or derivatives thereof:

MFVFLVLLPLVSIQCVNLTTRTQLPPAYTNSFTRGVYYPD
KVFRSSVLHSTQDLFLPFFSNVTWFHAIVSGTNGTKRFDN
PVLPFNDGVYFASTEKSNIIRGWIFGTTLDSKTQSLLIVN
NATNVVIKVCEFQFCNDPFLGVYYHKNNKSCMESEFRVYS
SANNCTFEYVSQPFLMDLEGKQGNFKNLREFVFKNIDGYF
KIYSKHTPINLVRDLPQGFSALEPLVDLPIGINITRFQTL
LALHRSYLTPGDSSSGWTAGAAAYYVGYLQPRTFLLKYNE
NGTITDAVDCALDPLSETKCTLKSFTVEKGIYQTSNFRVQ
PTESIVRFPNITNLCPFGEVFNATRFASVYAWNRKRISNC
VADYSVLYNSASFSTFKCYGVSPTKLNDLCFTNVYADSFV
IRGDEVRQIAPGQTGKIADYNYKLPDDFTGCVIAWNSNNL
DSKVGGNYNYRYRLFRKSNLKPFERDISTEIYQAGSTPCN
GVEGFNCYFPLQSYGFQPTNGVGYQPYRVVVLSFELLHAP
ATVCGPKKSTNLVKNKCVNFNFNGLTGTGVLTESNKKFLP
FQQFGRDIADTTDAVRDPQTLEILDITPCSFGGVSVITPG
TNTSNQVAVLYQDVNCTEVPVAIHADQLTPTWRVYSTGSN
VFQTRAGCLIGAEHVNNSYECDIPIGAGICASYQTQTNSR
SVASQSIIAYTMSLGAENSVAYSNNSIAIPTNFTISVTTE
ILPVSMTKTSVDCTMYICGDSTECSNLLLQYGSFCTQLNR
ALTGIAVEQDKNTQEVFAQVKQIYKTPPIKDFGGFNFSQI
LPDPSKPSKRSFIEDLLFNKVTLADAGFIKQYGDCLGDIA
ARDLICAQKFNGLTVLPPLLTDEMIAQYTSALLAGTITSG
WTFGAGAALQIPFAMQMAYRFNGIGVTQNVLYENQKLIAN
QFNSAIGKIQDSLSSTASALGKLQDVVNQNAQALNTLVKQ
LSSNFGAISSVLNDILSRLDKVEAEVQIDRLITGRLQSLQ
TYVTQQLIRAAEIRASANLAATKMSECVLGQSKRVDFCGK
GYHLMSFPQSAPHGVVFLHVTYVPAQEKNFTTAPAICHDG
KAHFPREGVFVSNGTHWFVTQRNFYEPQIITTDNTFVSGN
CDVVIGIVNNTVYDPLQPELDSFKEELDKYFKNHTSPDVD
LGDISGINASVVNIQKEIDRLNEVAKNLNESLIDLQELGK
YEQYIKWPWYIWLGFIAGLIAIVMVTIMLCCMTSCCSCLK
GCCSCGSCCKFDEDDSEPVLKGVKLHYTSRLEEELRRRLT
E*

In certain embodiments, the spike protein comprises the sequence as set forth below (SEQ ID NO:7), fragments, variants or derivatives thereof:

MFVFLVLLPLVSSQCVNFTNRTQLPSAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNYPFLGVYYHKNNKSWMESEFRVY
SSANNCTFEYVSQPFLMDLEGKQGNFKNLSEFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGNIADYNYKLPDDFTGCVIAWNSNN
LDSKVGGNYNYLYRLFRKSNLKPFERDISTEIYQAGSTPC
NGVKGFNCYFPLQSYGFQPTYGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQGVNCTEVPVAIHADQLTPTWRVYSTGS
NVFQTRAGCLIGAEYVNNSYECDIPIGAGICASYQTQTNS
RSVASQSIIAYTMSLGAENSVAYSNNSIAIPTNFTISVTT
EILPVSMTKTSVDCTMYICGDSTECSNLLLQYGSFCTQLN
RALTGIAVEQDKNTQEVFAQVKQIYKTPPIKDFGGFNFSQ
ILPDPSKPSKRSFIEDLLFNKVTLADAGFIKQYGDCLGDI
AARDLICAQKFNGLTVLPPLLTDEMIAQYTSALLAGTITS
GWTFGAGAALQIPFAMQMAYRFNGIGVTQNVLYENQKLIA
NQFNSAIGKIQDSLSSTASALGKLQDVVNQNAQALNTLVK
QLSSNFGAISSVLNDILSRLDKVEAEVQIDRLITGRLQSL
QTYVTQQLIRAAEIRASANLAAIKMSECVLGQSKRVDFCG
KGYHLMSFPQSAPHGVVFLHVTYVPAQEKNFTTAPAICHD
GKAHFPREGVFVSNGTHWFVTQRNFYEPQIITTDNTFVSG
NCDVVIGIVNNTVYDPLQPELDSFKEELDKYFKNHTSPDV
DLGDISGINASFVNIQKEIDRLNEVAKNLNESLIDLQELG
KYEQYIKWPWYIWLGFIAGLIAIVMVTIMLCCMTSCCSCL
KGCCSCGSCCKFDEDDSEPVLKGVKLHYTSRLEEELRRRL
TE*

In certain embodiments, the spike protein comprises the sequence as set forth below (SEQ ID NO:8), fragments, variants or derivatives thereof:

MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASIEKSNIIRGWIFGTTLDSKTQSLLIV
NNATNVVIKVCEFQFCNDPFLGVYYHKNNKSWMKSEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNK
LDSKVGGNYNYRYRLFRKSNLKPFERDISTEIYQAGSTPC
NGVQGFNCYFPLQSYGFQPTYGVGYQPYRVVVLSFELLHA
PATVCGPKKSTNLVKNKCVNFNFNGLTGTGVLTESNKKFL
PFQQFGRDIDDTTDAVRDPQTLEILDITPCSFGGVSVITP
GTNTSNQVAVLYQGVNCTEVPVAIHADQLTPTWRVYSTGS
NVFQTRAGCLIGAEHVNNSYECDIPIGAGICASYQTQTNS
SVASQSIIAYTMSLGAENSVAYSNNSIAIPINFTISVTTE
ILPVSMTKTSVDCTMYICGDSTECSNLLLQYGSFCTQLNR
ALTGIAVEQDKNTQEVFAQVKQIYKTPPIKDFGGFNFSQI
LPDPSKPSKRSFIEDLLFNKVTLADAGFIKQYGDCLGDIA
ARDLICAQKFNGLTVLPPLLTDEMIAQYTSALLAGTITSG
WTFGAGAALQIPFAMQMAYRFNGIGVTQNVLYENQKLIAN
QFNSAIGKIQDSLSSTASALGKLQDVVNQNAQALNTLVKQ
LSSNFGAISSVLNDILARLDKVEAEVQIDRLITGRLQSLQ
TYVTQQLIRAAEIRASANLAATKMSECVLGQSKRVDFCGK
GYHLMSFPQSAPHGVVFLHVTYVPAHEKNFTTAPAICHDG
KAHFPREGVFVSNGTHWFVTQRNFYEPQIITTHNTFVSGN
CDVVIGIVNNTVYDPLQPELDSFKEELDKYFKNHTSPDVD
LGDISGINASVVNIQKEIDRLNEVAKNLNESLIDLQELGK
YEQYIKWPWYIWLGFIAGLIAIVMVTIMLCCMTSCCSCLK
GCCSCGSCCKFDEDDSEPVLKGVKLHYTSRLEEELRRRLT
E*

In certain embodiments, the spike protein comprises the sequence as set forth below (SEQ ID NO:76), fragments, variants or derivatives thereof.

MFVFLVLLPLVSSQCVNFTTRTQLPPAYTNSFTRGVYYPD
KVFRSSVLHSTQDLFLPFFSNVTWFHAIHVSGTNVIKRFD
NPVLPFNDGVYFASTEKSNIIRGWIFGTTLDSKTQSLLIV
NNATNVVIKVCEFQFCNYPFLGVYYHKNNKSWMESEFRVY
SSANNCTFEYVSQPFLMDLEGKQGNFKNLSEFVFKNIDGY
FKIYSKHTPINLVRDLPQGFSALEPLVDLPIGINITRFQT
LLALHNSSSGWTAGAAAYYVGYLQPRTFLLKYNENGTITD
AVDCALDPLSETKCTLKSFTVEKGIYQTSNFRVQPTESIV
RFPNITNLCPFGEVFNATRFASVYAWNRKRISNCVADYSV
LYNSASFSTFKCYGVSPTKLNDLCFTNVYADSFVIRGDEV
RQIAPGQTGKIADYNYKLPDDFTGCVIAWNSNNLDSKVGG
NYNYQYRLFRKSNLKPFERDISTEIYQAGSTPCNGVKGFN
CYSPLQSYGFQPTYGVGYQPYRVVVLSFELLHAPATVCGP
KKSTNLVKNKCVNFNFNGLTGTGVLTESNKKFLPFQQFGR
DIADTTDAVRDPQTLEILDITPCSFGGVSVITPGTNTSNQ
VAVLYQGVNCTEVPVAIHADQLTPTWRVYSTGSNVFQTRA
GCLIGAEYVNNSYECDIPIGAGICASYQTQTNSSQSIIAY
TMSLGAENSVAYSNNSIAIPTNFTISVTTEILPVSMTKTS
VDCTMYICGDSTECSNLLLQYGSFCTQLNRALTGIAVEQD
KNTQEVFAQVKQIYKTPPIKDFGGFNFSQILPDPSKPSKR
SFIEDLLFNKVTLADAGFIKQYGDCLGDIAARDLICAQKF
NGLNVLPPLLTDEMIAQYTSALLAGTITSGWTFGAGAALQ
IPFAMQMAYRFNGIGVTQNVLYENQKLIANQFNSAIGKIQ
DSLSSTASALGKLQDVVNQNAQALNTLVKQLSSNFGAISS
VLNDILSRLDKVEAEVQIDRLITGRLQSLQTYVTQQLIRA
AEIRASANLAATKMSECVLGQSKRVDFCGKGYHLMSFPQS
APHGVVFLHVTYVPAQEKNFTTAPAICHDGKAHFPREGVF
VSNGTHWFVTQRNFYEPQIITTDNTFVSGNCDVVIGIVNN
TVYDPLQPELDSFKEELDKYFKNHTSPDVDLADISGINAS
VVNIQKEIDRLNEVAKNLNESLIDLQELGKYEQYIKWPWY
IWLGFIAGLIAIVMVTIMLCCMTSCCSCLKGCCSCGSCCK
FDEDDSEPVLKGVKLHYTSRLEEELRRRLTE*

In certain embodiments, the spike protein or fragments thereof is non-functional.

In certain embodiments, the one or more SARS-CoV2 immunogens comprise one or more spike proteins, or fragments thereof where the furin cleavage site is absent.

In certain embodiments, the spike proteins where the furin cleavage site is absent comprises the sequence as set forth below (SEQ ID NO: 9), fragments, variants or derivatives thereof:

MVNLTTRTQLPPAYTNSFTRGVYYPDKVFRSSVLHSTQDL
FLPFFSNVTWFHAIHVSGTNGTKRFDNPVLPFNDGVYFAS
TEKSKIIRGWIFGTTLDSKTQSLLIVNNATNVVIKVCEFQ
FCNDPFLGVYYHKNNKSWMESEFRVYSSANNCTFEYVSQP
FLMDLEGKQGNFKNLREFVFKNIDGYFKIYSKHTPINLVR
DLPQGFSALEPLVDLPIGINITRFQTLLALHRSYLTPGDS
SSGWTAGAAAYYVGYLQPRTFLLKYNENGTITDAVDCALD
PLSETKCTLKSFTVEKGIYQTSNFRVQPTESIVRFPNITN
LCPFGEVFNATRFASVYAWNRKRISNCVADYSVLYNSASL
STFKCYGVSPTKLNDLCFTNVYADSFVIRGDEVRQIAPGQ
TGKIADYNYKLPDDFTGCVIAWNSNNLDSKVGGNYNYLYR
LFRKSNLKPFERDISTEIYQAGSTPCNGVEGFNCYFPLQS
YGFQPTNGVGYQPYRVVVLSFELLHAPATVCGPKKSTNLV
KNKCVNFNFNGLTGTGVLTESNKKFLPFQQFGRDIADTTD
AVRDPQTLEILDITPCSFGGVSVITPGTNTSNQVAVLYQD
VNCTEVPVAIHADQLTPTWRVYSTGSNVFQTRAGCLIGAE
HVNNSYECDIPIGAGICASYQTQTNSASQSIIAYTMSLGA
ENSVAYSNNSIAIPTNFTISVTTEILPVSMTKTSVDCTMY
ICGDSTECSNLLLQYGSFCTQLNRALTGIAVEQDKNTQEV
FAQVKQIYKTPPIKDFGGFNFSQILPDPSKPSKRSFIEDL
LFNKVTLADAGFIKQYGDCLGDIAARDLICAQKFNGLTVL
PPLLTDEMIAQYTSALLAGTITSGWTFGAGAALQIPFAMQ
MAYRFNGIGVTQNVLYENQKLIANQFNSAIGKIQDSLSST
ASALGKLQDVVNQNAQALNTLVKQLSSNFGAISSVLNDIL
SRLDKVEAEVQIDRLITGRLQSLQTYVTQQLIRAAEIRAS
ANLAATKMSECVLGQSKRVDFCGKGYHLMSFPQSAPHGVV
FLHVTYVPAQEKNFTTAPAICHDGKAHFPREGVFVSNGTH
WFVTQRNFYEPQIITTDNTFVSGNCDVVIGIVNNTVYDPL
QPELDSFKEELDKYFKNHTSPDVDLGDISGINASVVNIQK
EIDRLNEVAKNLNESLIDLQELGKYEQYIKWPWYIWLGFI
AGLIAIVMVTIMLCCMTSCCSCLKGCCSCGSCCKFDEDDS
EPVLKGVKLHYT

In certain embodiments, the spike proteins where the furin cleavage site is absent comprises the sequence as set forth below (SEQ ID NO: 10), fragments, variants or derivatives thereof:

MVNLTTRTQLPPAYTNSFTRGVYYPDKVFRSSVLHSTQDL
FLPFFSNVTWFHAIHVSGTNGTKRFDNPVLPFNDGVYFAS
TEKSKIIRGWIFGTTLDSKTQSLLIVNNATNVVIKVCEFQ
FCNDPFLGVYYHKNNKSWMESEFRVYSSANNCTFEYVSQP
FLMDLEGKQGNFKNLREFVFKNIDGYFKIYSKHTPINLVR
DLPQGFSALEPLVDLPIGINITRFQTLLALHRSYLTPGDS
SSGWTAGAAAYYVGYLQPRTFLLKYNENGTITDAVDCALD
PLSETKCTLKSFTVEKGIYQTSNFRVQPTESIVRFPNITN
LCPFGEVFNATRFASVYAWNRKRISNCVADYSVLYNSASL
STFKCYGVSPTKLNDLCFTNVYADSFVIRGDEVRQIAPGQ
TGKIADYNYKLPDDFTGCVIAWNSNNLDSKVGGNYNYLYR
LFRKSNLKPFERDISTEIYQAGSTPCNGVEGFNCYFPLQS
YGFQPTNGVGYQPYRVVVLSFELLHAPATVCGPKKSTNLV
KNKCVNFNFNGLTGTGVLTESNKKFLPFQQFGRDIADTTD
AVRDPQTLEILDITPCSFGGVSVITPGTNTSNQVAVLYQD
VNCTEVPVAIHADQLTPTWRVYSTGSNVFQTRAGCLIGAE
HVNNSYECDIPIGAGICASYQTQTNSPRRARSVASQSIIA
YTMSLGAENSVAYSNNSIAIPTNFTISVTTEILPVSMTKT
SVDCTMYICGDSTECSNLLLQYGSFCTQLNRALTGIAVEQ
DKNTQEVFAQVKQIYKTPPIKDFGGFNFSQILPDPSKPSK
RSFIEDLLFNKVTLADAGFIKQYGDCLGDIAARDLICAQK
FNGLTVLPPLLTDEMIAQYTSALLAGTITSGWTFGAGAAL
QIPFAMQMAYRFNGIGVTQNVLYENQKLIANQFNSAIGKI
QDSLSSTASALGKLQDVVNQNAQALNTLVKQLSSNFGAIS
SVLNDILSRLDKVEAEVQIDRLITGRLQSLQTYVTQQLIR
AAEIRASANLAATKMSECVLGQSKRVDFCGKGYHLMSFPQ
SAPHGVVFLHVTYVPAQEKNFTTAPAICHDGKAHFPREGV
FVSNGTHWFVTQRNFYEPQIITTDNTFVSGNCDVVIGIVN
NTVYDPLQPELDSFKEELDKYFKNHTSPDVDLGDISGINA
SVVNIQKEIDRLNEVAKNLNESLIDLQELGKYEQYIKWP

In certain embodiments, the one or more SARS-CoV2 immunogens comprise the extracellular domain(s) of one or more spike proteins or fragments, variants or derivatives thereof. In certain embodiments, the one or more SARS-CoV2 immunogens comprise the extracellular domain(s) and the cytoplasmic domain(s) from one or spike proteins.

In certain embodiments, the spike protein extracellular domain comprises the sequence as set forth below (SEQ ID NO:11)

MVNLTTRTQLPPAYTNSFTRGVYYPDKVFRSSVLHSTQDL
FLPFFSNVTWFHAIHVSGTNGTKRFDNPVLPFNDGVYFAS
TEKSNIIRGWIFGTTLDSKTQSLLIVNNATNVVIKVCEFQ
FCNDPFLGVYYHKNNKSWMESEFRVYSSANNCTFEYVSQP
FLMDLEGKQGNFKNLREFVFKNIDGYFKIYSKHTPINLVR
DLPQGFSALEPLVDLPIGINITRFQTLLALHRSYLTPGDS
SSGWTAGAAAYYVGYLQPRTFLLKYNENGTITDAVDCALD
PLSETKCTLKSFTVEKGIYQTSNFRVQPTESIVRFPNITN
LCPFGEVFNATRFASVYAWNRKRISNCVADYSVLYNSASF
STFKCYGVSPTKLNDLCFTNVYADSFVIRGDEVRQIAPGQ
TGKIADYNYKLPDDFTGCVIAWNSNNLDSKVGGNYNYLYR
LFRKSNLKPFERDISTEIYQAGSTPCNGVEGFNCYFPLQS
YGFQPTNGVGYQPYRVVVLSFELLHAPATVCGPKKSTNLV
KNKCVNFNFNGLTGTGVLTESNKKFLPFQQFGRDIADTTD
AVRDPQTLEILDITPCSFGGVSVITPGTNTSNQVAVLYQD
VNCTEVPVAIHADQLTPTWRVYSTGSNVFQTRAGCLIGAE
HVNNSYECDIPIGAGICASYQTQTNSPRRARSVASQSIIA
YTMSLGAENSVAYSNNSIAIPTNFTISVTTEILPVSMTKT
SVDCTMYICGDSTECSNLLLQYGSFCTQLNRALTGIAVEQ
DKNTQEVFAQVKQIYKTPPIKDFGGFNFSQILPDPSKPSK
RSFIEDLLFNKVTLADAGFIKQYGDCLGDIAARDLICAQK
FNGLTVLPPLLTDEMIAQYTSALLAGTITSGWTFGAGAAL
QIPFAMQMAYRFNGIGVTQNVLYENQKLIANQFNSAIGKI
QDSLSSTASALGKLQDVVNQNAQALNTLVKQLSSNFGAIS
SVLNDILSRLDKVEAEVQIDRLITGRLQSLQTYVTQQLIR
AAEIRASANLAATKMSECVLGQSKRVDFCGKGYHLMSFPQ
SAPHGVVFLHVTYVPAQEKNFTTAPAICHDGKAHFPREGV
FVSNGTHWFVTQRNFYEPQIITTDNTFVSGNCDVVIGIVN
NTVYDPLQPELDSFKEELDKYFKNHTSPDVDLGDISGINA
SVVNIQKEIDRLNEVAKNLNESLIDLQELGKYEQYIKWP

In certain embodiments, the spike protein extracellular domain comprises the sequence as set forth below (SEQ ID NO:12)

MVNLTTRTQLPPAYTNSFTRGVYYPDKVFRSSVLHSTQDLFLPFFSNVTWF
HAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATV
CGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQ
TNSRSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKV
EAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAK
NLNESLIDLQELGKYEQYIKWPSRLEEELRRRLTE*

In certain embodiments, the spike protein extracellular domain comprises the sequence as set forth below (SEQ ID NO:13)

MVNLTTRTQLPPAYTNSFTRGVYYPDKVFRSSVLHSTQDLFLPFFSNVTWF
HAISGTNGTKRFDNPVLPFNDGVYFASTEKSNIIRGWIFGTTLDSKTQSLL
IVNNATNVVIKVCEFQFCNDPFLGVYHKNNKSWMESEFRVYSSANNCTFEY
VSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQP
RTFLLKYNENGTITDAVDCALDPLSETKCTLKSFTVEKGIYQTSNFRVQPT
ESIVRFPNITNLCPFGEVFNATRFASVYAWNRKRISNCVADYSVLYNSASF
STFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAG
STPCNGVEGFNCYFPLQSYGFQPTYGVGYQPYRVVVLSFELLHAPATVCGP
KKSTNLVKNKCVNFNFNGLTGTGVLTESNKKFLPFQQFGRDIDDTTDAVRD
PQTLEILDITPCSFGGVSVITPGTNTSNQVAVLYQGVNCTEVPVAIHADQL
TPTWRVYSTGSNVFQTRAGCLIGAEHVNNSYECDIPIGAGICASYQTQTNS
HRSVASQSIIAYTMSLGAENSVAYSNNSIAIPINFTISVTTEILPVSMTKT
SVDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDSL
SSTASALGKLQDVVNQNAQALNTLVKQLSSNFGAISSVLNDILARLDKVEA
EVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPR
EGVFVSNGTHWFVTQRNFYEPQIITTHNTFVSGNCDVVIGIVNNTVYDPLQ
PELDSFKEELDKYFKNHTSPDVDLGDISGINASVVNIQKEIDRLNEVAKNL
NESLIDLQELGKYEQYIKWPSRLEEELRRRLTE*

In certain embodiments, the spike protein extracellular domain comprises the sequence as set forth below (SEQ ID NO:14)

MVNFTTRTQLPPAYTNSFTRGVYYPDKVFRSSVLHSTQDLFLPFFSNVTWF
HAIHVSGTNGTKRFANPVLPFNDGVYFASTEKSNIIRGWIFGTTLDSKTQS
LLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRGLPQ
GFSALEPLVDLPIGINITRFQTLLALHISYLTPGDSSSGWTAGAAAYYVGY
LQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNS
ASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIY
QAGSTPCNGVKGFNCYFPLQSYGFQPTYGVGYQPYRVVVLSFELLHAPATV
CGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQ
TNSRSVASQSIIAYTMSLGVENSVAYSNNSIAIPTNFTISVTTEILPVSMT
KTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKV
EAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAK
NLNESLIDLQELGKYEQYIKWPSRLEEELRRRLTE*

In certain embodiments, the spike protein extracellular domain comprises the sequence as set forth below (SEQ ID NO:15)

MVNLTTRTQLPPAYTNSFTRGVYYPDKVFRSSVLHSTQDLFLPFFSNVTWF
HAIVSGTNGTKRFDNPVLPFNDGVYFASTEKSNIIRGWIFGTTLDSKTQSL
LIVNNATNVVIKVCEFQFCNDPFLGVYYHKNNKSCMESEFRVYSSANNCTF
EYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYL
QPRTFLLKYNENGTITDAVDCALDPLSETKCTLKSFTVEKGIYQTSNFRVQ
PTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNY
KLPDDFTGCVIAWNSNNLDSKVGGNYNYRYRLFRKSNLKPFERDISTEIYQ
AGSTPCNGVEGFNCYFPLQSYGFQPTNGVGYQPYRVVVLSFELLHAPATVC
GPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHAD
QLTPTWRVYSTGSNVFQTRAGCLIGAEHVNNSYECDIPIGAGICASYQTQT
NSRSVASQSIIAYTMSLGAENSVAYSNNSIAIPTNFTISVTTEILPVSMTK
TSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSGW
TFGAGAALQIPFAMQMAYRENGIGVTQNVLYENQKLIANQFNSAIGKIQDS
LSSTASALGKLQDVVNQNAQALNTLVKQLSSNFGAISSVLNDILSRLDKVE
AEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVAKN
LNESLIDLQELGKYEQYIKWPSRLEEELRRRLTE*

In certain embodiments, the spike protein extracellular domain comprises the sequence as set forth below (SEQ ID NO:16)

MVNFTNRTQLPSAYTNSFTRGVYYPDKVFRSSVLHSTQDLFLPFFSNVTWF
HAIHVSGTNGTKRFDNPVLPFNDGVYFASTEKSNIIRGWIFGTTLDSKTQS
LLIVNNATNVVIKVCEFQFCNYPFLGVYYHKNNKSWMESEFRVYSSANNCT
FEYVSQPFLMDLEGKQGNFKNLSEFVFKNIDGYFKIYSKHTPINLVRDLPQ
GFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNS
ASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIY
QAGSTPCNGVKGFNCYFPLQSYGFQPTYGVGYQPYRVVVLSFELLHAPATV
CGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHA
DQLTPTWRVYSTGSNVFQTRAGCLIGAEYVNNSYECDIPIGAGICASYQTQ
TNSRSVASQSIIAYTMSLGAENSVAYSNNSIAIPTNFTISVTTEILPVSMT
KTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKV
EAEVQIDRLITGRLQSLQTYVTQQLIRAAEIRASANLAAIKMSECVLGQSK
RVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDP
LQPELDSFKEELDKYFKNHTSPDVDLGDISGINASFVNIQKEIDRLNEVAK
NLNESLIDLQELGKYEQYIKWPSRLEEELRRRLTE*

In certain embodiments, the spike protein extracellular domain comprises the sequence as set forth below (SEQ ID NO:17)

MVNLTTRTQLPPAYTNSFTRGVYYPDKVFRSSVLHSTQDLFLPFFSNVTWF
HAIHVSGTNGTKRFDNPVLPFNDGVYFASIEKSNIIRGWIFGTTLDSKTQS
LLIVNNATNVVIKVCEFQFCNDPFLGVYYHKNNKSWMKSEFRVYSSANNCT
FEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYN
YKLPDDFTGCVIAWNSNKLDSKVGGNYNYRYRLFRKSNLKPFERDISTEIY
QAGSTPCNGVQGFNCYFPLQSYGFQPTYGVGYQPYRVVVLSFELLHAPATV
CGPKKSTNLVKNKCVNFNFNGLTGTGVLTESNKKFLPFQQFGRDIDDTTDA
VRDPQTLEILDITPCSFGGVSVITPGTNTSNQVAVLYQGVNCTEVPVAIHA
DQLTPTWRVYSTGSNVFQTRAGCLIGAEHVNNSYECDIPIGAGICASYQTQ
TNSSVASQSIIAYTMSLGAENSVAYSNNSIAIPINFTISVTTEILPVSMTK
TSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSGW
TFGAGAALQIPFAMQMAYRFNGIGVTQNVLYENQKLIANQFNSAIGKIQDS
LSSTASALGKLQDVVNQNAQALNTLVKQLSSNFGAISSVLNDILARLDKVE
AEVQIDRLITGRLQSLQTYVTQQLIRAAEIRASANLAATKMSECVLGQSKR
VDFCGKGYHLMSFPQSAPHGVVFLHVTYVPAHEKNFTTAPAICHDGKAHFP
REGVFVSNGTHWFVTQRNFYEPQIITTHNTFVSGNCDVVIGIVNNTVYDPL
QPELDSFKEELDKYFKNHTSPDVDLGDISGINASVVNIQKEIDRLNEVAKN
LNESLIDLQELGKYEQYIKWPSRLEEELRRRLTE*

In certain embodiments, the spike protein extracellular domain comprises the sequence as set forth below (SEQ ID NO:77)

MVNFTTRTQLPPAYTNSFTRGVYYPDKVFRSSVLHSTQDLFLPFFSNVTWF
HAIHVSGTNVIKRFDNPVLPFNDGVYFASTEKSNIIRGWIFGTTLDSKTQS
LLIVNNATNVVIKVCEFQFCNYPFLGVYYHKNNKSWMESEFRVYSSANNCT
FEYVSQPFLMDLEGKQGNFKNLSEFVFKNIDGYFKIYSKHTPINLVRDLPQ
GFSALEPLVDLPIGINITRFQTLLALHNSSSGWTAGAAAYYVGYLQPRTFL
LKYNENGTITDAVDCALDPLSETKCTLKSFTVEKGIYQTSNFRVQPTESIV
RFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDF
TGCVIAWNSNNLDSKVGGNYNYQYRLFRKSNLKPFERDISTEIYQAGSTPC
NGVKGFNCYSPLQSYGFQPTYGVGYQPYRVVVLSFELLHAPATVCGPKKST
NLVKNKCVNFNFNGLTGTGVLTESNKKFLPFQQFGRDIADTTDAVRDPQTL
EILDITPCSFGGVSVITPGTNTSNQVAVLYQGVNCTEVPVAIHADQLTPTW
RVYSTGSNVFQTRAGCLIGAEYVNNSYECDIPIGAGICASYQTQTNSSQSI
IAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPI
KDFGGFNFSQILPDPSKPSKRSFIEDLLFNKVTLADAGFIKQYGDCLGDIA
ARDLICAQKFNGLNVLPPLLTDEMIAQYTSALLAGTITSGWTFGAGAALQI
PFAMQMAYRFNGIGVTQNVLYENQKLIANQFNSAIGKIQDSLSSTASALGK
LQDVVNQNAQALNTLVKQLSSNFGAISSVLNDILSRLDKVEAEVQIDRLIT
GRLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEE
LDKYFKNHTSPDVDLADISGINASVVNIQKEIDRLNEVAKNLNESLIDLQE
LGKYEQYIKWPSRLEEELRRRLTE*

In specific embodiments, the one or more SARS-CoV2 immunogens comprise the amino sequence as any one of SEQ ID NOs described herein or immunogenic fragments, variants or derivatives thereof. In specific embodiments, the one or more SARS-CoV2 immunogens are encoded by the nucleic acid sequences as set forth in any one of SEQ ID NOs described herein or fragments thereof.

In certain embodiments, the vaccine comprises one or more nucleic acids encoding one or more viral immunogens. The one or more nucleic acids may be DNA or RNA. A worker skilled in the art would readily appreciate that for coding sequences represented as DNA the thymine (T) is replaced with uracil (U) in the corresponding RNA sequences.

The vaccine comprising or encoding the one or more viral immunogens may be virus-like particle-based vaccines, viral vector-based vaccines or nucleic acid-based vaccines. In nucleic acid-based vaccines, the nucleic acids may optionally include modifications including for example one or more modified nucleosides. In certain embodiments, the nucleic acid sequences are codon optimized. In certain embodiments, the nucleic acid sequences are codon optimized for expression in mammalian cells, optionally human cells. Also provided are nucleic acids comprising sequences having at least 50%, at least 60%, at least 70%, at least 75%, at least 80%, at least 85%, at least 90%, at least 95%, at least 98%, at least 99% sequence identity to a reference nucleic acid sequence. The reference sequence may be any of the nucleic acid sequences disclosed herein or known in the art.

In specific embodiments, the nucleic acid encoding the one or more SARS-CoV2 immunogens comprise a sequence encoding one or more immunogens comprising a sequence as set forth in any one of SEQ ID NOs: 1 to 17, immunogenic fragments, variants or derivatives thereof.

In specific embodiments, the nucleic acid encoding the one or more SARS-CoV2 immunogens comprise the sequence as set forth in any one of SEQ ID NOs: 18 to 33, fragments or derivatives thereof.

In certain embodiments, the sequence encoding the spike protein comprises the sequence as set forth below (SEQ ID NO:18):

GCCACCATGTTCGTATTTCTCGTCCTCCTCCCACTTGTTTCTAGTCAGTGT
GTGAACCTCACTACGCGAACTCAACTGCCGCCTGCTTACACTAATAGTTTT
GACACGAGGAGTCTACTACCCGACAAGGTGTTCCGCTCAAGTGTCTTGCAC
AGCACGCAAGACCTGTTCTTGCCATTTTTCAGCAATGTAACGTGGTTTCAT
GCCATCCACGTGTCAGGTACTAACGGAACGAAGAGATTTGATAACCCGGTC
CTCCCGTTCAACGACGGGGTTTATTTTGCCAGTACAGAAAAGTCCAATATT
ATAAGGGGATGGATTTTCGGCACGACTCTCGACTCTAAGACACAAAGCTTG
CTCATCGTTAATAATGCTACAAACGTGGTTATAAAAGTATGCGAGTTCCAA
TTCTGTAACGACCCCTTTCTGGGGGTTTACTACCACAAAAATAATAAGAGT
TGGATGGAGAGCGAATTCAGAGTTTACTCTTCCGCGAATAATTGCACCTTC
GAGTACGTATCCCAGCCTTTTCTTATGGATCTTGAGGGGAAACAAGGTAAT
TTCAAAAACCTTAGAGAATTCGTTTTTAAGAATATAGATGGATACTTCAAG
ATTTATTCTAAACATACCCCAATCAATCTGGTGCGCGATCTTCCTCAGGGT
TTCTCTGCGCTGGAGCCTCTCGTTGATCTGCCGATCGGAATTAATATAACC
AGATTCCAGACCCTTTTGGCATTGCACCGCTCCTATCTGACACCTGGGGAT
AGTAGTTCCGGCTGGACAGCGGGCGCCGCGGCATACTACGTTGGATATCTC
CAACCCCGCACTTTTCTTCTGAAATATAATGAGAATGGAACGATTACAGAC
GCAGTAGATTGCGCGCTTGACCCCCTTTCAGAAACAAAGTGCACGCTGAAA
TCTTTCACGGTCGAGAAGGGCATTTACCAAACGTCCAATTTCAGAGTTCAA
CCCACTGAGTCTATAGTCAGGTTTCCGAACATCACCAATCTCTGCCCATTT
GGAGAGGTTTTCAACGCAACACGGTTCGCCTCTGTATACGCATGGAATAGA
AAGAGAATATCCAATTGTGTCGCAGACTATAGTGTCTTGTACAACTCTGCG
TCATTTAGCACTTTTAAATGTTATGGGGTGAGTCCAACTAAATTGAACGAT
CTCTGCTTTACAAATGTTTACGCAGATAGTTTTGTGATACGAGGCGACGAG
GTACGACAAATTGCTCCTGGCCAGACCGGGAAAATAGCGGACTACAATTAT
AAACTGCCAGACGATTTCACCGGCTGTGTGATCGCGTGGAACTCAAACAAC
TTGGATAGTAAGGTAGGTGGTAACTATAACTACTTGTACCGGCTCTTCCGC
AAAAGTAATCTTAAACCGTTCGAGCGGGACATCTCTACCGAGATCTATCAA
GCAGGTAGCACTCCCTGCAATGGAGTCGAGGGTTTTAATTGCTATTTTCCC
CTCCAATCATACGGGTTTCAGCCCACAAATGGTGTTGGGTATCAGCCCTAT
CGAGTTGTGGTGCTTTCATTTGAATTGCTTCATGCACCGGCGACAGTTTGT
GGTCCTAAAAAGAGTACCAATTTGGTTAAAAACAAATGTGTCAACTTTAAC
TTTAACGGTCTGACTGGTACGGGGGTGCTTACGGAATCTAATAAAAAGTTT
CTTCCGTTCCAGCAATTCGGACGGGACATAGCGGACACGACTGACGCGGTT
AGAGACCCACAAACACTCGAGATTCTTGACATAACCCCCTGTTCTTTCGGC
GGAGTTTCTGTAATAACGCCTGGGACAAATACGTCAAACCAGGTTGCAGTA
TTGTACCAGGATGTCAACTGCACTGAAGTGCCAGTCGCGATCCACGCAGAT
CAGTTGACTCCAACATGGAGGGTCTACTCTACCGGTTCTAACGTGTTTCAG
ACCAGAGCCGGGTGCCTCATTGGCGCGGAACATGTGAACAATTCTTACGAA
TGCGACATACCCATCGGCGCCGGCATTTGTGCGTCATACCAAACTCAAACT
AATTCCCGGAGTGTGGCCTCCCAATCTATTATTGCTTATACTATGAGTCTT
GGCGCGGAAAACAGTGTAGCGTACTCAAATAACTCTATTGCGATCCCCACG
AATTTCACTATAAGCGTAACTACTGAAATTTTGCCGGTAAGCATGACGAAA
ACTTCAGTGGACTGCACAATGTACATTTGTGGTGACAGCACTGAGTGCTCA
AATCTCCTCCTTCAGTATGGTTCTTTCTGCACACAACTTAACAGGGCACTT
ACCGGAATCGCAGTAGAACAAGACAAAAACACGCAAGAAGTATTCGCACAG
GTCAAACAAATTTATAAAACCCCGCCGATCAAGGATTTTGGAGGGTTTAAC
TTCAGTCAGATCCTTCCAGACCCATCCAAACCCAGCAAACGGAGCTTTATT
GAAGACCTGCTGTTTAATAAAGTTACCCTTGCAGATGCCGGTTTCATCAAG
CAATACGGGGATTGTCTGGGCGACATTGCCGCCCGCGACTTGATCTGTGCC
CAGAAGTTCAACGGTCTCACAGTCTTGCCGCCACTTCTCACTGATGAAATG
ATAGCGCAGTATACATCCGCGCTGCTGGCTGGGACGATAACATCTGGTTGG
ACTTTCGGGGCCGGGGCGGCTTTGCAAATTCCTTTCGCCATGCAGATGGCC
TATAGGTTTAACGGCATAGGAGTGACTCAAAATGTTCTTTATGAGAATCAG
AAGCTTATCGCCAACCAATTTAACTCCGCGATTGGGAAGATACAGGACTCA
TTGAGCTCTACTGCTAGCGCCCTTGGCAAGCTTCAAGACGTAGTCAACCAA
AATGCTCAAGCGCTCAATACTCTCGTAAAGCAGCTCTCAAGCAACTTTGGC
GCGATATCTTCAGTTTTGAACGACATCTTGAGCAGACTCGACAAGGTGGAG
GCTGAAGTTCAAATTGATCGACTGATTACGGGCCGGCTCCAGTCACTCCAA
ACGTACGTGACCCAACAACTCATTAGAGCGGCAGAGATTAGAGCGAGCGCT
AATCTGGCGGCCACAAAGATGTCAGAATGTGTGCTTGGTCAATCCAAAAGG
GTGGATTTCTGCGGAAAGGGCTATCACTTGATGAGTTTCCCGCAGTCCGCG
CCTCATGGAGTTGTTTTCCTCCACGTAACCTACGTTCCCGCTCAGGAAAAG
AATTTCACGACAGCTCCGGCAATATGTCATGACGGTAAAGCACATTTCCCC
AGGGAAGGCGTTTTTGTCAGTAATGGAACCCACTGGTTCGTCACCCAGCGA
AACTTTTACGAGCCCCAAATAATAACTACTGACAATACCTTCGTCAGCGGC
AATTGCGACGTCGTCATAGGCATAGTAAATAATACGGTTTACGACCCTCTT
CAACCCGAGTTGGACAGCTTCAAAGAAGAACTTGATAAGTATTTTAAAAAC
CACACTAGCCCAGATGTAGACCTTGGCGACATCAGCGGTATCAATGCTTCC
GTTGTGAACATTCAAAAGGAAATTGACCGACTCAATGAAGTAGCGAAGAAC
TTGAATGAGTCTCTGATCGATCTTCAGGAGCTCGGGAAATATGAGCAATAC
ATCAAATGGCCGTGGTACATCTGGCTGGGTTTTATAGCTGGCCTGATCGCA
ATTGTAATGGTAACTATAATGTTGTGTTGCATGACCTCTTGCTGCAGCTGT
TTGAAGGGATGCTGTTCTTGCGGGAGTTGCTGTAAATTTGATGAGGATGAC
AGCGAGCCGGTGTTGAAAGGAGTGAAGCTTCATTATACTTCACGACTGGAG
GAAGAACTGCGCCGACGCCTGACTGAATGA

In certain embodiments, the sequence encoding the spike protein comprises the sequence as set forth below (SEQ ID NO:19)

GCCACCATGTTCGTCTTTCTCGTGCTGTTGCCTCTTGTATCTTCTCAGTGC
GTTAACCTGACAACACGCACCCAGTTGCCACCGGCCTATACGAACTCTTTC
ACGCGCGGAGTGTATTATCCCGATAAAGTCTTTCGGAGTTCTGTGCTGCAC
TCTACACAAGATCTTTTTCTGCCCTTTTTCTCCAACGTTACGTGGTTCCAT
GCAATAAGTGGTACGAATGGTACTAAAAGGTTCGATAATCCCGTACTTCCC
TTCAATGACGGCGTGTATTTCGCGAGCACAGAAAAGAGCAACATAATAAGA
GGTTGGATTTTTGGGACTACCCTGGACTCTAAAACACAAAGCCTCCTCATT
GTTAATAACGCAACTAATGTCGTAATAAAGGTATGCGAATTTCAGTTTTGT
AACGACCCTTTTTTGGGAGTCTATCATAAGAACAATAAGTCCTGGATGGAA
AGTGAATTTAGGGTGTATAGTTCAGCGAACAACTGTACATTTGAGTATGTG
TCACAGCCGTTCCTTATGGATCTTGAAGGGAAACAGGGGAACTTCAAGAAC
CTGCGGGAATTCGTGTTTAAAAATATAGACGGTTATTTTAAAATCTATAGC
AAACACACCCCAATAAACCTGGTTCGGGATCTTCCTCAGGGATTTTCAGCT
TTGGAACCGTTGGTTGACCTCCCCATCGGGATAAATATCACGCGATTTCAA
ACACTGCTGGCACTGCACCGGAGTTATCTGACTCCTGGAGATAGCAGCTCC
GGCTGGACGGCTGGCGCCGCGGCTTATTATGTCGGGTACCTTCAACCGCGA
ACGTTTCTTTTGAAGTATAACGAAAATGGCACTATAACCGATGCAGTAGAT
TGTGCGTTGGACCCTCTTTCCGAGACTAAGTGCACCCTGAAGTCATTTACA
GTAGAGAAGGGGATATACCAGACAAGCAACTTCCGCGTCCAACCAACGGAA
AGCATCGTGCGCTTTCCCAATATTACTAACCTCTGTCCGTTCGGCGAGGTT
TTTAATGCTACCAGATTTGCCTCAGTGTATGCCTGGAACCGCAAGAGGATT
AGCAACTGCGTTGCTGACTATAGCGTGCTTTACAATTCCGCCAGTTTTAGT
ACGTTCAAGTGTTATGGGGTAAGCCCTACCAAGCTTAACGATCTTTGCTTC
ACGAATGTTTACGCAGACTCATTCGTAATACGAGGTGATGAAGTGCGACAG
ATAGCCCCGGGTCAGACGGGGAAAATAGCCGACTACAATTACAAGCTCCCG
GACGACTTCACGGGCTGTGTTATTGCGTGGAACAGCAATAACCTGGACAGT
AAAGTTGGAGGGAATTATAACTACTTGTACAGGCTGTTCCGCAAATCTAAT
TTGAAACCGTTTGAAAGGGACATTTCCACCGAGATTTACCAAGCCGGCAGT
ACCCCATGTAACGGAGTTGAGGGATTTAATTGCTACTTTCCGCTCCAATCA
TATGGATTCCAGCCTACATACGGCGTCGGATATCAACCTTATAGGGTGGTC
GTTCTTTCTTTTGAGCTGCTTCACGCCCCAGCAACCGTTTGCGGCCCGAAA
AAATCAACGAACTTGGTCAAAAACAAGTGCGTCAACTTCAATTTCAACGGA
CTGACAGGCACCGGAGTGCTCACCGAGTCAAATAAAAAATTCCTCCCGTTT
CAACAATTCGGTAGAGATATAGACGACACCACAGATGCGGTACGCGACCCT
CAGACTCTTGAAATTTTGGACATTACACCATGTTCCTTCGGTGGCGTCTCA
GTGATTACGCCTGGCACTAACACTAGCAACCAAGTCGCAGTACTTTATCAG
GGCGTTAACTGTACAGAAGTACCAGTTGCGATACACGCGGACCAGCTCACC
CCAACCTGGCGCGTATATTCTACCGGATCTAATGTGTTTCAAACGCGGGCG
GGGTGCCTCATAGGTGCAGAGCATGTCAATAACTCCTACGAATGTGACATT
CCTATAGGAGCAGGAATATGTGCGAGCTACCAGACTCAAACCAATTCACAC
AGGAGCGTAGCTAGTCAGTCCATAATTGCCTACACTATGTCACTTGGTGCT
GAAAACAGTGTGGCTTATTCAAATAATTCCATAGCTATTCCAATCAATTTC
ACTATTAGCGTAACAACTGAGATACTTCCAGTCAGCATGACTAAGACTTCA
GTTGATTGTACGATGTATATTTGTGGCGATTCTACAGAATGTAGTAACCTT
CTGTTGCAATACGGCTCCTTTTGCACACAGCTCAATCGCGCATTGACGGGT
ATAGCTGTAGAGCAAGATAAAAACACCCAAGAAGTCTTCGCGCAGGTGAAG
CAAATATATAAGACACCACCGATTAAAGATTTCGGTGGATTCAATTTTAGC
CAAATTCTGCCTGATCCAAGCAAACCGTCAAAAAGATCCTTCATAGAGGAC
CTGCTTTTCAATAAGGTAACGCTGGCGGACGCAGGGTTTATTAAACAATAC
GGCGATTGCCTTGGTGACATTGCTGCACGAGACCTCATTTGCGCCCAGAAG
TTCAACGGACTCACAGTACTGCCTCCACTGCTCACAGACGAGATGATCGCC
CAGTACACATCTGCCTTGTTGGCTGGTACGATAACTTCTGGTTGGACCTTT
GGAGCGGGAGCAGCACTTCAGATTCCCTTTGCCATGCAGATGGCGTACAGA
TTCAACGGCATTGGTGTCACGCAGAACGTACTGTATGAGAACCAGAAGTTG
ATAGCTAATCAATTCAACTCCGCGATAGGTAAGATCCAAGACAGTCTCTCC
TCAACAGCCTCCGCACTCGGGAAACTCCAGGATGTGGTAAATCAGAATGCA
CAGGCATTGAATACGCTTGTTAAACAATTGTCCTCAAATTTTGGCGCCATA
TCATCAGTGCTCAACGACATCCTTGCCAGACTCGATAAAGTGGAGGCAGAA
GTGCAGATAGATCGCTTGATTACTGGGCGCCTCCAATCTCTCCAAACGTAC
GTGACACAACAGCTTATTCGGGCAGCGGAAATCCGCGCCTCTGCGAACCTT
GCAGCGACCAAGATGAGCGAATGTGTTCTCGGGCAGTCAAAACGAGTAGAT
TTCTGCGGTAAAGGATATCATCTTATGTCATTCCCCCAAAGCGCTCCACAT
GGTGTTGTATTTCTGCACGTGACTTACGTGCCGGCACAGGAGAAGAATTTC
ACAACGGCTCCCGCCATCTGTCACGACGGAAAGGCACACTTCCCTAGGGAG
GGGGTGTTTGTAAGCAATGGTACCCACTGGTTTGTCACACAACGAAACTTC
TATGAACCTCAGATAATTACAACGCATAATACTTTTGTAAGTGGGAACTGC
GACGTCGTTATCGGAATAGTTAACAATACTGTATATGATCCGTTGCAGCCG
GAGTTGGACTCATTTAAAGAAGAGCTGGACAAGTACTTCAAAAACCATACA
AGCCCAGATGTAGATCTTGGGGACATAAGTGGGATAAACGCCAGCGTTGTG
AACATACAGAAGGAAATAGACAGGCTGAATGAGGTAGCAAAGAATCTTAAC
GAAAGCCTTATAGATCTTCAGGAACTTGGGAAGTACGAACAGTACATAAAG
TGGCCTTGGTATATTTGGCTCGGTTTCATCGCTGGACTCATAGCGATAGTA
ATGGTGACTATCATGCTGTGTTGTATGACCAGTTGCTGCTCATGCCTGAAG
GGTTGCTGCTCTTGCGGCTCCTGCTGTAAATTCGACGAAGATGACTCCGAG
CCAGTGCTGAAGGGGGTGAAGCTGCATTATACTTCACGACTGGAGGAAGAA
CTGCGCCGACGCCTGACTGAATGA

In certain embodiments, the sequence encoding the spike protein comprises the sequence as set forth below (SEQ ID NO:20)

GCCACCATGTTCGTATTCCTTGTTCTTTTGCCGCTTGTGTCTTCTCAGTGT
GTCAATTTCACTACACGCACACAGCTGCCCCCGGCGTACACTAACAGTTTT
ACGAGGGGAGTCTACTACCCGGACAAAGTGTTCAGGTCTTCCGTACTCCAT
AGCACACAAGACCTCTTCTTGCCTTTTTTCAGCAATGTTACGTGGTTCCAT
GCAATCCATGTGAGCGGTACAAACGGGACTAAACGCTTTGCAAATCCAGTG
TTGCCCTTCAACGACGGTGTATACTTCGCGAGTACAGAGAAGAGCAACATA
ATACGCGGGTGGATATTTGGAACGACGCTTGATTCTAAGACACAGAGCTTG
CTCATCGTAAACAACGCAACGAATGTTGTAATCAAGGTCTGCGAATTCCAA
TTCTGCAATGACCCATTTCTTGGAGTTTATTACCATAAGAACAATAAGTCT
TGGATGGAATCCGAGTTCCGCGTATACTCTAGCGCGAACAACTGCACCTTC
GAGTATGTGTCCCAACCGTTTCTGATGGATCTTGAGGGAAAGCAGGGTAAT
TTTAAAAATCTCCGGGAGTTCGTTTTCAAAAATATTGACGGATATTTCAAA
ATCTACAGCAAGCATACCCCAATCAATTTGGTGCGGGGTCTGCCACAAGGA
TTTTCCGCTCTTGAACCTCTTGTTGACCTTCCCATAGGAATCAATATAACT
CGGTTCCAAACTCTTCTTGCGCTCCATATATCCTACTTGACGCCCGGAGAT
TCATCAAGTGGCTGGACGGCTGGGGCTGCCGCATACTACGTCGGCTATCTG
CAACCAAGGACGTTTCTCCTCAAATACAATGAAAATGGTACGATCACCGAC
GCTGTGGACTGCGCTCTCGACCCGTTGTCAGAGACGAAGTGCACCTTGAAG
TCCTTCACCGTGGAAAAGGGAATTTACCAGACTAGTAACTTCCGGGTGCAG
CCCACGGAATCAATTGTGCGATTTCCTAATATCACTAATCTCTGTCCCTTC
GGGGAAGTGTTCAATGCGACTCGATTCGCTTCAGTATACGCTTGGAATAGA
AAGAGAATCTCAAATTGCGTGGCTGACTATTCCGTACTGTATAATAGTGCG
TCTTTTAGCACGTTTAAGTGTTATGGCGTATCCCCGACAAAGCTCAACGAT
CTCTGCTTCACGAATGTCTATGCCGACTCCTTCGTGATACGCGGTGACGAG
GTACGACAAATTGCACCCGGCCAAACTGGAAATATTGCCGACTACAATTAT
AAGTTGCCGGACGATTTTACCGGTTGCGTAATAGCGTGGAACTCTAATAAC
CTCGACTCTAAGGTAGGAGGCAACTACAATTACTTGTACCGACTGTTCAGG
AAATCCAACCTGAAGCCCTTCGAAAGAGACATCAGTACAGAGATTTATCAA
GCCGGGTCCACCCCTTGCAATGGTGTCAAAGGCTTCAACTGCTATTTTCCG
TTGCAGTCTTACGGTTTCCAGCCGACGTACGGTGTGGGTTATCAACCCTAC
CGCGTTGTGGTGCTGAGCTTTGAGCTTCTCCACGCTCCAGCCACTGTCTGC
GGGCCTAAGAAATCCACAAACCTCGTTAAGAATAAATGCGTAAATTTCAAC
TTCAACGGGCTTACTGGCACGGGCGTGCTTACGGAGTCCAATAAGAAATTC
CTGCCTTTCCAGCAGTTCGGCCGCGACATCGCGGACACAACAGATGCTGTG
CGAGACCCACAAACCCTGGAGATTCTGGACATCACGCCTTGCTCTTTTGGC
GGTGTCAGCGTAATAACGCCAGGCACCAACACCAGTAATCAGGTCGCCGTG
CTTTATCAGGGTGTGAACTGTACGGAGGTACCGGTTGCCATACACGCAGAC
CAACTCACACCTACGTGGAGAGTTTACAGCACCGGGTCAAACGTATTTCAG
ACCCGCGCAGGCTGTCTGATCGGCGCAGAGCATGTCAATAACTCATACGAG
TGTGATATACCAATCGGAGCCGGCATCTGTGCGAGCTATCAAACCCAAACT
AATTCACGAAGCGTGGCCAGCCAGTCCATCATTGCCTACACGATGTCTTTG
GGAGTAGAGAATAGCGTGGCATACTCTAACAACAGTATCGCTATTCCTACC
AACTTCACAATATCAGTTACTACAGAAATCCTCCCGGTGAGCATGACTAAG
ACATCCGTAGACTGTACAATGTACATTTGTGGGGACAGCACGGAGTGTTCA
AACCTGCTGTTGCAATACGGAAGCTTCTGCACCCAGCTCAACCGAGCCCTG
ACCGGCATTGCCGTAGAACAAGATAAGAATACGCAGGAAGTGTTCGCCCAG
GTTAAGCAGATTTACAAGACTCCGCCTATCAAGGACTTCGGGGGTTTCAAC
TTTAGTCAGATTTTGCCTGACCCGTCAAAGCCTAGCAAACGAAGTTTTATC
GAAGATTTGTTGTTTAACAAGGTGACTTTGGCTGATGCGGGATTCATAAAA
CAATACGGCGATTGCCTTGGCGATATTGCGGCCCGAGATCTCATTTGCGCG
CAAAAGTTCAACGGTCTTACCGTCCTTCCGCCCCTGCTTACGGACGAGATG
ATCGCACAATACACTTCAGCACTGCTGGCTGGTACAATAACCAGTGGGTGG
ACATTCGGGGCTGGTGCAGCGCTCCAGATCCCCTTTGCGATGCAGATGGCG
TATAGATTCAATGGCATCGGGGTCACTCAGAACGTGCTTTATGAGAACCAA
AAGCTTATTGCAAATCAATTTAACTCTGCGATTGGAAAGATTCAAGACAGC
CTCTCATCCACAGCTAGTGCGCTTGGCAAGCTTCAGGACGTCGTAAACCAA
AACGCCCAAGCTCTCAACACACTCGTTAAACAGCTTTCCTCAAACTTTGGG
GCTATTAGCTCTGTGCTGAATGATATTCTTAGCAGACTCGACAAAGTCGAG
GCTGAAGTACAAATAGATAGACTGATAACAGGTAGACTTCAGTCTCTCCAA
ACCTACGTGACACAGCAGCTTATAAGAGCTGCTGAGATCAGAGCTTCTGCG
AACCTTGCGGCGACTAAAATGTCCGAGTGTGTACTCGGGCAGTCTAAACGC
GTCGACTTCTGTGGCAAGGGTTACCATCTCATGAGTTTTCCCCAATCAGCG
CCCCATGGGGTAGTTTTTCTGCACGTAACATACGTCCCAGCCCAAGAAAAG
AATTTTACCACCGCCCCTGCGATTTGTCACGACGGAAAAGCACACTTTCCA
AGAGAAGGCGTATTTGTTAGCAATGGCACCCATTGGTTCGTCACCCAGAGA
AACTTTTACGAACCGCAGATTATCACGACAGATAATACATTTGTATCTGGT
AATTGTGACGTCGTGATCGGCATCGTAAACAACACTGTTTACGACCCCCTC
CAGCCAGAGCTTGACTCATTTAAAGAGGAGCTCGACAAATATTTTAAGAAC
CACACAAGTCCAGACGTCGACTTGGGTGACATTTCCGGCATCAATGCAAGT
GTGGTCAACATCCAAAAGGAAATTGACAGATTGAACGAGGTTGCTAAGAAC
CTTAATGAGTCACTCATTGACCTCCAGGAACTGGGAAAATACGAACAGTAT
ATTAAGTGGCCCTGGTACATCTGGCTGGGATTTATCGCGGGACTTATCGCG
ATTGTTATGGTGACGATAATGCTTTGCTGCATGACCAGCTGTTGTAGTTGC
CTCAAGGGTTGCTGCTCATGCGGGAGCTGCTGTAAATTCGACGAGGACGAT
TCTGAGCCCGTTTTGAAGGGAGTCAAGCTTCACTACACGTCACGACTGGAG
GAAGAACTGCGCCGACGCCTGACTGAATGA

In certain embodiments, the sequence encoding the spike protein comprises the sequence as set forth below (SEQ ID NO:21)

GCCACCATGTTCGTGTTCCTGGTCCTTCTCCCCTTGGTGTCAATCCAGTGT
GTAAATTTGACAACGCGCACCCAATTGCCCCCAGCATATACGAACTCTTTC
ACGAGGGGCGTATATTATCCGGATAAGGTATTTCGGTCATCTGTTCTGCAC
AGCACCCAGGACCTCTTCCTTCCATTCTTTTCAAATGTAACTTGGTTCCAT
GCGATAGTATCAGGAACGAATGGGACAAAAAGGTTCGATAATCCGGTCTTG
CCGTTCAACGATGGGGTGTACTTCGCCAGTACCGAAAAGTCTAACATTATA
CGCGGTTGGATTTTTGGCACTACGCTTGACTCAAAGACACAGTCACTCCTC
ATTGTAAATAATGCTACTAATGTCGTGATCAAAGTTTGTGAGTTCCAATTC
TGCAACGACCCGTTCCTGGGAGTGTATTATCACAAAAATAATAAGTCCTGC
ATGGAGTCAGAGTTCAGGGTTTACTCAAGTGCGAACAATTGCACATTTGAG
TACGTTTCTCAACCATTTCTCATGGATTTGGAGGGGAAGCAGGGAAACTTT
AAAAACCTGAGAGAATTTGTTTTTAAGAACATTGATGGGTATTTCAAGATT
TATAGTAAACACACCCCTATCAACTTGGTTAGGGATCTCCCTCAAGGTTTC
TCTGCTCTCGAGCCCCTTGTAGATCTGCCAATAGGCATCAATATCACACGC
TTTCAGACACTCCTCGCACTTCATAGGAGCTACCTGACGCCAGGTGACTCT
TCCTCAGGTTGGACAGCCGGCGCAGCCGCATACTACGTTGGCTACCTCCAA
CCAAGGACATTTCTGTTGAAATACAACGAAAATGGGACCATCACCGACGCA
GTCGATTGTGCTCTCGACCCTCTTTCCGAGACTAAATGTACCCTCAAAAGC
TTTACTGTTGAGAAGGGTATCTATCAGACATCTAACTTTCGGGTGCAACCC
ACTGAGTCAATTGTGCGATTCCCAAATATTACGAACCTCTGTCCTTTTGGC
GAGGTTTTTAACGCCACTAGGTTCGCCAGTGTATATGCTTGGAACCGAAAA
CGGATAAGCAATTGTGTTGCTGACTACTCCGTCCTCTACAATAGCGCTAGT
TTCTCAACATTTAAGTGTTACGGTGTGAGCCCTACGAAACTTAACGATTTG
TGCTTCACTAACGTCTATGCCGACAGTTTCGTAATCCGAGGCGATGAGGTC
AGGCAAATTGCCCCGGGCCAAACGGGGAAAATCGCTGATTACAATTATAAG
TTGCCAGATGATTTTACGGGATGTGTCATTGCATGGAACAGTAATAACCTC
GATTCAAAGGTTGGCGGAAATTATAATTACAGATATCGGCTTTTTAGAAAA
TCTAACCTTAAACCATTTGAGCGGGACATAAGCACGGAGATTTACCAGGCT
GGTAGCACTCCGTGCAACGGTGTAGAAGGATTCAATTGCTATTTTCCATTG
CAGTCTTATGGATTCCAACCCACCAATGGGGTAGGGTACCAACCATACAGG
GTGGTAGTCCTTAGCTTTGAACTTTTGCATGCGCCAGCTACCGTCTGCGGT
CCCAAAAAGAGTACGAACTTGGTAAAAAATAAATGCGTCAACTTTAATTTT
AACGGTCTGACGGGAACGGGGGTTCTCACCGAGTCTAACAAAAAGTTTTTG
CCATTTCAGCAGTTCGGACGAGATATTGCCGACACTACCGACGCCGTGCGC
GATCCACAAACCTTGGAGATACTCGACATCACACCTTGCAGCTTCGGTGGT
GTTAGCGTTATTACGCCAGGAACGAACACTTCAAATCAGGTGGCCGTCTTG
TATCAAGATGTTAACTGTACAGAGGTGCCCGTCGCAATACACGCAGATCAG
CTCACCCCTACCTGGAGAGTTTACTCTACAGGCTCTAATGTGTTCCAAACT
AGGGCAGGCTGCTTGATTGGAGCTGAACACGTAAACAACTCATACGAGTGC
GATATCCCGATTGGTGCGGGTATTTGCGCCAGCTATCAGACGCAGACTAAC
TCACGATCAGTGGCGTCACAGTCAATAATTGCGTATACAATGAGTCTCGGT
GCAGAGAATTCCGTCGCATACTCAAACAACAGCATAGCGATTCCAACTAAT
TTTACCATTAGCGTTACAACGGAAATCCTCCCCGTTAGCATGACCAAGACC
TCCGTGGATTGCACAATGTACATCTGTGGAGACAGCACCGAGTGCTCCAAT
CTTCTTCTTCAATATGGGAGCTTTTGCACACAGCTGAACCGCGCGCTCACT
GGCATTGCGGTAGAACAAGACAAAAACACCCAAGAAGTGTTTGCGCAGGTC
AAACAGATTTACAAGACACCACCAATCAAAGACTTCGGGGGGTTCAATTTC
TCCCAAATTCTTCCTGATCCAAGCAAACCATCTAAAAGGTCCTTCATAGAA
GACCTCCTGTTCAATAAGGTGACACTCGCGGACGCAGGGTTTATTAAGCAA
TATGGAGATTGTCTCGGGGATATTGCAGCGAGAGATCTTATTTGTGCTCAA
AAGTTTAATGGCTTGACTGTCCTCCCGCCCTTGCTCACAGACGAGATGATA
GCACAATATACCAGTGCGCTGCTTGCGGGCACGATTACTTCCGGATGGACT
TTCGGGGCAGGAGCGGCGCTCCAGATTCCTTTCGCGATGCAAATGGCCTAC
CGATTTAACGGGATTGGCGTCACACAAAACGTTCTTTACGAAAACCAGAAA
CTTATTGCCAACCAATTCAATAGTGCAATTGGTAAAATTCAGGATTCACTC
TCCTCCACGGCGTCAGCTCTCGGCAAGCTCCAAGACGTGGTTAACCAAAAT
GCGCAGGCGCTTAACACGCTTGTAAAACAACTCTCTTCTAATTTTGGCGCG
ATTAGTAGCGTTCTCAACGATATCTTGTCACGCTTGGACAAAGTAGAAGCA
GAAGTCCAAATAGACCGGCTGATCACTGGTCGATTGCAATCTCTGCAGACA
TACGTGACGCAGCAGCTCATCCGGGCAGCCGAGATCCGGGCGTCCGCTAAT
CTGGCCGCAACCAAAATGAGCGAGTGTGTCCTTGGGCAAAGCAAACGCGTG
GATTTTTGCGGAAAGGGCTACCACCTCATGAGTTTCCCCCAATCTGCCCCG
CACGGTGTCGTCTTTTTGCATGTAACGTATGTCCCGGCGCAGGAGAAAAAT
TTCACTACAGCTCCCGCCATCTGTCACGACGGCAAGGCACACTTCCCTCGA
GAAGGGGTGTTTGTAAGCAATGGAACACACTGGTTCGTCACCCAGCGGAAT
TTTTATGAACCTCAGATAATCACAACAGACAACACTTTCGTTTCCGGAAAC
TGCGATGTCGTAATTGGGATAGTGAATAATACCGTATATGACCCATTGCAG
CCGGAGTTGGACTCCTTTAAAGAGGAATTGGACAAATACTTTAAAAATCAC
ACTTCACCCGATGTGGACCTGGGAGATATATCCGGGATTAACGCAAGTGTG
GTTAACATACAAAAGGAAATTGACCGCCTTAACGAGGTTGCTAAGAACTTG
AATGAGTCTCTCATAGATCTCCAGGAACTGGGGAAATACGAGCAATATATC
AAATGGCCGTGGTACATATGGCTCGGATTTATAGCCGGCCTCATAGCAATA
GTTATGGTCACTATAATGTTGTGCTGCATGACGTCCTGTTGTAGTTGCCTC
AAGGGGTGCTGTTCCTGCGGTAGTTGCTGCAAATTTGATGAAGATGACTCC
GAACCGGTTCTCAAGGGAGTCAAGCTTCATTATACCTCACGACTGGAGGAA
GAACTGCGCCGACGCCTGACTGAATGA

In certain embodiments, the sequence encoding the spike protein comprises the sequence as set forth below (SEQ ID NO:22)

GCCACCATGTTTGTGTTCCTGGTCCTTCTCCCGCTTGTAAGCTCCCAATGC
GTTAACTTTACTAATCGGACCCAACTCCCGAGTGCGTACACTAACTCTTTT
ACCCGGGGCGTCTATTATCCTGACAAAGTGTTTCGATCATCTGTTTTGCAC
TCCACCCAAGATCTCTTTCTTCCATTTTTCAGCAACGTTACGTGGTTCCAC
GCGATACATGTTAGTGGTACTAATGGCACAAAGAGATTTGACAACCCTGTG
CTCCCGTTTAACGATGGGGTCTATTTCGCTTCCACTGAGAAGTCCAACATT
ATTCGCGGTTGGATCTTCGGCACCACACTCGATTCAAAAACCCAAAGCTTG
CTCATAGTTAACAACGCCACAAACGTAGTCATTAAAGTGTGCGAATTTCAG
TTTTGTAACTATCCCTTTTTGGGCGTCTACTATCACAAGAACAACAAGTCC
TGGATGGAATCTGAGTTTCGGGTCTACAGTTCAGCCAACAACTGTACGTTT
GAGTACGTTAGCCAGCCCTTTCTGATGGATCTTGAAGGGAAGCAAGGAAAT
TTCAAGAATCTTTCCGAGTTTGTGTTTAAAAATATAGACGGATATTTCAAG
ATATACAGTAAGCACACACCGATCAATTTGGTACGGGATTTGCCGCAAGGA
TTTTCCGCCCTTGAGCCCCTTGTAGATCTCCCCATCGGAATTAACATAACC
AGGTTTCAAACACTGCTGGCGCTCCACAGGAGCTATCTGACCCCGGGCGAC
TCATCCAGTGGGTGGACCGCCGGAGCAGCCGCATACTACGTGGGGTATCTC
CAGCCCAGAACGTTTCTCCTCAAGTATAATGAAAACGGGACCATCACCGAC
GCGGTTGATTGTGCACTCGATCCCCTCAGCGAGACAAAGTGCACACTTAAA
TCATTTACCGTCGAGAAGGGGATCTACCAAACATCCAACTTTCGGGTGCAG
CCTACCGAGTCAATAGTACGGTTTCCTAACATCACCAATTTGTGCCCTTTT
GGGGAAGTATTTAATGCAACCCGGTTTGCGAGCGTATACGCTTGGAACCGA
AAACGGATAAGTAATTGTGTTGCAGATTATTCTGTGCTCTATAACTCAGCT
TCCTTCTCCACCTTTAAGTGTTACGGAGTATCTCCGACAAAATTGAATGAT
CTTTGTTTTACGAATGTTTACGCTGACTCATTTGTTATCCGAGGTGACGAA
GTGCGCCAGATAGCCCCCGGTCAAACAGGCAACATTGCTGATTATAACTAC
AAGTTGCCCGATGACTTCACGGGGTGTGTTATTGCATGGAATTCAAATAAT
CTTGATAGTAAGGTAGGGGGTAACTATAATTACCTTTATCGCTTGTTCCGC
AAATCCAATCTGAAACCCTTCGAAAGGGATATCAGTACCGAGATTTATCAA
GCCGGCTCCACACCTTGTAACGGAGTAAAAGGTTTTAACTGTTATTTCCCA
CTGCAGAGTTACGGCTTTCAACCCACATACGGGGTGGGATATCAGCCCTAT
CGCGTTGTGGTCTTGTCCTTTGAACTTCTCCACGCCCCTGCAACAGTTTGT
GGTCCTAAAAAGAGTACAAATCTGGTAAAAAATAAGTGCGTCAACTTTAAC
TTCAACGGACTGACTGGTACCGGGGTCCTCACTGAGAGCAACAAGAAATTT
TTGCCCTTCCAGCAATTCGGGAGGGATATTGCCGATACTACCGATGCTGTA
CGGGACCCCCAGACACTCGAAATATTGGACATAACCCCGTGCTCTTTTGGA
GGGGTCTCTGTGATTACCCCGGGTACGAACACGAGTAATCAGGTGGCAGTG
CTTTATCAGGGAGTGAATTGCACAGAAGTACCAGTCGCGATCCATGCCGAC
CAGCTCACGCCGACCTGGAGGGTTTATAGTACGGGTAGTAACGTTTTCCAA
ACACGGGCCGGTTGTCTCATCGGGGCAGAGTACGTTAATAACAGCTATGAA
TGTGATATACCCATAGGTGCCGGGATTTGTGCTTCTTATCAAACCCAAACC
AATAGCAGAAGCGTTGCGTCACAGTCTATAATCGCCTATACGATGAGCCTC
GGAGCGGAAAACTCAGTTGCCTACTCCAACAATAGCATTGCTATCCCAACG
AACTTCACTATTAGCGTAACCACGGAGATCCTTCCAGTTAGCATGACAAAA
ACTAGCGTCGATTGCACTATGTATATTTGCGGCGACAGTACAGAGTGTAGT
AATTTGCTCCTGCAGTACGGGTCTTTCTGCACCCAATTGAACCGAGCACTG
ACTGGCATCGCCGTGGAACAAGACAAAAACACGCAAGAAGTATTTGCGCAA
GTCAAGCAGATTTACAAGACTCCCCCGATCAAAGACTTTGGCGGATTTAAT
TTTAGCCAGATACTCCCCGATCCATCTAAACCATCAAAACGGTCCTTTATA
GAAGATCTCTTGTTCAATAAAGTGACACTCGCGGACGCGGGCTTTATCAAA
CAGTATGGGGACTGCCTTGGAGATATAGCTGCGAGGGACTTGATTTGCGCC
CAGAAATTCAACGGGCTGACGGTTCTGCCGCCGCTCTTGACAGACGAAATG
ATTGCACAGTATACGAGTGCCTTGCTGGCAGGGACTATCACTTCCGGTTGG
ACCTTCGGCGCAGGAGCAGCGCTCCAGATCCCATTCGCTATGCAAATGGCT
TACCGATTCAACGGAATTGGCGTCACACAGAACGTGCTTTACGAGAATCAA
AAGCTGATTGCCAATCAATTCAACTCAGCGATTGGCAAAATACAAGATAGT
CTCTCTTCAACTGCATCCGCGCTCGGTAAGTTGCAGGACGTAGTCAATCAG
AACGCACAGGCCCTGAACACGCTTGTTAAACAACTGTCTTCAAACTTCGGT
GCAATAAGTTCCGTACTTAATGATATCTTGTCAAGGTTGGATAAGGTCGAA
GCTGAGGTACAAATCGACCGCCTTATAACAGGCAGACTCCAATCCCTGCAG
ACATACGTGACCCAGCAACTCATTAGGGCAGCCGAGATCAGAGCAAGTGCC
AACCTCGCTGCTATTAAAATGTCTGAATGCGTTTTGGGGCAATCTAAGCGG
GTTGATTTCTGCGGTAAGGGATACCACTTGATGTCCTTTCCTCAGTCCGCT
CCTCATGGGGTAGTATTTCTTCATGTTACATATGTACCAGCACAAGAGAAA
AATTTTACTACTGCCCCGGCCATATGCCATGATGGCAAAGCGCACTTCCCT
CGGGAGGGGGTTTTTGTGTCTAATGGAACCCACTGGTTCGTGACTCAGCGG
AATTTCTATGAACCCCAGATTATAACTACTGACAACACTTTCGTTTCTGGA
AACTGCGATGTAGTAATAGGCATAGTAAATAATACAGTATATGATCCCTTG
CAACCAGAATTGGACTCTTTTAAAGAAGAGCTCGACAAATATTTTAAAAAC
CATACATCTCCAGACGTTGATCTTGGAGACATCTCTGGGATAAACGCTAGC
TTCGTAAACATACAAAAAGAAATCGATAGGCTTAACGAGGTCGCAAAAAAT
CTTAATGAAAGTCTTATCGATTTGCAAGAGCTTGGAAAGTATGAGCAGTAT
ATCAAATGGCCTTGGTATATATGGCTGGGATTTATTGCTGGTCTCATAGCC
ATAGTTATGGTGACAATAATGCTGTGCTGTATGACGTCATGTTGTTCCTGC
CTGAAAGGGTGCTGTAGTTGTGGGAGTTGTTGCAAGTTTGACGAGGACGAC
TCAGAGCCCGTTTTGAAAGGTGTGAAACTTCATTATACATCACGACTGGAG
GAAGAACTGCGCCGACGCCTGACTGAATGA

In certain embodiments, the sequence encoding the spike protein comprises the sequence as set forth below (SEQ ID NO:23)

CCACCATGTTCGTATTTCTCGTCCTCCTCCCACTTGTTTCTAGTCAGTGTG
TTAATCTTACAACCAGAACTCAATTACCCCCTGCATACACTAATTCTTTCA
CACGTGGTGTTTATTACCCTGACAAAGTTTTCAGATCCTCAGTTTTACATT
CAACTCAGGACTTGTTCTTACCTTTCTTTTCCAATGTTACTTGGTTCCATG
CTATACATGTCTCTGGGACCAATGGTACTAAGAGGTTTGATAACCCTGTCC
TACCATTTAATGATGGTGTTTATTTTGCTTCCATTGAGAAGTCTAACATAA
TAAGAGGCTGGATTTTTGGTACTACTTTAGATTCGAAGACCCAGTCCCTAC
TTATTGTTAATAACGCTACTAATGTTGTTATTAAAGTCTGTGAATTTCAAT
TTTGTAATGATCCATTTTTGGGTGTTTATTACCACAAAAACAACAAAAGTT
GGATGAAAAGTGAGTTCAGAGTTTATTCTAGTGCGAATAATTGCACTTTTG
AATATGTCTCTCAGCCTTTTCTTATGGACCTTGAAGGAAAACAGGGTAATT
TCAAAAATCTTAGGGAATTTGTGTTTAAGAATATTGATGGTTATTTTAAAA
TATATTCTAAGCACACGCCTATTAATTTAGTGCGTGATCTCCCTCAGGGTT
TTTCGGCTTTAGAACCATTGGTAGATTTGCCAATAGGTATTAACATCACTA
GGTTTCAAACTTTACTTGCTTTACATAGAAGTTATTTGACTCCTGGTGATT
CTTCTTCAGGTTGGACAGCTGGTGCTGCAGCTTATTATGTGGGTTATCTTC
AACCTAGGACTTTTCTATTAAAATATAATGAAAATGGAACCATTACAGATG
CTGTAGACTGTGCACTTGACCCTCTCTCAGAAACAAAGTGTACGTTGAAAT
CCTTCACTGTAGAAAAAGGAATCTATCAAACTTCTAACTTTAGAGTCCAAC
CAACAGAATCTATTGTTAGATTTCCTAATATTACAAACTTGTGCCCTTTTG
GTGAAGTTTTTAACGCCACCAGATTTGCATCTGTTTATGCTTGGAACAGGA
AGAGAATCAGCAACTGTGTTGCTGATTATTCTGTCCTATATAATTCCGCAT
CATTTTCCACTTTTAAGTGTTATGGAGTGTCTCCTACTAAATTAAATGATC
TCTGCTTTACTAATGTCTATGCAGATTCATTTGTAATTAGAGGTGATGAAG
TCAGACAAATCGCTCCAGGGCAAACTGGAAAGATTGCTGATTATAATTATA
AATTACCAGATGATTTTACAGGCTGCGTTATAGCTTGGAATTCTAACAAAC
TTGATTCTAAGGTTGGTGGTAATTATAATTACCGCTATAGATTGTTTAGGA
AGTCTAATCTCAAACCTTTTGAGAGAGATATTTCAACTGAAATCTATCAGG
CCGGTAGCACACCTTGTAATGGTGTTCAGGGTTTTAATTGTTACTTTCCTT
TACAATCATATGGTTTCCAACCCACTTATGGTGTTGGTTACCAACCATACA
GAGTAGTAGTACTTTCTTTTGAACTTCTACATGCACCAGCAACTGTTTGTG
GACCTAAAAAGTCTACTAATTTGGTTAAAAACAAATGTGTCAATTTCAACT
TCAATGGTTTAACAGGCACAGGTGTTCTTACTGAGTCTAACAAAAAGTTTC
TGCCTTTCCAACAATTTGGCAGAGACATTGATGACACTACTGATGCTGTCC
GTGATCCACAGACACTTGAGATTCTTGACATTACACCATGTTCTTTTGGTG
GTGTCAGTGTTATAACACCAGGAACAAATACTTCTAACCAGGTTGCTGTTC
TTTATCAGGGTGTTAACTGCACAGAAGTCCCTGTTGCTATTCATGCAGATC
AACTTACTCCTACTTGGCGTGTTTATTCTACAGGTTCTAATGTTTTTCAAA
CACGTGCAGGCTGTTTAATAGGGGCTGAACATGTCAACAACTCATATGAGT
GTGACATACCCATTGGTGCAGGTATATGCGCTAGTTATCAGACTCAGACTA
ATTCTAGTGTAGCTAGTCAATCCATCATTGCCTACACTATGTCACTTGGTG
CAGAAAATTCAGTTGCTTACTCTAATAACTCTATTGCCATACCCATTAATT
TTACTATTAGTGTTACCACAGAAATTCTACCAGTGTCTATGACCAAGACAT
CAGTAGATTGTACAATGTACATTTGTGGTGATTCAACTGAATGCAGCAATC
TTTTGTTGCAATATGGCAGTTTTTGTACACAATTAAACCGTGCTTTAACTG
GAATAGCTGTTGAACAAGACAAAAACACCCAAGAAGTTTTTGCACAAGTCA
AACAAATTTACAAAACACCACCAATTAAAGATTTTGGTGGTTTTAATTTTT
CACAAATATTACCAGATCCATCAAAACCAAGCAAGAGGTCATTTATTGAAG
ATCTACTTTTCAACAAAGTGACACTTGCAGATGCTGGCTTCATCAAACAAT
ATGGTGATTGCCTTGGTGATATTGCTGCTAGAGACCTCATTTGTGCACAAA
AGTTTAACGGCCTTACTGTTTTGCCACCTTTGCTCACAGATGAAATGATTG
CTCAATACACTTCTGCACTGTTAGCGGGTACAATCACTTCTGGTTGGACCT
TTGGTGCAGGTGCTGCATTACAAATACCATTTGCTATGCAAATGGCTTATA
GGTTTAATGGTATTGGAGTTACACAGAATGTTCTCTATGAGAACCAAAAAT
TGATTGCCAACCAATTTAATAGTGCTATTGGCAAAATTCAAGACTCACTTT
CTTCCACAGCAAGTGCACTTGGAAAACTTCAAGATGTGGTCAACCAAAATG
CACAAGCTTTAAACACGCTTGTTAAACAACTTAGCTCCAATTTTGGTGCAA
TTTCAAGTGTTTTAAATGATATCCTTGCACGTCTTGACAAAGTTGAGGCTG
AAGTGCAAATTGATAGGTTGATCACAGGCAGACTTCAAAGTTTGCAGACAT
ATGTGACTCAACAATTAATTAGAGCTGCAGAAATCAGAGCTTCTGCTAATC
TTGCTGCTACTAAAATGTCAGAGTGTGTACTTGGACAATCAAAAAGAGTTG
ATTTTTGTGGAAAGGGCTATCATCTTATGTCCTTCCCTCAGTCAGCACCTC
ATGGTGTAGTCTTCTTGCATGTGACTTATGTCCCTGCACACGAAAAGAACT
TCACAACTGCTCCTGCCATTTGTCATGATGGAAAAGCACACTTTCCTCGTG
AAGGTGTCTTTGTTTCAAATGGCACACACTGGTTTGTAACACAAAGGAATT
TTTATGAACCACAAATCATTACTACACACAACACATTTGTGTCTGGTAACT
GTGATGTTGTAATAGGAATTGTCAACAACACAGTTTATGATCCTTTGCAAC
CTGAATTAGACTCATTCAAGGAGGAGTTAGATAAATATTTTAAGAATCATA
CATCACCAGATGTTGATTTAGGTGACATCTCTGGCATTAATGCTTCAGTTG
TAAACATTCAAAAAGAAATTGACCGCCTCAATGAGGTTGCCAAGAATTTAA
ATGAATCTCTCATCGATCTCCAAGAACTTGGAAAGTATGAGCAGTATATAA
AATGGCCATGGTACATCTGGCTGGGTTTTATAGCTGGCCTGATCGCAATTG
TAATGGTAACTATAATGTTGTGTTGCATGACCTCTTGCTGCAGCTGTTTGA
AGGGATGCTGTTCTTGCGGGAGTTGCTGTAAATTTGATGAGGATGACAGCG
AGCCGGTGTTGAAAGGAGTGAAGCTTCATTATACTTCACGACTGGAGGAAG
AACTGCGCCGACGCCTGACTGAATAA

In certain embodiments, the sequence encoding the spike protein comprises the sequence as set forth below (SEQ ID NO:24)

GCCGCCACCATGGTGAACCTGACCACCCGGACCCAGCTACCTCCTGCCTAC
ACAAACAGCTTCACCCGCGGCGTGTACTACCCTGATAAGGTGTTCCGGAGC
TCTGTGCTCCACAGCACACAGGACCTGTTCCTGCCTTTCTTCAGCAACGTG
ACGTGGTTCCACGCCATCCACGTGTCCGGCACCAATGGTACAAAAAGGTTT
GACAACCCCGTGCTCCCCTTCAACGACGGCGTGTATTTTGCCAGCACTGAA
AAGTCCAATATCATCCGAGGATGGATCTTCGGCACAACGCTGGACTCTAAG
ACCCAGAGCCTGCTGATCGTGAACAACGCCACCAACGTGGTGATCAAGGTG
TGCGAGTTTCAGTTCTGCAACGATCCTTTTCTGGGCGTTTACTACCACAAG
AACAACAAGAGCTGGATGGAAAGCGAGTTCAGAGTGTATTCTAGCGCCAAC
AACTGTACCTTCGAATACGTCAGCCAGCCTTTCCTGATGGACCTGGAAGGC
AAGCAGGGCAACTTCAAGAACCTGAGAGAGTTCGTGTTCAAGAACATCGAC
GGCTACTTCAAGATCTACAGCAAGCACACACCTATCAACCTGGTGCGGGAC
CTTCCGCAGGGATTCTCCGCGCTGGAACCCCTGGTGGACCTGCCTATTGGC
ATCAACATCACCAGATTTCAGACCCTGCTGGCCCTGCACAGAAGCTACCTG
ACACCTGGCGACTCTTCTAGCGGCTGGACCGCCGGCGCCGCCGCTTATTAC
GTGGGGTACCTGCAGCCCAGAACCTTCCTGCTGAAATACAATGAGAACGGA
ACCATCACCGATGCCGTGGACTGCGCCCTGGACCCCCTGTCTGAAACCAAG
TGCACCCTGAAATCTTTTACAGTGGAAAAGGGAATTTACCAGACCTCCAAC
TTTAGAGTCCAGCCTACCGAAAGCATCGTGCGGTTCCCTAACATCACCAAC
CTGTGTCCTTTCGGCGAGGTTTTCAACGCCACACGGTTCGCCAGCGTATAC
GCCTGGAACCGGAAGCGGATCTCTAACTGTGTGGCAGACTACTCTGTGCTA
TACAACTCGGCCAGCTTTAGCACCTTCAAGTGCTACGGCGTGTCCCCAACC
AAGCTGAACGACCTGTGCTTCACCAACGTGTACGCCGATAGCTTCGTGATC
AGAGGCGATGAGGTGCGCCAGATCGCTCCTGGACAGACCGGAAAGATCGCT
GACTACAATTACAAGCTGCCCGACGACTTCACCGGCTGCGTGATCGCCTGG
AACAGCAACAACCTGGATTCCAAAGTGGGCGGCAACTACAACTATCTGTAC
CGGCTCTTCAGAAAGTCTAACCTGAAGCCCTTTGAGAGAGACATCTCCACA
GAAATCTACCAGGCCGGAAGCACACCTTGCAACGGCGTGGAAGGATTTAAT
TGCTATTTTCCTCTGCAGTCCTACGGCTTCCAACCTACCAACGGAGTGGGC
TACCAGCCTTACAGAGTGGTCGTGCTGAGCTTTGAGCTGCTGCATGCCCCT
GCTACCGTGTGTGGCCCTAAGAAAAGCACGAACCTGGTCAAGAACAAATGC
GTGAACTTCAACTTCAATGGACTGACAGGCACCGGCGTGCTCACAGAGAGC
AACAAGAAGTTCCTGCCATTTCAGCAGTTCGGCAGAGACATAGCTGACACC
ACCGACGCCGTGCGGGACCCCCAGACCCTGGAAATCCTGGATATCACACCC
TGCAGCTTTGGAGGCGTATCTGTGATCACACCTGGCACCAACACAAGCAAT
CAGGTCGCCGTGCTGTACCAAGACGTGAACTGCACAGAGGTGCCGGTGGCC
ATCCACGCCGATCAGCTGACCCCCACCTGGCGGGTGTACTCCACCGGCAGC
AACGTGTTCCAAACCCGGGCCGGCTGCCTGATCGGCGCCGAACATGTGAAT
AACAGCTACGAATGTGATATCCCTATTGGAGCCGGAATTTGCGCCAGCTAC
CAAACCCAAACCAACTCTCCAAGAAGAGCCAGAAGCGTGGCTAGCCAGTCT
ATCATCGCCTACACCATGAGCCTGGGCGCTGAAAACAGCGTGGCTTACAGC
AACAACAGCATCGCCATTCCTACTAACTTTACCATCAGCGTGACTACAGAG
ATCCTGCCCGTGAGCATGACCAAGACCAGCGTGGACTGCACCATGTACATC
TGTGGCGATTCTACCGAGTGCAGCAACTTGCTACTGCAGTATGGTAGCTTT
TGCACCCAACTGAACAGAGCCCTGACTGGCATCGCCGTTGAGCAGGACAAG
AACACCCAGGAGGTGTTCGCCCAGGTCAAGCAGATCTATAAGACCCCACCA
ATCAAGGACTTCGGCGGCTTCAACTTCAGCCAGATCCTGCCCGACCCGAGC
AAACCTAGCAAGAGAAGCTTCATCGAGGATCTTCTGTTCAACAAGGTGACG
CTGGCCGATGCCGGCTTCATCAAACAGTACGGCGATTGTCTGGGCGACATC
GCCGCCAGAGATCTGATCTGCGCCCAGAAGTTCAATGGCCTGACCGTGCTG
CCGCCTCTGCTCACAGACGAGATGATCGCCCAGTACACCTCTGCCCTGTTA
GCAGGCACAATCACGAGCGGCTGGACATTTGGCGCGGGCGCCGCTCTGCAG
ATCCCTTTCGCTATGCAGATGGCCTATAGATTTAATGGCATCGGCGTGACC
CAGAATGTGCTGTACGAGAACCAAAAGCTGATCGCTAATCAGTTCAATAGC
GCCATCGGAAAGATCCAGGACTCTCTGAGCAGCACCGCCTCCGCTCTGGGC
AAACTGCAGGACGTGGTCAATCAAAACGCTCAGGCTCTAAACACCCTGGTG
AAGCAGCTGTCAAGCAACTTCGGCGCCATCTCCTCAGTTCTTAACGACATC
TTGTCTAGACTGGACAAGGTGGAGGCTGAGGTGCAGATCGATAGACTGATC
ACAGGCAGATTACAGTCTCTGCAGACATACGTGACCCAACAGCTGATCCGG
GCCGCCGAGATCAGAGCCTCCGCCAACCTGGCTGCCACCAAGATGAGCGAG
TGTGTGCTTGGCCAGTCCAAGAGAGTGGACTTCTGCGGCAAAGGCTACCAC
CTGATGAGCTTCCCACAGAGCGCTCCTCACGGAGTTGTGTTCCTGCACGTG
ACCTACGTGCCTGCCCAGGAGAAGAATTTCACAACCGCCCCTGCCATCTGC
CACGACGGTAAGGCCCACTTCCCCAGAGAGGGCGTGTTCGTGTCTAATGGG
ACCCACTGGTTCGTGACACAGAGGAACTTCTACGAGCCCCAGATCATCACA
ACAGATAATACCTTCGTGTCTGGCAACTGCGATGTGGTGATCGGCATCGTG
AACAATACCGTGTACGACCCTCTGCAGCCCGAGCTGGACAGCTTCAAAGAG
GAACTGGACAAGTACTTCAAGAATCACACCTCCCCTGACGTGGACCTGGGA
GATATCTCCGGCATCAACGCCTCCGTTGTGAACATTCAAAAAGAAATCGAC
AGACTGAACGAGGTGGCCAAGAACCTAAACGAGTCTCTGATCGACCTGCAA
GAACTGGGCAAGTACGAGCAGTACATCAAGTGGCCTTGGTACATCTGGCTG
GGCTTTATCGCCGGCCTGATCGCCATTGTGATGGTGACCATCATGCTGTGC
TGCATGACAAGTTGCTGCAGCTGCCTGAAGGGCTGTTGTTCTTGTGGCAGC
TGCTGTAAATTCGACGAGGACGATAGCGAACCAGTGCTGAAAGGCGTCAAG
CTGCATTACACCTGA

In certain embodiments, the sequence encoding a spike protein comprises the sequence as set forth below (SEQ ID NO:78)

GCCACCATGTTTGTGTTCCTGGTCCTTCTCCCGCTTGTAAGCTCCCAATGC
GTTAACTTTACTACTCGGACCCAACTCCCGCCGGCGTACACTAACTCTTTT
GACCCGGGGCGTCTATTATCCTACAAAGTGTTTCGATCATCTGTTTTGCAC
TCCACCCAAGATCTCTTTCTTCCATTTTTCAGCAACGTTACGTGGTTCCAC
GCGATACATGTTAGTGGTACTAATGTTATAAAGAGATTTGACAACCCTGTG
CTCCCGTTTAACGATGGGGTCTATTTCGCTTCCACTGAGAAGTCCAACATT
ATTCGCGGTTGGATCTTCGGCACCACACTCGATTCAAAAACCCAAAGCTTG
CTCATAGTTAACAACGCCACAAACGTAGTCATTAAAGTGTGCGAATTTCAG
TTTTGTAACTATCCCTTTTTGGGCGTCTACTATCACAAGAACAACAAGTCC
TGGATGGAATCTGAGTTTCGGGTCTACAGTTCAGCCAACAACTGTACGTTT
GAGTACGTTAGCCAGCCCTTTCTGATGGATCTTGAAGGGAAGCAAGGAAAT
TTCAAGAATCTTTCCGAGTTTGTGTTTAAAAATATAGACGGATATTTCAAG
ATATACAGTAAGCACACACCGATCAATTTGGTACGGGATTTGCCGCAAGGA
TTTTCCGCCCTTGAGCCCCTTGTAGATCTCCCCATCGGAATTAACATAACC
AGGTTTCAAACACTGCTGGCGCTCCACAACTCATCCAGTGGGTGGACCGCC
GGAGCAGCCGCATACTACGTGGGGTATCTCCAGCCCAGAACGTTTCTCCTC
AAGTATAATGAAAACGGGACCATCACCGACGCGGTTGATTGTGCACTCGAT
CCCCTCAGCGAGACAAAGTGCACACTTAAATCATTTACCGTCGAGAAGGGG
ATCTACCAAACATCCAACTTTCGGGTGCAGCCTACCGAGTCAATAGTACGG
TTTCCTAACATCACCAATTTGTGCCCTTTTGGGGAAGTATTTAATGCAACC
CGGTTTGCGAGCGTATACGCTTGGAACCGAAAACGGATAAGTAATTGTGTT
GCAGATTATTCTGTGCTCTATAACTCAGCTTCCTTCTCCACCTTTAAGTGT
TACGGAGTATCTCCGACAAAATTGAATGATCTTTGTTTTACGAATGTTTAC
GCTGACTCATTTGTTATCCGAGGTGACGAAGTGCGCCAGATAGCCCCCGGT
CAAACAGGCAAAATTGCTGATTATAACTACAAGTTGCCCGATGACTTCACG
GGGTGTGTTATTGCATGGAATTCAAATAATCTTGATAGTAAGGTAGGGGGT
AACTATAATTACCAATATCGCTTGTTCCGCAAATCCAATCTGAAACCCTTC
GAAAGGGATATCAGTACCGAGATTTATCAAGCCGGCTCCACACCTTGTAAC
GGAGTAAAAGGTTTTAACTGTTATTCCCCACTGCAGAGTTACGGCTTTCAA
CCCACATACGGGGTGGGATATCAGCCCTATCGCGTTGTGGTCTTGTCCTTT
GAACTTCTCCACGCCCCTGCAACAGTTTGTGGTCCTAAAAAGAGTACAAAT
CTGGTAAAAAATAAGTGCGTCAACTTTAACTTCAACGGACTGACTGGTACC
GGGGTCCTCACTGAGAGCAACAAGAAATTTTTGCCCTTCCAGCAATTCGGG
AGGGATATTGCCGATACTACCGATGCTGTACGGGACCCCCAGACACTCGAA
ATATTGGACATAACCCCGTGCTCTTTTGGAGGGGTCTCTGTGATTACCCCG
GGTACGAACACGAGTAATCAGGTGGCAGTGCTTTATCAGGGAGTGAATTGC
ACAGAAGTACCAGTCGCGATCCATGCCGACCAGCTCACGCCGACCTGGAGG
GTTTATAGTACGGGTAGTAACGTTTTCCAAACACGGGCCGGTTGTCTCATC
GGGGCAGAGTACGTTAATAACAGCTATGAATGTGATATACCCATAGGTGCC
GGGATTTGTGCTTCTTATCAAACCCAAACCAATAGCTCACAGTCTATAATC
GCCTATACGATGAGCCTCGGAGCGGAAAACTCAGTTGCCTACTCCAACAAT
AGCATTGCTATCCCAACGAACTTCACTATTAGCGTAACCACGGAGATCCTT
CCAGTTAGCATGACAAAAACTAGCGTCGATTGCACTATGTATATTTGCGGC
GACAGTACAGAGTGTAGTAATTTGCTCCTGCAGTACGGGTCTTTCTGCACC
CAATTGAACCGAGCACTGACTGGCATCGCCGTGGAACAAGACAAAAACACG
CAAGAAGTATTTGCGCAAGTCAAGCAGATTTACAAGACTCCCCCGATCAAA
GACTTTGGCGGATTTAATTTTAGCCAGATACTCCCCGATCCATCTAAACCA
TCAAAACGGTCCTTTATAGAAGATCTCTTGTTCAATAAAGTGACACTCGCG
GACGCGGGCTTTATCAAACAGTATGGGGACTGCCTTGGAGATATAGCTGCG
AGGGACTTGATTTGCGCCCAGAAATTCAACGGGCTGAATGTTCTGCCGCCG
CTCTTGACAGACGAAATGATTGCACAGTATACGAGTGCCTTGCTGGCAGGG
ACTATCACTTCCGGTTGGACCTTCGGCGCAGGAGCAGCGCTCCAGATCCCA
TTCGCTATGCAAATGGCTTACCGATTCAACGGAATTGGCGTCACACAGAAC
GTGCTTTACGAGAATCAAAAGCTGATTGCCAATCAATTCAACTCAGCGATT
GGCAAAATACAAGATAGTCTCTCTTCAACTGCATCCGCGCTCGGTAAGTTG
CAGGACGTAGTCAATCAGAACGCACAGGCCCTGAACACGCTTGTTAAACAA
CTGTCTTCAAACTTCGGTGCAATAAGTTCCGTACTTAATGATATCTTGTCA
AGGTTGGATAAGGTCGAAGCTGAGGTACAAATCGACCGCCTTATAACAGGC
AGACTCCAATCCCTGCAGACATACGTGACCCAGCAACTCATTAGGGCAGCC
GAGATCAGAGCAAGTGCCAACCTCGCTGCTACAAAAATGTCTGAATGCGTT
TTGGGGCAATCTAAGCGGGTTGATTTCTGCGGTAAGGGATACCACTTGATG
TCCTTTCCTCAGTCCGCTCCTCATGGGGTAGTATTTCTTCATGTTACATAT
GTACCAGCACAAGAGAAAAATTTTACTACTGCCCCGGCCATATGCCATGAT
GGCAAAGCGCACTTCCCTCGGGAGGGGGTTTTTGTGTCTAATGGAACCCAC
TGGTTCGTGACTCAGCGGAATTTCTATGAACCCCAGATTATAACTACTGAC
AACACTTTCGTTTCTGGAAACTGCGATGTAGTAATAGGCATAGTAAATAAT
ACAGTATATGATCCCTTGCAACCAGAATTGGACTCTTTTAAAGAAGAGCTC
GACAAATATTTTAAAAACCATACATCTCCAGACGTTGATCTTGCTGACATC
TCTGGGATAAACGCTAGCGTAGTAAACATACAAAAAGAAATCGATAGGCTT
AACGAGGTCGCAAAAAATCTTAATGAAAGTCTTATCGATTTGCAAGAGCTT
GGAAAGTATGAGCAGTATATCAAATGGCCTTGGTATATATGGCTGGGATTT
ATTGCTGGTCTCATAGCCATAGTTATGGTGACAATAATGCTGTGCTGTATG
ACGTCATGTTGTTCCTGCCTGAAAGGGTGCTGTAGTTGTGGGAGTTGTTGC
AAGTTTGACGAGGACGACTCAGAGCCCGTTTTGAAAGGTGTGAAACTTCAT
TATACATCACGACTGGAGGAAGAACTGCGCCGACGCCTGACTGAATGA

In certain embodiments, the sequence encoding the spike protein without furin cleavage site comprises the sequence as set forth below (SEQ ID NO:25):

GCCGCCACCATGGTGAACCTGACCACCCGGACCCAGCTACCACCAGCCT
ACACAAACAGCTTCACCCGCGGCGTGTACTACCCAGACAAGGTGTTCCG
GAGCTCAGTGCTCCACAGCACACAGGACCTGTTCCTGCCATTCTTCAGC
AACGTGACGTGGTTCCACGCCATCCACGTGTCCGGCACCAACGGAACAA
AAAGGTTCGACAACCCCGTGCTCCCCTTCAACGACGGCGTGTACTTCGC
CAGCACAGAAAAGTCCAAAATCATCCGAGGATGGATCTTCGGCACAACG
CTGGACTCAAAGACCCAGAGCCTGCTGATCGTGAACAACGCCACCAACG
TGGTGATCAAGGTGTGCGAGTTCCAGTTCTGCAACGATCCATTCCTGGG
CGTATACTACCACAAGAACAACAAGAGCTGGATGGAAAGCGAGTTCAGA
GTGTACTCAAGCGCCAACAACTGCACCTTCGAATACGTCAGCCAGCCAT
TCCTGATGGACCTGGAAGGCAAGCAGGGCAACTTCAAGAACCTGAGAGA
GTTCGTGTTCAAGAACATCGACGGCTACTTCAAGATCTACAGCAAGCAC
ACACCCATCAACCTGGTGCGGGACCTACCGCAGGGATTCTCCGCGCTGG
AACCCCTGGTGGACCTGCCAATAGGCATCAACATCACCAGATTCCAGAC
CCTGCTGGCCCTGCACAGAAGCTACCTGACACCAGGCGACAGCAGCAGC
GGCTGGACCGCCGGCGCCGCCGCATACTACGTGGGGTACCTGCAGCCCA
GAACCTTCCTGCTGAAATACAACGAGAACGGAACCATCACCGACGCCGT
GGACTGCGCCCTGGACCCCCTGAGCGAAACCAAGTGCACCCTGAAAAGC
TTCACAGTGGAAAAGGGAATATACCAGACCTCCAACTTCAGAGTCCAGC
CAACCGAAAGCATCGTGCGGTTCCCAAACATCACCAACCTGTGCCCATT
CGGCGAGGTATTCAACGCCACACGGTTCGCCAGCGTATACGCCTGGAAC
CGGAAGCGGATCAGCAACTGCGTGGCAGACTACAGCGTGCTATACAACA
GCGCCAGCTTAAGCACCTTCAAGTGCTACGGCGTGTCCCCAACCAAGCT
GAACGACCTGTGCTTCACCAACGTGTACGCCGACAGCTTCGTGATCAGA
GGCGACGAGGTGCGCCAGATCGCACCAGGACAGACCGGAAAGATCGCAG
ACTACAACTACAAGCTGCCCGACGACTTCACCGGCTGCGTGATCGCCTG
GAACAGCAACAACCTGGACTCCAAAGTGGGCGGCAACTACAACTACCTG
TACCGGCTCTTCAGAAAGAGCAACCTGAAGCCCTTCGAGAGAGACATCA
GCACAGAAATCTACCAGGCCGGAAGCACACCATGCAACGGCGTGGAAGG
ATTCAACTGCTATTTCCCACTGCAGTCCTACGGCTTCCAACCAACCAAC
GGAGTGGGCTACCAGCCATACAGAGTGGTCGTGCTGAGCTTCGAGCTGC
TGCACGCCCCAGCAACCGTGTGCGGCCCAAAGAAAAGCACGAACCTGGT
CAAGAACAAATGCGTGAACTTCAACTTCAACGGACTGACAGGCACCGGC
GTGCTCACAGAGAGCAACAAGAAGTTCCTGCCATTCCAGCAGTTCGGCA
GAGACATAGCAGACACCACCGACGCCGTGCGGGACCCCCAGACCCTGGA
AATCCTGGACATCACACCCTGCAGCTTCGGAGGCGTAAGCGTGATCACA
CCAGGCACCAACACAAGCAACCAGGTCGCCGTGCTGTACCAAGACGTGA
ACTGCACAGAGGTGCCGGTGGCCATCCACGCCGACCAGCTGACCCCCAC
CTGGCGGGTGTACTCCACCGGCAGCAACGTGTTCCAAACCCGGGCCGGC
TGCCTGATCGGCGCCGAACACGTGAACAACAGCTACGAATGCGACATCC
CAATCGGAGCCGGAATCTGCGCCAGCTACCAAACCCAAACCAACAGCGC
AAGCCAGAGCATCATCGCCTACACCATGAGCCTGGGCGCAGAAAACAGC
GTGGCATACAGCAACAACAGCATCGCCATCCCAACCAACTTCACCATCA
GCGTGACCACAGAGATCCTGCCCGTGAGCATGACCAAGACCAGCGTGGA
CTGCACCATGTACATCTGCGGCGACAGCACCGAGTGCAGCAACTTGCTA
CTGCAGTACGGAAGCTTCTGCACCCAACTGAACAGAGCCCTGACCGGCA
TCGCCGTCGAGCAGGACAAGAACACCCAGGAGGTGTTCGCCCAGGTCAA
GCAGATCTACAAGACCCCACCAATCAAGGACTTCGGCGGCTTCAACTTC
AGCCAGATCCTGCCCGACCCGAGCAAACCAAGCAAGAGAAGCTTCATCG
AGGACCTACTGTTCAACAAGGTGACGCTGGCCGACGCCGGCTTCATCAA
ACAGTACGGCGACTGCCTGGGCGACATCGCCGCCAGAGACCTGATCTGC
GCCCAGAAGTTCAATGGCCTGACCGTGCTGCCGCCACTGCTCACAGACG
AGATGATCGCCCAGTACACCAGCGCCCTGTTAGCAGGCACAATCACGAG
CGGCTGGACATTCGGCGCGGGCGCCGCACTGCAGATCCCATTCGCAATG
CAGATGGCCTACAGATTCAATGGCATCGGCGTGACCCAGAACGTGCTGT
ACGAGAACCAAAAGCTGATCGCAAACCAGTTCAACAGCGCCATCGGAAA
GATCCAGGACAGCCTGAGCAGCACCGCCTCCGCACTGGGCAAACTGCAG
GACGTGGTCAACCAAAACGCACAGGCACTAAACACCCTGGTGAAGCAGC
TGTCAAGCAACTTCGGCGCCATCAGCTCAGTCCTAAACGACATCTTGAG
CAGACTGGACAAGGTGGAGGCAGAGGTGCAGATCGACAGACTGATCACA
GGCAGATTACAGAGCCTGCAGACATACGTGACCCAACAGCTGATCCGGG
CCGCCGAGATCAGAGCCTCCGCCAACCTGGCAGCCACCAAGATGAGCGA
GTGCGTGCTAGGCCAGAGCAAGAGAGTGGACTTCTGCGGCAAAGGCTAC
CACCTGATGAGCTTCCCACAGAGCGCACCACACGGAGTAGTGTTCCTGC
ACGTGACCTACGTGCCAGCCCAGGAGAAGAACTTCACAACCGCCCCAGC
CATCTGCCACGACGGAAAGGCCCACTTCCCCAGAGAGGGCGTGTTCGTG
AGCAACGGGACCCACTGGTTCGTGACACAGAGGAACTTCTACGAGCCCC
AGATCATCACAACAGACAACACCTTCGTGAGCGGCAACTGCGACGTGGT
GATCGGCATCGTGAACAACACCGTGTACGACCCACTGCAGCCCGAGCTG
GACAGCTTCAAAGAGGAACTGGACAAGTACTTCAAGAACCACACCTCCC
CAGACGTGGACCTGGGAGACATCAGCGGCATCAACGCCTCCGTAGTGAA
CATCCAAAAAGAAATCGACAGACTGAACGAGGTGGCCAAGAACCTAAAC
GAGAGCCTGATCGACCTGCAAGAACTGGGCAAGTACGAGCAGTACATCA
AGTGGCCATGGTACATCTGGCTGGGCTTCATCGCCGGCCTGATCGCCAT
CGTGATGGTGACCATCATGCTGTGCTGCATGACAAGCTGCTGCAGCTGC
CTGAAGGGCTGCTGCAGCTGCGGCAGCTGCTGCAAATTCGACGAGGACG
ACAGCGAACCAGTGCTGAAAGGCGTCAAGCTGCACTACACCTGA

In certain embodiments, the sequence encoding the spike protein extracellular domain comprises the sequence as set forth below (SEQ ID NO:26)

GCCACCATGGTGAACCTCACTACGCGAACTCAACTGCCGCCTGCTTACA
CTAATAGTTTTACACGAGGAGTCTACTACCCGGACAAGGTGTTCCGCTC
AAGTGTCTTGCACAGCACGCAAGACCTGTTCTTGCCATTTTTCAGCAAT
GTAACGTGGTTTCATGCCATCCACGTGTCAGGTACTAACGGAACGAAGA
GATTTGATAACCCGGTCCTCCCGTTCAACGACGGGGTTTATTTTGCCAG
TACAGAAAAGTCCAATATTATAAGGGGATGGATTTTCGGCACGACTCTC
GACTCTAAGACACAAAGCTTGCTCATCGTTAATAATGCTACAAACGTGG
TTATAAAAGTATGCGAGTTCCAATTCTGTAACGACCCCTTTCTGGGGGT
TTACTACCACAAAAATAATAAGAGTTGGATGGAGAGCGAATTCAGAGTT
TACTCTTCCGCGAATAATTGCACCTTCGAGTACGTATCCCAGCCTTTTC
TTATGGATCTTGAGGGGAAACAAGGTAATTTCAAAAACCTTAGAGAATT
CGTTTTTAAGAATATAGATGGATACTTCAAGATTTATTCTAAACATACC
CCAATCAATCTGGTGCGCGATCTTCCTCAGGGTTTCTCTGCGCTGGAGC
CTCTCGTTGATCTGCCGATCGGAATTAATATAACCAGATTCCAGACCCT
TTTGGCATTGCACCGCTCCTATCTGACACCTGGGGATAGTAGTTCCGGC
TGGACAGCGGGCGCCGCGGCATACTACGTTGGATATCTCCAACCCCGCA
CTTTTCTTCTGAAATATAATGAGAATGGAACGATTACAGACGCAGTAGA
TTGCGCGCTTGACCCCCTTTCAGAAACAAAGTGCACGCTGAAATCTTTC
ACGGTCGAGAAGGGCATTTACCAAACGTCCAATTTCAGAGTTCAACCCA
CTGAGTCTATAGTCAGGTTTCCGAACATCACCAATCTCTGCCCATTTGG
AGAGGTTTTCAACGCAACACGGTTCGCCTCTGTATACGCATGGAATAGA
AAGAGAATATCCAATTGTGTCGCAGACTATAGTGTCTTGTACAACTCTG
CGTCATTTAGCACTTTTAAATGTTATGGGGTGAGTCCAACTAAATTGAA
CGATCTCTGCTTTACAAATGTTTACGCAGATAGTTTTGTGATACGAGGC
GACGAGGTACGACAAATTGCTCCTGGCCAGACCGGGAAAATAGCGGACT
ACAATTATAAACTGCCAGACGATTTCACCGGCTGTGTGATCGCGTGGAA
CTCAAACAACTTGGATAGTAAGGTAGGTGGTAACTATAACTACTTGTAC
CGGCTCTTCCGCAAAAGTAATCTTAAACCGTTCGAGCGGGACATCTCTA
CCGAGATCTATCAAGCAGGTAGCACTCCCTGCAATGGAGTCGAGGGTTT
TAATTGCTATTTTCCCCTCCAATCATACGGGTTTCAGCCCACAAATGGT
GTTGGGTATCAGCCCTATCGAGTTGTGGTGCTTTCATTTGAATTGCTTC
ATGCACCGGCGACAGTTTGTGGTCCTAAAAAGAGTACCAATTTGGTTAA
AAACAAATGTGTCAACTTTAACTTTAACGGTCTGACTGGTACGGGGGTG
CTTACGGAATCTAATAAAAAGTTTCTTCCGTTCCAGCAATTCGGACGGG
ACATAGCGGACACGACTGACGCGGTTAGAGACCCACAAACACTCGAGAT
TCTTGACATAACCCCCTGTTCTTTCGGCGGAGTTTCTGTAATAACGCCT
GGGACAAATACGTCAAACCAGGTTGCAGTATTGTACCAGGATGTCAACT
GCACTGAAGTGCCAGTCGCGATCCACGCAGATCAGTTGACTCCAACATG
GAGGGTCTACTCTACCGGTTCTAACGTGTTTCAGACCAGAGCCGGGTGC
CTCATTGGCGCGGAACATGTGAACAATTCTTACGAATGCGACATACCCA
TCGGCGCCGGCATTTGTGCGTCATACCAAACTCAAACTAATTCCCGGAG
TGTGGCCTCCCAATCTATTATTGCTTATACTATGAGTCTTGGCGCGGAA
AACAGTGTAGCGTACTCAAATAACTCTATTGCGATCCCCACGAATTTCA
CTATAAGCGTAACTACTGAAATTTTGCCGGTAAGCATGACGAAAACTTC
AGTGGACTGCACAATGTACATTTGTGGTGACAGCACTGAGTGCTCAAAT
CTCCTCCTTCAGTATGGTTCTTTCTGCACACAACTTAACAGGGCACTTA
CCGGAATCGCAGTAGAACAAGACAAAAACACGCAAGAAGTATTCGCACA
GGTCAAACAAATTTATAAAACCCCGCCGATCAAGGATTTTGGAGGGTTT
AACTTCAGTCAGATCCTTCCAGACCCATCCAAACCCAGCAAACGGAGCT
TTATTGAAGACCTGCTGTTTAATAAAGTTACCCTTGCAGATGCCGGTTT
CATCAAGCAATACGGGGATTGTCTGGGCGACATTGCCGCCCGCGACTTG
ATCTGTGCCCAGAAGTTCAACGGTCTCACAGTCTTGCCGCCACTTCTCA
CTGATGAAATGATAGCGCAGTATACATCCGCGCTGCTGGCTGGGACGAT
AACATCTGGTTGGACTTTCGGGGCCGGGGCGGCTTTGCAAATTCCTTTC
GCCATGCAGATGGCCTATAGGTTTAACGGCATAGGAGTGACTCAAAATG
TTCTTTATGAGAATCAGAAGCTTATCGCCAACCAATTTAACTCCGCGAT
TGGGAAGATACAGGACTCATTGAGCTCTACTGCTAGCGCCCTTGGCAAG
CTTCAAGACGTAGTCAACCAAAATGCTCAAGCGCTCAATACTCTCGTAA
AGCAGCTCTCAAGCAACTTTGGCGCGATATCTTCAGTTTTGAACGACAT
CTTGAGCAGACTCGACAAGGTGGAGGCTGAAGTTCAAATTGATCGACTG
ATTACGGGCCGGCTCCAGTCACTCCAAACGTACGTGACCCAACAACTCA
TTAGAGCGGCAGAGATTAGAGCGAGCGCTAATCTGGCGGCCACAAAGAT
GTCAGAATGTGTGCTTGGTCAATCCAAAAGGGTGGATTTCTGCGGAAAG
GGCTATCACTTGATGAGTTTCCCGCAGTCCGCGCCTCATGGAGTTGTTT
TCCTCCACGTAACCTACGTTCCCGCTCAGGAAAAGAATTTCACGACAGC
TCCGGCAATATGTCATGACGGTAAAGCACATTTCCCCAGGGAAGGCGTT
TTTGTCAGTAATGGAACCCACTGGTTCGTCACCCAGCGAAACTTTTACG
AGCCCCAAATAATAACTACTGACAATACCTTCGTCAGCGGCAATTGCGA
CGTCGTCATAGGCATAGTAAATAATACGGTTTACGACCCTCTTCAACCC
GAGTTGGACAGCTTCAAAGAAGAACTTGATAAGTATTTTAAAAACCACA
CTAGCCCAGATGTAGACCTTGGCGACATCAGCGGTATCAATGCTTCCGT
TGTGAACATTCAAAAGGAAATTGACCGACTCAATGAAGTAGCGAAGAAC
TTGAATGAGTCTCTGATCGATCTTCAGGAGCTCGGGAAATATGAGCAAT
ACATCAAATGGCCGTCACGACTGGAGGAAGAACTGCGCCGACGCCTGAC
TGAATGA

In certain embodiments, the sequence encoding the spike protein extracellular domain comprises the sequence as set forth below (SEQ ID NO:27)

GCCACCATGGTTAACCTGACAACACGCACCCAGTTGCCACCGGCCTATA
CGAACTCTTTCACGCGCGGAGTGTATTATCCCGATAAAGTCTTTCGGAG
TTCTGTGCTGCACTCTACACAAGATCTTTTTCTGCCCTTTTTCTCCAAC
GTTACGTGGTTCCATGCAATAAGTGGTACGAATGGTACTAAAAGGTTCG
ATAATCCCGTACTTCCCTTCAATGACGGCGTGTATTTCGCGAGCACAGA
AAAGAGCAACATAATAAGAGGTTGGATTTTTGGGACTACCCTGGACTCT
AAAACACAAAGCCTCCTCATTGTTAATAACGCAACTAATGTCGTAATAA
AGGTATGCGAATTTCAGTTTTGTAACGACCCTTTTTTGGGAGTCTATCA
TAAGAACAATAAGTCCTGGATGGAAAGTGAATTTAGGGTGTATAGTTCA
GCGAACAACTGTACATTTGAGTATGTGTCACAGCCGTTCCTTATGGATC
TTGAAGGGAAACAGGGGAACTTCAAGAACCTGCGGGAATTCGTGTTTAA
AAATATAGACGGTTATTTTAAAATCTATAGCAAACACACCCCAATAAAC
CTGGTTCGGGATCTTCCTCAGGGATTTTCAGCTTTGGAACCGTTGGTTG
ACCTCCCCATCGGGATAAATATCACGCGATTTCAAACACTGCTGGCACT
GCACCGGAGTTATCTGACTCCTGGAGATAGCAGCTCCGGCTGGACGGCT
GGCGCCGCGGCTTATTATGTCGGGTACCTTCAACCGCGAACGTTTCTTT
TGAAGTATAACGAAAATGGCACTATAACCGATGCAGTAGATTGTGCGTT
GGACCCTCTTTCCGAGACTAAGTGCACCCTGAAGTCATTTACAGTAGAG
AAGGGGATATACCAGACAAGCAACTTCCGCGTCCAACCAACGGAAAGCA
TCGTGCGCTTTCCCAATATTACTAACCTCTGTCCGTTCGGCGAGGTTTT
TAATGCTACCAGATTTGCCTCAGTGTATGCCTGGAACCGCAAGAGGATT
AGCAACTGCGTTGCTGACTATAGCGTGCTTTACAATTCCGCCAGTTTTA
GTACGTTCAAGTGTTATGGGGTAAGCCCTACCAAGCTTAACGATCTTTG
CTTCACGAATGTTTACGCAGACTCATTCGTAATACGAGGTGATGAAGTG
CGACAGATAGCCCCGGGTCAGACGGGGAAAATAGCCGACTACAATTACA
AGCTCCCGGACGACTTCACGGGCTGTGTTATTGCGTGGAACAGCAATAA
CCTGGACAGTAAAGTTGGAGGGAATTATAACTACTTGTACAGGCTGTTC
CGCAAATCTAATTTGAAACCGTTTGAAAGGGACATTTCCACCGAGATTT
ACCAAGCCGGCAGTACCCCATGTAACGGAGTTGAGGGATTTAATTGCTA
CTTTCCGCTCCAATCATATGGATTCCAGCCTACATACGGCGTCGGATAT
CAACCTTATAGGGTGGTCGTTCTTTCTTTTGAGCTGCTTCACGCCCCAG
CAACCGTTTGCGGCCCGAAAAAATCAACGAACTTGGTCAAAAACAAGTG
CGTCAACTTCAATTTCAACGGACTGACAGGCACCGGAGTGCTCACCGAG
TCAAATAAAAAATTCCTCCCGTTTCAACAATTCGGTAGAGATATAGACG
ACACCACAGATGCGGTACGCGACCCTCAGACTCTTGAAATTTTGGACAT
TACACCATGTTCCTTCGGTGGCGTCTCAGTGATTACGCCTGGCACTAAC
ACTAGCAACCAAGTCGCAGTACTTTATCAGGGCGTTAACTGTACAGAAG
TACCAGTTGCGATACACGCGGACCAGCTCACCCCAACCTGGCGCGTATA
TTCTACCGGATCTAATGTGTTTCAAACGCGGGCGGGGTGCCTCATAGGT
GCAGAGCATGTCAATAACTCCTACGAATGTGACATTCCTATAGGAGCAG
GAATATGTGCGAGCTACCAGACTCAAACCAATTCACACAGGAGCGTAGC
TAGTCAGTCCATAATTGCCTACACTATGTCACTTGGTGCTGAAAACAGT
GTGGCTTATTCAAATAATTCCATAGCTATTCCAATCAATTTCACTATTA
GCGTAACAACTGAGATACTTCCAGTCAGCATGACTAAGACTTCAGTTGA
TTGTACGATGTATATTTGTGGCGATTCTACAGAATGTAGTAACCTTCTG
TTGCAATACGGCTCCTTTTGCACACAGCTCAATCGCGCATTGACGGGTA
TAGCTGTAGAGCAAGATAAAAACACCCAAGAAGTCTTCGCGCAGGTGAA
GCAAATATATAAGACACCACCGATTAAAGATTTCGGTGGATTCAATTTT
AGCCAAATTCTGCCTGATCCAAGCAAACCGTCAAAAAGATCCTTCATAG
AGGACCTGCTTTTCAATAAGGTAACGCTGGCGGACGCAGGGTTTATTAA
ACAATACGGCGATTGCCTTGGTGACATTGCTGCACGAGACCTCATTTGC
GCCCAGAAGTTCAACGGACTCACAGTACTGCCTCCACTGCTCACAGACG
AGATGATCGCCCAGTACACATCTGCCTTGTTGGCTGGTACGATAACTTC
TGGTTGGACCTTTGGAGCGGGAGCAGCACTTCAGATTCCCTTTGCCATG
CAGATGGCGTACAGATTCAACGGCATTGGTGTCACGCAGAACGTACTGT
ATGAGAACCAGAAGTTGATAGCTAATCAATTCAACTCCGCGATAGGTAA
GATCCAAGACAGTCTCTCCTCAACAGCCTCCGCACTCGGGAAACTCCAG
GATGTGGTAAATCAGAATGCACAGGCATTGAATACGCTTGTTAAACAAT
TGTCCTCAAATTTTGGCGCCATATCATCAGTGCTCAACGACATCCTTGC
CAGACTCGATAAAGTGGAGGCAGAAGTGCAGATAGATCGCTTGATTACT
GGGCGCCTCCAATCTCTCCAAACGTACGTGACACAACAGCTTATTCGGG
CAGCGGAAATCCGCGCCTCTGCGAACCTTGCAGCGACCAAGATGAGCGA
ATGTGTTCTCGGGCAGTCAAAACGAGTAGATTTCTGCGGTAAAGGATAT
CATCTTATGTCATTCCCCCAAAGCGCTCCACATGGTGTTGTATTTCTGC
ACGTGACTTACGTGCCGGCACAGGAGAAGAATTTCACAACGGCTCCCGC
CATCTGTCACGACGGAAAGGCACACTTCCCTAGGGAGGGGGTGTTTGTA
AGCAATGGTACCCACTGGTTTGTCACACAACGAAACTTCTATGAACCTC
AGATAATTACAACGCATAATACTTTTGTAAGTGGGAACTGCGACGTCGT
TATCGGAATAGTTAACAATACTGTATATGATCCGTTGCAGCCGGAGTTG
GACTCATTTAAAGAAGAGCTGGACAAGTACTTCAAAAACCATACAAGCC
CAGATGTAGATCTTGGGGACATAAGTGGGATAAACGCCAGCGTTGTGAA
CATACAGAAGGAAATAGACAGGCTGAATGAGGTAGCAAAGAATCTTAAC
GAAAGCCTTATAGATCTTCAGGAACTTGGGAAGTACGAACAGTACATAA
AGTGGCCTTCACGACTGGAGGAAGAACTGCGCCGACGCCTGACTGAATG
A

In certain embodiments, the sequence encoding the spike protein extracellular domain comprises the sequence as set forth below (SEQ ID NO:28)

GCCACCATGGTCAATTTCACTACACGCACACAGCTGCCCCCGGCGTACA
CTAACAGTTTTACGAGGGGAGTCTACTACCCGGACAAAGTGTTCAGGTC
TTCCGTACTCCATAGCACACAAGACCTCTTCTTGCCTTTTTTCAGCAAT
GTTACGTGGTTCCATGCAATCCATGTGAGCGGTACAAACGGGACTAAAC
GCTTTGCAAATCCAGTGTTGCCCTTCAACGACGGTGTATACTTCGCGAG
TACAGAGAAGAGCAACATAATACGCGGGTGGATATTTGGAACGACGCTT
GATTCTAAGACACAGAGCTTGCTCATCGTAAACAACGCAACGAATGTTG
TAATCAAGGTCTGCGAATTCCAATTCTGCAATGACCCATTTCTTGGAGT
TTATTACCATAAGAACAATAAGTCTTGGATGGAATCCGAGTTCCGCGTA
TACTCTAGCGCGAACAACTGCACCTTCGAGTATGTGTCCCAACCGTTTC
TGATGGATCTTGAGGGAAAGCAGGGTAATTTTAAAAATCTCCGGGAGTT
CGTTTTCAAAAATATTGACGGATATTTCAAAATCTACAGCAAGCATACC
CCAATCAATTTGGTGCGGGGTCTGCCACAAGGATTTTCCGCTCTTGAAC
CTCTTGTTGACCTTCCCATAGGAATCAATATAACTCGGTTCCAAACTCT
TCTTGCGCTCCATATATCCTACTTGACGCCCGGAGATTCATCAAGTGGC
TGGACGGCTGGGGCTGCCGCATACTACGTCGGCTATCTGCAACCAAGGA
CGTTTCTCCTCAAATACAATGAAAATGGTACGATCACCGACGCTGTGGA
CTGCGCTCTCGACCCGTTGTCAGAGACGAAGTGCACCTTGAAGTCCTTC
ACCGTGGAAAAGGGAATTTACCAGACTAGTAACTTCCGGGTGCAGCCCA
CGGAATCAATTGTGCGATTTCCTAATATCACTAATCTCTGTCCCTTCGG
GGAAGTGTTCAATGCGACTCGATTCGCTTCAGTATACGCTTGGAATAGA
AAGAGAATCTCAAATTGCGTGGCTGACTATTCCGTACTGTATAATAGTG
CGTCTTTTAGCACGTTTAAGTGTTATGGCGTATCCCCGACAAAGCTCAA
CGATCTCTGCTTCACGAATGTCTATGCCGACTCCTTCGTGATACGCGGT
GACGAGGTACGACAAATTGCACCCGGCCAAACTGGAAATATTGCCGACT
ACAATTATAAGTTGCCGGACGATTTTACCGGTTGCGTAATAGCGTGGAA
CTCTAATAACCTCGACTCTAAGGTAGGAGGCAACTACAATTACTTGTAC
CGACTGTTCAGGAAATCCAACCTGAAGCCCTTCGAAAGAGACATCAGTA
CAGAGATTTATCAAGCCGGGTCCACCCCTTGCAATGGTGTCAAAGGCTT
CAACTGCTATTTTCCGTTGCAGTCTTACGGTTTCCAGCCGACGTACGGT
GTGGGTTATCAACCCTACCGCGTTGTGGTGCTGAGCTTTGAGCTTCTCC
ACGCTCCAGCCACTGTCTGCGGGCCTAAGAAATCCACAAACCTCGTTAA
GAATAAATGCGTAAATTTCAACTTCAACGGGCTTACTGGCACGGGCGTG
CTTACGGAGTCCAATAAGAAATTCCTGCCTTTCCAGCAGTTCGGCCGCG
ACATCGCGGACACAACAGATGCTGTGCGAGACCCACAAACCCTGGAGAT
TCTGGACATCACGCCTTGCTCTTTTGGCGGTGTCAGCGTAATAACGCCA
GGCACCAACACCAGTAATCAGGTCGCCGTGCTTTATCAGGGTGTGAACT
GTACGGAGGTACCGGTTGCCATACACGCAGACCAACTCACACCTACGTG
GAGAGTTTACAGCACCGGGTCAAACGTATTTCAGACCCGCGCAGGCTGT
CTGATCGGCGCAGAGCATGTCAATAACTCATACGAGTGTGATATACCAA
TCGGAGCCGGCATCTGTGCGAGCTATCAAACCCAAACTAATTCACGAAG
CGTGGCCAGCCAGTCCATCATTGCCTACACGATGTCTTTGGGAGTAGAG
AATAGCGTGGCATACTCTAACAACAGTATCGCTATTCCTACCAACTTCA
CAATATCAGTTACTACAGAAATCCTCCCGGTGAGCATGACTAAGACATC
CGTAGACTGTACAATGTACATTTGTGGGGACAGCACGGAGTGTTCAAAC
CTGCTGTTGCAATACGGAAGCTTCTGCACCCAGCTCAACCGAGCCCTGA
CCGGCATTGCCGTAGAACAAGATAAGAATACGCAGGAAGTGTTCGCCCA
GGTTAAGCAGATTTACAAGACTCCGCCTATCAAGGACTTCGGGGGTTTC
AACTTTAGTCAGATTTTGCCTGACCCGTCAAAGCCTAGCAAACGAAGTT
TTATCGAAGATTTGTTGTTTAACAAGGTGACTTTGGCTGATGCGGGATT
CATAAAACAATACGGCGATTGCCTTGGCGATATTGCGGCCCGAGATCTC
ATTTGCGCGCAAAAGTTCAACGGTCTTACCGTCCTTCCGCCCCTGCTTA
CGGACGAGATGATCGCACAATACACTTCAGCACTGCTGGCTGGTACAAT
AACCAGTGGGTGGACATTCGGGGCTGGTGCAGCGCTCCAGATCCCCTTT
GCGATGCAGATGGCGTATAGATTCAATGGCATCGGGGTCACTCAGAACG
TGCTTTATGAGAACCAAAAGCTTATTGCAAATCAATTTAACTCTGCGAT
TGGAAAGATTCAAGACAGCCTCTCATCCACAGCTAGTGCGCTTGGCAAG
CTTCAGGACGTCGTAAACCAAAACGCCCAAGCTCTCAACACACTCGTTA
AACAGCTTTCCTCAAACTTTGGGGCTATTAGCTCTGTGCTGAATGATAT
TCTTAGCAGACTCGACAAAGTCGAGGCTGAAGTACAAATAGATAGACTG
ATAACAGGTAGACTTCAGTCTCTCCAAACCTACGTGACACAGCAGCTTA
TAAGAGCTGCTGAGATCAGAGCTTCTGCGAACCTTGCGGCGACTAAAAT
GTCCGAGTGTGTACTCGGGCAGTCTAAACGCGTCGACTTCTGTGGCAAG
GGTTACCATCTCATGAGTTTTCCCCAATCAGCGCCCCATGGGGTAGTTT
TTCTGCACGTAACATACGTCCCAGCCCAAGAAAAGAATTTTACCACCGC
CCCTGCGATTTGTCACGACGGAAAAGCACACTTTCCAAGAGAAGGCGTA
TTTGTTAGCAATGGCACCCATTGGTTCGTCACCCAGAGAAACTTTTACG
AACCGCAGATTATCACGACAGATAATACATTTGTATCTGGTAATTGTGA
CGTCGTGATCGGCATCGTAAACAACACTGTTTACGACCCCCTCCAGCCA
GAGCTTGACTCATTTAAAGAGGAGCTCGACAAATATTTTAAGAACCACA
CAAGTCCAGACGTCGACTTGGGTGACATTTCCGGCATCAATGCAAGTGT
GGTCAACATCCAAAAGGAAATTGACAGATTGAACGAGGTTGCTAAGAAC
CTTAATGAGTCACTCATTGACCTCCAGGAACTGGGAAAATACGAACAGT
ATATTAAGTGGCCCTCACGACTGGAGGAAGAACTGCGCCGACGCCTGAC
TGAATGA

In certain embodiments, the sequence encoding the spike protein extracellular domain comprises the sequence as set forth below (SEQ ID NO:29)

GCCACCATGGTAAATTTGACAACGCGCACCCAATTGCCCCCAGCATATA
CGAACTCTTTCACGAGGGGCGTATATTATCCGGATAAGGTATTTCGGTC
ATCTGTTCTGCACAGCACCCAGGACCTCTTCCTTCCATTCTTTTCAAAT
GTAACTTGGTTCCATGCGATAGTATCAGGAACGAATGGGACAAAAAGGT
TCGATAATCCGGTCTTGCCGTTCAACGATGGGGTGTACTTCGCCAGTAC
CGAAAAGTCTAACATTATACGCGGTTGGATTTTTGGCACTACGCTTGAC
TCAAAGACACAGTCACTCCTCATTGTAAATAATGCTACTAATGTCGTGA
TCAAAGTTTGTGAGTTCCAATTCTGCAACGACCCGTTCCTGGGAGTGTA
TTATCACAAAAATAATAAGTCCTGCATGGAGTCAGAGTTCAGGGTTTAC
TCAAGTGCGAACAATTGCACATTTGAGTACGTTTCTCAACCATTTCTCA
TGGATTTGGAGGGGAAGCAGGGAAACTTTAAAAACCTGAGAGAATTTGT
TTTTAAGAACATTGATGGGTATTTCAAGATTTATAGTAAACACACCCCT
ATCAACTTGGTTAGGGATCTCCCTCAAGGTTTCTCTGCTCTCGAGCCCC
TTGTAGATCTGCCAATAGGCATCAATATCACACGCTTTCAGACACTCCT
CGCACTTCATAGGAGCTACCTGACGCCAGGTGACTCTTCCTCAGGTTGG
ACAGCCGGCGCAGCCGCATACTACGTTGGCTACCTCCAACCAAGGACAT
TTCTGTTGAAATACAACGAAAATGGGACCATCACCGACGCAGTCGATTG
TGCTCTCGACCCTCTTTCCGAGACTAAATGTACCCTCAAAAGCTTTACT
GTTGAGAAGGGTATCTATCAGACATCTAACTTTCGGGTGCAACCCACTG
AGTCAATTGTGCGATTCCCAAATATTACGAACCTCTGTCCTTTTGGCGA
GGTTTTTAACGCCACTAGGTTCGCCAGTGTATATGCTTGGAACCGAAAA
CGGATAAGCAATTGTGTTGCTGACTACTCCGTCCTCTACAATAGCGCTA
GTTTCTCAACATTTAAGTGTTACGGTGTGAGCCCTACGAAACTTAACGA
TTTGTGCTTCACTAACGTCTATGCCGACAGTTTCGTAATCCGAGGCGAT
GAGGTCAGGCAAATTGCCCCGGGCCAAACGGGGAAAATCGCTGATTACA
ATTATAAGTTGCCAGATGATTTTACGGGATGTGTCATTGCATGGAACAG
TAATAACCTCGATTCAAAGGTTGGCGGAAATTATAATTACAGATATCGG
CTTTTTAGAAAATCTAACCTTAAACCATTTGAGCGGGACATAAGCACGG
AGATTTACCAGGCTGGTAGCACTCCGTGCAACGGTGTAGAAGGATTCAA
TTGCTATTTTCCATTGCAGTCTTATGGATTCCAACCCACCAATGGGGTA
GGGTACCAACCATACAGGGTGGTAGTCCTTAGCTTTGAACTTTTGCATG
CGCCAGCTACCGTCTGCGGTCCCAAAAAGAGTACGAACTTGGTAAAAAA
TAAATGCGTCAACTTTAATTTTAACGGTCTGACGGGAACGGGGGTTCTC
ACCGAGTCTAACAAAAAGTTTTTGCCATTTCAGCAGTTCGGACGAGATA
TTGCCGACACTACCGACGCCGTGCGCGATCCACAAACCTTGGAGATACT
CGACATCACACCTTGCAGCTTCGGTGGTGTTAGCGTTATTACGCCAGGA
ACGAACACTTCAAATCAGGTGGCCGTCTTGTATCAAGATGTTAACTGTA
CAGAGGTGCCCGTCGCAATACACGCAGATCAGCTCACCCCTACCTGGAG
AGTTTACTCTACAGGCTCTAATGTGTTCCAAACTAGGGCAGGCTGCTTG
ATTGGAGCTGAACACGTAAACAACTCATACGAGTGCGATATCCCGATTG
GTGCGGGTATTTGCGCCAGCTATCAGACGCAGACTAACTCACGATCAGT
GGCGTCACAGTCAATAATTGCGTATACAATGAGTCTCGGTGCAGAGAAT
TCCGTCGCATACTCAAACAACAGCATAGCGATTCCAACTAATTTTACCA
TTAGCGTTACAACGGAAATCCTCCCCGTTAGCATGACCAAGACCTCCGT
GGATTGCACAATGTACATCTGTGGAGACAGCACCGAGTGCTCCAATCTT
CTTCTTCAATATGGGAGCTTTTGCACACAGCTGAACCGCGCGCTCACTG
GCATTGCGGTAGAACAAGACAAAAACACCCAAGAAGTGTTTGCGCAGGT
CAAACAGATTTACAAGACACCACCAATCAAAGACTTCGGGGGGTTCAAT
TTCTCCCAAATTCTTCCTGATCCAAGCAAACCATCTAAAAGGTCCTTCA
TAGAAGACCTCCTGTTCAATAAGGTGACACTCGCGGACGCAGGGTTTAT
TAAGCAATATGGAGATTGTCTCGGGGATATTGCAGCGAGAGATCTTATT
TGTGCTCAAAAGTTTAATGGCTTGACTGTCCTCCCGCCCTTGCTCACAG
ACGAGATGATAGCACAATATACCAGTGCGCTGCTTGCGGGCACGATTAC
TTCCGGATGGACTTTCGGGGCAGGAGCGGCGCTCCAGATTCCTTTCGCG
ATGCAAATGGCCTACCGATTTAACGGGATTGGCGTCACACAAAACGTTC
TTTACGAAAACCAGAAACTTATTGCCAACCAATTCAATAGTGCAATTGG
TAAAATTCAGGATTCACTCTCCTCCACGGCGTCAGCTCTCGGCAAGCTC
CAAGACGTGGTTAACCAAAATGCGCAGGCGCTTAACACGCTTGTAAAAC
AACTCTCTTCTAATTTTGGCGCGATTAGTAGCGTTCTCAACGATATCTT
GTCACGCTTGGACAAAGTAGAAGCAGAAGTCCAAATAGACCGGCTGATC
ACTGGTCGATTGCAATCTCTGCAGACATACGTGACGCAGCAGCTCATCC
GGGCAGCCGAGATCCGGGCGTCCGCTAATCTGGCCGCAACCAAAATGAG
CGAGTGTGTCCTTGGGCAAAGCAAACGCGTGGATTTTTGCGGAAAGGGC
TACCACCTCATGAGTTTCCCCCAATCTGCCCCGCACGGTGTCGTCTTTT
TGCATGTAACGTATGTCCCGGCGCAGGAGAAAAATTTCACTACAGCTCC
CGCCATCTGTCACGACGGCAAGGCACACTTCCCTCGAGAAGGGGTGTTT
GTAAGCAATGGAACACACTGGTTCGTCACCCAGCGGAATTTTTATGAAC
CTCAGATAATCACAACAGACAACACTTTCGTTTCCGGAAACTGCGATGT
CGTAATTGGGATAGTGAATAATACCGTATATGACCCATTGCAGCCGGAG
TTGGACTCCTTTAAAGAGGAATTGGACAAATACTTTAAAAATCACACTT
CACCCGATGTGGACCTGGGAGATATATCCGGGATTAACGCAAGTGTGGT
TAACATACAAAAGGAAATTGACCGCCTTAACGAGGTTGCTAAGAACTTG
AATGAGTCTCTCATAGATCTCCAGGAACTGGGGAAATACGAGCAATATA
TCAAATGGCCGTCACGACTGGAGGAAGAACTGCGCCGACGCCTGACTGA
ATGA

In certain embodiments, the sequence encoding the spike protein extracellular domain comprises the sequence as set forth below (SEQ ID NO:30)

GCCACCATGGTTAACTTTACTAATCGGACCCAACTCCCGAGTGCGTACA
CTAACTCTTTTACCCGGGGCGTCTATTATCCTGACAAAGTGTTTCGATC
ATCTGTTTTGCACTCCACCCAAGATCTCTTTCTTCCATTTTTCAGCAAC
GTTACGTGGTTCCACGCGATACATGTTAGTGGTACTAATGGCACAAAGA
GATTTGACAACCCTGTGCTCCCGTTTAACGATGGGGTCTATTTCGCTTC
CACTGAGAAGTCCAACATTATTCGCGGTTGGATCTTCGGCACCACACTC
GATTCAAAAACCCAAAGCTTGCTCATAGTTAACAACGCCACAAACGTAG
TCATTAAAGTGTGCGAATTTCAGTTTTGTAACTATCCCTTTTTGGGCGT
CTACTATCACAAGAACAACAAGTCCTGGATGGAATCTGAGTTTCGGGTC
TACAGTTCAGCCAACAACTGTACGTTTGAGTACGTTAGCCAGCCCTTTC
TGATGGATCTTGAAGGGAAGCAAGGAAATTTCAAGAATCTTTCCGAGTT
TGTGTTTAAAAATATAGACGGATATTTCAAGATATACAGTAAGCACACA
CCGATCAATTTGGTACGGGATTTGCCGCAAGGATTTTCCGCCCTTGAGC
CCCTTGTAGATCTCCCCATCGGAATTAACATAACCAGGTTTCAAACACT
GCTGGCGCTCCACAGGAGCTATCTGACCCCGGGCGACTCATCCAGTGGG
TGGACCGCCGGAGCAGCCGCATACTACGTGGGGTATCTCCAGCCCAGAA
CGTTTCTCCTCAAGTATAATGAAAACGGGACCATCACCGACGCGGTTGA
TTGTGCACTCGATCCCCTCAGCGAGACAAAGTGCACACTTAAATCATTT
ACCGTCGAGAAGGGGATCTACCAAACATCCAACTTTCGGGTGCAGCCTA
CCGAGTCAATAGTACGGTTTCCTAACATCACCAATTTGTGCCCTTTTGG
GGAAGTATTTAATGCAACCCGGTTTGCGAGCGTATACGCTTGGAACCGA
AAACGGATAAGTAATTGTGTTGCAGATTATTCTGTGCTCTATAACTCAG
CTTCCTTCTCCACCTTTAAGTGTTACGGAGTATCTCCGACAAAATTGAA
TGATCTTTGTTTTACGAATGTTTACGCTGACTCATTTGTTATCCGAGGT
GACGAAGTGCGCCAGATAGCCCCCGGTCAAACAGGCAACATTGCTGATT
ATAACTACAAGTTGCCCGATGACTTCACGGGGTGTGTTATTGCATGGAA
TTCAAATAATCTTGATAGTAAGGTAGGGGGTAACTATAATTACCTTTAT
CGCTTGTTCCGCAAATCCAATCTGAAACCCTTCGAAAGGGATATCAGTA
CCGAGATTTATCAAGCCGGCTCCACACCTTGTAACGGAGTAAAAGGTTT
TAACTGTTATTTCCCACTGCAGAGTTACGGCTTTCAACCCACATACGGG
GTGGGATATCAGCCCTATCGCGTTGTGGTCTTGTCCTTTGAACTTCTCC
ACGCCCCTGCAACAGTTTGTGGTCCTAAAAAGAGTACAAATCTGGTAAA
AAATAAGTGCGTCAACTTTAACTTCAACGGACTGACTGGTACCGGGGTC
CTCACTGAGAGCAACAAGAAATTTTTGCCCTTCCAGCAATTCGGGAGGG
ATATTGCCGATACTACCGATGCTGTACGGGACCCCCAGACACTCGAAAT
ATTGGACATAACCCCGTGCTCTTTTGGAGGGGTCTCTGTGATTACCCCG
GGTACGAACACGAGTAATCAGGTGGCAGTGCTTTATCAGGGAGTGAATT
GCACAGAAGTACCAGTCGCGATCCATGCCGACCAGCTCACGCCGACCTG
GAGGGTTTATAGTACGGGTAGTAACGTTTTCCAAACACGGGCCGGTTGT
CTCATCGGGGCAGAGTACGTTAATAACAGCTATGAATGTGATATACCCA
TAGGTGCCGGGATTTGTGCTTCTTATCAAACCCAAACCAATAGCAGAAG
CGTTGCGTCACAGTCTATAATCGCCTATACGATGAGCCTCGGAGCGGAA
AACTCAGTTGCCTACTCCAACAATAGCATTGCTATCCCAACGAACTTCA
CTATTAGCGTAACCACGGAGATCCTTCCAGTTAGCATGACAAAAACTAG
CGTCGATTGCACTATGTATATTTGCGGCGACAGTACAGAGTGTAGTAAT
TTGCTCCTGCAGTACGGGTCTTTCTGCACCCAATTGAACCGAGCACTGA
CTGGCATCGCCGTGGAACAAGACAAAAACACGCAAGAAGTATTTGCGCA
AGTCAAGCAGATTTACAAGACTCCCCCGATCAAAGACTTTGGCGGATTT
AATTTTAGCCAGATACTCCCCGATCCATCTAAACCATCAAAACGGTCCT
TTATAGAAGATCTCTTGTTCAATAAAGTGACACTCGCGGACGCGGGCTT
TATCAAACAGTATGGGGACTGCCTTGGAGATATAGCTGCGAGGGACTTG
ATTTGCGCCCAGAAATTCAACGGGCTGACGGTTCTGCCGCCGCTCTTGA
CAGACGAAATGATTGCACAGTATACGAGTGCCTTGCTGGCAGGGACTAT
CACTTCCGGTTGGACCTTCGGCGCAGGAGCAGCGCTCCAGATCCCATTC
GCTATGCAAATGGCTTACCGATTCAACGGAATTGGCGTCACACAGAACG
TGCTTTACGAGAATCAAAAGCTGATTGCCAATCAATTCAACTCAGCGAT
TGGCAAAATACAAGATAGTCTCTCTTCAACTGCATCCGCGCTCGGTAAG
TTGCAGGACGTAGTCAATCAGAACGCACAGGCCCTGAACACGCTTGTTA
AACAACTGTCTTCAAACTTCGGTGCAATAAGTTCCGTACTTAATGATAT
CTTGTCAAGGTTGGATAAGGTCGAAGCTGAGGTACAAATCGACCGCCTT
ATAACAGGCAGACTCCAATCCCTGCAGACATACGTGACCCAGCAACTCA
TTAGGGCAGCCGAGATCAGAGCAAGTGCCAACCTCGCTGCTATTAAAAT
GTCTGAATGCGTTTTGGGGCAATCTAAGCGGGTTGATTTCTGCGGTAAG
GGATACCACTTGATGTCCTTTCCTCAGTCCGCTCCTCATGGGGTAGTAT
TTCTTCATGTTACATATGTACCAGCACAAGAGAAAAATTTTACTACTGC
CCCGGCCATATGCCATGATGGCAAAGCGCACTTCCCTCGGGAGGGGGTT
TTTGTGTCTAATGGAACCCACTGGTTCGTGACTCAGCGGAATTTCTATG
AACCCCAGATTATAACTACTGACAACACTTTCGTTTCTGGAAACTGCGA
TGTAGTAATAGGCATAGTAAATAATACAGTATATGATCCCTTGCAACCA
GAATTGGACTCTTTTAAAGAAGAGCTCGACAAATATTTTAAAAACCATA
CATCTCCAGACGTTGATCTTGGAGACATCTCTGGGATAAACGCTAGCTT
CGTAAACATACAAAAAGAAATCGATAGGCTTAACGAGGTCGCAAAAAAT
CTTAATGAAAGTCTTATCGATTTGCAAGAGCTTGGAAAGTATGAGCAGT
ATATCAAATGGCCTTCACGACTGGAGGAAGAACTGCGCCGACGCCTGAC
TGAATGA

In certain embodiments, the sequence encoding the spike protein extracellular domain comprises the sequence as set forth below (SEQ ID NO:31)

GCCACCATGGTTAATCTTACAACCAGAACTCAATTACCCCCTGCATACA
CTAATTCTTTCACACGTGGTGTTTATTACCCTGACAAAGTTTTCAGATC
CTCAGTTTTACATTCAACTCAGGACTTGTTCTTACCTTTCTTTTCCAAT
GTTACTTGGTTCCATGCTATACATGTCTCTGGGACCAATGGTACTAAGA
GGTTTGATAACCCTGTCCTACCATTTAATGATGGTGTTTATTTTGCTTC
CATTGAGAAGTCTAACATAATAAGAGGCTGGATTTTTGGTACTACTTTA
GATTCGAAGACCCAGTCCCTACTTATTGTTAATAACGCTACTAATGTTG
TTATTAAAGTCTGTGAATTTCAATTTTGTAATGATCCATTTTTGGGTGT
TTATTACCACAAAAACAACAAAAGTTGGATGAAAAGTGAGTTCAGAGTT
TATTCTAGTGCGAATAATTGCACTTTTGAATATGTCTCTCAGCCTTTTC
TTATGGACCTTGAAGGAAAACAGGGTAATTTCAAAAATCTTAGGGAATT
TGTGTTTAAGAATATTGATGGTTATTTTAAAATATATTCTAAGCACACG
CCTATTAATTTAGTGCGTGATCTCCCTCAGGGTTTTTCGGCTTTAGAAC
CATTGGTAGATTTGCCAATAGGTATTAACATCACTAGGTTTCAAACTTT
ACTTGCTTTACATAGAAGTTATTTGACTCCTGGTGATTCTTCTTCAGGT
TGGACAGCTGGTGCTGCAGCTTATTATGTGGGTTATCTTCAACCTAGGA
CTTTTCTATTAAAATATAATGAAAATGGAACCATTACAGATGCTGTAGA
CTGTGCACTTGACCCTCTCTCAGAAACAAAGTGTACGTTGAAATCCTTC
ACTGTAGAAAAAGGAATCTATCAAACTTCTAACTTTAGAGTCCAACCAA
CAGAATCTATTGTTAGATTTCCTAATATTACAAACTTGTGCCCTTTTGG
TGAAGTTTTTAACGCCACCAGATTTGCATCTGTTTATGCTTGGAACAGG
AAGAGAATCAGCAACTGTGTTGCTGATTATTCTGTCCTATATAATTCCG
CATCATTTTCCACTTTTAAGTGTTATGGAGTGTCTCCTACTAAATTAAA
TGATCTCTGCTTTACTAATGTCTATGCAGATTCATTTGTAATTAGAGGT
GATGAAGTCAGACAAATCGCTCCAGGGCAAACTGGAAAGATTGCTGATT
ATAATTATAAATTACCAGATGATTTTACAGGCTGCGTTATAGCTTGGAA
TTCTAACAAACTTGATTCTAAGGTTGGTGGTAATTATAATTACCGCTAT
AGATTGTTTAGGAAGTCTAATCTCAAACCTTTTGAGAGAGATATTTCAA
CTGAAATCTATCAGGCCGGTAGCACACCTTGTAATGGTGTTCAGGGTTT
TAATTGTTACTTTCCTTTACAATCATATGGTTTCCAACCCACTTATGGT
GTTGGTTACCAACCATACAGAGTAGTAGTACTTTCTTTTGAACTTCTAC
ATGCACCAGCAACTGTTTGTGGACCTAAAAAGTCTACTAATTTGGTTAA
AAACAAATGTGTCAATTTCAACTTCAATGGTTTAACAGGCACAGGTGTT
CTTACTGAGTCTAACAAAAAGTTTCTGCCTTTCCAACAATTTGGCAGAG
ACATTGATGACACTACTGATGCTGTCCGTGATCCACAGACACTTGAGAT
TCTTGACATTACACCATGTTCTTTTGGTGGTGTCAGTGTTATAACACCA
GGAACAAATACTTCTAACCAGGTTGCTGTTCTTTATCAGGGTGTTAACT
GCACAGAAGTCCCTGTTGCTATTCATGCAGATCAACTTACTCCTACTTG
GCGTGTTTATTCTACAGGTTCTAATGTTTTTCAAACACGTGCAGGCTGT
TTAATAGGGGCTGAACATGTCAACAACTCATATGAGTGTGACATACCCA
TTGGTGCAGGTATATGCGCTAGTTATCAGACTCAGACTAATTCTAGTGT
AGCTAGTCAATCCATCATTGCCTACACTATGTCACTTGGTGCAGAAAAT
TCAGTTGCTTACTCTAATAACTCTATTGCCATACCCATTAATTTTACTA
TTAGTGTTACCACAGAAATTCTACCAGTGTCTATGACCAAGACATCAGT
AGATTGTACAATGTACATTTGTGGTGATTCAACTGAATGCAGCAATCTT
TTGTTGCAATATGGCAGTTTTTGTACACAATTAAACCGTGCTTTAACTG
GAATAGCTGTTGAACAAGACAAAAACACCCAAGAAGTTTTTGCACAAGT
CAAACAAATTTACAAAACACCACCAATTAAAGATTTTGGTGGTTTTAAT
TTTTCACAAATATTACCAGATCCATCAAAACCAAGCAAGAGGTCATTTA
TTGAAGATCTACTTTTCAACAAAGTGACACTTGCAGATGCTGGCTTCAT
CAAACAATATGGTGATTGCCTTGGTGATATTGCTGCTAGAGACCTCATT
TGTGCACAAAAGTTTAACGGCCTTACTGTTTTGCCACCTTTGCTCACAG
ATGAAATGATTGCTCAATACACTTCTGCACTGTTAGCGGGTACAATCAC
TTCTGGTTGGACCTTTGGTGCAGGTGCTGCATTACAAATACCATTTGCT
ATGCAAATGGCTTATAGGTTTAATGGTATTGGAGTTACACAGAATGTTC
TCTATGAGAACCAAAAATTGATTGCCAACCAATTTAATAGTGCTATTGG
CAAAATTCAAGACTCACTTTCTTCCACAGCAAGTGCACTTGGAAAACTT
CAAGATGTGGTCAACCAAAATGCACAAGCTTTAAACACGCTTGTTAAAC
AACTTAGCTCCAATTTTGGTGCAATTTCAAGTGTTTTAAATGATATCCT
TGCACGTCTTGACAAAGTTGAGGCTGAAGTGCAAATTGATAGGTTGATC
ACAGGCAGACTTCAAAGTTTGCAGACATATGTGACTCAACAATTAATTA
GAGCTGCAGAAATCAGAGCTTCTGCTAATCTTGCTGCTACTAAAATGTC
AGAGTGTGTACTTGGACAATCAAAAAGAGTTGATTTTTGTGGAAAGGGC
TATCATCTTATGTCCTTCCCTCAGTCAGCACCTCATGGTGTAGTCTTCT
TGCATGTGACTTATGTCCCTGCACACGAAAAGAACTTCACAACTGCTCC
TGCCATTTGTCATGATGGAAAAGCACACTTTCCTCGTGAAGGTGTCTTT
GTTTCAAATGGCACACACTGGTTTGTAACACAAAGGAATTTTTATGAAC
CACAAATCATTACTACACACAACACATTTGTGTCTGGTAACTGTGATGT
TGTAATAGGAATTGTCAACAACACAGTTTATGATCCTTTGCAACCTGAA
TTAGACTCATTCAAGGAGGAGTTAGATAAATATTTTAAGAATCATACAT
CACCAGATGTTGATTTAGGTGACATCTCTGGCATTAATGCTTCAGTTGT
AAACATTCAAAAAGAAATTGACCGCCTCAATGAGGTTGCCAAGAATTTA
AATGAATCTCTCATCGATCTCCAAGAACTTGGAAAGTATGAGCAGTATA
TAAAATGGCCATCACGACTGGAGGAAGAACTGCGCCGACGCCTGACTGA
ATAA

In certain embodiments, the sequence encoding the spike protein extracellular domain comprises the sequence as set forth below (SEQ ID NO:32)

GCCGCCACCATGGTGAACCTGACCACCCGGACCCAGCTACCTCCTGCCT
ACACAAACAGCTTCACCCGCGGCGTGTACTACCCTGATAAGGTGTTCCG
GAGCTCTGTGCTCCACAGCACACAGGACCTGTTCCTGCCTTTCTTCAGC
AACGTGACGTGGTTCCACGCCATCCACGTGTCCGGCACCAATGGTACAA
AAAGGTTTGACAACCCCGTGCTCCCCTTCAACGACGGCGTGTATTTTGC
CAGCACTGAAAAGTCCAATATCATCCGAGGATGGATCTTCGGCACAACG
CTGGACTCTAAGACCCAGAGCCTGCTGATCGTGAACAACGCCACCAACG
TGGTGATCAAGGTGTGCGAGTTTCAGTTCTGCAACGATCCTTTTCTGGG
CGTTTACTACCACAAGAACAACAAGAGCTGGATGGAAAGCGAGTTCAGA
GTGTATTCTAGCGCCAACAACTGTACCTTCGAATACGTCAGCCAGCCTT
TCCTGATGGACCTGGAAGGCAAGCAGGGCAACTTCAAGAACCTGAGAGA
GTTCGTGTTCAAGAACATCGACGGCTACTTCAAGATCTACAGCAAGCAC
ACACCTATCAACCTGGTGCGGGACCTTCCGCAGGGATTCTCCGCGCTGG
AACCCCTGGTGGACCTGCCTATTGGCATCAACATCACCAGATTTCAGAC
CCTGCTGGCCCTGCACAGAAGCTACCTGACACCTGGCGACTCTTCTAGC
GGCTGGACCGCCGGCGCCGCCGCTTATTACGTGGGGTACCTGCAGCCCA
GAACCTTCCTGCTGAAATACAATGAGAACGGAACCATCACCGATGCCGT
GGACTGCGCCCTGGACCCCCTGTCTGAAACCAAGTGCACCCTGAAATCT
TTTACAGTGGAAAAGGGAATTTACCAGACCTCCAACTTTAGAGTCCAGC
CTACCGAAAGCATCGTGCGGTTCCCTAACATCACCAACCTGTGTCCTTT
CGGCGAGGTTTTCAACGCCACACGGTTCGCCAGCGTATACGCCTGGAAC
CGGAAGCGGATCTCTAACTGTGTGGCAGACTACTCTGTGCTATACAACT
CGGCCAGCTTTAGCACCTTCAAGTGCTACGGCGTGTCCCCAACCAAGCT
GAACGACCTGTGCTTCACCAACGTGTACGCCGATAGCTTCGTGATCAGA
GGCGATGAGGTGCGCCAGATCGCTCCTGGACAGACCGGAAAGATCGCTG
ACTACAATTACAAGCTGCCCGACGACTTCACCGGCTGCGTGATCGCCTG
GAACAGCAACAACCTGGATTCCAAAGTGGGCGGCAACTACAACTATCTG
TACCGGCTCTTCAGAAAGTCTAACCTGAAGCCCTTTGAGAGAGACATCT
CCACAGAAATCTACCAGGCCGGAAGCACACCTTGCAACGGCGTGGAAGG
ATTTAATTGCTATTTTCCTCTGCAGTCCTACGGCTTCCAACCTACCAAC
GGAGTGGGCTACCAGCCTTACAGAGTGGTCGTGCTGAGCTTTGAGCTGC
TGCATGCCCCTGCTACCGTGTGTGGCCCTAAGAAAAGCACGAACCTGGT
CAAGAACAAATGCGTGAACTTCAACTTCAATGGACTGACAGGCACCGGC
GTGCTCACAGAGAGCAACAAGAAGTTCCTGCCATTTCAGCAGTTCGGCA
GAGACATAGCTGACACCACCGACGCCGTGCGGGACCCCCAGACCCTGGA
AATCCTGGATATCACACCCTGCAGCTTTGGAGGCGTATCTGTGATCACA
CCTGGCACCAACACAAGCAATCAGGTCGCCGTGCTGTACCAAGACGTGA
ACTGCACAGAGGTGCCGGTGGCCATCCACGCCGATCAGCTGACCCCCAC
CTGGCGGGTGTACTCCACCGGCAGCAACGTGTTCCAAACCCGGGCCGGC
TGCCTGATCGGCGCCGAACATGTGAATAACAGCTACGAATGTGATATCC
CTATTGGAGCCGGAATTTGCGCCAGCTACCAAACCCAAACCAACTCTCC
AAGAAGAGCCAGAAGCGTGGCTAGCCAGTCTATCATCGCCTACACCATG
AGCCTGGGCGCTGAAAACAGCGTGGCTTACAGCAACAACAGCATCGCCA
TTCCTACTAACTTTACCATCAGCGTGACTACAGAGATCCTGCCCGTGAG
CATGACCAAGACCAGCGTGGACTGCACCATGTACATCTGTGGCGATTCT
ACCGAGTGCAGCAACTTGCTACTGCAGTATGGTAGCTTTTGCACCCAAC
TGAACAGAGCCCTGACTGGCATCGCCGTTGAGCAGGACAAGAACACCCA
GGAGGTGTTCGCCCAGGTCAAGCAGATCTATAAGACCCCACCAATCAAG
GACTTCGGCGGCTTCAACTTCAGCCAGATCCTGCCCGACCCGAGCAAAC
CTAGCAAGAGAAGCTTCATCGAGGATCTTCTGTTCAACAAGGTGACGCT
GGCCGATGCCGGCTTCATCAAACAGTACGGCGATTGTCTGGGCGACATC
GCCGCCAGAGATCTGATCTGCGCCCAGAAGTTCAATGGCCTGACCGTGC
TGCCGCCTCTGCTCACAGACGAGATGATCGCCCAGTACACCTCTGCCCT
GTTAGCAGGCACAATCACGAGCGGCTGGACATTTGGCGCGGGCGCCGCT
CTGCAGATCCCTTTCGCTATGCAGATGGCCTATAGATTTAATGGCATCG
GCGTGACCCAGAATGTGCTGTACGAGAACCAAAAGCTGATCGCTAATCA
GTTCAATAGCGCCATCGGAAAGATCCAGGACTCTCTGAGCAGCACCGCC
TCCGCTCTGGGCAAACTGCAGGACGTGGTCAATCAAAACGCTCAGGCTC
TAAACACCCTGGTGAAGCAGCTGTCAAGCAACTTCGGCGCCATCTCCTC
AGTTCTTAACGACATCTTGTCTAGACTGGACAAGGTGGAGGCTGAGGTG
CAGATCGATAGACTGATCACAGGCAGATTACAGTCTCTGCAGACATACG
TGACCCAACAGCTGATCCGGGCCGCCGAGATCAGAGCCTCCGCCAACCT
GGCTGCCACCAAGATGAGCGAGTGTGTGCTTGGCCAGTCCAAGAGAGTG
GACTTCTGCGGCAAAGGCTACCACCTGATGAGCTTCCCACAGAGCGCTC
CTCACGGAGTTGTGTTCCTGCACGTGACCTACGTGCCTGCCCAGGAGAA
GAATTTCACAACCGCCCCTGCCATCTGCCACGACGGTAAGGCCCACTTC
CCCAGAGAGGGCGTGTTCGTGTCTAATGGGACCCACTGGTTCGTGACAC
AGAGGAACTTCTACGAGCCCCAGATCATCACAACAGATAATACCTTCGT
GTCTGGCAACTGCGATGTGGTGATCGGCATCGTGAACAATACCGTGTAC
GACCCTCTGCAGCCCGAGCTGGACAGCTTCAAAGAGGAACTGGACAAGT
ACTTCAAGAATCACACCTCCCCTGACGTGGACCTGGGAGATATCTCCGG
CATCAACGCCTCCGTTGTGAACATTCAAAAAGAAATCGACAGACTGAAC
GAGGTGGCCAAGAACCTAAACGAGTCTCTGATCGACCTGCAAGAACTGG
GCAAGTACGAGCAGTACATCAAGTGGCCataa

In certain embodiments, the sequence encoding the spike protein extracellular domain without the furin sequence comprises the sequence as set forth below (SEQ ID NO:33)

GCCGCCACCATGGTGAACCTGACCACCCGGACCCAGCTACCaCCaGCCT
ACACAAACAGCTTCACCCGCGGCGTGTACTACCCaGAcAAGGTGTTCCG
GAGCTCaGTGCTCCACAGCACACAGGACCTGTTCCTGCCaTTCTTCAGC
AACGTGACGTGGTTCCACGCCATCCACGTGTCCGGCACCAAcGGaACAA
AAAGGTTcGACAACCCCGTGCTCCCCTTCAACGACGGCGTGTAcTTcGC
CAGCACaGAAAAGTCCAAaATCATCCGAGGATGGATCTTCGGCACAACG
CTGGACTCaAAGACCCAGAGCCTGCTGATCGTGAACAACGCCACCAACG
TGGTGATCAAGGTGTGCGAGTTcCAGTTCTGCAACGATCCaTTcCTGGG
CGTaTACTACCACAAGAACAACAAGAGCTGGATGGAAAGCGAGTTCAGA
GTGTAcTCaAGCGCCAACAACTGcACCTTCGAATACGTCAGCCAGCCaT
TCCTGATGGACCTGGAAGGCAAGCAGGGCAACTTCAAGAACCTGAGAGA
GTTCGTGTTCAAGAACATCGACGGCTACTTCAAGATCTACAGCAAGCAC
ACACCcATCAACCTGGTGCGGGACCTaCCGCAGGGATTCTCCGCGCTGG
AACCCCTGGTGGACCTGCCaATaGGCATCAACATCACCAGATTcCAGAC
CCTGCTGGCCCTGCACAGAAGCTACCTGACACCaGGCGACagcagcAGC
GGCTGGACCGCCGGCGCCGCCGCaTAcTACGTGGGGTACCTGCAGCCCA
GAACCTTCCTGCTGAAATACAAcGAGAACGGAACCATCACCGAcGCCGT
GGACTGCGCCCTGGACCCCCTGagcGAAACCAAGTGCACCCTGAAAagc
TTcACAGTGGAAAAGGGAATaTACCAGACCTCCAACTTcAGAGTCCAGC
CaACCGAAAGCATCGTGCGGTTCCCaAACATCACCAACCTGTGcCCaTT
CGGCGAGGTaTTCAACGCCACACGGTTCGCCAGCGTATACGCCTGGAAC
CGGAAGCGGATCagcAACTGcGTGGCAGACTACagcGTGCTATACAACa
gcGCCAGCTTaAGCACCTTCAAGTGCTACGGCGTGTCCCCAACCAAGCT
GAACGACCTGTGCTTCACCAACGTGTACGCCGAcAGCTTCGTGATCAGA
GGCGAcGAGGTGCGCCAGATCGCaCCaGGACAGACCGGAAAGATCGCaG
ACTACAAcTACAAGCTGCCCGACGACTTCACCGGCTGCGTGATCGCCTG
GAACAGCAACAACCTGGAcTCCAAAGTGGGCGGCAACTACAACTAcCTG
TACCGGCTCTTCAGAAAGagcAACCTGAAGCCCTTcGAGAGAGACATCa
gcACAGAAATCTACCAGGCCGGAAGCACACCaTGCAACGGCGTGGAAGG
ATTcAAcTGCTATTTcCCaCTGCAGTCCTACGGCTTCCAACCaACCAAC
GGAGTGGGCTACCAGCCaTACAGAGTGGTCGTGCTGAGCTTcGAGCTGC
TGCAcGCCCCaGCaACCGTGTGcGGCCCaAAGAAAAGCACGAACCTGGT
CAAGAACAAATGCGTGAACTTCAACTTCAAcGGACTGACAGGCACCGGC
GTGCTCACAGAGAGCAACAAGAAGTTCCTGCCATTcCAGCAGTTCGGCA
GAGACATAGCaGACACCACCGACGCCGTGCGGGACCCCCAGACCCTGGA
AATCCTGGAcATCACACCCTGCAGCTTcGGAGGCGTAagcGTGATCACA
CCaGGCACCAACACAAGCAAcCAGGTCGCCGTGCTGTACCAAGACGTGA
ACTGCACAGAGGTGCCGGTGGCCATCCACGCCGAcCAGCTGACCCCCAC
CTGGCGGGTGTACTCCACCGGCAGCAACGTGTTCCAAACCCGGGCCGGC
TGCCTGATCGGCGCCGAACAcGTGAAcAACAGCTACGAATGcGAcATCC
CaATcGGAGCCGGAATcTGCGCCAGCTACCAAACCCAAACCAACagcCC
AAGAAGAGCCAGAAGCGTGGCaAGCCAGagcATCATCGCCTACACCATG
AGCCTGGGCGCaGAAAACAGCGTGGCaTACAGCAACAACAGCATCGCCA
TcCCaACcAACTTcACCATCAGCGTGACcACAGAGATCCTGCCCGTGAG
CATGACCAAGACCAGCGTGGACTGCACCATGTACATCTGcGGCGAcagc
ACCGAGTGCAGCAACTTGCTACTGCAGTAcGGaAGCTTcTGCACCCAAC
TGAACAGAGCCCTGACcGGCATCGCCGTcGAGCAGGACAAGAACACCCA
GGAGGTGTTCGCCCAGGTCAAGCAGATCTAcAAGACCCCACCAATCAAG
GACTTCGGCGGCTTCAACTTCAGCCAGATCCTGCCCGACCCGAGCAAAC
CaAGCAAGAGAAGCTTCATCGAGGAcCTaCTGTTCAACAAGGTGACGCT
GGCCGAcGCCGGCTTCATCAAACAGTACGGCGAcTGcCTGGGCGACATC
GCCGCCAGAGAcCTGATCTGCGCCCAGAAGTTCAATGGCCTGACCGTGC
TGCCGCCaCTGCTCACAGACGAGATGATCGCCCAGTACACCagcGCCCT
GTTAGCAGGCACAATCACGAGCGGCTGGACATTcGGCGCGGGCGCCGCa
CTGCAGATCCCaTTCGCaATGCAGATGGCCTAcAGATTcAATGGCATCG
GCGTGACCCAGAAcGTGCTGTACGAGAACCAAAAGCTGATCGCaAAcCA
GTTCAAcAGCGCCATCGGAAAGATCCAGGACagcCTGAGCAGCACCGCC
TCCGCaCTGGGCAAACTGCAGGACGTGGTCAAcCAAAACGCaCAGGCaC
TAAACACCCTGGTGAAGCAGCTGTCAAGCAACTTCGGCGCCATCagcTC
AGTcCTaAACGACATCTTGagcAGACTGGACAAGGTGGAGGCaGAGGTG
CAGATCGAcAGACTGATCACAGGCAGATTACAGagcCTGCAGACATACG
TGACCCAACAGCTGATCCGGGCCGCCGAGATCAGAGCCTCCGCCAACCT
GGCaGCCACCAAGATGAGCGAGTGcGTGCTaGGCCAGagcAAGAGAGTG
GACTTCTGCGGCAAAGGCTACCACCTGATGAGCTTCCCACAGAGCGCaC
CaCACGGAGTaGTGTTCCTGCACGTGACCTACGTGCCaGCCCAGGAGAA
GAAcTTCACAACCGCCCCaGCCATCTGCCACGACGGaAAGGCCCACTTC
CCCAGAGAGGGCGTGTTCGTGagcAAcGGGACCCACTGGTTCGTGACAC
AGAGGAACTTCTACGAGCCCCAGATCATCACAACAGAcAAcACCTTCGT
GagcGGCAACTGCGAcGTGGTGATCGGCATCGTGAACAAcACCGTGTAC
GACCCaCTGCAGCCCGAGCTGGACAGCTTCAAAGAGGAACTGGACAAGT
ACTTCAAGAAcCACACCTCCCCaGACGTGGACCTGGGAGAcATCagcGG
CATCAACGCCTCCGTaGTGAACATcCAAAAAGAAATCGACAGACTGAAC
GAGGTGGCCAAGAACCTAAACGAGagcCTGATCGACCTGCAAGAACTGG
GCAAGTACGAGCAGTACATCAAGTGGCCaTAA

In certain embodiments, the sequence encoding a spike protein extracellular domain comprises the sequence as set forth below (SEQ ID NO:79)

GCCACCATGGTTAACTTTACTACTCGGACCCAACTCCCGCCGGCGTACA
CTAACTCTTTTACCCGGGGCGTCTATTATCCTGACAAAGTGTTTCGATC
ATCTGTTTTGCACTCCACCCAAGATCTCTTTCTTCCATTTTTCAGCAAC
GTTACGTGGTTCCACGCGATACATGTTAGTGGTACTAATGTTATAAAGA
GATTTGACAACCCTGTGCTCCCGTTTAACGATGGGGTCTATTTCGCTTC
CACTGAGAAGTCCAACATTATTCGCGGTTGGATCTTCGGCACCACACTC
GATTCAAAAACCCAAAGCTTGCTCATAGTTAACAACGCCACAAACGTAG
TCATTAAAGTGTGCGAATTTCAGTTTTGTAACTATCCCTTTTTGGGCGT
CTACTATCACAAGAACAACAAGTCCTGGATGGAATCTGAGTTTCGGGTC
TACAGTTCAGCCAACAACTGTACGTTTGAGTACGTTAGCCAGCCCTTTC
TGATGGATCTTGAAGGGAAGCAAGGAAATTTCAAGAATCTTTCCGAGTT
TGTGTTTAAAAATATAGACGGATATTTCAAGATATACAGTAAGCACACA
CCGATCAATTTGGTACGGGATTTGCCGCAAGGATTTTCCGCCCTTGAGC
CCCTTGTAGATCTCCCCATCGGAATTAACATAACCAGGTTTCAAACACT
GCTGGCGCTCCACAACTCATCCAGTGGGTGGACCGCCGGAGCAGCCGCA
TACTACGTGGGGTATCTCCAGCCCAGAACGTTTCTCCTCAAGTATAATG
AAAACGGGACCATCACCGACGCGGTTGATTGTGCACTCGATCCCCTCAG
CGAGACAAAGTGCACACTTAAATCATTTACCGTCGAGAAGGGGATCTAC
CAAACATCCAACTTTCGGGTGCAGCCTACCGAGTCAATAGTACGGTTTC
CTAACATCACCAATTTGTGCCCTTTTGGGGAAGTATTTAATGCAACCCG
GTTTGCGAGCGTATACGCTTGGAACCGAAAACGGATAAGTAATTGTGTT
GCAGATTATTCTGTGCTCTATAACTCAGCTTCCTTCTCCACCTTTAAGT
GTTACGGAGTATCTCCGACAAAATTGAATGATCTTTGTTTTACGAATGT
TTACGCTGACTCATTTGTTATCCGAGGTGACGAAGTGCGCCAGATAGCC
CCCGGTCAAACAGGCAAAATTGCTGATTATAACTACAAGTTGCCCGATG
ACTTCACGGGGTGTGTTATTGCATGGAATTCAAATAATCTTGATAGTAA
GGTAGGGGGTAACTATAATTACCAATATCGCTTGTTCCGCAAATCCAAT
CTGAAACCCTTCGAAAGGGATATCAGTACCGAGATTTATCAAGCCGGCT
CCACACCTTGTAACGGAGTAAAAGGTTTTAACTGTTATTCCCCACTGCA
GAGTTACGGCTTTCAACCCACATACGGGGTGGGATATCAGCCCTATCGC
GTTGTGGTCTTGTCCTTTGAACTTCTCCACGCCCCTGCAACAGTTTGTG
GTCCTAAAAAGAGTACAAATCTGGTAAAAAATAAGTGCGTCAACTTTAA
CTTCAACGGACTGACTGGTACCGGGGTCCTCACTGAGAGCAACAAGAAA
TTTTTGCCCTTCCAGCAATTCGGGAGGGATATTGCCGATACTACCGATG
CTGTACGGGACCCCCAGACACTCGAAATATTGGACATAACCCCGTGCTC
TTTTGGAGGGGTCTCTGTGATTACCCCGGGTACGAACACGAGTAATCAG
GTGGCAGTGCTTTATCAGGGAGTGAATTGCACAGAAGTACCAGTCGCGA
TCCATGCCGACCAGCTCACGCCGACCTGGAGGGTTTATAGTACGGGTAG
TAACGTTTTCCAAACACGGGCCGGTTGTCTCATCGGGGCAGAGTACGTT
AATAACAGCTATGAATGTGATATACCCATAGGTGCCGGGATTTGTGCTT
CTTATCAAACCCAAACCAATAGCTCACAGTCTATAATCGCCTATACGAT
GAGCCTCGGAGCGGAAAACTCAGTTGCCTACTCCAACAATAGCATTGCT
ATCCCAACGAACTTCACTATTAGCGTAACCACGGAGATCCTTCCAGTTA
GCATGACAAAAACTAGCGTCGATTGCACTATGTATATTTGCGGCGACAG
TACAGAGTGTAGTAATTTGCTCCTGCAGTACGGGTCTTTCTGCACCCAA
TTGAACCGAGCACTGACTGGCATCGCCGTGGAACAAGACAAAAACACGC
AAGAAGTATTTGCGCAAGTCAAGCAGATTTACAAGACTCCCCCGATCAA
AGACTTTGGCGGATTTAATTTTAGCCAGATACTCCCCGATCCATCTAAA
CCATCAAAACGGTCCTTTATAGAAGATCTCTTGTTCAATAAAGTGACAC
TCGCGGACGCGGGCTTTATCAAACAGTATGGGGACTGCCTTGGAGATAT
AGCTGCGAGGGACTTGATTTGCGCCCAGAAATTCAACGGGCTGAATGTT
CTGCCGCCGCTCTTGACAGACGAAATGATTGCACAGTATACGAGTGCCT
TGCTGGCAGGGACTATCACTTCCGGTTGGACCTTCGGCGCAGGAGCAGC
GCTCCAGATCCCATTCGCTATGCAAATGGCTTACCGATTCAACGGAATT
GGCGTCACACAGAACGTGCTTTACGAGAATCAAAAGCTGATTGCCAATC
AATTCAACTCAGCGATTGGCAAAATACAAGATAGTCTCTCTTCAACTGC
ATCCGCGCTCGGTAAGTTGCAGGACGTAGTCAATCAGAACGCACAGGCC
CTGAACACGCTTGTTAAACAACTGTCTTCAAACTTCGGTGCAATAAGTT
CCGTACTTAATGATATCTTGTCAAGGTTGGATAAGGTCGAAGCTGAGGT
ACAAATCGACCGCCTTATAACAGGCAGACTCCAATCCCTGCAGACATAC
GTGACCCAGCAACTCATTAGGGCAGCCGAGATCAGAGCAAGTGCCAACC
TCGCTGCTACAAAAATGTCTGAATGCGTTTTGGGGCAATCTAAGCGGGT
TGATTTCTGCGGTAAGGGATACCACTTGATGTCCTTTCCTCAGTCCGCT
CCTCATGGGGTAGTATTTCTTCATGTTACATATGTACCAGCACAAGAGA
AAAATTTTACTACTGCCCCGGCCATATGCCATGATGGCAAAGCGCACTT
CCCTCGGGAGGGGGTTTTTGTGTCTAATGGAACCCACTGGTTCGTGACT
CAGCGGAATTTCTATGAACCCCAGATTATAACTACTGACAACACTTTCG
TTTCTGGAAACTGCGATGTAGTAATAGGCATAGTAAATAATACAGTATA
TGATCCCTTGCAACCAGAATTGGACTCTTTTAAAGAAGAGCTCGACAAA
TATTTTAAAAACCATACATCTCCAGACGTTGATCTTGCTGACATCTCTG
GGATAAACGCTAGCGTAGTAAACATACAAAAAGAAATCGATAGGCTTAA
CGAGGTCGCAAAAAATCTTAATGAAAGTCTTATCGATTTGCAAGAGCTT
GGAAAGTATGAGCAGTATATCAAATGGCCTTCACGACTGGAGGAAGAAC
TGCGCCGACGCCTGACTGAATGA

Immune Response Enhancing Components:

In certain embodiments, the vaccines comprise or encode one or more additional components to enhance the immune response to the one or more viral immunogens. These components may include, for example, targeting molecules, elements which enhance antigen processing, immunostimulatory molecules such as cytokines and other adjuvants. In certain embodiments, the vaccine comprises or encodes one or more fusion polypeptides comprising the one or more additional components and one or more viral immunogens. The additional components include but are not limited to one or more targeting molecules/motifs. Exemplary targeting motifs include but are not limited to endosome/lysosome (i.e. endolysosomal) motif. In certain embodiments where the vaccine comprises or encodes more than more immunogen, each immunogen may be fused to one or more targeting molecules. The targeting molecules may be the same for all immunogens in the vaccine or different.

In certain embodiments, the targeting molecule comprises a sequence or encoded by a sequence as set forth below. In other embodiments, the targeting molecule comprises or is encoded by a sequence having at least 50%, at least 60%, at least 70%, at least 75%, at least 80%, at least 85%, at least 90%, at least 95%, at least 98%, at least 99% sequence identity to a sequence set forth below.

It has been found that vaccine performance is enhanced when the immunogen is delivered to the endolysosomal compartment of dendritic cells for antigen processing and loading on MHC I and II molecules. Various endolysosomal targeting sequence molecules are known in the art. In certain embodiments, the vaccine comprises or is capable of expressing one or more fusion proteins comprises one or more endolysosomal targeting molecules and one or more immunogens. Lysosomal targeting motifs typically have the consensus sequences YXXφ (tyrosine motif, where φ is a hydrophobic amino acid) or EXXXLL (dileucine motif; E may be replaced with D, and L with I or V).

TABLE 1
Dileucine- and tyrosine-based sorting signals in the cytoplasmic
domains of human antigen-presenting molecules

| | Protein | |
|---|---|---|
| | desig- | |
| Classification | nation | Cytoplasmic tail sequence |
| Invariant chain | Ii | MDDQRDLISNNEQLPMLGRRPGAPESKCSR-Tm^{a} |
| Classical^{b} MHC II x/β chains | DRx1^{c} | Tm-RKSNAAERRGPL |
| | DQx1 | Tm-RSVGASRHQGPL |
| | DPx1 | Tm-RSGHDPRAQGTL |
| | DRβ1 | Tm-RNQKGHSGLQPTGFLS |
| | DRβ4 | Tm-RNQKGHSGLQPTGLLS |
| | DRβ5 | Tm-KNQKGHSGLHPTGLVS |
| | DQβ1 | Tm-RSRKGLLH |
| | DPβ1 | Tm-RSKKVQRGSA |
| Non-classical MHC II x/β | DQx1 | Tm-YVSSVPR |
| chains | DMx1 | Tm-KPCSGD |
| | DQβ1 | Tm-RAQKGYVRTQMSGNEVSRAVLLPQSC |
| | DMβ1 | Tm-RRAGHSSYTPLPGSNYSEGWHIS |
| Classical MHC Ia x chains | HLA-A | Tm-RRKSSDRKGGSYTQAASSDSAQGSDVSLTACKV |
| | HLA-B | Tm-RRKSSGGKGGSYSQAACSDSAQGSDVSLTA |
| | HLA-C | Tm-RRKSSGGKGGSCSQAASSNSAQGSDESLIACKA |
| Non-classical MHC Ib x | HLA-E | Tm-RKKSSGGKGGSYSKAEWSDSAQGSESHSL |
| chains | HLA-F | Tm-RKKSSDRNRGSYSQAAVTDSAQGSGVSLTANKV |
| | HLA-G | Tm-RKKSSD |
| | HLA-H | Tm-KRQGSRGAMGHYVLAERE |
| CD1 x chains | CD1a | Tm-RKRCFC |
| | CD1b | Tm-RRRSYQNIP |
| | CD1c | Tm-KHCSYQDIL |
| | CD1d | Tm-RQTSYQGVL |
| | CD1e | Tm-KQ |

In specific embodiments, the fusion protein comprises one or more CD74 or fragments thereof and one or more the immunogen or fragments thereof. In certain embodiments, the fusion protein comprises the CD74 transmembrane and/or CD74 cytoplasmic and the one or immunogen or fragment thereof.

In certain embodiments, the CD74 cytoplasmic domain comprises the sequence as set forth below:

(SEQ ID NO: 67)
MHRRRSRSCREDQNPVMDDQRDLISNNEQLPMLGRRPGAPESKCSR

In certain embodiments, the CD74 cytoplasmic domain is encoded by the sequence as set forth below:

(SEQ ID NO: 75)
CACCGGAGGAGATCCAGAAGTTGCAGAGAAGACCAAAATCCAGTAATGG
ATGACCAAAGGGATCTTATTTCTAATAACGAGCAGCTCCCTATGCTGGG
ACGCCGACCGGGGGCACCCGAATCAAAGTGCAGTCGC

In certain embodiments, the fusion protein comprises the CD74 transmembrane and cytoplasmic domain and the one or immunogen or fragment thereof. In certain embodiments, the CD74 transmembrane and cytoplasmic domain has the sequence set forth below:

(SEQ ID NO: 34)
HRRRSRSCREDQKPVMDDQRDLISNNEQLPMLGRRPGAPESKCSRGAL
YTGFSILVTLLLAGQATTAYFLY

In certain embodiments, the vaccine comprises or encodes the CD74 Cytoplasmic domain with a spike protein, variant or fragment thereof. In certain embodiments, the vaccine comprises or encodes the CD74 Cytoplasmic domain with an influenza immunogen, variant or fragment thereof.

In certain embodiments, the vaccine comprises or encodes the CD74 Cytoplasmic domain and transmembrane domain with a spike protein, variant or fragment thereof. In certain embodiments, the vaccine comprises or encodes the CD74 Cytoplasmic domain and transmembrane domain with an influenza immunogen, variant or fragment thereof.

In certain embodiments, the vaccine comprises or encodes the CD74 Cytoplasmic domain and transmembrane domain with full length SARS-Cov-2 Spike Protein (without furin cleavage site).

In certain embodiments, the vaccine comprises or encodes the CD74 Cytoplasmic domain and transmembrane domain with the extracellular domain of SARS-Cov-2 Spike Protein (without furin cleavage site).

In certain embodiments, the fusion protein comprises one or more human leukocyte antigen (HLA) sequences or fragment thereof and the one or more immunogen or fragment thereof.

In certain embodiments, the fusion protein comprises the HLA signal sequence with a spike protein, variant or fragment thereof. In certain embodiments, the fusion protein comprises the HLA signal sequence with an influenza immunogen, variant or fragment thereof.

In certain embodiments, the HLA signal sequence comprises the sequence as set forth below:

(SEQ ID NO: 68)
MAVMAPRTLLLLLSGALALTQTWA

In certain embodiments, the HLA signal sequence is encoded by the sequence as set forth below:

(SEQ ID NO: 69)
ATGGCGGTCATGGCACCACGCACGCTCCTTCTTTTGTTGTCTGGTGCC
CTTGCACTCACCCAAACGTGGGCA

In certain embodiments, the fusion protein comprises the HLA transmembrane domain. In certain embodiments, the HLA transmembrane domain comprises the sequence as set forth below:

(SEQ ID NO: 70)
WMVAAVVAGTIVAGLLVLGAIIGV

In certain embodiments, the HLA transmembrane domain is encoded by the sequence as set forth below:

(SEQ ID NO: 71)
TGGATGGTGGCTGCGGTCGTGGCAGGTACGATTGTGGCTGGCCTCCTC
GTTCTTGGGGCTATTATCGGTGTA

In certain embodiments, the fusion protein comprises the HLA cytoplasmic domain, fragment or fragment thereof. In certain embodiments, the HLA cytoplasmic domain comprises the sequence as set forth or fragment thereof below:

(SEQ ID NO: 72)
VGIIAGLVLLGAVITGAVVAAVMWRRKSSDRKGGSYTQAASSDSAQGS
DVSLTACKV

In certain embodiments, the fusion protein comprises the HLA cytoplasmic domain comprises

(SEQ ID NO: 80)
RRKSSDRKGGSYTQAASSDSAQGS

In certain embodiments, the fusion protein comprises a sequence from a HLA cytoplasmic domain comprising RRKSSDRKGGSYTQAAV (SEQ ID NO: 81)

In certain embodiments, the HLA cytoplasmic domain is encoded by the sequence as set forth below:

(SEQ ID NO: 73)
GTAGGTATTATAGCCGGGCTCGTGTTGTTGGGTGCAGTAATCACAGGAG
CCGTCGTGGCAGCGGTTATGTGGAGACGGAAATCTTCAGACCGCAAAGG
TGGTAGTTACACTCAAGCGGCTTCCAGTGATTCTGCCCAGGGCTCCGAC
GTATCCCTCACTGCGTGTAAGGTT

In specific embodiments, the fusion protein comprises the HLA transmembrane and cytoplasmic domain and immunogen or fragment thereof.

In specific embodiment, the HLA fragment comprises the following sequence:

(SEQ ID NO: 35)
MVKCATLSVDSGQASDSSAAQTYSGGKRDSSKRRWMVAAVVAGTIVAGL
LVLGAIIGV

In certain embodiments, the targeting moiety is a chimeric targeting moiety comprising portions of different molecules. For example, the targeting moiety may comprise the cytoplasmic domain from one molecule fused to the transmembrane domain of another molecule. In certain embodiments, the targeting moiety comprises the CD74 cytoplasmic domain fused to an HLA transmembrane sequence. In specific embodiments, the targeting moiety comprises the following sequence:

(SEQ ID NO: 36)
HRRRSRSCREDQKPVMDDQRDLISNNEQLPMLGRRPGAPESKCSRWMVA
AVVAGTIVAGLLVLGAIIGV

In specific embodiments, the targeting moiety comprises the following sequence:

(SEQ ID NO: 74)
MHRRRSRSCREDQNPVMDDQRDLISNNEQLPMLGRRPGAPESKCSRWMV
AAVVAGTIVAGLLVLGAIIGV

Other Elements:

The proteins of the present invention may also include tags. Appropriate tags are known in the art and include but are not limited to HA-, FLAG®- or myc- or alpha tags.

Exemplary Vaccines

In certain embodiments, the vaccine comprises or encodes one or more coronavirus immunogens including but not limited to SARS-CoV immunogens including but not limited to SARS-CoV2 immunogens. In certain embodiments, the vaccine comprises or encodes a SARS-CoV-2 spike protein, fragments, variants or derivatives thereof. Non-limiting exemplary spike protein sequences are set forth in SEQ ID NOs:1 to 18. In alternate embodiments, the vaccine comprises or encodes SARS-CoV-2 spike protein with the carboxy-terminal transmembrane region deleted. In certain embodiments, the vaccine comprises or encodes a fusion protein comprising a targeting molecule and a spike protein, fragment, variant or derivative thereof. Exemplary targeting molecules include for example, CD74 or fragments thereof, HLA or fragments there of or CD74-HLA chimeric molecules. Accordingly, in certain embodiments, the vaccine comprises or encodes a fusion protein comprising CD74 Cytoplasmic domain and HLA transmembrane domain with a SARS-Cov-2 Spike protein, fragment, variant or derivative thereof.

In certain embodiments, the vaccine comprises or encodes a fusion protein CD74 Cytoplasmic domain and transmembrane domain with a SARS-CoV-2 spike protein with the carboxy-terminal transmembrane region deleted.

In certain embodiments, the vaccine comprises or encodes a fusion protein comprising CD74 Cytoplasmic domain and HLA transmembrane domain with a SARS-Cov-2 spike protein, fragment, variant or derivative thereof. In certain embodiments, the vaccine comprises or encodes CD74 Cytoplasmic domain and HLA transmembrane domain with a SARS-CoV-2 spike protein with the carboxy-terminal transmembrane region deleted.

In certain embodiments the vaccine comprises or encodes CD74 Cytoplasmic domain and transmembrane domain with a spike protein. In specific embodiments, the vaccine comprises or encodes the sequence set forth below (SEQ ID NO:37):

MHRRRSRSCREDQKPVMDDQRDLISNNEQLPMLGRRPGAPESKCSRGAL
YTGFSILVTLLLAGQATTAYFLYVNLTTRTQLPPAYTNSFTRGVYYPDK
VFRSSVLHSTQDLFLPFFSNVTWFHAIHVSGTNGTKRFDNPVLPFNDGV
YFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKN
LREFVFKNIDGYFKIYSKHTPINLVRDLPQGFSALEPLVDLPIGINITR
FQTLLALHRSYLTPGDSSSGWTAGAAAYYVGYLQPRTFLLKYNENGTIT
DAVDCALDPLSETKCTLKSFTVEKGIYQTSNFRVQPTESIVRFPNITNL
CPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQT
NSPRRARSVASQSIIAYTMSLGAENSVAYSNNSIAIPTNFTISVTTEIL
PVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNK
VTLADAGFIKQYGDCLGDIAARDLICAQKFNGLTVLPPLLTDEMIAQYT
SALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGA
ISSVLNDILSRLDKVEAEVQIDRLITGRLQSLQTYVTQQLIRAAEIRAS
ANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDN
TFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIW
LGFIAGLIAIVMVTIMLCCMTSCCSCLKGCCSCGSCCKFDEDDSEPVLK
GVKLHYT

In certain embodiments the vaccine comprises or encodes CD74 cytoplasmic domain and transmembrane domain with the extracellular domain of a spike protein. In specific embodiments, the vaccine comprises or encodes the sequence set forth below (SEQ ID NO:38):

MAATMAHRRRSRSCREDQKPVMDDQRDLISNNEQLPMLGRRPGAPESKC
SRGALYTGFSILVTLLLAGQATTAYFLYVNLTTRTQLPPAYTNSFTRGV
YYPDKVFRSSVLHSTQDLFLPFFSNVTWFHAIHVSGTNGTKRFDNPVLP
FNDGVYFASTEKSKIIRGWIFGTTLDSKTQSLLIVNNATNVVIKVCEFQ
FCNDPFLGVYYHKNNKSWMESEFRVYSSANNCTFEYVSQPFLMDLEGKQ
GNFKNLREFTAGAAAYYVGYLQPRTFLLKYNENGTITDAVDCALDPLSE
TKCTLKSFTVEKGIYQTSNFRVQPTESIVRFPNITNLCPFGEVFNATRF
ASVYAWNRKRISNCVADYSVLYNSASLSTFKCYGVSPTKLNDLCFTNVY
ADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQS
YGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSF
GGVSVITPGTNTSNQVAVLYQDVNCTEVPVAIHADQLTPTWRVYSTGSN
VFQTRAGCLIGAEHVNNSYECDIPIGAGICASYQTQTNSPRRARSVASQ
SIIAYTMSLGAENSVAYSNNSIAIPTNFTISVTTEILPVSMTKTSVDCT
MYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGW
TFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRL
DKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQ
KEIDRLNEVAKNLNESLIDLQELGKYEQYIKWP*

In certain embodiments the vaccine comprises or encodes CD74 Cytoplasmic domain and transmembrane domain with a full length SARS-Cov-2 Spike Protein without a furin cleavage site. In specific embodiments, the vaccine comprises or encodes comprising the sequence as set forth below (SEQ ID NO:39):

MHRRRSRSCREDQKPVMDDQRDLISNNEQLPMLGRRPGAPESKCSRGAL
YTGFSILVTLLLAGQATTAYFLYVNLTTRTQLPPAYTNSFTRGVYYPDK
VFRSSVLHSTQDLFLPFFSNVTWFHAIHVSGTNGTKRFDNPVLPFNDGV
YFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKN
LREFVFKNIDGYFKIYSKHTPINLVRDLPQGFSALEPLVDLPIGINITR
FQTLLALHRSYLTPGDSSSGWTAGAAAYYVGYLQPRTFLLKYNENGTIT
DAVDCALDPLSETKCTLKSFTVEKGIYQTSNFRVQPTESIVRFPNITNL
CPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQT
NSASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

In certain embodiments, the vaccine comprises the sequence encoding the CD74 Cytoplasmic domain and transmembrane domain with full length SARS-Cov-2 Spike Protein as set forth below (SEQ ID NO:40);

GCCGCCACCAUGCACAGGAGGAGAAGCAGGAGCUGUCGGGAAGAUCAGAAGCCAGUCA
UGGAUGACCAGCGCGACCUUAUCUCCAACAAUGAGCAACUGCCCAUGCUGGGCCGGCG
CCCUGGGGCCCCGGAGAGCAAGUGCAGCCGCGGAGCCCUGUACACAGGCUUUUCCAUC
CUGGUGACUCUGCUCCUCGCUGGCCAGGCCACCACCGCCUACUUCCUGUACGUGAACC
UGACCACCCGGACCCAGCUACCUCCUGCCUACACAAACAGCUUCACCCGCGGCGUGUAC
UACCCUGAUAAGGUGUUCCGGAGCUCUGUGCUCCACAGCACACAGGACCUGUUCCUGC
CUUUCUUCAGCAACGUGACGUGGUUCCACGCCAUCCACGUGUCCGGCACCAAUGGUAC
AAAAAGGUUUGACAACCCCGUGCUCCCCUUCAACGACGGCGUGUAUUUUGCCAGCACU
GAAAAGUCCAAUAUCAUCCGAGGAUGGAUCUUCGGCACAACGCUGGACUCUAAGACCCA
GAGCCUGCUGAUCGUGAACAACGCCACCAACGUGGUGAUCAAGGUGUGCGAGUUUCAG
UUCUGCAACGAUCCUUUUCUGGGCGUUUACUACCACAAGAACAACAAGAGCUGGAUGGA
AAGCGAGUUCAGAGUGUAUUCUAGCGCCAACAACUGUACCUUCGAAUACGUCAGCCAGC
CUUUCCUGAUGGACCUGGAAGGCAAGCAGGGCAACUUCAAGAACCUGAGAGAGUUCGU
GUUCAAGAACAUCGACGGCUACUUCAAGAUCUACAGCAAGCACACACCUAUCAACCUGG
UGCGGGACCUUCCGCAGGGAUUCUCCGCGCUGGAACCCCUGGUGGACCUGCCUAUUG
GCAUCAACAUCACCAGAUUUCAGACCCUGCUGGCCCUGCACAGAAGCUACCUGACACCU
GGCGACUCUUCUAGCGGCUGGACCGCCGGCGCCGCCGCUUAUUACGUGGGGUACCUG
CAGCCCAGAACCUUCCUGCUGAAAUACAAUGAGAACGGAACCAUCACCGAUGCCGUGGA
CUGCGCCCUGGACCCCCUGUCUGAAACCAAGUGCACCCUGAAAUCUUUUACAGUGGAAA
AGGGAAUUUACCAGACCUCCAACUUUAGAGUCCAGCCUACCGAAAGCAUCGUGCGGUUC
CCUAACAUCACCAACCUGUGUCCUUUCGGCGAGGUUUUCAACGCCACACGGUUCGCCA
GCGUAUACGCCUGGAACCGGAAGCGGAUCUCUAACUGUGUGGCAGACUACUCUGUGCU
AUACAACUCGGCCAGCUUUAGCACCUUCAAGUGCUACGGCGUGUCCCCAACCAAGCUGA
ACGACCUGUGCUUCACCAACGUGUACGCCGAUAGCUUCGUGAUCAGAGGCGAUGAGGU
GCGCCAGAUCGCUCCUGGACAGACCGGAAAGAUCGCUGACUACAAUUACAAGCUGCCC
GACGACUUCACCGGCUGCGUGAUCGCCUGGAACAGCAACAACCUGGAUUCCAAAGUGG
GCGGCAACUACAACUAUCUGUACCGGCUCUUCAGAAAGUCUAACCUGAAGCCCUUUGAG
AGAGACAUCUCCACAGAAAUCUACCAGGCCGGAAGCACACCUUGCAACGGCGUGGAAGG
AUUUAAUUGCUAUUUUCCUCUGCAGUCCUACGGCUUCCAACCUACCAACGGAGUGGGC
UACCAGCCUUACAGAGUGGUCGUGCUGAGCUUUGAGCUGCUGCAUGCCCCUGCUACCG
UGUGUGGCCCUAAGAAAAGCACGAACCUGGUCAAGAACAAAUGCGUGAACUUCAACUUC
AAUGGACUGACAGGCACCGGCGUGCUCACAGAGAGCAACAAGAAGUUCCUGCCAUUUCA
GCAGUUCGGCAGAGACAUAGCUGACACCACCGACGCCGUGCGGGACCCCCAGACCCUG
GAAAUCCUGGAUAUCACACCCUGCAGCUUUGGAGGCGUAUCUGUGAUCACACCUGGCA
CCAACACAAGCAAUCAGGUCGCCGUGCUGUACCAAGACGUGAACUGCACAGAGGUGCC
GGUGGCCAUCCACGCCGAUCAGCUGACCCCCACCUGGCGGGUGUACUCCACCGGCAGC
AACGUGUUCCAAACCCGGGCCGGCUGCCUGAUCGGCGCCGAACAUGUGAAUAACAGCU
ACGAAUGUGAUAUCCCUAUUGGAGCCGGAAUUUGCGCCAGCUACCAAACCCAAACCAAC
UCUCCAAGAAGAGCCAGAAGCGUGGCUAGCCAGUCUAUCAUCGCCUACACCAUGAGCCU
GGGCGCUGAAAACAGCGUGGCUUACAGCAACAACAGCAUCGCCAUUCCUACUAACUUUA
CCAUCAGCGUGACUACAGAGAUCCUGCCCGUGAGCAUGACCAAGACCAGCGUGGACUG
CACCAUGUACAUCUGUGGCGAUUCUACCGAGUGCAGCAACUUGCUACUGCAGUAUGGU
AGCUUUUGCACCCAACUGAACAGAGCCCUGACUGGCAUCGCCGUUGAGCAGGACAAGAA
CACCCAGGAGGUGUUCGCCCAGGUCAAGCAGAUCUAUAAGACCCCACCAAUCAAGGACU
UCGGCGGCUUCAACUUCAGCCAGAUCCUGCCCGACCCGAGCAAACCUAGCAAGAGAAGC
UUCAUCGAGGAUCUUCUGUUCAACAAGGUGACGCUGGCCGAUGCCGGCUUCAUCAAAC
AGUACGGCGAUUGUCUGGGCGACAUCGCCGCCAGAGAUCUGAUCUGCGCCCAGAAGUU
CAAUGGCCUGACCGUGCUGCCGCCUCUGCUCACAGACGAGAUGAUCGCCCAGUACACC
UCUGCCCUGUUAGCAGGCACAAUCACGAGCGGCUGGACAUUUGGCGCGGGCGCCGCUC
UGCAGAUCCCUUUCGCUAUGCAGAUGGCCUAUAGAUUUAAUGGCAUCGGCGUGACCCA
GAAUGUGCUGUACGAGAACCAAAAGCUGAUCGCUAAUCAGUUCAAUAGCGCCAUCGGAA
AGAUCCAGGACUCUCUGAGCAGCACCGCCUCCGCUCUGGGCAAACUGCAGGACGUGGU
CAAUCAAAACGCUCAGGCUCUAAACACCCUGGUGAAGCAGCUGUCAAGCAACUUCGGCG
CCAUCUCCUCAGUUCUUAACGACAUCUUGUCUAGACUGGACAAGGUGGAGGCUGAGGU
GCAGAUCGAUAGACUGAUCACAGGCAGAUUACAGUCUCUGCAGACAUACGUGACCCAAC
AGCUGAUCCGGGCCGCCGAGAUCAGAGCCUCCGCCAACCUGGCUGCCACCAAGAUGAG
CGAGUGUGUGCUUGGCCAGUCCAAGAGAGUGGACUUCUGCGGCAAAGGCUACCACCUG
AUGAGCUUCCCACAGAGCGCUCCUCACGGAGUUGUGUUCCUGCACGUGACCUACGUGC
CUGCCCAGGAGAAGAAUUUCACAACCGCCCCUGCCAUCUGCCACGACGGUAAGGCCCAC
UUCCCCAGAGAGGGCGUGUUCGUGUCUAAUGGGACCCACUGGUUCGUGACACAGAGGA
ACUUCUACGAGCCCCAGAUCAUCACAACAGAUAAUACCUUCGUGUCUGGCAACUGCGAU
GUGGUGAUCGGCAUCGUGAACAAUACCGUGUACGACCCUCUGCAGCCCGAGCUGGACA
GCUUCAAAGAGGAACUGGACAAGUACUUCAAGAAUCACACCUCCCCUGACGUGGACCUG
GGAGAUAUCUCCGGCAUCAACGCCUCCGUUGUGAACAUUCAAAAAGAAAUCGACAGACU
GAACGAGGUGGCCAAGAACCUAAACGAGUCUCUGAUCGACCUGCAAGAACUGGGCAAGU
ACGAGCAGUACAUCAAGUGGCCUUGGUACAUCUGGCUGGGCUUUAUCGCCGGCCUGAU
CGCCAUUGUGAUGGUGACCAUCAUGCUGUGCUGCAUGACAAGUUGCUGCAGCUGCCUG
AAGGGCUGUUGUUCUUGUGGCAGCUGCUGUAAAUUCGACGAGGACGAUAGCGAACCAG
UGCUGAAAGGCGUCAAGCUGCAUUACACCUGA

In certain embodiments, the vaccine comprises the sequence encoding CD74 Cytoplasmic domain and transmembrane domain with the extracellular domain of SARS-Cov-2 Spike Protein as set forth below (SEQ ID NO:41):

GCCGCCACCATGgcaCACAGGAGGAGAAGCAGGAGCTGcCGGGAAGAcCA
GAAGCCAGTCATGGAcGACCAGCGCGACCTCATCTCCAACAAcGAGCAAC
TGCCCATGCTGGGCCGGCGCCCaGGGGCCCCGGAGAGCAAGTGCAGCCGC
GGAGCCCTGTACACAGGCTTCTCCATCCTGGTGACcCTGCTCCTCGCcGG
CCAGGCCACCACCGCCTACTTCCTGTACGTGAACCTGACCACCCGGACCC
AGCTACCaCCaGCCTACACAAACAGCTTCACCCGCGGCGTGTACTACCCa
GAcAAGGTGTTCCGGAGCTCaGTGCTCCACAGCACACAGGACCTGTTCCT
GCCaTTCTTCAGCAACGTGACGTGGTTCCACGCCATCCACGTGTCCGGCA
CCAAcGGaACAAAAAGGTTcGACAACCCCGTGCTCCCCTTCAACGACGGC
GTGTAcTTcGCCAGCACaGAAAAGTCCAAaATCATCCGAGGATGGATCTT
CGGCACAACGCTGGACTCaAAGACCCAGAGCCTGCTGATCGTGAACAACG
CCACCAACGTGGTGATCAAGGTGTGCGAGTTCCAGTTCTGCAACGATCCa
TTcCTGGGCGTaTACTACCACAAGAACAACAAGAGCTGGATGGAAAGCGA
GTTCAGAGTGTACTCaAGCGCCAACAACTGcACCTTCGAATACGTCAGCC
AGCCaTTCCTGATGGACCTGGAAGGCAAGCAGGGCAACTTCAAGAACCTG
AGAGAGTTCGTGTTCAAGAACATCGACGGCTACTTCAAGATCTACAGCAA
GCACACACCCATCAACCTGGTGCGGGACCTaCCGCAGGGATTCTCCGCGC
TGGAACCCCTGGTGGACCTGCCaATaGGCATCAACATCACCAGATTcCAG
ACCCTGCTGGCCCTGCACAGAAGCTACCTGACACCaGGCGACagcagcAG
CGGCTGGACCGCCGGCGCCGCCGCaTACTACGTGGGGTACCTGCAGCCCA
GAACCTTCCTGCTGAAATACAAcGAGAACGGAACCATCACCGAcGCCGTG
GACTGCGCCCTGGACCCCCTGagcGAAACCAAGTGCACCCTGAAAagcTT
cACAGTGGAAAAGGGAATaTACCAGACCTCCAACTTcAGAGTCCAGCCaA
CCGAAAGCATCGTGCGGTTCCCaAACATCACCAACCTGTGcCCaTTCGGC
GAGGTaTTCAACGCCACACGGTTCGCCAGCGTATACGCCTGGAACCGGAA
GCGGATCagcAACTGcGTGGCAGACTACagcGTGCTATACAACagcGCCA
GCTTaAGCACCTTCAAGTGCTACGGCGTGTCCCCAACCAAGCTGAACGAC
CTGTGCTTCACCAACGTGTACGCCGAcAGCTTCGTGATCAGAGGCGAcGA
GGTGCGCCAGATCGCaCCaGGACAGACCGGAAAGATCGCaGACTACAAcT
ACAAGCTGCCCGACGACTTCACCGGCTGCGTGATCGCCTGGAACAGCAAC
AACCTGGACTCCAAAGTGGGCGGCAACTACAACTAcCTGTACCGGCTCTT
CAGAAAGagcAACCTGAAGCCCTTCGAGAGAGACATCagcACAGAAATCT
ACCAGGCCGGAAGCACACCaTGCAACGGCGTGGAAGGATTCAAcTGCTAT
TTcCCaCTGCAGTCCTACGGCTTCCAACCaACCAACGGAGTGGGCTACCA
GCCaTACAGAGTGGTCGTGCTGAGCTTcGAGCTGCTGCAcGCCCCaGCaA
CCGTGTGcGGCCCaAAGAAAAGCACGAACCTGGTCAAGAACAAATGCGTG
AACTTCAACTTCAAcGGACTGACAGGCACCGGCGTGCTCACAGAGAGCAA
CAAGAAGTTCCTGCCATTCCAGCAGTTCGGCAGAGACATAGCaGACACCA
CCGACGCCGTGCGGGACCCCCAGACCCTGGAAATCCTGGAcATCACACCC
TGCAGCTTcGGAGGCGTAagcGTGATCACACCaGGCACCAACACAAGCAA
cCAGGTCGCCGTGCTGTACCAAGACGTGAACTGCACAGAGGTGCCGGTGG
CCATCCACGCCGAcCAGCTGACCCCCACCTGGCGGGTGTACTCCACCGGC
AGCAACGTGTTCCAAACCCGGGCCGGCTGCCTGATCGGCGCCGAACAcGT
GAAcAACAGCTACGAATGcGAcATCCCaATcGGAGCCGGAATCTGCGCCA
GCTACCAAACCCAAACCAACagcCCAAGAAGAGCCAGAAGCGTGGCaAGC
CAGagcATCATCGCCTACACCATGAGCCTGGGCGCaGAAAACAGCGTGGC
aTACAGCAACAACAGCATCGCCATcCCaACCAACTTcACCATCAGCGTGA
CCACAGAGATCCTGCCCGTGAGCATGACCAAGACCAGCGTGGACTGCACC
ATGTACATCTGcGGCGAcagcACCGAGTGCAGCAACTTGCTACTGCAGTA
cGGaAGCTTCTGCACCCAACTGAACAGAGCCCTGACcGGCATCGCCGTcG
AGCAGGACAAGAACACCCAGGAGGTGTTCGCCCAGGTCAAGCAGATCTAC
AAGACCCCACCAATCAAGGACTTCGGCGGCTTCAACTTCAGCCAGATCCT
GCCCGACCCGAGCAAACCaAGCAAGAGAAGCTTCATCGAGGAcCTaCTGT
TCAACAAGGTGACGCTGGCCGAcGCCGGCTTCATCAAACAGTACGGCGAC
TGcCTGGGCGACATCGCCGCCAGAGAcCTGATCTGCGCCCAGAAGTTCAA
TGGCCTGACCGTGCTGCCGCCaCTGCTCACAGACGAGATGATCGCCCAGT
ACACCagcGCCCTGTTAGCAGGCACAATCACGAGCGGCTGGACATTcGGC
GCGGGCGCCGCaCTGCAGATCCCaTTCGCaATGCAGATGGCCTAcAGATT
cAATGGCATCGGCGTGACCCAGAAcGTGCTGTACGAGAACCAAAAGCTGA
TCGCaAAcCAGTTCAAcAGCGCCATCGGAAAGATCCAGGACagcCTGAGC
AGCACCGCCTCCGCaCTGGGCAAACTGCAGGACGTGGTCAAcCAAAACGC
aCAGGCaCTAAACACCCTGGTGAAGCAGCTGTCAAGCAACTTCGGCGCCA
TCagcTCAGTcCTaAACGACATCTTGagcAGACTGGACAAGGTGGAGGCa
GAGGTGCAGATCGAcAGACTGATCACAGGCAGATTACAGagcCTGCAGAC
ATACGTGACCCAACAGCTGATCCGGGCCGCCGAGATCAGAGCCTCCGCCA
ACCTGGCaGCCACCAAGATGAGCGAGTGcGTGCTaGGCCAGagcAAGAGA
GTGGACTTCTGCGGCAAAGGCTACCACCTGATGAGCTTCCCACAGAGCGC
aCCaCACGGAGTaGTGTTCCTGCACGTGACCTACGTGCCaGCCCAGGAGA
AGAAcTTCACAACCGCCCCaGCCATCTGCCACGACGGaAAGGCCCACTTC
CCCAGAGAGGGCGTGTTCGTGagcAAcGGGACCCACTGGTTCGTGACACA
GAGGAACTTCTACGAGCCCCAGATCATCACAACAGACAAcACCTTCGTGa
gcGGCAACTGCGAcGTGGTGATCGGCATCGTGAACAAcACCGTGTACGAC
CCaCTGCAGCCCGAGCTGGACAGCTTCAAAGAGGAACTGGACAAGTACTT
CAAGAAcCACACCTCCCCaGACGTGGACCTGGGAGAcATCagcGGCATCA
ACGCCTCCGTaGTGAACATcCAAAAAGAAATCGACAGACTGAACGAGGTG
GCCAAGAACCTAAACGAGagcCTGATCGACCTGCAAGAACTGGGCAAGTA
CGAGCAGTACATCAAGTGGCCaTAA

In certain embodiments, the vaccine comprises the sequence encoding CD74 Cytoplasmic domain and transmembrane domain with full length SARS-Cov-2 Spike Protein (without furin cleavage site) as set forth below (SEQ ID NO:42):

GCCGCCACCAUGCACAGGAGGAGAAGCAGGAGCUGUCGGGAAGAUCAGAA
GCCAGUCAUGGAUGACCAGCGCGACCUUAUCUCCAACAAUGAGCAACUGC
CCAUGCUGGGCCGGCGCCCUGGGGCCCCGGAGAGCAAGUGCAGCCGCGGA
GCCCUGUACACAGGCUUUUCCAUCCUGGUGACUCUGCUCCUCGCUGGCCA
GGCCACCACCGCCUACUUCCUGUACGUGAACCUGACCACCCGGACCCAGC
UACCUCCUGCCUACACAAACAGCUUCACCCGCGGCGUGUACUACCCUGAU
AAGGUGUUCCGGAGCUCUGUGCUCCACAGCACACAGGACCUGUUCCUGCC
UUUCUUCAGCAACGUGACGUGGUUCCACGCCAUCCACGUGUCCGGCACCA
AUGGUACAAAAAGGUUUGACAACCCCGUGCUCCCCUUCAACGACGGCGUG
UAUUUUGCCAGCACUGAAAAGUCCAAUAUCAUCCGAGGAUGGAUCUUCGG
CACAACGCUGGACUCUAAGACCCAGAGCCUGCUGAUCGUGAACAACGCCA
CCAACGUGGUGAUCAAGGUGUGCGAGUUUCAGUUCUGCAACGAUCCUUUU
CUGGGCGUUUACUACCACAAGAACAACAAGAGCUGGAUGGAAAGCGAGUU
CAGAGUGUAUUCUAGCGCCAACAACUGUACCUUCGAAUACGUCAGCCAGC
CUUUCCUGAUGGACCUGGAAGGCAAGCAGGGCAACUUCAAGAACCUGAGA
GAGUUCGUGUUCAAGAACAUCGACGGCUACUUCAAGAUCUACAGCAAGCA
CACACCUAUCAACCUGGUGCGGGACCUUCCGCAGGGAUUCUCCGCGCUGG
AACCCCUGGUGGACCUGCCUAUUGGCAUCAACAUCACCAGAUUUCAGACC
CUGCUGGCCCUGCACAGAAGCUACCUGACACCUGGCGACUCUUCUAGCGG
CUGGACCGCCGGCGCCGCCGCUUAUUACGUGGGGUACCUGCAGCCCAGAA
CCUUCCUGCUGAAAUACAAUGAGAACGGAACCAUCACCGAUGCCGUGGAC
UGCGCCCUGGACCCCCUGUCUGAAACCAAGUGCACCCUGAAAUCUUUUAC
AGUGGAAAAGGGAAUUUACCAGACCUCCAACUUUAGAGUCCAGCCUACCG
AAAGCAUCGUGCGGUUCCCUAACAUCACCAACCUGUGUCCUUUCGGCGAG
GUUUUCAACGCCACACGGUUCGCCAGCGUAUACGCCUGGAACCGGAAGCG
GAUCUCUAACUGUGUGGCAGACUACUCUGUGCUAUACAACUCGGCCAGCU
UUAGCACCUUCAAGUGCUACGGCGUGUCCCCAACCAAGCUGAACGACCUG
UGCUUCACCAACGUGUACGCCGAUAGCUUCGUGAUCAGAGGCGAUGAGGU
GCGCCAGAUCGCUCCUGGACAGACCGGAAAGAUCGCUGACUACAAUUACA
AGCUGCCCGACGACUUCACCGGCUGCGUGAUCGCCUGGAACAGCAACAAC
CUGGAUUCCAAAGUGGGCGGCAACUACAACUAUCUGUACCGGCUCUUCAG
AAAGUCUAACCUGAAGCCCUUUGAGAGAGACAUCUCCACAGAAAUCUACC
AGGCCGGAAGCACACCUUGCAACGGCGUGGAAGGAUUUAAUUGCUAUUUU
CCUCUGCAGUCCUACGGCUUCCAACCUACCAACGGAGUGGGCUACCAGCC
UUACAGAGUGGUCGUGCUGAGCUUUGAGCUGCUGCAUGCCCCUGCUACCG
UGUGUGGCCCUAAGAAAAGCACGAACCUGGUCAAGAACAAAUGCGUGAAC
UUCAACUUCAAUGGACUGACAGGCACCGGCGUGCUCACAGAGAGCAACAA
GAAGUUCCUGCCAUUUCAGCAGUUCGGCAGAGACAUAGCUGACACCACCG
ACGCCGUGCGGGACCCCCAGACCCUGGAAAUCCUGGAUAUCACACCCUGC
AGCUUUGGAGGCGUAUCUGUGAUCACACCUGGCACCAACACAAGCAAUCA
GGUCGCCGUGCUGUACCAAGACGUGAACUGCACAGAGGUGCCGGUGGCCA
UCCACGCCGAUCAGCUGACCCCCACCUGGCGGGUGUACUCCACCGGCAGC
AACGUGUUCCAAACCCGGGCCGGCUGCCUGAUCGGCGCCGAACAUGUGAA
UAACAGCUACGAAUGUGAUAUCCCUAUUGGAGCCGGAAUUUGCGCCAGCU
ACCAAACCCAAACCAACUCUGCUAGCCAGUCUAUCAUCGCCUACACCAUG
AGCCUGGGCGCUGAAAACAGCGUGGCUUACAGCAACAACAGCAUCGCCAU
UCCUACUAACUUUACCAUCAGCGUGACUACAGAGAUCCUGCCCGUGAGCA
UGACCAAGACCAGCGUGGACUGCACCAUGUACAUCUGUGGCGAUUCUACC
GAGUGCAGCAACUUGCUACUGCAGUAUGGUAGCUUUUGCACCCAACUGAA
CAGAGCCCUGACUGGCAUCGCCGUUGAGCAGGACAAGAACACCCAGGAGG
UGUUCGCCCAGGUCAAGCAGAUCUAUAAGACCCCACCAAUCAAGGACUUC
GGCGGCUUCAACUUCAGCCAGAUCCUGCCCGACCCGAGCAAACCUAGCAA
GAGAAGCUUCAUCGAGGAUCUUCUGUUCAACAAGGUGACGCUGGCCGAUG
CCGGCUUCAUCAAACAGUACGGCGAUUGUCUGGGCGACAUCGCCGCCAGA
GAUCUGAUCUGCGCCCAGAAGUUCAAUGGCCUGACCGUGCUGCCGCCUCU
GCUCACAGACGAGAUGAUCGCCCAGUACACCUCUGCCCUGUUAGCAGGCA
CAAUCACGAGCGGCUGGACAUUUGGCGCGGGCGCCGCUCUGCAGAUCCCU
UUCGCUAUGCAGAUGGCCUAUAGAUUUAAUGGCAUCGGCGUGACCCAGAA
UGUGCUGUACGAGAACCAAAAGCUGAUCGCUAAUCAGUUCAAUAGCGCCA
UCGGAAAGAUCCAGGACUCUCUGAGCAGCACCGCCUCCGCUCUGGGCAAA
CUGCAGGACGUGGUCAAUCAAAACGCUCAGGCUCUAAACACCCUGGUGAA
GCAGCUGUCAAGCAACUUCGGCGCCAUCUCCUCAGUUCUUAACGACAUCU
UGUCUAGACUGGACAAGGUGGAGGCUGAGGUGCAGAUCGAUAGACUGAUC
ACAGGCAGAUUACAGUCUCUGCAGACAUACGUGACCCAACAGCUGAUCCG
GGCCGCCGAGAUCAGAGCCUCCGCCAACCUGGCUGCCACCAAGAUGAGCG
AGUGUGUGCUUGGCCAGUCCAAGAGAGUGGACUUCUGCGGCAAAGGCUAC
CACCUGAUGAGCUUCCCACAGAGCGCUCCUCACGGAGUUGUGUUCCUGCA
CGUGACCUACGUGCCUGCCCAGGAGAAGAAUUUCACAACCGCCCCUGCCA
UCUGCCACGACGGUAAGGCCCACUUCCCCAGAGAGGGCGUGUUCGUGUCU
AAUGGGACCCACUGGUUCGUGACACAGAGGAACUUCUACGAGCCCCAGAU
CAUCACAACAGAUAAUACCUUCGUGUCUGGCAACUGCGAUGUGGUGAUCG
GCAUCGUGAACAAUACCGUGUACGACCCUCUGCAGCCCGAGCUGGACAGC
UUCAAAGAGGAACUGGACAAGUACUUCAAGAAUCACACCUCCCCUGACGU
GGACCUGGGAGAUAUCUCCGGCAUCAACGCCUCCGUUGUGAACAUUCAAA
AAGAAAUCGACAGACUGAACGAGGUGGCCAAGAACCUAAACGAGUCUCUG
AUCGACCUGCAAGAACUGGGCAAGUACGAGCAGUACAUCAAGUGGCCUUG
GUACAUCUGGCUGGGCUUUAUCGCCGGCCUGAUCGCCAUUGUGAUGGUGA
CCAUCAUGCUGUGCUGCAUGACAAGUUGCUGCAGCUGCCUGAAGGGCUGU
UGUUCUUGUGGCAGCUGCUGUAAAUUCGACGAGGACGAUAGCGAACCAGU
GCUGAAAGGCGUCAAGCUGCAUUACACCUGA

In certain embodiments, the vaccine comprises the sequence encoding a Spike S1+S2 ECD_CD74+HLA as set forth below (SEQ ID NO: 43).

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTGAACCTCACTACGCGAACTCAACTGCCGCCTG
CTTACACTAATAGTTTTACACGAGGAGTCTACTACCCGGACAAGGTGTTC
CGCTCAAGTGTCTTGCACAGCACGCAAGACCTGTTCTTGCCATTTTTCAG
CAATGTAACGTGGTTTCATGCCATCCACGTGTCAGGTACTAACGGAACGA
AGAGATTTGATAACCCGGTCCTCCCGTTCAACGACGGGGTTTATTTTGCC
AGTACAGAAAAGTCCAATATTATAAGGGGATGGATTTTCGGCACGACTCT
CGACTCTAAGACACAAAGCTTGCTCATCGTTAATAATGCTACAAACGTGG
TTATAAAAGTATGCGAGTTCCAATTCTGTAACGACCCCTTTCTGGGGGTT
TACTACCACAAAAATAATAAGAGTTGGATGGAGAGCGAATTCAGAGTTTA
CTCTTCCGCGAATAATTGCACCTTCGAGTACGTATCCCAGCCTTTTCTTA
TGGATCTTGAGGGGAAACAAGGTAATTTCAAAAACCTTAGAGAATTCGTT
TTTAAGAATATAGATGGATACTTCAAGATTTATTCTAAACATACCCCAAT
CAATCTGGTGCGCGATCTTCCTCAGGGTTTCTCTGCGCTGGAGCCTCTCG
TTGATCTGCCGATCGGAATTAATATAACCAGATTCCAGACCCTTTTGGCA
TTGCACCGCTCCTATCTGACACCTGGGGATAGTAGTTCCGGCTGGACAGC
GGGCGCCGCGGCATACTACGTTGGATATCTCCAACCCCGCACTTTTCTTC
TGAAATATAATGAGAATGGAACGATTACAGACGCAGTAGATTGCGCGCTT
GACCCCCTTTCAGAAACAAAGTGCACGCTGAAATCTTTCACGGTCGAGAA
GGGCATTTACCAAACGTCCAATTTCAGAGTTCAACCCACTGAGTCTATAG
TCAGGTTTCCGAACATCACCAATCTCTGCCCATTTGGAGAGGTTTTCAAC
GCAACACGGTTCGCCTCTGTATACGCATGGAATAGAAAGAGAATATCCAA
TTGTGTCGCAGACTATAGTGTCTTGTACAACTCTGCGTCATTTAGCACTT
TTAAATGTTATGGGGTGAGTCCAACTAAATTGAACGATCTCTGCTTTACA
AATGTTTACGCAGATAGTTTTGTGATACGAGGCGACGAGGTACGACAAAT
TGCTCCTGGCCAGACCGGGAAAATAGCGGACTACAATTATAAACTGCCAG
ACGATTTCACCGGCTGTGTGATCGCGTGGAACTCAAACAACTTGGATAGT
AAGGTAGGTGGTAACTATAACTACTTGTACCGGCTCTTCCGCAAAAGTAA
TCTTAAACCGTTCGAGCGGGACATCTCTACCGAGATCTATCAAGCAGGTA
GCACTCCCTGCAATGGAGTCGAGGGTTTTAATTGCTATTTTCCCCTCCAA
TCATACGGGTTTCAGCCCACAAATGGTGTTGGGTATCAGCCCTATCGAGT
TGTGGTGCTTTCATTTGAATTGCTTCATGCACCGGCGACAGTTTGTGGTC
CTAAAAAGAGTACCAATTTGGTTAAAAACAAATGTGTCAACTTTAACTTT
AACGGTCTGACTGGTACGGGGGTGCTTACGGAATCTAATAAAAAGTTTCT
TCCGTTCCAGCAATTCGGACGGGACATAGCGGACACGACTGACGCGGTTA
GAGACCCACAAACACTCGAGATTCTTGACATAACCCCCTGTTCTTTCGGC
GGAGTTTCTGTAATAACGCCTGGGACAAATACGTCAAACCAGGTTGCAGT
ATTGTACCAGGATGTCAACTGCACTGAAGTGCCAGTCGCGATCCACGCAG
ATCAGTTGACTCCAACATGGAGGGTCTACTCTACCGGTTCTAACGTGTTT
CAGACCAGAGCCGGGTGCCTCATTGGCGCGGAACATGTGAACAATTCTTA
CGAATGCGACATACCCATCGGCGCCGGCATTTGTGCGTCATACCAAACTC
AAACTAATTCCCGGAGTGTGGCCTCCCAATCTATTATTGCTTATACTATG
AGTCTTGGCGCGGAAAACAGTGTAGCGTACTCAAATAACTCTATTGCGAT
CCCCACGAATTTCACTATAAGCGTAACTACTGAAATTTTGCCGGTAAGCA
TGACGAAAACTTCAGTGGACTGCACAATGTACATTTGTGGTGACAGCACT
GAGTGCTCAAATCTCCTCCTTCAGTATGGTTCTTTCTGCACACAACTTAA
CAGGGCACTTACCGGAATCGCAGTAGAACAAGACAAAAACACGCAAGAAG
TATTCGCACAGGTCAAACAAATTTATAAAACCCCGCCGATCAAGGATTTT
GGAGGGTTTAACTTCAGTCAGATCCTTCCAGACCCATCCAAACCCAGCAA
ACGGAGCTTTATTGAAGACCTGCTGTTTAATAAAGTTACCCTTGCAGATG
CCGGTTTCATCAAGCAATACGGGGATTGTCTGGGCGACATTGCCGCCCGC
GACTTGATCTGTGCCCAGAAGTTCAACGGTCTCACAGTCTTGCCGCCACT
TCTCACTGATGAAATGATAGCGCAGTATACATCCGCGCTGCTGGCTGGGA
CGATAACATCTGGTTGGACTTTCGGGGCCGGGGCGGCTTTGCAAATTCCT
TTCGCCATGCAGATGGCCTATAGGTTTAACGGCATAGGAGTGACTCAAAA
TGTTCTTTATGAGAATCAGAAGCTTATCGCCAACCAATTTAACTCCGCGA
TTGGGAAGATACAGGACTCATTGAGCTCTACTGCTAGCGCCCTTGGCAAG
CTTCAAGACGTAGTCAACCAAAATGCTCAAGCGCTCAATACTCTCGTAAA
GCAGCTCTCAAGCAACTTTGGCGCGATATCTTCAGTTTTGAACGACATCT
TGAGCAGACTCGACAAGGTGGAGGCTGAAGTTCAAATTGATCGACTGATT
ACGGGCCGGCTCCAGTCACTCCAAACGTACGTGACCCAACAACTCATTAG
AGCGGCAGAGATTAGAGCGAGCGCTAATCTGGCGGCCACAAAGATGTCAG
AATGTGTGCTTGGTCAATCCAAAAGGGTGGATTTCTGCGGAAAGGGCTAT
CACTTGATGAGTTTCCCGCAGTCCGCGCCTCATGGAGTTGTTTTCCTCCA
CGTAACCTACGTTCCCGCTCAGGAAAAGAATTTCACGACAGCTCCGGCAA
TATGTCATGACGGTAAAGCACATTTCCCCAGGGAAGGCGTTTTTGTCAGT
AATGGAACCCACTGGTTCGTCACCCAGCGAAACTTTTACGAGCCCCAAAT
AATAACTACTGACAATACCTTCGTCAGCGGCAATTGCGACGTCGTCATAG
GCATAGTAAATAATACGGTTTACGACCCTCTTCAACCCGAGTTGGACAGC
TTCAAAGAAGAACTTGATAAGTATTTTAAAAACCACACTAGCCCAGATGT
AGACCTTGGCGACATCAGCGGTATCAATGCTTCCGTTGTGAACATTCAAA
AGGAAATTGACCGACTCAATGAAGTAGCGAAGAACTTGAATGAGTCTCTG
ATCGATCTTCAGGAGCTCGGGAAATATGAGCAATACATCAAATGGCCGTC
ACGACTGGAGGAAGAACTGCGCCGACGCCTGACTGAATGA

In certain embodiments the vaccine comprises or encodes Spike S1+S2 ECD_CD74+HLA. In specific embodiments the vaccine comprises or encodes the sequence as set forth below (SEQ ID NO:44).

MHRRRSRSCREDQNPVMDDQRDLISNNEQLPMLGRRPGAPESKCSRWMVA
AVVAGTIVAGLLVLGAIIGVVNLTTRTQLPPAYTNSFTRGVYYPDKVFRS
SVLHSTQDLFLPFFSNVTWFHAIHVSGTNGTKRFDNPVLPFNDGVYFAST
EKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFK
NIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNAT
RFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSY
GFQPTNGVGYQPYRVVVLSFELLHAPATVCGPKKSTNLVKNKCVNFNFNG
LTGTGVLTESNKKFLPFQQFGRDIADTTDAVRDPQTLEILDITPCSFGGV
SVITPGTNTSNQVAVLYQDVNCTEVPVAIHADQLTPTWRVYSTGSNVFQT
RAGCLIGAEHVNNSYECDIPIGAGICASYQTQTNSRSVASQSIIAYTMSL
GAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAARDL
ICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITG
RLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLID
LQELGKYEQYIKWPSRLEEELRRRLTE*

In certain embodiments, the vaccine comprises the sequence encoding Kent Spike S1+S2 ECD_CD74+HLA as set forth below (SEQ ID NO:45).

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTTAACCTGACAACACGCACCCAGTTGCCACCGG
CCTATACGAACTCTTTCACGCGCGGAGTGTATTATCCCGATAAAGTCTTT
CGGAGTTCTGTGCTGCACTCTACACAAGATCTTTTTCTGCCCTTTTTCTC
CAACGTTACGTGGTTCCATGCAATAAGTGGTACGAATGGTACTAAAAGGT
TCGATAATCCCGTACTTCCCTTCAATGACGGCGTGTATTTCGCGAGCACA
GAAAAGAGCAACATAATAAGAGGTTGGATTTTTGGGACTACCCTGGACTC
TAAAACACAAAGCCTCCTCATTGTTAATAACGCAACTAATGTCGTAATAA
AGGTATGCGAATTTCAGTTTTGTAACGACCCTTTTTTGGGAGTCTATCAT
AAGAACAATAAGTCCTGGATGGAAAGTGAATTTAGGGTGTATAGTTCAGC
GAACAACTGTACATTTGAGTATGTGTCACAGCCGTTCCTTATGGATCTTG
AAGGGAAACAGGGGAACTTCAAGAACCTGCGGGAATTCGTGTTTAAAAAT
ATAGACGGTTATTTTAAAATCTATAGCAAACACACCCCAATAAACCTGGT
TCGGGATCTTCCTCAGGGATTTTCAGCTTTGGAACCGTTGGTTGACCTCC
CCATCGGGATAAATATCACGCGATTTCAAACACTGCTGGCACTGCACCGG
AGTTATCTGACTCCTGGAGATAGCAGCTCCGGCTGGACGGCTGGCGCCGC
GGCTTATTATGTCGGGTACCTTCAACCGCGAACGTTTCTTTTGAAGTATA
ACGAAAATGGCACTATAACCGATGCAGTAGATTGTGCGTTGGACCCTCTT
TCCGAGACTAAGTGCACCCTGAAGTCATTTACAGTAGAGAAGGGGATATA
CCAGACAAGCAACTTCCGCGTCCAACCAACGGAAAGCATCGTGCGCTTTC
CCAATATTACTAACCTCTGTCCGTTCGGCGAGGTTTTTAATGCTACCAGA
TTTGCCTCAGTGTATGCCTGGAACCGCAAGAGGATTAGCAACTGCGTTGC
TGACTATAGCGTGCTTTACAATTCCGCCAGTTTTAGTACGTTCAAGTGTT
ATGGGGTAAGCCCTACCAAGCTTAACGATCTTTGCTTCACGAATGTTTAC
GCAGACTCATTCGTAATACGAGGTGATGAAGTGCGACAGATAGCCCCGGG
TCAGACGGGGAAAATAGCCGACTACAATTACAAGCTCCCGGACGACTTCA
CGGGCTGTGTTATTGCGTGGAACAGCAATAACCTGGACAGTAAAGTTGGA
GGGAATTATAACTACTTGTACAGGCTGTTCCGCAAATCTAATTTGAAACC
GTTTGAAAGGGACATTTCCACCGAGATTTACCAAGCCGGCAGTACCCCAT
GTAACGGAGTTGAGGGATTTAATTGCTACTTTCCGCTCCAATCATATGGA
TTCCAGCCTACATACGGCGTCGGATATCAACCTTATAGGGTGGTCGTTCT
TTCTTTTGAGCTGCTTCACGCCCCAGCAACCGTTTGCGGCCCGAAAAAAT
CAACGAACTTGGTCAAAAACAAGTGCGTCAACTTCAATTTCAACGGACTG
ACAGGCACCGGAGTGCTCACCGAGTCAAATAAAAAATTCCTCCCGTTTCA
ACAATTCGGTAGAGATATAGACGACACCACAGATGCGGTACGCGACCCTC
AGACTCTTGAAATTTTGGACATTACACCATGTTCCTTCGGTGGCGTCTCA
GTGATTACGCCTGGCACTAACACTAGCAACCAAGTCGCAGTACTTTATCA
GGGCGTTAACTGTACAGAAGTACCAGTTGCGATACACGCGGACCAGCTCA
CCCCAACCTGGCGCGTATATTCTACCGGATCTAATGTGTTTCAAACGCGG
GCGGGGTGCCTCATAGGTGCAGAGCATGTCAATAACTCCTACGAATGTGA
CATTCCTATAGGAGCAGGAATATGTGCGAGCTACCAGACTCAAACCAATT
CACACAGGAGCGTAGCTAGTCAGTCCATAATTGCCTACACTATGTCACTT
GGTGCTGAAAACAGTGTGGCTTATTCAAATAATTCCATAGCTATTCCAAT
CAATTTCACTATTAGCGTAACAACTGAGATACTTCCAGTCAGCATGACTA
AGACTTCAGTTGATTGTACGATGTATATTTGTGGCGATTCTACAGAATGT
AGTAACCTTCTGTTGCAATACGGCTCCTTTTGCACACAGCTCAATCGCGC
ATTGACGGGTATAGCTGTAGAGCAAGATAAAAACACCCAAGAAGTCTTCG
CGCAGGTGAAGCAAATATATAAGACACCACCGATTAAAGATTTCGGTGGA
TTCAATTTTAGCCAAATTCTGCCTGATCCAAGCAAACCGTCAAAAAGATC
CTTCATAGAGGACCTGCTTTTCAATAAGGTAACGCTGGCGGACGCAGGGT
TTATTAAACAATACGGCGATTGCCTTGGTGACATTGCTGCACGAGACCTC
ATTTGCGCCCAGAAGTTCAACGGACTCACAGTACTGCCTCCACTGCTCAC
AGACGAGATGATCGCCCAGTACACATCTGCCTTGTTGGCTGGTACGATAA
CTTCTGGTTGGACCTTTGGAGCGGGAGCAGCACTTCAGATTCCCTTTGCC
ATGCAGATGGCGTACAGATTCAACGGCATTGGTGTCACGCAGAACGTACT
GTATGAGAACCAGAAGTTGATAGCTAATCAATTCAACTCCGCGATAGGTA
AGATCCAAGACAGTCTCTCCTCAACAGCCTCCGCACTCGGGAAACTCCAG
GATGTGGTAAATCAGAATGCACAGGCATTGAATACGCTTGTTAAACAATT
GTCCTCAAATTTTGGCGCCATATCATCAGTGCTCAACGACATCCTTGCCA
GACTCGATAAAGTGGAGGCAGAAGTGCAGATAGATCGCTTGATTACTGGG
CGCCTCCAATCTCTCCAAACGTACGTGACACAACAGCTTATTCGGGCAGC
GGAAATCCGCGCCTCTGCGAACCTTGCAGCGACCAAGATGAGCGAATGTG
TTCTCGGGCAGTCAAAACGAGTAGATTTCTGCGGTAAAGGATATCATCTT
ATGTCATTCCCCCAAAGCGCTCCACATGGTGTTGTATTTCTGCACGTGAC
TTACGTGCCGGCACAGGAGAAGAATTTCACAACGGCTCCCGCCATCTGTC
ACGACGGAAAGGCACACTTCCCTAGGGAGGGGGTGTTTGTAAGCAATGGT
ACCCACTGGTTTGTCACACAACGAAACTTCTATGAACCTCAGATAATTAC
AACGCATAATACTTTTGTAAGTGGGAACTGCGACGTCGTTATCGGAATAG
TTAACAATACTGTATATGATCCGTTGCAGCCGGAGTTGGACTCATTTAAA
GAAGAGCTGGACAAGTACTTCAAAAACCATACAAGCCCAGATGTAGATCT
TGGGGACATAAGTGGGATAAACGCCAGCGTTGTGAACATACAGAAGGAAA
TAGACAGGCTGAATGAGGTAGCAAAGAATCTTAACGAAAGCCTTATAGAT
CTTCAGGAACTTGGGAAGTACGAACAGTACATAAAGTGGCCTTCACGACT
GGAGGAAGAACTGCGCCGACGCCTGACTGAATGA

In certain embodiments the vaccine comprises or encodes Kent Spike S1+S2 ECD_CD74+HLA. In specific embodiments, the vaccine comprises or encodes the sequence as set forth below (SEQ ID NO:46):

MHRRRSRSCREDQNPVMDDQRDLISNNEQLPMLGRRPGAPESKCSRWMVA
AVVAGTIVAGLLVLGAIIGVVNLTTRTQLPPAYTNSFTRGVYYPDKVFRS
SVLHSTQDLFLPFFSNVTWFHAISGTNGTKRFDNPVLPFNDGVYFASTEK
SNIIRGWIFGTTLDSKTQSLLIVNNATNVVIKVCEFQFCNDPFLGVYHKN
NKSWMESEFRVYSSANNCTFEYVSQPFLMDLEGKQGNFKNLREFVFKNID
GYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSE
TKCTLKSFTVEKGIYQTSNFRVQPTESIVRFPNITNLCPFGEVFNATRFA
SVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQ
PTYGVGYQPYRVVVLSFELLHAPATVCGPKKSTNLVKNKCVNFNFNGLTG
TGVLTESNKKFLPFQQFGRDIDDTTDAVRDPQTLEILDITPCSFGGVSVI
TPGTNTSNQVAVLYQGVNCTEVPVAIHADQLTPTWRVYSTGSNVFQTRAG
CLIGAEHVNNSYECDIPIGAGICASYQTQTNSHRSVASQSIIAYTMSLGA
ENSVAYSNNSIAIPINFTISVTTEILPVSMTKTSVDCTMYICGDSTECSN
LLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDV
VNQNAQALNTLVKQLSSNFGAISSVLNDILARLDKVEAEVQIDRLITGRL
QSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTH
WFVTQRNFYEPQIITTHNTFVSGNCDVVIGIVNNTVYDPLQPELDSFKEE
LDKYFKNHTSPDVDLGDISGINASVVNIQKEIDRLNEVAKNLNESLIDLQ
ELGKYEQYIKWPSRLEEELRRRLTE*

In certain embodiments, the vaccine comprises the sequence encoding South Africa Spike ECD_CD74+HLA as set forth below (SEQ ID NO:47).

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTCAATTTCACTACACGCACACAGCTGCCCCCGG
CGTACACTAACAGTTTTACGAGGGGAGTCTACTACCCGGACAAAGTGTTC
AGGTCTTCCGTACTCCATAGCACACAAGACCTCTTCTTGCCTTTTTTCAG
CAATGTTACGTGGTTCCATGCAATCCATGTGAGCGGTACAAACGGGACTA
AACGCTTTGCAAATCCAGTGTTGCCCTTCAACGACGGTGTATACTTCGCG
AGTACAGAGAAGAGCAACATAATACGCGGGTGGATATTTGGAACGACGCT
TGATTCTAAGACACAGAGCTTGCTCATCGTAAACAACGCAACGAATGTTG
TAATCAAGGTCTGCGAATTCCAATTCTGCAATGACCCATTTCTTGGAGTT
TATTACCATAAGAACAATAAGTCTTGGATGGAATCCGAGTTCCGCGTATA
CTCTAGCGCGAACAACTGCACCTTCGAGTATGTGTCCCAACCGTTTCTGA
TGGATCTTGAGGGAAAGCAGGGTAATTTTAAAAATCTCCGGGAGTTCGTT
TTCAAAAATATTGACGGATATTTCAAAATCTACAGCAAGCATACCCCAAT
CAATTTGGTGCGGGGTCTGCCACAAGGATTTTCCGCTCTTGAACCTCTTG
TTGACCTTCCCATAGGAATCAATATAACTCGGTTCCAAACTCTTCTTGCG
CTCCATATATCCTACTTGACGCCCGGAGATTCATCAAGTGGCTGGACGGC
TGGGGCTGCCGCATACTACGTCGGCTATCTGCAACCAAGGACGTTTCTCC
TCAAATACAATGAAAATGGTACGATCACCGACGCTGTGGACTGCGCTCTC
GACCCGTTGTCAGAGACGAAGTGCACCTTGAAGTCCTTCACCGTGGAAAA
GGGAATTTACCAGACTAGTAACTTCCGGGTGCAGCCCACGGAATCAATTG
TGCGATTTCCTAATATCACTAATCTCTGTCCCTTCGGGGAAGTGTTCAAT
GCGACTCGATTCGCTTCAGTATACGCTTGGAATAGAAAGAGAATCTCAAA
TTGCGTGGCTGACTATTCCGTACTGTATAATAGTGCGTCTTTTAGCACGT
TTAAGTGTTATGGCGTATCCCCGACAAAGCTCAACGATCTCTGCTTCACG
AATGTCTATGCCGACTCCTTCGTGATACGCGGTGACGAGGTACGACAAAT
TGCACCCGGCCAAACTGGAAATATTGCCGACTACAATTATAAGTTGCCGG
ACGATTTTACCGGTTGCGTAATAGCGTGGAACTCTAATAACCTCGACTCT
AAGGTAGGAGGCAACTACAATTACTTGTACCGACTGTTCAGGAAATCCAA
CCTGAAGCCCTTCGAAAGAGACATCAGTACAGAGATTTATCAAGCCGGGT
CCACCCCTTGCAATGGTGTCAAAGGCTTCAACTGCTATTTTCCGTTGCAG
TCTTACGGTTTCCAGCCGACGTACGGTGTGGGTTATCAACCCTACCGCGT
TGTGGTGCTGAGCTTTGAGCTTCTCCACGCTCCAGCCACTGTCTGCGGGC
CTAAGAAATCCACAAACCTCGTTAAGAATAAATGCGTAAATTTCAACTTC
AACGGGCTTACTGGCACGGGCGTGCTTACGGAGTCCAATAAGAAATTCCT
GCCTTTCCAGCAGTTCGGCCGCGACATCGCGGACACAACAGATGCTGTGC
GAGACCCACAAACCCTGGAGATTCTGGACATCACGCCTTGCTCTTTTGGC
GGTGTCAGCGTAATAACGCCAGGCACCAACACCAGTAATCAGGTCGCCGT
GCTTTATCAGGGTGTGAACTGTACGGAGGTACCGGTTGCCATACACGCAG
ACCAACTCACACCTACGTGGAGAGTTTACAGCACCGGGTCAAACGTATTT
CAGACCCGCGCAGGCTGTCTGATCGGCGCAGAGCATGTCAATAACTCATA
CGAGTGTGATATACCAATCGGAGCCGGCATCTGTGCGAGCTATCAAACCC
AAACTAATTCACGAAGCGTGGCCAGCCAGTCCATCATTGCCTACACGATG
TCTTTGGGAGTAGAGAATAGCGTGGCATACTCTAACAACAGTATCGCTAT
TCCTACCAACTTCACAATATCAGTTACTACAGAAATCCTCCCGGTGAGCA
TGACTAAGACATCCGTAGACTGTACAATGTACATTTGTGGGGACAGCACG
GAGTGTTCAAACCTGCTGTTGCAATACGGAAGCTTCTGCACCCAGCTCAA
CCGAGCCCTGACCGGCATTGCCGTAGAACAAGATAAGAATACGCAGGAAG
TGTTCGCCCAGGTTAAGCAGATTTACAAGACTCCGCCTATCAAGGACTTC
GGGGGTTTCAACTTTAGTCAGATTTTGCCTGACCCGTCAAAGCCTAGCAA
ACGAAGTTTTATCGAAGATTTGTTGTTTAACAAGGTGACTTTGGCTGATG
CGGGATTCATAAAACAATACGGCGATTGCCTTGGCGATATTGCGGCCCGA
GATCTCATTTGCGCGCAAAAGTTCAACGGTCTTACCGTCCTTCCGCCCCT
GCTTACGGACGAGATGATCGCACAATACACTTCAGCACTGCTGGCTGGTA
CAATAACCAGTGGGTGGACATTCGGGGCTGGTGCAGCGCTCCAGATCCCC
TTTGCGATGCAGATGGCGTATAGATTCAATGGCATCGGGGTCACTCAGAA
CGTGCTTTATGAGAACCAAAAGCTTATTGCAAATCAATTTAACTCTGCGA
TTGGAAAGATTCAAGACAGCCTCTCATCCACAGCTAGTGCGCTTGGCAAG
CTTCAGGACGTCGTAAACCAAAACGCCCAAGCTCTCAACACACTCGTTAA
ACAGCTTTCCTCAAACTTTGGGGCTATTAGCTCTGTGCTGAATGATATTC
TTAGCAGACTCGACAAAGTCGAGGCTGAAGTACAAATAGATAGACTGATA
ACAGGTAGACTTCAGTCTCTCCAAACCTACGTGACACAGCAGCTTATAAG
AGCTGCTGAGATCAGAGCTTCTGCGAACCTTGCGGCGACTAAAATGTCCG
AGTGTGTACTCGGGCAGTCTAAACGCGTCGACTTCTGTGGCAAGGGTTAC
CATCTCATGAGTTTTCCCCAATCAGCGCCCCATGGGGTAGTTTTTCTGCA
CGTAACATACGTCCCAGCCCAAGAAAAGAATTTTACCACCGCCCCTGCGA
TTTGTCACGACGGAAAAGCACACTTTCCAAGAGAAGGCGTATTTGTTAGC
AATGGCACCCATTGGTTCGTCACCCAGAGAAACTTTTACGAACCGCAGAT
TATCACGACAGATAATACATTTGTATCTGGTAATTGTGACGTCGTGATCG
GCATCGTAAACAACACTGTTTACGACCCCCTCCAGCCAGAGCTTGACTCA
TTTAAAGAGGAGCTCGACAAATATTTTAAGAACCACACAAGTCCAGACGT
CGACTTGGGTGACATTTCCGGCATCAATGCAAGTGTGGTCAACATCCAAA
AGGAAATTGACAGATTGAACGAGGTTGCTAAGAACCTTAATGAGTCACTC
ATTGACCTCCAGGAACTGGGAAAATACGAACAGTATATTAAGTGGCCCTC
ACGACTGGAGGAAGAACTGCGCCGACGCCTGACTGAATGA

In certain embodiments the vaccine comprises or encodes South Africa Spike S1+S2 ECD_CD74+HLA. In specific embodiments, the vaccine comprises or encodes the sequence as set forth below (SEQ ID NO:48).

MHRRRSRSCREDQNPVMDDQRDLISNNEQLPMLGRRPGAPESKCSRWMVA
AVVAGTIVAGLLVLGAIIGVVNFTTRTQLPPAYTNSFTRGVYYPDKVFRS
SVLHSTQDLFLPFFSNVTWFHAIHVSGTNGTKRFANPVLPFNDGVYFAST
EKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFK
NIDGYFKIYSKHTPINLVRGLPQGFSALEPLVDLPIGINITRFQTLLALH
ISYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNAT
RFASVYAWNRKRISNCVADYSVLYNSASFSTFKCYGVSPTKLNDLCFTNV
YADSFVIRGDEVRQIAPGQTGNIADYNYKLPDDFTGCVIAWNSNNLDSKV
GGNYNYLYRLFRKSNLKPFERDISTEIYQAGSTPCNGVKGFNCYFPLQSY
GFQPTYGVGYQPYRVVVLSFELLHAPATVCGPKKSTNLVKNKCVNFNFNG
LTGTGVLTESNKKFLPFQQFGRDIADTTDAVRDPQTLEILDITPCSFGGV
SVITPGTNTSNQVAVLYQGVNCTEVPVAIHADQLTPTWRVYSTGSNVFQT
RAGCLIGAEHVNNSYECDIPIGAGICASYQTQTNSRSVASQSIIAYTMSL
GVENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAARDL
ICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITG
RLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLID
LQELGKYEQYIKWPSRLEEELRRRLTE*

In certain embodiments, the vaccine comprises the sequence encoding California Spike S1+S2 ECD_CD74+HLA as set forth below (SEQ ID NO:49).

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTAAATTTGACAACGCGCACCCAATTGCCCCCAG
CATATACGAACTCTTTCACGAGGGGCGTATATTATCCGGATAAGGTATTT
CGGTCATCTGTTCTGCACAGCACCCAGGACCTCTTCCTTCCATTCTTTTC
AAATGTAACTTGGTTCCATGCGATAGTATCAGGAACGAATGGGACAAAAA
GGTTCGATAATCCGGTCTTGCCGTTCAACGATGGGGTGTACTTCGCCAGT
ACCGAAAAGTCTAACATTATACGCGGTTGGATTTTTGGCACTACGCTTGA
CTCAAAGACACAGTCACTCCTCATTGTAAATAATGCTACTAATGTCGTGA
TCAAAGTTTGTGAGTTCCAATTCTGCAACGACCCGTTCCTGGGAGTGTAT
TATCACAAAAATAATAAGTCCTGCATGGAGTCAGAGTTCAGGGTTTACTC
AAGTGCGAACAATTGCACATTTGAGTACGTTTCTCAACCATTTCTCATGG
ATTTGGAGGGGAAGCAGGGAAACTTTAAAAACCTGAGAGAATTTGTTTTT
AAGAACATTGATGGGTATTTCAAGATTTATAGTAAACACACCCCTATCAA
CTTGGTTAGGGATCTCCCTCAAGGTTTCTCTGCTCTCGAGCCCCTTGTAG
ATCTGCCAATAGGCATCAATATCACACGCTTTCAGACACTCCTCGCACTT
CATAGGAGCTACCTGACGCCAGGTGACTCTTCCTCAGGTTGGACAGCCGG
CGCAGCCGCATACTACGTTGGCTACCTCCAACCAAGGACATTTCTGTTGA
AATACAACGAAAATGGGACCATCACCGACGCAGTCGATTGTGCTCTCGAC
CCTCTTTCCGAGACTAAATGTACCCTCAAAAGCTTTACTGTTGAGAAGGG
TATCTATCAGACATCTAACTTTCGGGTGCAACCCACTGAGTCAATTGTGC
GATTCCCAAATATTACGAACCTCTGTCCTTTTGGCGAGGTTTTTAACGCC
ACTAGGTTCGCCAGTGTATATGCTTGGAACCGAAAACGGATAAGCAATTG
TGTTGCTGACTACTCCGTCCTCTACAATAGCGCTAGTTTCTCAACATTTA
AGTGTTACGGTGTGAGCCCTACGAAACTTAACGATTTGTGCTTCACTAAC
GTCTATGCCGACAGTTTCGTAATCCGAGGCGATGAGGTCAGGCAAATTGC
CCCGGGCCAAACGGGGAAAATCGCTGATTACAATTATAAGTTGCCAGATG
ATTTTACGGGATGTGTCATTGCATGGAACAGTAATAACCTCGATTCAAAG
GTTGGCGGAAATTATAATTACAGATATCGGCTTTTTAGAAAATCTAACCT
TAAACCATTTGAGCGGGACATAAGCACGGAGATTTACCAGGCTGGTAGCA
CTCCGTGCAACGGTGTAGAAGGATTCAATTGCTATTTTCCATTGCAGTCT
TATGGATTCCAACCCACCAATGGGGTAGGGTACCAACCATACAGGGTGGT
AGTCCTTAGCTTTGAACTTTTGCATGCGCCAGCTACCGTCTGCGGTCCCA
AAAAGAGTACGAACTTGGTAAAAAATAAATGCGTCAACTTTAATTTTAAC
GGTCTGACGGGAACGGGGGTTCTCACCGAGTCTAACAAAAAGTTTTTGCC
ATTTCAGCAGTTCGGACGAGATATTGCCGACACTACCGACGCCGTGCGCG
ATCCACAAACCTTGGAGATACTCGACATCACACCTTGCAGCTTCGGTGGT
GTTAGCGTTATTACGCCAGGAACGAACACTTCAAATCAGGTGGCCGTCTT
GTATCAAGATGTTAACTGTACAGAGGTGCCCGTCGCAATACACGCAGATC
AGCTCACCCCTACCTGGAGAGTTTACTCTACAGGCTCTAATGTGTTCCAA
ACTAGGGCAGGCTGCTTGATTGGAGCTGAACACGTAAACAACTCATACGA
GTGCGATATCCCGATTGGTGCGGGTATTTGCGCCAGCTATCAGACGCAGA
CTAACTCACGATCAGTGGCGTCACAGTCAATAATTGCGTATACAATGAGT
CTCGGTGCAGAGAATTCCGTCGCATACTCAAACAACAGCATAGCGATTCC
AACTAATTTTACCATTAGCGTTACAACGGAAATCCTCCCCGTTAGCATGA
CCAAGACCTCCGTGGATTGCACAATGTACATCTGTGGAGACAGCACCGAG
TGCTCCAATCTTCTTCTTCAATATGGGAGCTTTTGCACACAGCTGAACCG
CGCGCTCACTGGCATTGCGGTAGAACAAGACAAAAACACCCAAGAAGTGT
TTGCGCAGGTCAAACAGATTTACAAGACACCACCAATCAAAGACTTCGGG
GGGTTCAATTTCTCCCAAATTCTTCCTGATCCAAGCAAACCATCTAAAAG
GTCCTTCATAGAAGACCTCCTGTTCAATAAGGTGACACTCGCGGACGCAG
GGTTTATTAAGCAATATGGAGATTGTCTCGGGGATATTGCAGCGAGAGAT
CTTATTTGTGCTCAAAAGTTTAATGGCTTGACTGTCCTCCCGCCCTTGCT
CACAGACGAGATGATAGCACAATATACCAGTGCGCTGCTTGCGGGCACGA
TTACTTCCGGATGGACTTTCGGGGCAGGAGCGGCGCTCCAGATTCCTTTC
GCGATGCAAATGGCCTACCGATTTAACGGGATTGGCGTCACACAAAACGT
TCTTTACGAAAACCAGAAACTTATTGCCAACCAATTCAATAGTGCAATTG
GTAAAATTCAGGATTCACTCTCCTCCACGGCGTCAGCTCTCGGCAAGCTC
CAAGACGTGGTTAACCAAAATGCGCAGGCGCTTAACACGCTTGTAAAACA
ACTCTCTTCTAATTTTGGCGCGATTAGTAGCGTTCTCAACGATATCTTGT
CACGCTTGGACAAAGTAGAAGCAGAAGTCCAAATAGACCGGCTGATCACT
GGTCGATTGCAATCTCTGCAGACATACGTGACGCAGCAGCTCATCCGGGC
AGCCGAGATCCGGGCGTCCGCTAATCTGGCCGCAACCAAAATGAGCGAGT
GTGTCCTTGGGCAAAGCAAACGCGTGGATTTTTGCGGAAAGGGCTACCAC
CTCATGAGTTTCCCCCAATCTGCCCCGCACGGTGTCGTCTTTTTGCATGT
AACGTATGTCCCGGCGCAGGAGAAAAATTTCACTACAGCTCCCGCCATCT
GTCACGACGGCAAGGCACACTTCCCTCGAGAAGGGGTGTTTGTAAGCAAT
GGAACACACTGGTTCGTCACCCAGCGGAATTTTTATGAACCTCAGATAAT
CACAACAGACAACACTTTCGTTTCCGGAAACTGCGATGTCGTAATTGGGA
TAGTGAATAATACCGTATATGACCCATTGCAGCCGGAGTTGGACTCCTTT
AAAGAGGAATTGGACAAATACTTTAAAAATCACACTTCACCCGATGTGGA
CCTGGGAGATATATCCGGGATTAACGCAAGTGTGGTTAACATACAAAAGG
AAATTGACCGCCTTAACGAGGTTGCTAAGAACTTGAATGAGTCTCTCATA
GATCTCCAGGAACTGGGGAAATACGAGCAATATATCAAATGGCCGTCACG
ACTGGAGGAAGAACTGCGCCGACGCCTGACTGAATGA

In certain embodiments the vaccine comprises or encodes California Spike S1+S2 ECD_CD74+HLA. In specific embodiments, the vaccine comprises or encodes the sequence as set forth below (SEQ ID NO:50).

MHRRRSRSCREDQNPVMDDQRDLISNNEQLPMLGRRPGAPESKCSRWMVA
AVVAGTIVAGLLVLGAIIGVVNLTTRTQLPPAYTNSFTRGVYYPDKVFRS
SVLHSTQDLFLPFFSNVTWFHAIVSGTNGTKRFDNPVLPFNDGVYFASTE
KSNIIRGWIFGTTLDSKTQSLLIVNNATNVVIKVCEFQFCNDPFLGVYYH
KNNKSCMESEFRVYSSANNCTFEYVSQPFLMDLEGKQGNFKNLREFVFKN
IDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPL
SETKCTLKSFTVEKGIYQTSNFRVQPTESIVRFPNITNLCPFGEVFNATR
FASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVG
GNYNYRYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGL
TGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTR
AGCLIGAEHVNNSYECDIPIGAGICASYQTQTNSRSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPSRLEEELRRRLTE*

In certain embodiments, the vaccine comprises the sequence encoding Brazil Spike S1+S2 ECD_CD74+HLA as set for the below (SEQ ID NO:51):

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTTAACTTTACTAATCGGACCCAACTCCCGAGTG
CGTACACTAACTCTTTTACCCGGGGCGTCTATTATCCTGACAAAGTGTTT
CGATCATCTGTTTTGCACTCCACCCAAGATCTCTTTCTTCCATTTTTCAG
CAACGTTACGTGGTTCCACGCGATACATGTTAGTGGTACTAATGGCACAA
AGAGATTTGACAACCCTGTGCTCCCGTTTAACGATGGGGTCTATTTCGCT
TCCACTGAGAAGTCCAACATTATTCGCGGTTGGATCTTCGGCACCACACT
CGATTCAAAAACCCAAAGCTTGCTCATAGTTAACAACGCCACAAACGTAG
TCATTAAAGTGTGCGAATTTCAGTTTTGTAACTATCCCTTTTTGGGCGTC
TACTATCACAAGAACAACAAGTCCTGGATGGAATCTGAGTTTCGGGTCTA
CAGTTCAGCCAACAACTGTACGTTTGAGTACGTTAGCCAGCCCTTTCTGA
TGGATCTTGAAGGGAAGCAAGGAAATTTCAAGAATCTTTCCGAGTTTGTG
TTTAAAAATATAGACGGATATTTCAAGATATACAGTAAGCACACACCGAT
CAATTTGGTACGGGATTTGCCGCAAGGATTTTCCGCCCTTGAGCCCCTTG
TAGATCTCCCCATCGGAATTAACATAACCAGGTTTCAAACACTGCTGGCG
CTCCACAGGAGCTATCTGACCCCGGGCGACTCATCCAGTGGGTGGACCGC
CGGAGCAGCCGCATACTACGTGGGGTATCTCCAGCCCAGAACGTTTCTCC
TCAAGTATAATGAAAACGGGACCATCACCGACGCGGTTGATTGTGCACTC
GATCCCCTCAGCGAGACAAAGTGCACACTTAAATCATTTACCGTCGAGAA
GGGGATCTACCAAACATCCAACTTTCGGGTGCAGCCTACCGAGTCAATAG
TACGGTTTCCTAACATCACCAATTTGTGCCCTTTTGGGGAAGTATTTAAT
GCAACCCGGTTTGCGAGCGTATACGCTTGGAACCGAAAACGGATAAGTAA
TTGTGTTGCAGATTATTCTGTGCTCTATAACTCAGCTTCCTTCTCCACCT
TTAAGTGTTACGGAGTATCTCCGACAAAATTGAATGATCTTTGTTTTACG
AATGTTTACGCTGACTCATTTGTTATCCGAGGTGACGAAGTGCGCCAGAT
AGCCCCCGGTCAAACAGGCAACATTGCTGATTATAACTACAAGTTGCCCG
ATGACTTCACGGGGTGTGTTATTGCATGGAATTCAAATAATCTTGATAGT
AAGGTAGGGGGTAACTATAATTACCTTTATCGCTTGTTCCGCAAATCCAA
TCTGAAACCCTTCGAAAGGGATATCAGTACCGAGATTTATCAAGCCGGCT
CCACACCTTGTAACGGAGTAAAAGGTTTTAACTGTTATTTCCCACTGCAG
AGTTACGGCTTTCAACCCACATACGGGGTGGGATATCAGCCCTATCGCGT
TGTGGTCTTGTCCTTTGAACTTCTCCACGCCCCTGCAACAGTTTGTGGTC
CTAAAAAGAGTACAAATCTGGTAAAAAATAAGTGCGTCAACTTTAACTTC
AACGGACTGACTGGTACCGGGGTCCTCACTGAGAGCAACAAGAAATTTTT
GCCCTTCCAGCAATTCGGGAGGGATATTGCCGATACTACCGATGCTGTAC
GGGACCCCCAGACACTCGAAATATTGGACATAACCCCGTGCTCTTTTGGA
GGGGTCTCTGTGATTACCCCGGGTACGAACACGAGTAATCAGGTGGCAGT
GCTTTATCAGGGAGTGAATTGCACAGAAGTACCAGTCGCGATCCATGCCG
ACCAGCTCACGCCGACCTGGAGGGTTTATAGTACGGGTAGTAACGTTTTC
CAAACACGGGCCGGTTGTCTCATCGGGGCAGAGTACGTTAATAACAGCTA
TGAATGTGATATACCCATAGGTGCCGGGATTTGTGCTTCTTATCAAACCC
AAACCAATAGCAGAAGCGTTGCGTCACAGTCTATAATCGCCTATACGATG
AGCCTCGGAGCGGAAAACTCAGTTGCCTACTCCAACAATAGCATTGCTAT
CCCAACGAACTTCACTATTAGCGTAACCACGGAGATCCTTCCAGTTAGCA
TGACAAAAACTAGCGTCGATTGCACTATGTATATTTGCGGCGACAGTACA
GAGTGTAGTAATTTGCTCCTGCAGTACGGGTCTTTCTGCACCCAATTGAA
CCGAGCACTGACTGGCATCGCCGTGGAACAAGACAAAAACACGCAAGAAG
TATTTGCGCAAGTCAAGCAGATTTACAAGACTCCCCCGATCAAAGACTTT
GGCGGATTTAATTTTAGCCAGATACTCCCCGATCCATCTAAACCATCAAA
ACGGTCCTTTATAGAAGATCTCTTGTTCAATAAAGTGACACTCGCGGACG
CGGGCTTTATCAAACAGTATGGGGACTGCCTTGGAGATATAGCTGCGAGG
GACTTGATTTGCGCCCAGAAATTCAACGGGCTGACGGTTCTGCCGCCGCT
CTTGACAGACGAAATGATTGCACAGTATACGAGTGCCTTGCTGGCAGGGA
CTATCACTTCCGGTTGGACCTTCGGCGCAGGAGCAGCGCTCCAGATCCCA
TTCGCTATGCAAATGGCTTACCGATTCAACGGAATTGGCGTCACACAGAA
CGTGCTTTACGAGAATCAAAAGCTGATTGCCAATCAATTCAACTCAGCGA
TTGGCAAAATACAAGATAGTCTCTCTTCAACTGCATCCGCGCTCGGTAAG
TTGCAGGACGTAGTCAATCAGAACGCACAGGCCCTGAACACGCTTGTTAA
ACAACTGTCTTCAAACTTCGGTGCAATAAGTTCCGTACTTAATGATATCT
TGTCAAGGTTGGATAAGGTCGAAGCTGAGGTACAAATCGACCGCCTTATA
ACAGGCAGACTCCAATCCCTGCAGACATACGTGACCCAGCAACTCATTAG
GGCAGCCGAGATCAGAGCAAGTGCCAACCTCGCTGCTATTAAAATGTCTG
AATGCGTTTTGGGGCAATCTAAGCGGGTTGATTTCTGCGGTAAGGGATAC
CACTTGATGTCCTTTCCTCAGTCCGCTCCTCATGGGGTAGTATTTCTTCA
TGTTACATATGTACCAGCACAAGAGAAAAATTTTACTACTGCCCCGGCCA
TATGCCATGATGGCAAAGCGCACTTCCCTCGGGAGGGGGTTTTTGTGTCT
AATGGAACCCACTGGTTCGTGACTCAGCGGAATTTCTATGAACCCCAGAT
TATAACTACTGACAACACTTTCGTTTCTGGAAACTGCGATGTAGTAATAG
GCATAGTAAATAATACAGTATATGATCCCTTGCAACCAGAATTGGACTCT
TTTAAAGAAGAGCTCGACAAATATTTTAAAAACCATACATCTCCAGACGT
TGATCTTGGAGACATCTCTGGGATAAACGCTAGCTTCGTAAACATACAAA
AAGAAATCGATAGGCTTAACGAGGTCGCAAAAAATCTTAATGAAAGTCTT
ATCGATTTGCAAGAGCTTGGAAAGTATGAGCAGTATATCAAATGGCCTTC
ACGACTGGAGGAAGAACTGCGCCGACGCCTGACTGAATGA

In certain embodiments the vaccine comprises or encodes Brazil Spike S1+S2 ECD_CD74+HLA. In specific embodiments, the vaccine comprises or encodes the sequence as set for the below (SEQ ID NO:52):

MHRRRSRSCREDQNPVMDDQRDLISNNEQLPMLGRRPGAPESKCSRWMVA
AVVAGTIVAGLLVLGAIIGVVNFTNRTQLPSAYTNSFTRGVYYPDKVFRS
SVLHSTQDLFLPFFSNVTWFHAIHVSGTNGTKRFDNPVLPFNDGVYFAST
EKSNIIRGWIFGTTLDSKTQSLLIVNNATNVVIKVCEFQFCNYPFLGVYY
HKNNKSWMESEFRVYSSANNCTFEYVSQPFLMDLEGKQGNFKNLSEFVFK
NIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNAT
RFASVYAWNRKRISNCVADYSVLYNSASFSTFKCYGVSPTKLNDLCFTNV
YADSFVIRGDEVRQIAPGQTGNIADYNYKLPDDFTGCVIAWNSNNLDSKV
GGNYNYLYRLFRKSNLKPFERDISTEIYQAGSTPCNGVKGFNCYFPLQSY
GFQPTYGVGYQPYRVVVLSFELLHAPATVCGPKKSTNLVKNKCVNFNFNG
LTGTGVLTESNKKFLPFQQFGRDIADTTDAVRDPQTLEILDITPCSFGGV
SVITPGTNTSNQVAVLYQGVNCTEVPVAIHADQLTPTWRVYSTGSNVFQT
RAGCLIGAEYVNNSYECDIPIGAGICASYQTQTNSRSVASQSIIAYTMSL
GAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGG
FNFSQILPDPSKPSKRSFIEDLLFNKVTLADAGFIKQYGDCLGDIAARDL
ICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQ
DVVNQNAQALNTLVKQLSSNFGAISSVLNDILSRLDKVEAEVQIDRLITG
RLQSLQTYVTQQLIRAAEIRASANLAAIKMSECVLGQSKRVDFCGKGYHL
MSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFK
EELDKYFKNHTSPDVDLGDISGINASFVNIQKEIDRLNEVAKNLNESLID
LQELGKYEQYIKWPSRLEEELRRRLTE*

In certain embodiments, the vaccine comprises the sequence encoding Delta Spike S1+S2 ECD_CD74+HLA as set forth below (SEQ ID NO:53):

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTTAATCTTACAACCAGAACTCAATTACCCCCTG
CATACACTAATTCTTTCACACGTGGTGTTTATTACCCTGACAAAGTTTTC
AGATCCTCAGTTTTACATTCAACTCAGGACTTGTTCTTACCTTTCTTTTC
CAATGTTACTTGGTTCCATGCTATACATGTCTCTGGGACCAATGGTACTA
AGAGGTTTGATAACCCTGTCCTACCATTTAATGATGGTGTTTATTTTGCT
TCCATTGAGAAGTCTAACATAATAAGAGGCTGGATTTTTGGTACTACTTT
AGATTCGAAGACCCAGTCCCTACTTATTGTTAATAACGCTACTAATGTTG
TTATTAAAGTCTGTGAATTTCAATTTTGTAATGATCCATTTTTGGGTGTT
TATTACCACAAAAACAACAAAAGTTGGATGAAAAGTGAGTTCAGAGTTTA
TTCTAGTGCGAATAATTGCACTTTTGAATATGTCTCTCAGCCTTTTCTTA
TGGACCTTGAAGGAAAACAGGGTAATTTCAAAAATCTTAGGGAATTTGTG
TTTAAGAATATTGATGGTTATTTTAAAATATATTCTAAGCACACGCCTAT
TAATTTAGTGCGTGATCTCCCTCAGGGTTTTTCGGCTTTAGAACCATTGG
TAGATTTGCCAATAGGTATTAACATCACTAGGTTTCAAACTTTACTTGCT
TTACATAGAAGTTATTTGACTCCTGGTGATTCTTCTTCAGGTTGGACAGC
TGGTGCTGCAGCTTATTATGTGGGTTATCTTCAACCTAGGACTTTTCTAT
TAAAATATAATGAAAATGGAACCATTACAGATGCTGTAGACTGTGCACTT
GACCCTCTCTCAGAAACAAAGTGTACGTTGAAATCCTTCACTGTAGAAAA
AGGAATCTATCAAACTTCTAACTTTAGAGTCCAACCAACAGAATCTATTG
TTAGATTTCCTAATATTACAAACTTGTGCCCTTTTGGTGAAGTTTTTAAC
GCCACCAGATTTGCATCTGTTTATGCTTGGAACAGGAAGAGAATCAGCAA
CTGTGTTGCTGATTATTCTGTCCTATATAATTCCGCATCATTTTCCACTT
TTAAGTGTTATGGAGTGTCTCCTACTAAATTAAATGATCTCTGCTTTACT
AATGTCTATGCAGATTCATTTGTAATTAGAGGTGATGAAGTCAGACAAAT
CGCTCCAGGGCAAACTGGAAAGATTGCTGATTATAATTATAAATTACCAG
ATGATTTTACAGGCTGCGTTATAGCTTGGAATTCTAACAAACTTGATTCT
AAGGTTGGTGGTAATTATAATTACCGCTATAGATTGTTTAGGAAGTCTAA
TCTCAAACCTTTTGAGAGAGATATTTCAACTGAAATCTATCAGGCCGGTA
GCACACCTTGTAATGGTGTTCAGGGTTTTAATTGTTACTTTCCTTTACAA
TCATATGGTTTCCAACCCACTTATGGTGTTGGTTACCAACCATACAGAGT
AGTAGTACTTTCTTTTGAACTTCTACATGCACCAGCAACTGTTTGTGGAC
CTAAAAAGTCTACTAATTTGGTTAAAAACAAATGTGTCAATTTCAACTTC
AATGGTTTAACAGGCACAGGTGTTCTTACTGAGTCTAACAAAAAGTTTCT
GCCTTTCCAACAATTTGGCAGAGACATTGATGACACTACTGATGCTGTCC
GTGATCCACAGACACTTGAGATTCTTGACATTACACCATGTTCTTTTGGT
GGTGTCAGTGTTATAACACCAGGAACAAATACTTCTAACCAGGTTGCTGT
TCTTTATCAGGGTGTTAACTGCACAGAAGTCCCTGTTGCTATTCATGCAG
ATCAACTTACTCCTACTTGGCGTGTTTATTCTACAGGTTCTAATGTTTTT
CAAACACGTGCAGGCTGTTTAATAGGGGCTGAACATGTCAACAACTCATA
TGAGTGTGACATACCCATTGGTGCAGGTATATGCGCTAGTTATCAGACTC
AGACTAATTCTAGTGTAGCTAGTCAATCCATCATTGCCTACACTATGTCA
CTTGGTGCAGAAAATTCAGTTGCTTACTCTAATAACTCTATTGCCATACC
CATTAATTTTACTATTAGTGTTACCACAGAAATTCTACCAGTGTCTATGA
CCAAGACATCAGTAGATTGTACAATGTACATTTGTGGTGATTCAACTGAA
TGCAGCAATCTTTTGTTGCAATATGGCAGTTTTTGTACACAATTAAACCG
TGCTTTAACTGGAATAGCTGTTGAACAAGACAAAAACACCCAAGAAGTTT
TTGCACAAGTCAAACAAATTTACAAAACACCACCAATTAAAGATTTTGGT
GGTTTTAATTTTTCACAAATATTACCAGATCCATCAAAACCAAGCAAGAG
GTCATTTATTGAAGATCTACTTTTCAACAAAGTGACACTTGCAGATGCTG
GCTTCATCAAACAATATGGTGATTGCCTTGGTGATATTGCTGCTAGAGAC
CTCATTTGTGCACAAAAGTTTAACGGCCTTACTGTTTTGCCACCTTTGCT
CACAGATGAAATGATTGCTCAATACACTTCTGCACTGTTAGCGGGTACAA
TCACTTCTGGTTGGACCTTTGGTGCAGGTGCTGCATTACAAATACCATTT
GCTATGCAAATGGCTTATAGGTTTAATGGTATTGGAGTTACACAGAATGT
TCTCTATGAGAACCAAAAATTGATTGCCAACCAATTTAATAGTGCTATTG
GCAAAATTCAAGACTCACTTTCTTCCACAGCAAGTGCACTTGGAAAACTT
CAAGATGTGGTCAACCAAAATGCACAAGCTTTAAACACGCTTGTTAAACA
ACTTAGCTCCAATTTTGGTGCAATTTCAAGTGTTTTAAATGATATCCTTG
CACGTCTTGACAAAGTTGAGGCTGAAGTGCAAATTGATAGGTTGATCACA
GGCAGACTTCAAAGTTTGCAGACATATGTGACTCAACAATTAATTAGAGC
TGCAGAAATCAGAGCTTCTGCTAATCTTGCTGCTACTAAAATGTCAGAGT
GTGTACTTGGACAATCAAAAAGAGTTGATTTTTGTGGAAAGGGCTATCAT
CTTATGTCCTTCCCTCAGTCAGCACCTCATGGTGTAGTCTTCTTGCATGT
GACTTATGTCCCTGCACACGAAAAGAACTTCACAACTGCTCCTGCCATTT
GTCATGATGGAAAAGCACACTTTCCTCGTGAAGGTGTCTTTGTTTCAAAT
GGCACACACTGGTTTGTAACACAAAGGAATTTTTATGAACCACAAATCAT
TACTACACACAACACATTTGTGTCTGGTAACTGTGATGTTGTAATAGGAA
TTGTCAACAACACAGTTTATGATCCTTTGCAACCTGAATTAGACTCATTC
AAGGAGGAGTTAGATAAATATTTTAAGAATCATACATCACCAGATGTTGA
TTTAGGTGACATCTCTGGCATTAATGCTTCAGTTGTAAACATTCAAAAAG
AAATTGACCGCCTCAATGAGGTTGCCAAGAATTTAAATGAATCTCTCATC
GATCTCCAAGAACTTGGAAAGTATGAGCAGTATATAAAATGGCCATCACG
ACTGGAGGAAGAACTGCGCCGACGCCTGACTGAATAA

In certain embodiments the vaccine comprises or encodes Delta Spike S1+S2 ECD_CD74+HLA. In specific embodiments, the vaccine comprises or encodes the sequence as set forth below (SEQ ID NO:54).

MHRRRSRSCREDQNPVMDDQRDLISNNEQLPMLGRRPGAPESKCSRWMVA
AVVAGTIVAGLLVLGAIIGVVNLTTRTQLPPAYTNSFTRGVYYPDKVFRS
SVLHSTQDLFLPFFSNVTWFHAIHVSGTNGTKRFDNPVLPFNDGVYFASI
EKSNIIRGWIFGTTLDSKTQSLLIVNNATNVVIKVCEFQFCNDPFLGVYY
HKNNKSWMKSEFRVYSSANNCTFEYVSQPFLMDLEGKQGNFKNLREFVFK
NIDGYFKIYSKHTPINLVRDLPQGFSALEPLVDLPIGINITRFQTLLALH
RSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNAT
RFASVYAWNRKRISNCVADYSVLYNSASFSTFKCYGVSPTKLNDLCFTNV
YADSFVIRGDEVRQIAPGQTGKIADYNYKLPDDFTGCVIAWNSNKLDSKV
GGNYNYRYRLFRKSNLKPFERDISTEIYQAGSTPCNGVQGFNCYFPLQSY
GFQPTYGVGYQPYRVVVLSFELLHAPATVCGPKKSTNLVKNKCVNFNFNG
LTGTGVLTESNKKFLPFQQFGRDIDDTTDAVRDPQTLEILDITPCSFGGV
SVITPGTNTSNQVAVLYQGVNCTEVPVAIHADQLTPTWRVYSTGSNVFQT
RAGCLIGAEHVNNSYECDIPIGAGICASYQTQTNSSVASQSIIAYTMSLG
AENSVAYSNNSIAIPINFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILARLDKVEAEVQIDRLITGR
LQSLQTYVTQQLIRAAEIRASANLAATKMSECVLGQSKRVDFCGKGYHLM
SFPQSAPHGVVFLHVTYVPAHEKNFTTAPAICHDGKAHFPREGVFVSNGT
HWFVTQRNFYEPQIITTHNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPSRLEEELRRRLTE*

In certain embodiments, the vaccine comprises the sequence encoding Wuhan Spike S1+S2 ECD_HLA as set forth below (SEQ ID NO:55):

GCCACCATGGCGGTCATGGCACCACGCACGCTCCTTCTTTTGTTGTCTGG
TGCCCTTGCACTCACCCAAACGTGGGCAGTGAACCTCACTACGCGAACTC
AACTGCCGCCTGCTTACACTAATAGTTTTACACGAGGAGTCTACTACCCG
GACAAGGTGTTCCGCTCAAGTGTCTTGCACAGCACGCAAGACCTGTTCTT
GCCATTTTTCAGCAATGTAACGTGGTTTCATGCCATCCACGTGTCAGGTA
CTAACGGAACGAAGAGATTTGATAACCCGGTCCTCCCGTTCAACGACGGG
GTTTATTTTGCCAGTACAGAAAAGTCCAATATTATAAGGGGATGGATTTT
CGGCACGACTCTCGACTCTAAGACACAAAGCTTGCTCATCGTTAATAATG
CTACAAACGTGGTTATAAAAGTATGCGAGTTCCAATTCTGTAACGACCCC
TTTCTGGGGGTTTACTACCACAAAAATAATAAGAGTTGGATGGAGAGCGA
ATTCAGAGTTTACTCTTCCGCGAATAATTGCACCTTCGAGTACGTATCCC
AGCCTTTTCTTATGGATCTTGAGGGGAAACAAGGTAATTTCAAAAACCTT
AGAGAATTCGTTTTTAAGAATATAGATGGATACTTCAAGATTTATTCTAA
ACATACCCCAATCAATCTGGTGCGCGATCTTCCTCAGGGTTTCTCTGCGC
TGGAGCCTCTCGTTGATCTGCCGATCGGAATTAATATAACCAGATTCCAG
ACCCTTTTGGCATTGCACCGCTCCTATCTGACACCTGGGGATAGTAGTTC
CGGCTGGACAGCGGGCGCCGCGGCATACTACGTTGGATATCTCCAACCCC
GCACTTTTCTTCTGAAATATAATGAGAATGGAACGATTACAGACGCAGTA
GATTGCGCGCTTGACCCCCTTTCAGAAACAAAGTGCACGCTGAAATCTTT
CACGGTCGAGAAGGGCATTTACCAAACGTCCAATTTCAGAGTTCAACCCA
CTGAGTCTATAGTCAGGTTTCCGAACATCACCAATCTCTGCCCATTTGGA
GAGGTTTTCAACGCAACACGGTTCGCCTCTGTATACGCATGGAATAGAAA
GAGAATATCCAATTGTGTCGCAGACTATAGTGTCTTGTACAACTCTGCGT
CATTTAGCACTTTTAAATGTTATGGGGTGAGTCCAACTAAATTGAACGAT
CTCTGCTTTACAAATGTTTACGCAGATAGTTTTGTGATACGAGGCGACGA
GGTACGACAAATTGCTCCTGGCCAGACCGGGAAAATAGCGGACTACAATT
ATAAACTGCCAGACGATTTCACCGGCTGTGTGATCGCGTGGAACTCAAAC
AACTTGGATAGTAAGGTAGGTGGTAACTATAACTACTTGTACCGGCTCTT
CCGCAAAAGTAATCTTAAACCGTTCGAGCGGGACATCTCTACCGAGATCT
ATCAAGCAGGTAGCACTCCCTGCAATGGAGTCGAGGGTTTTAATTGCTAT
TTTCCCCTCCAATCATACGGGTTTCAGCCCACAAATGGTGTTGGGTATCA
GCCCTATCGAGTTGTGGTGCTTTCATTTGAATTGCTTCATGCACCGGCGA
CAGTTTGTGGTCCTAAAAAGAGTACCAATTTGGTTAAAAACAAATGTGTC
AACTTTAACTTTAACGGTCTGACTGGTACGGGGGTGCTTACGGAATCTAA
TAAAAAGTTTCTTCCGTTCCAGCAATTCGGACGGGACATAGCGGACACGA
CTGACGCGGTTAGAGACCCACAAACACTCGAGATTCTTGACATAACCCCC
TGTTCTTTCGGCGGAGTTTCTGTAATAACGCCTGGGACAAATACGTCAAA
CCAGGTTGCAGTATTGTACCAGGATGTCAACTGCACTGAAGTGCCAGTCG
CGATCCACGCAGATCAGTTGACTCCAACATGGAGGGTCTACTCTACCGGT
TCTAACGTGTTTCAGACCAGAGCCGGGTGCCTCATTGGCGCGGAACATGT
GAACAATTCTTACGAATGCGACATACCCATCGGCGCCGGCATTTGTGCGT
CATACCAAACTCAAACTAATTCCCGGAGTGTGGCCTCCCAATCTATTATT
GCTTATACTATGAGTCTTGGCGCGGAAAACAGTGTAGCGTACTCAAATAA
CTCTATTGCGATCCCCACGAATTTCACTATAAGCGTAACTACTGAAATTT
TGCCGGTAAGCATGACGAAAACTTCAGTGGACTGCACAATGTACATTTGT
GGTGACAGCACTGAGTGCTCAAATCTCCTCCTTCAGTATGGTTCTTTCTG
CACACAACTTAACAGGGCACTTACCGGAATCGCAGTAGAACAAGACAAAA
ACACGCAAGAAGTATTCGCACAGGTCAAACAAATTTATAAAACCCCGCCG
ATCAAGGATTTTGGAGGGTTTAACTTCAGTCAGATCCTTCCAGACCCATC
CAAACCCAGCAAACGGAGCTTTATTGAAGACCTGCTGTTTAATAAAGTTA
CCCTTGCAGATGCCGGTTTCATCAAGCAATACGGGGATTGTCTGGGCGAC
ATTGCCGCCCGCGACTTGATCTGTGCCCAGAAGTTCAACGGTCTCACAGT
CTTGCCGCCACTTCTCACTGATGAAATGATAGCGCAGTATACATCCGCGC
TGCTGGCTGGGACGATAACATCTGGTTGGACTTTCGGGGCCGGGGCGGCT
TTGCAAATTCCTTTCGCCATGCAGATGGCCTATAGGTTTAACGGCATAGG
AGTGACTCAAAATGTTCTTTATGAGAATCAGAAGCTTATCGCCAACCAAT
TTAACTCCGCGATTGGGAAGATACAGGACTCATTGAGCTCTACTGCTAGC
GCCCTTGGCAAGCTTCAAGACGTAGTCAACCAAAATGCTCAAGCGCTCAA
TACTCTCGTAAAGCAGCTCTCAAGCAACTTTGGCGCGATATCTTCAGTTT
TGAACGACATCTTGAGCAGACTCGACAAGGTGGAGGCTGAAGTTCAAATT
GATCGACTGATTACGGGCCGGCTCCAGTCACTCCAAACGTACGTGACCCA
ACAACTCATTAGAGCGGCAGAGATTAGAGCGAGCGCTAATCTGGCGGCCA
CAAAGATGTCAGAATGTGTGCTTGGTCAATCCAAAAGGGTGGATTTCTGC
GGAAAGGGCTATCACTTGATGAGTTTCCCGCAGTCCGCGCCTCATGGAGT
TGTTTTCCTCCACGTAACCTACGTTCCCGCTCAGGAAAAGAATTTCACGA
CAGCTCCGGCAATATGTCATGACGGTAAAGCACATTTCCCCAGGGAAGGC
GTTTTTGTCAGTAATGGAACCCACTGGTTCGTCACCCAGCGAAACTTTTA
CGAGCCCCAAATAATAACTACTGACAATACCTTCGTCAGCGGCAATTGCG
ACGTCGTCATAGGCATAGTAAATAATACGGTTTACGACCCTCTTCAACCC
GAGTTGGACAGCTTCAAAGAAGAACTTGATAAGTATTTTAAAAACCACAC
TAGCCCAGATGTAGACCTTGGCGACATCAGCGGTATCAATGCTTCCGTTG
TGAACATTCAAAAGGAAATTGACCGACTCAATGAAGTAGCGAAGAACTTG
AATGAGTCTCTGATCGATCTTCAGGAGCTCGGGAAATATGAGCAATACAT
CAAATGGCCGGTAGGTATTATAGCCGGGCTCGTGTTGTTGGGTGCAGTAA
TCACAGGAGCCGTCGTGGCAGCGGTTATGTGGAGACGGAAATCTTCAGAC
CGCAAAGGTGGTAGTTACACTCAAGCGGCTTCCAGTGATTCTGCCCAGGG
CTCCGACGTATCCCTCACTGCGTGTAAGGTTTCACGACTGGAGGAAGAAC
TGCGCCGACGCCTGACTGAATGA

In certain embodiments the vaccine comprises or encodes Wuhan Spike S1+S2 ECD_HLA. In specific embodiments, the vaccine comprises or encodes the sequence as set forth below (SEQ ID NO:56):

MAVMAPRTLLLLLSGALALTQTWAVNLTTRTQLPPAYTNSFTRGVYYPDK
VFRSSVLHSTQDLFLPFFSNVTWFHAIHVSGTNGTKRFDNPVLPFNDGVY
FASTEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKNLRE
FVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFP
LQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSN
VFQTRAGCLIGAEHVNNSYECDIPIGAGICASYQTQTNSRSVASQSIIAY
TMSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQ
IPFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQIDR
LITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNE
SLIDLQELGKYEQYIKWPVGIIAGLVLLGAVITGAVVAAVMWRRKSSDRK
GGSYTQAASSDSAQGSDVSLTACKVSRLEEELRRRLTE*

In certain embodiments, the vaccine comprises the sequence encoding Kent Spike S1+S2 ECD_HLA as set forth below (SEQ ID NO:57)

GCCACCATGGCGGTCATGGCACCACGCACGCTCCTTCTTTTGTTGTCTGG
TGCCCTTGCACTCACCCAAACGTGGGCAGTTAACCTGACAACACGCACCC
AGTTGCCACCGGCCTATACGAACTCTTTCACGCGCGGAGTGTATTATCCC
GATAAAGTCTTTCGGAGTTCTGTGCTGCACTCTACACAAGATCTTTTTCT
GCCCTTTTTCTCCAACGTTACGTGGTTCCATGCAATAAGTGGTACGAATG
GTACTAAAAGGTTCGATAATCCCGTACTTCCCTTCAATGACGGCGTGTAT
TTCGCGAGCACAGAAAAGAGCAACATAATAAGAGGTTGGATTTTTGGGAC
TACCCTGGACTCTAAAACACAAAGCCTCCTCATTGTTAATAACGCAACTA
ATGTCGTAATAAAGGTATGCGAATTTCAGTTTTGTAACGACCCTTTTTTG
GGAGTCTATCATAAGAACAATAAGTCCTGGATGGAAAGTGAATTTAGGGT
GTATAGTTCAGCGAACAACTGTACATTTGAGTATGTGTCACAGCCGTTCC
TTATGGATCTTGAAGGGAAACAGGGGAACTTCAAGAACCTGCGGGAATTC
GTGTTTAAAAATATAGACGGTTATTTTAAAATCTATAGCAAACACACCCC
AATAAACCTGGTTCGGGATCTTCCTCAGGGATTTTCAGCTTTGGAACCGT
TGGTTGACCTCCCCATCGGGATAAATATCACGCGATTTCAAACACTGCTG
GCACTGCACCGGAGTTATCTGACTCCTGGAGATAGCAGCTCCGGCTGGAC
GGCTGGCGCCGCGGCTTATTATGTCGGGTACCTTCAACCGCGAACGTTTC
TTTTGAAGTATAACGAAAATGGCACTATAACCGATGCAGTAGATTGTGCG
TTGGACCCTCTTTCCGAGACTAAGTGCACCCTGAAGTCATTTACAGTAGA
GAAGGGGATATACCAGACAAGCAACTTCCGCGTCCAACCAACGGAAAGCA
TCGTGCGCTTTCCCAATATTACTAACCTCTGTCCGTTCGGCGAGGTTTTT
AATGCTACCAGATTTGCCTCAGTGTATGCCTGGAACCGCAAGAGGATTAG
CAACTGCGTTGCTGACTATAGCGTGCTTTACAATTCCGCCAGTTTTAGTA
CGTTCAAGTGTTATGGGGTAAGCCCTACCAAGCTTAACGATCTTTGCTTC
ACGAATGTTTACGCAGACTCATTCGTAATACGAGGTGATGAAGTGCGACA
GATAGCCCCGGGTCAGACGGGGAAAATAGCCGACTACAATTACAAGCTCC
CGGACGACTTCACGGGCTGTGTTATTGCGTGGAACAGCAATAACCTGGAC
AGTAAAGTTGGAGGGAATTATAACTACTTGTACAGGCTGTTCCGCAAATC
TAATTTGAAACCGTTTGAAAGGGACATTTCCACCGAGATTTACCAAGCCG
GCAGTACCCCATGTAACGGAGTTGAGGGATTTAATTGCTACTTTCCGCTC
CAATCATATGGATTCCAGCCTACATACGGCGTCGGATATCAACCTTATAG
GGTGGTCGTTCTTTCTTTTGAGCTGCTTCACGCCCCAGCAACCGTTTGCG
GCCCGAAAAAATCAACGAACTTGGTCAAAAACAAGTGCGTCAACTTCAAT
TTCAACGGACTGACAGGCACCGGAGTGCTCACCGAGTCAAATAAAAAATT
CCTCCCGTTTCAACAATTCGGTAGAGATATAGACGACACCACAGATGCGG
TACGCGACCCTCAGACTCTTGAAATTTTGGACATTACACCATGTTCCTTC
GGTGGCGTCTCAGTGATTACGCCTGGCACTAACACTAGCAACCAAGTCGC
AGTACTTTATCAGGGCGTTAACTGTACAGAAGTACCAGTTGCGATACACG
CGGACCAGCTCACCCCAACCTGGCGCGTATATTCTACCGGATCTAATGTG
TTTCAAACGCGGGCGGGGTGCCTCATAGGTGCAGAGCATGTCAATAACTC
CTACGAATGTGACATTCCTATAGGAGCAGGAATATGTGCGAGCTACCAGA
CTCAAACCAATTCACACAGGAGCGTAGCTAGTCAGTCCATAATTGCCTAC
ACTATGTCACTTGGTGCTGAAAACAGTGTGGCTTATTCAAATAATTCCAT
AGCTATTCCAATCAATTTCACTATTAGCGTAACAACTGAGATACTTCCAG
TCAGCATGACTAAGACTTCAGTTGATTGTACGATGTATATTTGTGGCGAT
TCTACAGAATGTAGTAACCTTCTGTTGCAATACGGCTCCTTTTGCACACA
GCTCAATCGCGCATTGACGGGTATAGCTGTAGAGCAAGATAAAAACACCC
AAGAAGTCTTCGCGCAGGTGAAGCAAATATATAAGACACCACCGATTAAA
GATTTCGGTGGATTCAATTTTAGCCAAATTCTGCCTGATCCAAGCAAACC
GTCAAAAAGATCCTTCATAGAGGACCTGCTTTTCAATAAGGTAACGCTGG
CGGACGCAGGGTTTATTAAACAATACGGCGATTGCCTTGGTGACATTGCT
GCACGAGACCTCATTTGCGCCCAGAAGTTCAACGGACTCACAGTACTGCC
TCCACTGCTCACAGACGAGATGATCGCCCAGTACACATCTGCCTTGTTGG
CTGGTACGATAACTTCTGGTTGGACCTTTGGAGCGGGAGCAGCACTTCAG
ATTCCCTTTGCCATGCAGATGGCGTACAGATTCAACGGCATTGGTGTCAC
GCAGAACGTACTGTATGAGAACCAGAAGTTGATAGCTAATCAATTCAACT
CCGCGATAGGTAAGATCCAAGACAGTCTCTCCTCAACAGCCTCCGCACTC
GGGAAACTCCAGGATGTGGTAAATCAGAATGCACAGGCATTGAATACGCT
TGTTAAACAATTGTCCTCAAATTTTGGCGCCATATCATCAGTGCTCAACG
ACATCCTTGCCAGACTCGATAAAGTGGAGGCAGAAGTGCAGATAGATCGC
TTGATTACTGGGCGCCTCCAATCTCTCCAAACGTACGTGACACAACAGCT
TATTCGGGCAGCGGAAATCCGCGCCTCTGCGAACCTTGCAGCGACCAAGA
TGAGCGAATGTGTTCTCGGGCAGTCAAAACGAGTAGATTTCTGCGGTAAA
GGATATCATCTTATGTCATTCCCCCAAAGCGCTCCACATGGTGTTGTATT
TCTGCACGTGACTTACGTGCCGGCACAGGAGAAGAATTTCACAACGGCTC
CCGCCATCTGTCACGACGGAAAGGCACACTTCCCTAGGGAGGGGGTGTTT
GTAAGCAATGGTACCCACTGGTTTGTCACACAACGAAACTTCTATGAACC
TCAGATAATTACAACGCATAATACTTTTGTAAGTGGGAACTGCGACGTCG
TTATCGGAATAGTTAACAATACTGTATATGATCCGTTGCAGCCGGAGTTG
GACTCATTTAAAGAAGAGCTGGACAAGTACTTCAAAAACCATACAAGCCC
AGATGTAGATCTTGGGGACATAAGTGGGATAAACGCCAGCGTTGTGAACA
TACAGAAGGAAATAGACAGGCTGAATGAGGTAGCAAAGAATCTTAACGAA
AGCCTTATAGATCTTCAGGAACTTGGGAAGTACGAACAGTACATAAAGTG
GCCTGTAGGTATTATAGCCGGGCTCGTGTTGTTGGGTGCAGTAATCACAG
GAGCCGTCGTGGCAGCGGTTATGTGGAGACGGAAATCTTCAGACCGCAAA
GGTGGTAGTTACACTCAAGCGGCTTCCAGTGATTCTGCCCAGGGCTCCGA
CGTATCCCTCACTGCGTGTAAGGTTTCACGACTGGAGGAAGAACTGCGCC
GACGCCTGACTGAATGA

In certain embodiments the vaccine comprises or encodes Kent Spike S1+S2 ECD_HLA. In specific embodiments, the vaccine comprises or encodes the sequence as set forth below (SEQ ID NO:58).

MAVMAPRTLLLLLSGALALTQTWAVNLTTRTQLPPAYTNSFTRGVYYPDK
VFRSSVLHSTQDLFLPFFSNVTWFHAISGTNGTKRFDNPVLPFNDGVYFA
STEKSNIIRGWIFGTTLDSKTQSLLIVNNATNVVIKVCEFQFCNDPFLGV
YHKNNKSWMESEFRVYSSANNCTFEYVSQPFLMDLEGKQGNFKNLREFVF
KNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFNA
TRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQS
YGFQPTYGVGYQPYRVVVLSFELLHAPATVCGPKKSTNLVKNKCVNFNFN
GLTGTGVLTESNKKFLPFQQFGRDIDDTTDAVRDPQTLEILDITPCSFGG
VSVITPGTNTSNQVAVLYQGVNCTEVPVAIHADQLTPTWRVYSTGSNVFQ
TRAGCLIGAEHVNNSYECDIPIGAGICASYQTQTNSHRSVASQSIIAYTM
SLGAENSVAYSNNSIAIPINFTISVTTEILPVSMTKTSVDCTMYICGDST
ECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAAR
DLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGK
LQDVVNQNAQALNTLVKQLSSNFGAISSVLNDILARLDKVEAEVQIDRLI
TGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVS
NGTHWFVTQRNFYEPQIITTHNTFVSGNCDVVIGIVNNTVYDPLQPELDS
FKEELDKYFKNHTSPDVDLGDISGINASVVNIQKEIDRLNEVAKNLNESL
IDLQELGKYEQYIKWPVGIIAGLVLLGAVITGAVVAAVMWRRKSSDRKGG
SYTQAASSDSAQGSDVSLTACKVSRLEEELRRRLTE*

In certain embodiments, the vaccine comprises the sequence encoding South Africa Spike S1+S2 ECD_HLA as set forth below (SEQ ID NO:59).

GCCACCATGGCGGTCATGGCACCACGCACGCTCCTTCTTTTGTTGTCTGG
TGCCCTTGCACTCACCCAAACGTGGGCAGTCAATTTCACTACACGCACAC
AGCTGCCCCCGGCGTACACTAACAGTTTTACGAGGGGAGTCTACTACCCG
GACAAAGTGTTCAGGTCTTCCGTACTCCATAGCACACAAGACCTCTTCTT
GCCTTTTTTCAGCAATGTTACGTGGTTCCATGCAATCCATGTGAGCGGTA
CAAACGGGACTAAACGCTTTGCAAATCCAGTGTTGCCCTTCAACGACGGT
GTATACTTCGCGAGTACAGAGAAGAGCAACATAATACGCGGGTGGATATT
TGGAACGACGCTTGATTCTAAGACACAGAGCTTGCTCATCGTAAACAACG
CAACGAATGTTGTAATCAAGGTCTGCGAATTCCAATTCTGCAATGACCCA
TTTCTTGGAGTTTATTACCATAAGAACAATAAGTCTTGGATGGAATCCGA
GTTCCGCGTATACTCTAGCGCGAACAACTGCACCTTCGAGTATGTGTCCC
AACCGTTTCTGATGGATCTTGAGGGAAAGCAGGGTAATTTTAAAAATCTC
CGGGAGTTCGTTTTCAAAAATATTGACGGATATTTCAAAATCTACAGCAA
GCATACCCCAATCAATTTGGTGCGGGGTCTGCCACAAGGATTTTCCGCTC
TTGAACCTCTTGTTGACCTTCCCATAGGAATCAATATAACTCGGTTCCAA
ACTCTTCTTGCGCTCCATATATCCTACTTGACGCCCGGAGATTCATCAAG
TGGCTGGACGGCTGGGGCTGCCGCATACTACGTCGGCTATCTGCAACCAA
GGACGTTTCTCCTCAAATACAATGAAAATGGTACGATCACCGACGCTGTG
GACTGCGCTCTCGACCCGTTGTCAGAGACGAAGTGCACCTTGAAGTCCTT
CACCGTGGAAAAGGGAATTTACCAGACTAGTAACTTCCGGGTGCAGCCCA
CGGAATCAATTGTGCGATTTCCTAATATCACTAATCTCTGTCCCTTCGGG
GAAGTGTTCAATGCGACTCGATTCGCTTCAGTATACGCTTGGAATAGAAA
GAGAATCTCAAATTGCGTGGCTGACTATTCCGTACTGTATAATAGTGCGT
CTTTTAGCACGTTTAAGTGTTATGGCGTATCCCCGACAAAGCTCAACGAT
CTCTGCTTCACGAATGTCTATGCCGACTCCTTCGTGATACGCGGTGACGA
GGTACGACAAATTGCACCCGGCCAAACTGGAAATATTGCCGACTACAATT
ATAAGTTGCCGGACGATTTTACCGGTTGCGTAATAGCGTGGAACTCTAAT
AACCTCGACTCTAAGGTAGGAGGCAACTACAATTACTTGTACCGACTGTT
CAGGAAATCCAACCTGAAGCCCTTCGAAAGAGACATCAGTACAGAGATTT
ATCAAGCCGGGTCCACCCCTTGCAATGGTGTCAAAGGCTTCAACTGCTAT
TTTCCGTTGCAGTCTTACGGTTTCCAGCCGACGTACGGTGTGGGTTATCA
ACCCTACCGCGTTGTGGTGCTGAGCTTTGAGCTTCTCCACGCTCCAGCCA
CTGTCTGCGGGCCTAAGAAATCCACAAACCTCGTTAAGAATAAATGCGTA
AATTTCAACTTCAACGGGCTTACTGGCACGGGCGTGCTTACGGAGTCCAA
TAAGAAATTCCTGCCTTTCCAGCAGTTCGGCCGCGACATCGCGGACACAA
CAGATGCTGTGCGAGACCCACAAACCCTGGAGATTCTGGACATCACGCCT
TGCTCTTTTGGCGGTGTCAGCGTAATAACGCCAGGCACCAACACCAGTAA
TCAGGTCGCCGTGCTTTATCAGGGTGTGAACTGTACGGAGGTACCGGTTG
CCATACACGCAGACCAACTCACACCTACGTGGAGAGTTTACAGCACCGGG
TCAAACGTATTTCAGACCCGCGCAGGCTGTCTGATCGGCGCAGAGCATGT
CAATAACTCATACGAGTGTGATATACCAATCGGAGCCGGCATCTGTGCGA
GCTATCAAACCCAAACTAATTCACGAAGCGTGGCCAGCCAGTCCATCATT
GCCTACACGATGTCTTTGGGAGTAGAGAATAGCGTGGCATACTCTAACAA
CAGTATCGCTATTCCTACCAACTTCACAATATCAGTTACTACAGAAATCC
TCCCGGTGAGCATGACTAAGACATCCGTAGACTGTACAATGTACATTTGT
GGGGACAGCACGGAGTGTTCAAACCTGCTGTTGCAATACGGAAGCTTCTG
CACCCAGCTCAACCGAGCCCTGACCGGCATTGCCGTAGAACAAGATAAGA
ATACGCAGGAAGTGTTCGCCCAGGTTAAGCAGATTTACAAGACTCCGCCT
ATCAAGGACTTCGGGGGTTTCAACTTTAGTCAGATTTTGCCTGACCCGTC
AAAGCCTAGCAAACGAAGTTTTATCGAAGATTTGTTGTTTAACAAGGTGA
CTTTGGCTGATGCGGGATTCATAAAACAATACGGCGATTGCCTTGGCGAT
ATTGCGGCCCGAGATCTCATTTGCGCGCAAAAGTTCAACGGTCTTACCGT
CCTTCCGCCCCTGCTTACGGACGAGATGATCGCACAATACACTTCAGCAC
TGCTGGCTGGTACAATAACCAGTGGGTGGACATTCGGGGCTGGTGCAGCG
CTCCAGATCCCCTTTGCGATGCAGATGGCGTATAGATTCAATGGCATCGG
GGTCACTCAGAACGTGCTTTATGAGAACCAAAAGCTTATTGCAAATCAAT
TTAACTCTGCGATTGGAAAGATTCAAGACAGCCTCTCATCCACAGCTAGT
GCGCTTGGCAAGCTTCAGGACGTCGTAAACCAAAACGCCCAAGCTCTCAA
CACACTCGTTAAACAGCTTTCCTCAAACTTTGGGGCTATTAGCTCTGTGC
TGAATGATATTCTTAGCAGACTCGACAAAGTCGAGGCTGAAGTACAAATA
GATAGACTGATAACAGGTAGACTTCAGTCTCTCCAAACCTACGTGACACA
GCAGCTTATAAGAGCTGCTGAGATCAGAGCTTCTGCGAACCTTGCGGCGA
CTAAAATGTCCGAGTGTGTACTCGGGCAGTCTAAACGCGTCGACTTCTGT
GGCAAGGGTTACCATCTCATGAGTTTTCCCCAATCAGCGCCCCATGGGGT
AGTTTTTCTGCACGTAACATACGTCCCAGCCCAAGAAAAGAATTTTACCA
CCGCCCCTGCGATTTGTCACGACGGAAAAGCACACTTTCCAAGAGAAGGC
GTATTTGTTAGCAATGGCACCCATTGGTTCGTCACCCAGAGAAACTTTTA
CGAACCGCAGATTATCACGACAGATAATACATTTGTATCTGGTAATTGTG
ACGTCGTGATCGGCATCGTAAACAACACTGTTTACGACCCCCTCCAGCCA
GAGCTTGACTCATTTAAAGAGGAGCTCGACAAATATTTTAAGAACCACAC
AAGTCCAGACGTCGACTTGGGTGACATTTCCGGCATCAATGCAAGTGTGG
TCAACATCCAAAAGGAAATTGACAGATTGAACGAGGTTGCTAAGAACCTT
AATGAGTCACTCATTGACCTCCAGGAACTGGGAAAATACGAACAGTATAT
TAAGTGGCCCGTAGGTATTATAGCCGGGCTCGTGTTGTTGGGTGCAGTAA
TCACAGGAGCCGTCGTGGCAGCGGTTATGTGGAGACGGAAATCTTCAGAC
CGCAAAGGTGGTAGTTACACTCAAGCGGCTTCCAGTGATTCTGCCCAGGG
CTCCGACGTATCCCTCACTGCGTGTAAGGTTTCACGACTGGAGGAAGAAC
TGCGCCGACGCCTGACTGAATGA

In certain embodiments the vaccine comprises or encodes South Africa Spike S1+S2 ECD_HLA. In specific embodiments, the vaccine comprises or encodes the sequence as set forth below (SEQ ID NO:60).

MAVMAPRTLLLLLSGALALTQTWAVNFTTRTQLPPAYTNSFTRGVYYPDK
VFRSSVLHSTQDLFLPFFSNVTWFHAIHVSGTNGTKRFANPVLPFNDGVY
FASTEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKNLRE
FVFKNIDGYFKIYSKHTPINLVRGLPQGFSALEPLVDLPIGINITRFQTL
LALHISYLTPGDSSSGWTAGAAAYYVGYLQPRTFLLKYNENGTITDAVDC
ALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLC
FTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFP
LQSYGFQPTYGVGYQPYRVVVLSFELLHAPATVCGPKKSTNLVKNKCVNF
NFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSN
VFQTRAGCLIGAEHVNNSYECDIPIGAGICASYQTQTNSRSVASQSIIAY
TMSLGVENSVAYSNNSIAIPTNFTISVTTEILPVSMTKTSVDCTMYICGD
STECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQ
IPFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQIDR
LITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNE
SLIDLQELGKYEQYIKWPVGIIAGLVLLGAVITGAVVAAVMWRRKSSDRK
GGSYTQAASSDSAQGSDVSLTACKVSRLEEELRRRLTE*

In certain embodiments, the vaccine comprises the sequence encoding California Spike S1+S2 ECD_HLA as set forth below (SEQ ID NO:61).

GCCACCATGGCGGTCATGGCACCACGCACGCTCCTTCTTTTGTTGTCTGG
TGCCCTTGCACTCACCCAAACGTGGGCAGTAAATTTGACAACGCGCACCC
AATTGCCCCCAGCATATACGAACTCTTTCACGAGGGGCGTATATTATCCG
GATAAGGTATTTCGGTCATCTGTTCTGCACAGCACCCAGGACCTCTTCCT
TCCATTCTTTTCAAATGTAACTTGGTTCCATGCGATAGTATCAGGAACGA
ATGGGACAAAAAGGTTCGATAATCCGGTCTTGCCGTTCAACGATGGGGTG
TACTTCGCCAGTACCGAAAAGTCTAACATTATACGCGGTTGGATTTTTGG
CACTACGCTTGACTCAAAGACACAGTCACTCCTCATTGTAAATAATGCTA
CTAATGTCGTGATCAAAGTTTGTGAGTTCCAATTCTGCAACGACCCGTTC
CTGGGAGTGTATTATCACAAAAATAATAAGTCCTGCATGGAGTCAGAGTT
CAGGGTTTACTCAAGTGCGAACAATTGCACATTTGAGTACGTTTCTCAAC
CATTTCTCATGGATTTGGAGGGGAAGCAGGGAAACTTTAAAAACCTGAGA
GAATTTGTTTTTAAGAACATTGATGGGTATTTCAAGATTTATAGTAAACA
CACCCCTATCAACTTGGTTAGGGATCTCCCTCAAGGTTTCTCTGCTCTCG
AGCCCCTTGTAGATCTGCCAATAGGCATCAATATCACACGCTTTCAGACA
CTCCTCGCACTTCATAGGAGCTACCTGACGCCAGGTGACTCTTCCTCAGG
TTGGACAGCCGGCGCAGCCGCATACTACGTTGGCTACCTCCAACCAAGGA
CATTTCTGTTGAAATACAACGAAAATGGGACCATCACCGACGCAGTCGAT
TGTGCTCTCGACCCTCTTTCCGAGACTAAATGTACCCTCAAAAGCTTTAC
TGTTGAGAAGGGTATCTATCAGACATCTAACTTTCGGGTGCAACCCACTG
AGTCAATTGTGCGATTCCCAAATATTACGAACCTCTGTCCTTTTGGCGAG
GTTTTTAACGCCACTAGGTTCGCCAGTGTATATGCTTGGAACCGAAAACG
GATAAGCAATTGTGTTGCTGACTACTCCGTCCTCTACAATAGCGCTAGTT
TCTCAACATTTAAGTGTTACGGTGTGAGCCCTACGAAACTTAACGATTTG
TGCTTCACTAACGTCTATGCCGACAGTTTCGTAATCCGAGGCGATGAGGT
CAGGCAAATTGCCCCGGGCCAAACGGGGAAAATCGCTGATTACAATTATA
AGTTGCCAGATGATTTTACGGGATGTGTCATTGCATGGAACAGTAATAAC
CTCGATTCAAAGGTTGGCGGAAATTATAATTACAGATATCGGCTTTTTAG
AAAATCTAACCTTAAACCATTTGAGCGGGACATAAGCACGGAGATTTACC
AGGCTGGTAGCACTCCGTGCAACGGTGTAGAAGGATTCAATTGCTATTTT
CCATTGCAGTCTTATGGATTCCAACCCACCAATGGGGTAGGGTACCAACC
ATACAGGGTGGTAGTCCTTAGCTTTGAACTTTTGCATGCGCCAGCTACCG
TCTGCGGTCCCAAAAAGAGTACGAACTTGGTAAAAAATAAATGCGTCAAC
TTTAATTTTAACGGTCTGACGGGAACGGGGGTTCTCACCGAGTCTAACAA
AAAGTTTTTGCCATTTCAGCAGTTCGGACGAGATATTGCCGACACTACCG
ACGCCGTGCGCGATCCACAAACCTTGGAGATACTCGACATCACACCTTGC
AGCTTCGGTGGTGTTAGCGTTATTACGCCAGGAACGAACACTTCAAATCA
GGTGGCCGTCTTGTATCAAGATGTTAACTGTACAGAGGTGCCCGTCGCAA
TACACGCAGATCAGCTCACCCCTACCTGGAGAGTTTACTCTACAGGCTCT
AATGTGTTCCAAACTAGGGCAGGCTGCTTGATTGGAGCTGAACACGTAAA
CAACTCATACGAGTGCGATATCCCGATTGGTGCGGGTATTTGCGCCAGCT
ATCAGACGCAGACTAACTCACGATCAGTGGCGTCACAGTCAATAATTGCG
TATACAATGAGTCTCGGTGCAGAGAATTCCGTCGCATACTCAAACAACAG
CATAGCGATTCCAACTAATTTTACCATTAGCGTTACAACGGAAATCCTCC
CCGTTAGCATGACCAAGACCTCCGTGGATTGCACAATGTACATCTGTGGA
GACAGCACCGAGTGCTCCAATCTTCTTCTTCAATATGGGAGCTTTTGCAC
ACAGCTGAACCGCGCGCTCACTGGCATTGCGGTAGAACAAGACAAAAACA
CCCAAGAAGTGTTTGCGCAGGTCAAACAGATTTACAAGACACCACCAATC
AAAGACTTCGGGGGGTTCAATTTCTCCCAAATTCTTCCTGATCCAAGCAA
ACCATCTAAAAGGTCCTTCATAGAAGACCTCCTGTTCAATAAGGTGACAC
TCGCGGACGCAGGGTTTATTAAGCAATATGGAGATTGTCTCGGGGATATT
GCAGCGAGAGATCTTATTTGTGCTCAAAAGTTTAATGGCTTGACTGTCCT
CCCGCCCTTGCTCACAGACGAGATGATAGCACAATATACCAGTGCGCTGC
TTGCGGGCACGATTACTTCCGGATGGACTTTCGGGGCAGGAGCGGCGCTC
CAGATTCCTTTCGCGATGCAAATGGCCTACCGATTTAACGGGATTGGCGT
CACACAAAACGTTCTTTACGAAAACCAGAAACTTATTGCCAACCAATTCA
ATAGTGCAATTGGTAAAATTCAGGATTCACTCTCCTCCACGGCGTCAGCT
CTCGGCAAGCTCCAAGACGTGGTTAACCAAAATGCGCAGGCGCTTAACAC
GCTTGTAAAACAACTCTCTTCTAATTTTGGCGCGATTAGTAGCGTTCTCA
ACGATATCTTGTCACGCTTGGACAAAGTAGAAGCAGAAGTCCAAATAGAC
CGGCTGATCACTGGTCGATTGCAATCTCTGCAGACATACGTGACGCAGCA
GCTCATCCGGGCAGCCGAGATCCGGGCGTCCGCTAATCTGGCCGCAACCA
AAATGAGCGAGTGTGTCCTTGGGCAAAGCAAACGCGTGGATTTTTGCGGA
AAGGGCTACCACCTCATGAGTTTCCCCCAATCTGCCCCGCACGGTGTCGT
CTTTTTGCATGTAACGTATGTCCCGGCGCAGGAGAAAAATTTCACTACAG
CTCCCGCCATCTGTCACGACGGCAAGGCACACTTCCCTCGAGAAGGGGTG
TTTGTAAGCAATGGAACACACTGGTTCGTCACCCAGCGGAATTTTTATGA
ACCTCAGATAATCACAACAGACAACACTTTCGTTTCCGGAAACTGCGATG
TCGTAATTGGGATAGTGAATAATACCGTATATGACCCATTGCAGCCGGAG
TTGGACTCCTTTAAAGAGGAATTGGACAAATACTTTAAAAATCACACTTC
ACCCGATGTGGACCTGGGAGATATATCCGGGATTAACGCAAGTGTGGTTA
ACATACAAAAGGAAATTGACCGCCTTAACGAGGTTGCTAAGAACTTGAAT
GAGTCTCTCATAGATCTCCAGGAACTGGGGAAATACGAGCAATATATCAA
ATGGCCGGTAGGTATTATAGCCGGGCTCGTGTTGTTGGGTGCAGTAATCA
CAGGAGCCGTCGTGGCAGCGGTTATGTGGAGACGGAAATCTTCAGACCGC
AAAGGTGGTAGTTACACTCAAGCGGCTTCCAGTGATTCTGCCCAGGGCTC
CGACGTATCCCTCACTGCGTGTAAGGTTTCACGACTGGAGGAAGAACTGC
GCCGACGCCTGACTGAATGA

In certain embodiments the vaccine comprises or encodes California Spike S1+S2 ECD_HLA. In specific embodiments, the vaccine comprises or encodes the sequence as set forth below (SEQ ID NO:62).

MAVMAPRTLLLLLSGALALTQTWAVNLTTRTQLPPAYTNSFTRGVYYPDK
VFRSSVLHSTQDLFLPFFSNVTWFHAIVSGTNGTKRFDNPVLPFNDGVYF
ASTEKSNIIRGWIFGTTLDSKTQSLLIVNNATNVVIKVCEFQFCNDPFLG
VYYHKNNKSCMESEFRVYSSANNCTFEYVSQPFLMDLEGKQGNFKNLREF
VFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFGEVF
NATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLD
SKVGGNYNYRYRLFRKSNLKPFERDISTEIYQAGSTPCNGVEGFNCYFPL
QSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSF
GGVSVITPGTNTSNQVAVLYQDVNCTEVPVAIHADQLTPTWRVYSTGSNV
FQTRAGCLIGAEHVNNSYECDIPIGAGICASYQTQTNSRSVASQSIIAYT
MSLGAENSVAYSNNSIAIPTNFTISVTTEILPVSMTKTSVDCTMYICGDS
TECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALG
KLQDVVNQNAQALNTLVKQLSSNFGAISSVLNDILSRLDKVEAEVQIDRL
ITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFV
SNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNES
LIDLQELGKYEQYIKWPVGIIAGLVLLGAVITGAVVAAVMWRRKSSDRKG
GSYTQAASSDSAQGSDVSLTACKVSRLEEELRRRLTE*

In certain embodiments, the vaccine comprises the sequence encoding Brazil Spike S1+S2 ECD_HLA as set forth below (SEQ ID NO:63).

GCCACCATGGCGGTCATGGCACCACGCACGCTCCTTCTTTTGTTGTCTGG
TGCCCTTGCACTCACCCAAACGTGGGCAGTTAACTTTACTAATCGGACCC
AACTCCCGAGTGCGTACACTAACTCTTTTACCCGGGGCGTCTATTATCCT
GACAAAGTGTTTCGATCATCTGTTTTGCACTCCACCCAAGATCTCTTTCT
TCCATTTTTCAGCAACGTTACGTGGTTCCACGCGATACATGTTAGTGGTA
CTAATGGCACAAAGAGATTTGACAACCCTGTGCTCCCGTTTAACGATGGG
GTCTATTTCGCTTCCACTGAGAAGTCCAACATTATTCGCGGTTGGATCTT
CGGCACCACACTCGATTCAAAAACCCAAAGCTTGCTCATAGTTAACAACG
CCACAAACGTAGTCATTAAAGTGTGCGAATTTCAGTTTTGTAACTATCCC
TTTTTGGGCGTCTACTATCACAAGAACAACAAGTCCTGGATGGAATCTGA
GTTTCGGGTCTACAGTTCAGCCAACAACTGTACGTTTGAGTACGTTAGCC
AGCCCTTTCTGATGGATCTTGAAGGGAAGCAAGGAAATTTCAAGAATCTT
TCCGAGTTTGTGTTTAAAAATATAGACGGATATTTCAAGATATACAGTAA
GCACACACCGATCAATTTGGTACGGGATTTGCCGCAAGGATTTTCCGCCC
TTGAGCCCCTTGTAGATCTCCCCATCGGAATTAACATAACCAGGTTTCAA
ACACTGCTGGCGCTCCACAGGAGCTATCTGACCCCGGGCGACTCATCCAG
TGGGTGGACCGCCGGAGCAGCCGCATACTACGTGGGGTATCTCCAGCCCA
GAACGTTTCTCCTCAAGTATAATGAAAACGGGACCATCACCGACGCGGTT
GATTGTGCACTCGATCCCCTCAGCGAGACAAAGTGCACACTTAAATCATT
TACCGTCGAGAAGGGGATCTACCAAACATCCAACTTTCGGGTGCAGCCTA
CCGAGTCAATAGTACGGTTTCCTAACATCACCAATTTGTGCCCTTTTGGG
GAAGTATTTAATGCAACCCGGTTTGCGAGCGTATACGCTTGGAACCGAAA
ACGGATAAGTAATTGTGTTGCAGATTATTCTGTGCTCTATAACTCAGCTT
CCTTCTCCACCTTTAAGTGTTACGGAGTATCTCCGACAAAATTGAATGAT
CTTTGTTTTACGAATGTTTACGCTGACTCATTTGTTATCCGAGGTGACGA
AGTGCGCCAGATAGCCCCCGGTCAAACAGGCAACATTGCTGATTATAACT
ACAAGTTGCCCGATGACTTCACGGGGTGTGTTATTGCATGGAATTCAAAT
AATCTTGATAGTAAGGTAGGGGGTAACTATAATTACCTTTATCGCTTGTT
CCGCAAATCCAATCTGAAACCCTTCGAAAGGGATATCAGTACCGAGATTT
ATCAAGCCGGCTCCACACCTTGTAACGGAGTAAAAGGTTTTAACTGTTAT
TTCCCACTGCAGAGTTACGGCTTTCAACCCACATACGGGGTGGGATATCA
GCCCTATCGCGTTGTGGTCTTGTCCTTTGAACTTCTCCACGCCCCTGCAA
CAGTTTGTGGTCCTAAAAAGAGTACAAATCTGGTAAAAAATAAGTGCGTC
AACTTTAACTTCAACGGACTGACTGGTACCGGGGTCCTCACTGAGAGCAA
CAAGAAATTTTTGCCCTTCCAGCAATTCGGGAGGGATATTGCCGATACTA
CCGATGCTGTACGGGACCCCCAGACACTCGAAATATTGGACATAACCCCG
TGCTCTTTTGGAGGGGTCTCTGTGATTACCCCGGGTACGAACACGAGTAA
TCAGGTGGCAGTGCTTTATCAGGGAGTGAATTGCACAGAAGTACCAGTCG
CGATCCATGCCGACCAGCTCACGCCGACCTGGAGGGTTTATAGTACGGGT
AGTAACGTTTTCCAAACACGGGCCGGTTGTCTCATCGGGGCAGAGTACGT
TAATAACAGCTATGAATGTGATATACCCATAGGTGCCGGGATTTGTGCTT
CTTATCAAACCCAAACCAATAGCAGAAGCGTTGCGTCACAGTCTATAATC
GCCTATACGATGAGCCTCGGAGCGGAAAACTCAGTTGCCTACTCCAACAA
TAGCATTGCTATCCCAACGAACTTCACTATTAGCGTAACCACGGAGATCC
TTCCAGTTAGCATGACAAAAACTAGCGTCGATTGCACTATGTATATTTGC
GGCGACAGTACAGAGTGTAGTAATTTGCTCCTGCAGTACGGGTCTTTCTG
CACCCAATTGAACCGAGCACTGACTGGCATCGCCGTGGAACAAGACAAAA
ACACGCAAGAAGTATTTGCGCAAGTCAAGCAGATTTACAAGACTCCCCCG
ATCAAAGACTTTGGCGGATTTAATTTTAGCCAGATACTCCCCGATCCATC
TAAACCATCAAAACGGTCCTTTATAGAAGATCTCTTGTTCAATAAAGTGA
CACTCGCGGACGCGGGCTTTATCAAACAGTATGGGGACTGCCTTGGAGAT
ATAGCTGCGAGGGACTTGATTTGCGCCCAGAAATTCAACGGGCTGACGGT
TCTGCCGCCGCTCTTGACAGACGAAATGATTGCACAGTATACGAGTGCCT
TGCTGGCAGGGACTATCACTTCCGGTTGGACCTTCGGCGCAGGAGCAGCG
CTCCAGATCCCATTCGCTATGCAAATGGCTTACCGATTCAACGGAATTGG
CGTCACACAGAACGTGCTTTACGAGAATCAAAAGCTGATTGCCAATCAAT
TCAACTCAGCGATTGGCAAAATACAAGATAGTCTCTCTTCAACTGCATCC
GCGCTCGGTAAGTTGCAGGACGTAGTCAATCAGAACGCACAGGCCCTGAA
CACGCTTGTTAAACAACTGTCTTCAAACTTCGGTGCAATAAGTTCCGTAC
TTAATGATATCTTGTCAAGGTTGGATAAGGTCGAAGCTGAGGTACAAATC
GACCGCCTTATAACAGGCAGACTCCAATCCCTGCAGACATACGTGACCCA
GCAACTCATTAGGGCAGCCGAGATCAGAGCAAGTGCCAACCTCGCTGCTA
TTAAAATGTCTGAATGCGTTTTGGGGCAATCTAAGCGGGTTGATTTCTGC
GGTAAGGGATACCACTTGATGTCCTTTCCTCAGTCCGCTCCTCATGGGGT
AGTATTTCTTCATGTTACATATGTACCAGCACAAGAGAAAAATTTTACTA
CTGCCCCGGCCATATGCCATGATGGCAAAGCGCACTTCCCTCGGGAGGGG
GTTTTTGTGTCTAATGGAACCCACTGGTTCGTGACTCAGCGGAATTTCTA
TGAACCCCAGATTATAACTACTGACAACACTTTCGTTTCTGGAAACTGCG
ATGTAGTAATAGGCATAGTAAATAATACAGTATATGATCCCTTGCAACCA
GAATTGGACTCTTTTAAAGAAGAGCTCGACAAATATTTTAAAAACCATAC
ATCTCCAGACGTTGATCTTGGAGACATCTCTGGGATAAACGCTAGCTTCG
TAAACATACAAAAAGAAATCGATAGGCTTAACGAGGTCGCAAAAAATCTT
AATGAAAGTCTTATCGATTTGCAAGAGCTTGGAAAGTATGAGCAGTATAT
CAAATGGCCTGTAGGTATTATAGCCGGGCTCGTGTTGTTGGGTGCAGTAA
TCACAGGAGCCGTCGTGGCAGCGGTTATGTGGAGACGGAAATCTTCAGAC
CGCAAAGGTGGTAGTTACACTCAAGCGGCTTCCAGTGATTCTGCCCAGGG
CTCCGACGTATCCCTCACTGCGTGTAAGGTTTCACGACTGGAGGAAGAAC
TGCGCCGACGCCTGACTGAATGA

In certain embodiments the vaccine comprises or encodes Brazil Spike S1+S2 ECD_HLA. In specific embodiments, the vaccine comprises or encodes the sequence as set forth below (SEQ ID NO:64).

MAVMAPRTLLLLLSGALALTQTWAVNFTNRTQLPSAYTNSFTRGVYYPDK
VFRSSVLHSTQDLFLPFFSNVTWFHAIHVSGTNGTKRFDNPVLPFNDGVY
FASTEKSNIIRGWIFGTTLDSKTQSLLIVNNATNVVIKVCEFQFCNYPFL
GVYYHKNNKSWMESEFRVYSSANNCTFEYVSQPFLMDLEGKQGNFKNLSE
FVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLC
FTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFP
LQSYGFQPTYGVGYQPYRVVVLSFELLHAPATVCGPKKSTNLVKNKCVNF
NFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSN
VFQTRAGCLIGAEYVNNSYECDIPIGAGICASYQTQTNSRSVASQSIIAY
TMSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQ
IPFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQIDR
LITGRLQSLQTYVTQQLIRAAEIRASANLAAIKMSECVLGQSKRVDFCGK
GYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPEL
DSFKEELDKYFKNHTSPDVDLGDISGINASFVNIQKEIDRLNEVAKNLNE
SLIDLQELGKYEQYIKWPVGIIAGLVLLGAVITGAVVAAVMWRRKSSDRK
GGSYTQAASSDSAQGSDVSLTACKVSRLEEELRRRLTE*

In certain embodiments, the vaccine comprises the sequence encoding Delta Spike S1+S2 ECD_HLA as set forth below (SEQ ID NO:65).

GCCACCATGGCGGTCATGGCACCACGCACGCTCCTTCTTTTGTTGTCTGG
TGCCCTTGCACTCACCCAAACGTGGGCAGTTAATCTTACAACCAGAACTC
AATTACCCCCTGCATACACTAATTCTTTCACACGTGGTGTTTATTACCCT
GACAAAGTTTTCAGATCCTCAGTTTTACATTCAACTCAGGACTTGTTCTT
ACCTTTCTTTTCCAATGTTACTTGGTTCCATGCTATACATGTCTCTGGGA
CCAATGGTACTAAGAGGTTTGATAACCCTGTCCTACCATTTAATGATGGT
GTTTATTTTGCTTCCATTGAGAAGTCTAACATAATAAGAGGCTGGATTTT
TGGTACTACTTTAGATTCGAAGACCCAGTCCCTACTTATTGTTAATAACG
CTACTAATGTTGTTATTAAAGTCTGTGAATTTCAATTTTGTAATGATCCA
TTTTTGGGTGTTTATTACCACAAAAACAACAAAAGTTGGATGAAAAGTGA
GTTCAGAGTTTATTCTAGTGCGAATAATTGCACTTTTGAATATGTCTCTC
AGCCTTTTCTTATGGACCTTGAAGGAAAACAGGGTAATTTCAAAAATCTT
AGGGAATTTGTGTTTAAGAATATTGATGGTTATTTTAAAATATATTCTAA
GCACACGCCTATTAATTTAGTGCGTGATCTCCCTCAGGGTTTTTCGGCTT
TAGAACCATTGGTAGATTTGCCAATAGGTATTAACATCACTAGGTTTCAA
ACTTTACTTGCTTTACATAGAAGTTATTTGACTCCTGGTGATTCTTCTTC
AGGTTGGACAGCTGGTGCTGCAGCTTATTATGTGGGTTATCTTCAACCTA
GGACTTTTCTATTAAAATATAATGAAAATGGAACCATTACAGATGCTGTA
GACTGTGCACTTGACCCTCTCTCAGAAACAAAGTGTACGTTGAAATCCTT
CACTGTAGAAAAAGGAATCTATCAAACTTCTAACTTTAGAGTCCAACCAA
CAGAATCTATTGTTAGATTTCCTAATATTACAAACTTGTGCCCTTTTGGT
GAAGTTTTTAACGCCACCAGATTTGCATCTGTTTATGCTTGGAACAGGAA
GAGAATCAGCAACTGTGTTGCTGATTATTCTGTCCTATATAATTCCGCAT
CATTTTCCACTTTTAAGTGTTATGGAGTGTCTCCTACTAAATTAAATGAT
CTCTGCTTTACTAATGTCTATGCAGATTCATTTGTAATTAGAGGTGATGA
AGTCAGACAAATCGCTCCAGGGCAAACTGGAAAGATTGCTGATTATAATT
ATAAATTACCAGATGATTTTACAGGCTGCGTTATAGCTTGGAATTCTAAC
AAACTTGATTCTAAGGTTGGTGGTAATTATAATTACCGCTATAGATTGTT
TAGGAAGTCTAATCTCAAACCTTTTGAGAGAGATATTTCAACTGAAATCT
ATCAGGCCGGTAGCACACCTTGTAATGGTGTTCAGGGTTTTAATTGTTAC
TTTCCTTTACAATCATATGGTTTCCAACCCACTTATGGTGTTGGTTACCA
ACCATACAGAGTAGTAGTACTTTCTTTTGAACTTCTACATGCACCAGCAA
CTGTTTGTGGACCTAAAAAGTCTACTAATTTGGTTAAAAACAAATGTGTC
AATTTCAACTTCAATGGTTTAACAGGCACAGGTGTTCTTACTGAGTCTAA
CAAAAAGTTTCTGCCTTTCCAACAATTTGGCAGAGACATTGATGACACTA
CTGATGCTGTCCGTGATCCACAGACACTTGAGATTCTTGACATTACACCA
TGTTCTTTTGGTGGTGTCAGTGTTATAACACCAGGAACAAATACTTCTAA
CCAGGTTGCTGTTCTTTATCAGGGTGTTAACTGCACAGAAGTCCCTGTTG
CTATTCATGCAGATCAACTTACTCCTACTTGGCGTGTTTATTCTACAGGT
TCTAATGTTTTTCAAACACGTGCAGGCTGTTTAATAGGGGCTGAACATGT
CAACAACTCATATGAGTGTGACATACCCATTGGTGCAGGTATATGCGCTA
GTTATCAGACTCAGACTAATTCTAGTGTAGCTAGTCAATCCATCATTGCC
TACACTATGTCACTTGGTGCAGAAAATTCAGTTGCTTACTCTAATAACTC
TATTGCCATACCCATTAATTTTACTATTAGTGTTACCACAGAAATTCTAC
CAGTGTCTATGACCAAGACATCAGTAGATTGTACAATGTACATTTGTGGT
GATTCAACTGAATGCAGCAATCTTTTGTTGCAATATGGCAGTTTTTGTAC
ACAATTAAACCGTGCTTTAACTGGAATAGCTGTTGAACAAGACAAAAACA
CCCAAGAAGTTTTTGCACAAGTCAAACAAATTTACAAAACACCACCAATT
AAAGATTTTGGTGGTTTTAATTTTTCACAAATATTACCAGATCCATCAAA
ACCAAGCAAGAGGTCATTTATTGAAGATCTACTTTTCAACAAAGTGACAC
TTGCAGATGCTGGCTTCATCAAACAATATGGTGATTGCCTTGGTGATATT
GCTGCTAGAGACCTCATTTGTGCACAAAAGTTTAACGGCCTTACTGTTTT
GCCACCTTTGCTCACAGATGAAATGATTGCTCAATACACTTCTGCACTGT
TAGCGGGTACAATCACTTCTGGTTGGACCTTTGGTGCAGGTGCTGCATTA
CAAATACCATTTGCTATGCAAATGGCTTATAGGTTTAATGGTATTGGAGT
TACACAGAATGTTCTCTATGAGAACCAAAAATTGATTGCCAACCAATTTA
ATAGTGCTATTGGCAAAATTCAAGACTCACTTTCTTCCACAGCAAGTGCA
CTTGGAAAACTTCAAGATGTGGTCAACCAAAATGCACAAGCTTTAAACAC
GCTTGTTAAACAACTTAGCTCCAATTTTGGTGCAATTTCAAGTGTTTTAA
ATGATATCCTTGCACGTCTTGACAAAGTTGAGGCTGAAGTGCAAATTGAT
AGGTTGATCACAGGCAGACTTCAAAGTTTGCAGACATATGTGACTCAACA
ATTAATTAGAGCTGCAGAAATCAGAGCTTCTGCTAATCTTGCTGCTACTA
AAATGTCAGAGTGTGTACTTGGACAATCAAAAAGAGTTGATTTTTGTGGA
AAGGGCTATCATCTTATGTCCTTCCCTCAGTCAGCACCTCATGGTGTAGT
CTTCTTGCATGTGACTTATGTCCCTGCACACGAAAAGAACTTCACAACTG
CTCCTGCCATTTGTCATGATGGAAAAGCACACTTTCCTCGTGAAGGTGTC
TTTGTTTCAAATGGCACACACTGGTTTGTAACACAAAGGAATTTTTATGA
ACCACAAATCATTACTACACACAACACATTTGTGTCTGGTAACTGTGATG
TTGTAATAGGAATTGTCAACAACACAGTTTATGATCCTTTGCAACCTGAA
TTAGACTCATTCAAGGAGGAGTTAGATAAATATTTTAAGAATCATACATC
ACCAGATGTTGATTTAGGTGACATCTCTGGCATTAATGCTTCAGTTGTAA
ACATTCAAAAAGAAATTGACCGCCTCAATGAGGTTGCCAAGAATTTAAAT
GAATCTCTCATCGATCTCCAAGAACTTGGAAAGTATGAGCAGTATATAAA
ATGGCCAGTAGGTATTATAGCCGGGCTCGTGTTGTTGGGTGCAGTAATCA
CAGGAGCCGTCGTGGCAGCGGTTATGTGGAGACGGAAATCTTCAGACCGC
AAAGGTGGTAGTTACACTCAAGCGGCTTCCAGTGATTCTGCCCAGGGCTC
CGACGTATCCCTCACTGCGTGTAAGGTTTCACGACTGGAGGAAGAACTGC
GCCGACGCCTGACTGAATAA

In certain embodiments the vaccine comprises or encodes Delta Spike S1+S2 ECD_HLA. In certain embodiments the vaccine comprises or encodes the sequence as set forth below (SEQ ID NO:66).

MAVMAPRTLLLLLSGALALTQTWAVNLTTRTQLPPAYTNSFTRGVYYPDK
VFRSSVLHSTQDLFLPFFSNVTWFHAIHVSGTNGTKRFDNPVLPFNDGVY
FASIEKSNIIRGWIFGTTLDSKTQSLLIVNNATNVVIKVCEFQFCNDPFL
GVYYHKNNKSWMKSEFRVYSSANNCTFEYVSQPFLMDLEGKQGNFKNLRE
FVFKNIDGYFKIYSKHTPINLVRDLPQGFSALEPLVDLPIGINITRFQTL
LALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEV
FNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNKL
DSKVGGNYNYRYRLFRKSNLKPFERDISTEIYQAGSTPCNGVQGFNCYFP
LQSYGFQPTYGVGYQPYRVVVLSFELLHAPATVCGPKKSTNLVKNKCVNF
NFNGLTGTGVLTESNKKFLPFQQFGRDIDDTTDAVRDPQTLEILDITPCS
FGGVSVITPGTNTSNQVAVLYQGVNCTEVPVAIHADQLTPTWRVYSTGSN
VFQTRAGCLIGAEHVNNSYECDIPIGAGICASYQTQTNSSVASQSIIAYT
MSLGAENSVAYSNNSIAIPINFTISVTTEILPVSMTKTSVDCTMYICGDS
TECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALG
KLQDVVNQNAQALNTLVKQLSSNFGAISSVLNDILARLDKVEAEVQIDRL
ITGRLQSLQTYVTQQLIRAAEIRASANLAATKMSECVLGQSKRVDFCGKG
YHLMSFPQSAPHGVVFLHVTYVPAHEKNFTTAPAICHDGKAHFPREGVFV
SNGTHWFVTQRNFYEPQIITTHNTFVSGNCDVVIGIVNNTVYDPLQPELD
SFKEELDKYFKNHTSPDVDLGDISGINASVVNIQKEIDRLNEVAKNLNES
LIDLQELGKYEQYIKWPVGIIAGLVLLGAVITGAVVAAVMWRRKSSDRKG
GSYTQAASSDSAQGSDVSLTACKVSRLEEELRRRLTE*

In certain embodiments, the vaccine comprises the sequence encoding a Lambda Spike S1+S2ECD_CD74CD+HLATM (SEQ ID NO: #)

GCCACCATGCACCGGAGGAGATCCAGAAGTTGCAGAGAAGACCAAAATCC
AGTAATGGATGACCAAAGGGATCTTATTTCTAATAACGAGCAGCTCCCTA
TGCTGGGACGCCGACCGGGGGCACCCGAATCAAAGTGCAGTCGCTGGATG
GTGGCTGCGGTCGTGGCAGGTACGATTGTGGCTGGCCTCCTCGTTCTTGG
GGCTATTATCGGTGTAGTTAACTTTACTACTCGGACCCAACTCCCGCCGG
CGTACACTAACTCTTTTACCCGGGGCGTCTATTATCCTGACAAAGTGTTT
CGATCATCTGTTTTGCACTCCACCCAAGATCTCTTTCTTCCATTTTTCAG
CAACGTTACGTGGTTCCACGCGATACATGTTAGTGGTACTAATGTTATAA
AGAGATTTGACAACCCTGTGCTCCCGTTTAACGATGGGGTCTATTTCGCT
TCCACTGAGAAGTCCAACATTATTCGCGGTTGGATCTTCGGCACCACACT
CGATTCAAAAACCCAAAGCTTGCTCATAGTTAACAACGCCACAAACGTAG
TCATTAAAGTGTGCGAATTTCAGTTTTGTAACTATCCCTTTTTGGGCGTC
TACTATCACAAGAACAACAAGTCCTGGATGGAATCTGAGTTTCGGGTCTA
CAGTTCAGCCAACAACTGTACGTTTGAGTACGTTAGCCAGCCCTTTCTGA
TGGATCTTGAAGGGAAGCAAGGAAATTTCAAGAATCTTTCCGAGTTTGTG
TTTAAAAATATAGACGGATATTTCAAGATATACAGTAAGCACACACCGAT
CAATTTGGTACGGGATTTGCCGCAAGGATTTTCCGCCCTTGAGCCCCTTG
TAGATCTCCCCATCGGAATTAACATAACCAGGTTTCAAACACTGCTGGCG
CTCCACAACTCATCCAGTGGGTGGACCGCCGGAGCAGCCGCATACTACGT
GGGGTATCTCCAGCCCAGAACGTTTCTCCTCAAGTATAATGAAAACGGGA
CCATCACCGACGCGGTTGATTGTGCACTCGATCCCCTCAGCGAGACAAAG
TGCACACTTAAATCATTTACCGTCGAGAAGGGGATCTACCAAACATCCAA
CTTTCGGGTGCAGCCTACCGAGTCAATAGTACGGTTTCCTAACATCACCA
ATTTGTGCCCTTTTGGGGAAGTATTTAATGCAACCCGGTTTGCGAGCGTA
TACGCTTGGAACCGAAAACGGATAAGTAATTGTGTTGCAGATTATTCTGT
GCTCTATAACTCAGCTTCCTTCTCCACCTTTAAGTGTTACGGAGTATCTC
CGACAAAATTGAATGATCTTTGTTTTACGAATGTTTACGCTGACTCATTT
GTTATCCGAGGTGACGAAGTGCGCCAGATAGCCCCCGGTCAAACAGGCAA
AATTGCTGATTATAACTACAAGTTGCCCGATGACTTCACGGGGTGTGTTA
TTGCATGGAATTCAAATAATCTTGATAGTAAGGTAGGGGGTAACTATAAT
TACCAATATCGCTTGTTCCGCAAATCCAATCTGAAACCCTTCGAAAGGGA
TATCAGTACCGAGATTTATCAAGCCGGCTCCACACCTTGTAACGGAGTAA
AAGGTTTTAACTGTTATTCCCCACTGCAGAGTTACGGCTTTCAACCCACA
TACGGGGTGGGATATCAGCCCTATCGCGTTGTGGTCTTGTCCTTTGAACT
TCTCCACGCCCCTGCAACAGTTTGTGGTCCTAAAAAGAGTACAAATCTGG
TAAAAAATAAGTGCGTCAACTTTAACTTCAACGGACTGACTGGTACCGGG
GTCCTCACTGAGAGCAACAAGAAATTTTTGCCCTTCCAGCAATTCGGGAG
GGATATTGCCGATACTACCGATGCTGTACGGGACCCCCAGACACTCGAAA
TATTGGACATAACCCCGTGCTCTTTTGGAGGGGTCTCTGTGATTACCCCG
GGTACGAACACGAGTAATCAGGTGGCAGTGCTTTATCAGGGAGTGAATTG
CACAGAAGTACCAGTCGCGATCCATGCCGACCAGCTCACGCCGACCTGGA
GGGTTTATAGTACGGGTAGTAACGTTTTCCAAACACGGGCCGGTTGTCTC
ATCGGGGCAGAGTACGTTAATAACAGCTATGAATGTGATATACCCATAGG
TGCCGGGATTTGTGCTTCTTATCAAACCCAAACCAATAGCTCACAGTCTA
TAATCGCCTATACGATGAGCCTCGGAGCGGAAAACTCAGTTGCCTACTCC
AACAATAGCATTGCTATCCCAACGAACTTCACTATTAGCGTAACCACGGA
GATCCTTCCAGTTAGCATGACAAAAACTAGCGTCGATTGCACTATGTATA
TTTGCGGCGACAGTACAGAGTGTAGTAATTTGCTCCTGCAGTACGGGTCT
TTCTGCACCCAATTGAACCGAGCACTGACTGGCATCGCCGTGGAACAAGA
CAAAAACACGCAAGAAGTATTTGCGCAAGTCAAGCAGATTTACAAGACTC
CCCCGATCAAAGACTTTGGCGGATTTAATTTTAGCCAGATACTCCCCGAT
CCATCTAAACCATCAAAACGGTCCTTTATAGAAGATCTCTTGTTCAATAA
AGTGACACTCGCGGACGCGGGCTTTATCAAACAGTATGGGGACTGCCTTG
GAGATATAGCTGCGAGGGACTTGATTTGCGCCCAGAAATTCAACGGGCTG
AATGTTCTGCCGCCGCTCTTGACAGACGAAATGATTGCACAGTATACGAG
TGCCTTGCTGGCAGGGACTATCACTTCCGGTTGGACCTTCGGCGCAGGAG
CAGCGCTCCAGATCCCATTCGCTATGCAAATGGCTTACCGATTCAACGGA
ATTGGCGTCACACAGAACGTGCTTTACGAGAATCAAAAGCTGATTGCCAA
TCAATTCAACTCAGCGATTGGCAAAATACAAGATAGTCTCTCTTCAACTG
CATCCGCGCTCGGTAAGTTGCAGGACGTAGTCAATCAGAACGCACAGGCC
CTGAACACGCTTGTTAAACAACTGTCTTCAAACTTCGGTGCAATAAGTTC
CGTACTTAATGATATCTTGTCAAGGTTGGATAAGGTCGAAGCTGAGGTAC
AAATCGACCGCCTTATAACAGGCAGACTCCAATCCCTGCAGACATACGTG
ACCCAGCAACTCATTAGGGCAGCCGAGATCAGAGCAAGTGCCAACCTCGC
TGCTACAAAAATGTCTGAATGCGTTTTGGGGCAATCTAAGCGGGTTGATT
TCTGCGGTAAGGGATACCACTTGATGTCCTTTCCTCAGTCCGCTCCTCAT
GGGGTAGTATTTCTTCATGTTACATATGTACCAGCACAAGAGAAAAATTT
TACTACTGCCCCGGCCATATGCCATGATGGCAAAGCGCACTTCCCTCGGG
AGGGGGTTTTTGTGTCTAATGGAACCCACTGGTTCGTGACTCAGCGGAAT
TTCTATGAACCCCAGATTATAACTACTGACAACACTTTCGTTTCTGGAAA
CTGCGATGTAGTAATAGGCATAGTAAATAATACAGTATATGATCCCTTGC
AACCAGAATTGGACTCTTTTAAAGAAGAGCTCGACAAATATTTTAAAAAC
CATACATCTCCAGACGTTGATCTTGCTGACATCTCTGGGATAAACGCTAG
CGTAGTAAACATACAAAAAGAAATCGATAGGCTTAACGAGGTCGCAAAAA
ATCTTAATGAAAGTCTTATCGATTTGCAAGAGCTTGGAAAGTATGAGCAG
TATATCAAATGGCCTTCACGACTGGAGGAAGAACTGCGCCGACGCCTGAC
TGAATGA

In certain embodiments the vaccine comprises or encodes Lambda Spike S1+S2ECD_CD74CD+HLATM. In specific embodiments the vaccine comprises or encodes the sequence as set forth below (SEQ ID NO:83).

MHRRRSRSCREDQNPVMDDQRDLISNNEQLPMLGRRPGAPESKCSRWMVA
AVVAGTIVAGLLVLGAIIGVVNFTTRTQLPPAYTNSFTRGVYYPDKVFRS
SVLHSTQDLFLPFFSNVTWFHAIHVSGTNVIKRFDNPVLPFNDGVYFAST
EKSNIIRGWIFGTTLDSKTQSLLIVNNATNVVIKVCEFQFCNYPFLGVYY
HKNNKSWMESEFRVYSSANNCTFEYVSQPFLMDLEGKQGNFKNLSEFVFK
NIDGYFKIYSKHTPINLVRDLPQGFSALEPLVDLPIGINITRFQTLLALH
NSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYQ
YRLFRKSNLKPFERDISTEIYQAGSTPCNGVKGFNCYSPLQSYGFQPTYG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIG
AEYVNNSYECDIPIGAGICASYQTQTNSSQSIIAYTMSLGAENSVAYSNN
SIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLNV
LPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQ
QLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHT
SPDVDLADISGINASVVNIQKEIDRLNEVAKNLNESLIDLQELGKYEQYI
KWPSRLEEELRRRLTE*

In certain embodiments, the vaccine comprises the sequence encoding a Lambda Spike S1+S2ECD_HLA SS/TM/CD. In specific embodiments the vaccine comprises or encodes the sequence as set forth below (SEQ ID NO:84).

GCCACCATGGCGGTCATGGCACCACGCACGCTCCTTCTTTTGTTGTCTGG
TGCCCTTGCACTCACCCAAACGTGGGCAGTTAACTTTACTACTCGGACCC
AACTCCCGCCGGCGTACACTAACTCTTTTACCCGGGGCGTCTATTATCCT
GACAAAGTGTTTCGATCATCTGTTTTGCACTCCACCCAAGATCTCTTTCT
TCCATTTTTCAGCAACGTTACGTGGTTCCACGCGATACATGTTAGTGGTA
CTAATGTTATAAAGAGATTTGACAACCCTGTGCTCCCGTTTAACGATGGG
GTCTATTTCGCTTCCACTGAGAAGTCCAACATTATTCGCGGTTGGATCTT
CGGCACCACACTCGATTCAAAAACCCAAAGCTTGCTCATAGTTAACAACG
CCACAAACGTAGTCATTAAAGTGTGCGAATTTCAGTTTTGTAACTATCCC
TTTTTGGGCGTCTACTATCACAAGAACAACAAGTCCTGGATGGAATCTGA
GTTTCGGGTCTACAGTTCAGCCAACAACTGTACGTTTGAGTACGTTAGCC
AGCCCTTTCTGATGGATCTTGAAGGGAAGCAAGGAAATTTCAAGAATCTT
TCCGAGTTTGTGTTTAAAAATATAGACGGATATTTCAAGATATACAGTAA
GCACACACCGATCAATTTGGTACGGGATTTGCCGCAAGGATTTTCCGCCC
TTGAGCCCCTTGTAGATCTCCCCATCGGAATTAACATAACCAGGTTTCAA
ACACTGCTGGCGCTCCACAACTCATCCAGTGGGTGGACCGCCGGAGCAGC
CGCATACTACGTGGGGTATCTCCAGCCCAGAACGTTTCTCCTCAAGTATA
ATGAAAACGGGACCATCACCGACGCGGTTGATTGTGCACTCGATCCCCTC
AGCGAGACAAAGTGCACACTTAAATCATTTACCGTCGAGAAGGGGATCTA
CCAAACATCCAACTTTCGGGTGCAGCCTACCGAGTCAATAGTACGGTTTC
CTAACATCACCAATTTGTGCCCTTTTGGGGAAGTATTTAATGCAACCCGG
TTTGCGAGCGTATACGCTTGGAACCGAAAACGGATAAGTAATTGTGTTGC
AGATTATTCTGTGCTCTATAACTCAGCTTCCTTCTCCACCTTTAAGTGTT
ACGGAGTATCTCCGACAAAATTGAATGATCTTTGTTTTACGAATGTTTAC
GCTGACTCATTTGTTATCCGAGGTGACGAAGTGCGCCAGATAGCCCCCGG
TCAAACAGGCAAAATTGCTGATTATAACTACAAGTTGCCCGATGACTTCA
CGGGGTGTGTTATTGCATGGAATTCAAATAATCTTGATAGTAAGGTAGGG
GGTAACTATAATTACCAATATCGCTTGTTCCGCAAATCCAATCTGAAACC
CTTCGAAAGGGATATCAGTACCGAGATTTATCAAGCCGGCTCCACACCTT
GTAACGGAGTAAAAGGTTTTAACTGTTATTCCCCACTGCAGAGTTACGGC
TTTCAACCCACATACGGGGTGGGATATCAGCCCTATCGCGTTGTGGTCTT
GTCCTTTGAACTTCTCCACGCCCCTGCAACAGTTTGTGGTCCTAAAAAGA
GTACAAATCTGGTAAAAAATAAGTGCGTCAACTTTAACTTCAACGGACTG
ACTGGTACCGGGGTCCTCACTGAGAGCAACAAGAAATTTTTGCCCTTCCA
GCAATTCGGGAGGGATATTGCCGATACTACCGATGCTGTACGGGACCCCC
AGACACTCGAAATATTGGACATAACCCCGTGCTCTTTTGGAGGGGTCTCT
GTGATTACCCCGGGTACGAACACGAGTAATCAGGTGGCAGTGCTTTATCA
GGGAGTGAATTGCACAGAAGTACCAGTCGCGATCCATGCCGACCAGCTCA
CGCCGACCTGGAGGGTTTATAGTACGGGTAGTAACGTTTTCCAAACACGG
GCCGGTTGTCTCATCGGGGCAGAGTACGTTAATAACAGCTATGAATGTGA
TATACCCATAGGTGCCGGGATTTGTGCTTCTTATCAAACCCAAACCAATA
GCTCACAGTCTATAATCGCCTATACGATGAGCCTCGGAGCGGAAAACTCA
GTTGCCTACTCCAACAATAGCATTGCTATCCCAACGAACTTCACTATTAG
CGTAACCACGGAGATCCTTCCAGTTAGCATGACAAAAACTAGCGTCGATT
GCACTATGTATATTTGCGGCGACAGTACAGAGTGTAGTAATTTGCTCCTG
CAGTACGGGTCTTTCTGCACCCAATTGAACCGAGCACTGACTGGCATCGC
CGTGGAACAAGACAAAAACACGCAAGAAGTATTTGCGCAAGTCAAGCAGA
TTTACAAGACTCCCCCGATCAAAGACTTTGGCGGATTTAATTTTAGCCAG
ATACTCCCCGATCCATCTAAACCATCAAAACGGTCCTTTATAGAAGATCT
CTTGTTCAATAAAGTGACACTCGCGGACGCGGGCTTTATCAAACAGTATG
GGGACTGCCTTGGAGATATAGCTGCGAGGGACTTGATTTGCGCCCAGAAA
TTCAACGGGCTGAATGTTCTGCCGCCGCTCTTGACAGACGAAATGATTGC
ACAGTATACGAGTGCCTTGCTGGCAGGGACTATCACTTCCGGTTGGACCT
TCGGCGCAGGAGCAGCGCTCCAGATCCCATTCGCTATGCAAATGGCTTAC
CGATTCAACGGAATTGGCGTCACACAGAACGTGCTTTACGAGAATCAAAA
GCTGATTGCCAATCAATTCAACTCAGCGATTGGCAAAATACAAGATAGTC
TCTCTTCAACTGCATCCGCGCTCGGTAAGTTGCAGGACGTAGTCAATCAG
AACGCACAGGCCCTGAACACGCTTGTTAAACAACTGTCTTCAAACTTCGG
TGCAATAAGTTCCGTACTTAATGATATCTTGTCAAGGTTGGATAAGGTCG
AAGCTGAGGTACAAATCGACCGCCTTATAACAGGCAGACTCCAATCCCTG
CAGACATACGTGACCCAGCAACTCATTAGGGCAGCCGAGATCAGAGCAAG
TGCCAACCTCGCTGCTACAAAAATGTCTGAATGCGTTTTGGGGCAATCTA
AGCGGGTTGATTTCTGCGGTAAGGGATACCACTTGATGTCCTTTCCTCAG
TCCGCTCCTCATGGGGTAGTATTTCTTCATGTTACATATGTACCAGCACA
AGAGAAAAATTTTACTACTGCCCCGGCCATATGCCATGATGGCAAAGCGC
ACTTCCCTCGGGAGGGGGTTTTTGTGTCTAATGGAACCCACTGGTTCGTG
ACTCAGCGGAATTTCTATGAACCCCAGATTATAACTACTGACAACACTTT
CGTTTCTGGAAACTGCGATGTAGTAATAGGCATAGTAAATAATACAGTAT
ATGATCCCTTGCAACCAGAATTGGACTCTTTTAAAGAAGAGCTCGACAAA
TATTTTAAAAACCATACATCTCCAGACGTTGATCTTGCTGACATCTCTGG
GATAAACGCTAGCGTAGTAAACATACAAAAAGAAATCGATAGGCTTAACG
AGGTCGCAAAAAATCTTAATGAAAGTCTTATCGATTTGCAAGAGCTTGGA
AAGTATGAGCAGTATATCAAATGGCCTGTAGGTATTATAGCCGGGCTCGT
GTTGTTGGGTGCAGTAATCACAGGAGCCGTCGTGGCAGCGGTTATGTGGA
GACGGAAATCTTCAGACCGCAAAGGTGGTAGTTACACTCAAGCGGCTTCC
AGTGATTCTGCCCAGGGCTCCGACGTATCCCTCACTGCGTGTAAGGTTTC
ACGACTGGAGGAAGAACTGCGCCGACGCCTGACTGAATGA

In certain embodiments the vaccine comprises or encodes Lambda Spike S1+S2ECD_HLA SS/TM/CD (SEQ ID NO:85)

MAVMAPRTLLLLLSGALALTQTWAVNFTTRTQLPPAYTNSFTRGVYYPDK
VFRSSVLHSTQDLFLPFFSNVTWFHAIHVSGTNVIKRFDNPVLPFNDGVY
FASTEKSNIIRGWIFGTTLDSKTQSLLIVNNATNVVIKVCEFQFCNYPFL
GVYYHKNNKSWMESEFRVYSSANNCTFEYVSQPFLMDLEGKQGNFKNLSE
FVFKNIDGYFKIYSKHTPINLVRDLPQGFSALEPLVDLPIGINITRFQTL
LALHNSSSGWTAGAAAYYVGYLQPRTFLLKYNENGTITDAVDCALDPLSE
TKCTLKSFTVEKGIYQTSNFRVQPTESIVRFPNITNLCPFGEVFNATRFA
SVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGN
YNYQYRLFRKSNLKPFERDISTEIYQAGSTPCNGVKGFNCYSPLQSYGFQ
PTYGVGYQPYRVVVLSFELLHAPATVCGPKKSTNLVKNKCVNFNFNGLTG
TGVLTESNKKFLPFQQFGRDIADTTDAVRDPQTLEILDITPCSFGGVSVI
TPGTNTSNQVAVLYQGVNCTEVPVAIHADQLTPTWRVYSTGSNVFQTRAG
CLIGAEYVNNSYECDIPIGAGICASYQTQTNSSQSIIAYTMSLGAENSVA
YSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFN
GLNVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNA
QALNTLVKQLSSNFGAISSVLNDILSRLDKVEAEVQIDRLITGRLQSLQT
YVTQQLIRAAEIRASANLAATKMSECVLGQSKRVDFCGKGYHLMSFPQSA
PHGVVFLHVTYVPAQEKNFTTAPAICHDGKAHFPREGVFVSNGTHWFVTQ
RNFYEPQIITTDNTFVSGNCDVVIGIVNNTVYDPLQPELDSFKEELDKYF
KNHTSPDVDLADISGINASVVNIQKEIDRLNEVAKNLNESLIDLQELGKY
EQYIKWPVGIIAGLVLLGAVITGAVVAAVMWRRKSSDRKGGSYTQAASSD
SAQGSDVSLTACKVSRLEEELRRRLTE*

In certain embodiments, the vaccines viral vector-based vaccines or nucleic acid-based vaccines.

In certain embodiments, the vaccines target more than one strain of SARs-CoV, including but not limited to SARs-CoV2. In certain embodiments, the vaccines encode spike proteins, fragments thereof or fusion proteins comprising spike proteins or fragments thereof from more than one strain of SARs-CoV2. In specific embodiments, the vaccines encode more than one polypeptide selected from the group consisting of SEQ ID NOs: 44, 46, 48, 50, 52 and 54. In specific embodiments, the vaccines encode more than one polypeptide selected from the group consisting of 56, 58, 60, 62, 64 and 66.

In certain embodiments, vaccine target one or more influenza viruses. In certain embodiments the vaccine comprises or encodes an influenza immunogen. In certain embodiments the vaccine comprises or encodes a targeting domain and an influenza immunogen. Optionally, the targeting domain comprises CD74 Cytoplasmic domain and a HLA transmembrane domain. Optionally the influenza immunogen is selected from the group consisting of one or more of M, N, HA, fragments thereof, variants thereof and combinations thereof.

In specific embodiments, the vaccines are SAM RNA-based vaccines. Optionally, the SAM vaccines are in lipid nanoparticle formulations.

Vaccine Formulations

The vaccines formulations may also comprise pharmaceutically acceptable carriers, excipients and/or adjuvants. Adjuvants and carriers suitable for administering genetic vaccines and immunogens are known in the art. Conventional carriers and adjuvants are for example reviewed in Kiyono et al. 1996.

A vaccine adjuvant is a component that potentiates the immune responses to an antigen and/or modulates it towards the desired immune responses. A vaccine may include one or more adjuvants. Exemplary adjuvants include mineral salts including but not limited to aluminium salts (such as amorphous aluminum hydroxyphosphate sulfate (AAHS), aluminum hydroxide, aluminum phosphate, potassium aluminum sulfate (Alum)) and calcium phosphate gels; Oil emulsions and surfactant based formulations, including but not limited to MF59 (microfluidised detergent stabilised oil-in-water emulsion), QS21 (purified saponin), AS02 [SBAS2] (oil-in-water emulsion+MPL+QS-21), Montanide ISA-51 and ISA-720 (stabilised water-in-oil emulsion); Particulate adjuvants, including but not limited to virosomes (unilamellar liposomal vehicles incorporating influenza haemagglutinin), AS04 ([SBAS4] Al salt with MPL), ISCOMS (structured complex of saponins and lipids), polylactide co-glycolide (PLG) and; microbial derivatives (natural and synthetic), including but not limited to monophosphoryl lipid A (MPL), Detox (MPL+M. Phlei cell wall skeleton), AGP [RC-529] (synthetic acylated monosaccharide), DC_Chol (lipoidal immunostimulators able to self organise into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, including but not limited to hGM-CSF or hIL-12 (cytokines that can be administered either as protein or plasmid encoded), Immudaptin (C3d tandem array) and inert vehicles, such as gold particles.

The vaccine formulations may also comprise a stabilizer. Suitable stabilizer are known in the art and include but are not limited to amino acids, antioxidants, cyclodextrins, proteins, sugars/sugar alcohols, and surfactants. See for example Morefield, AAPS J. 2011 June; 13(2): 191-200; https://www.ncbi.nlm.nih.gov/pmc/articles/PMC3085699/).

The vaccine can be incorporated into liposomes, microspheres or other polymer matrices. Liposomes can consist of phospholipids or other lipids, and can be nontoxic, physiologically acceptable and metabolizable carriers that are relatively simple to make and administer.

Previously, it has been found that a SARS-CoV-2 SAM lipid nanoparticle (LNP) vaccine induced high neutralizing antibody titers in mice (McKay et al., Nat Commun 11, 3523 (2020). https://doi.org/10.1038/s41467-020-17409-9). Briefly, the LNP (described in US patent U.S. Pat. No. 10,221,127) contains an ionizable cationic lipid phosphatidylcholine/cholesterol/PEG-lipid. The SAM RNA were encapsulated in LNP using a self-assembly process in which an aqueous solution of SAM RNA at pH=4.0 is rapidly mixed with an ethanolic lipid mixture. LNP.

Accordingly, in certain embodiments, the vaccines formulations comprise lipid nanoparticle delivery formulations of nucleic acid-based vaccines. Optionally, the lipid is cationic. Appropriate cationic lipids are known in the art. Non-limiting examples include phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA). Also see for example, U.S. Pat. No. 10,221,127 (incorporated by reference) and Reichmuth A M et al. (Therapeutic Delivery. 2016; 7(5):319-334. DOI: 10.4155/tde-2016-0006). In specific embodiments, the vaccines formulations comprise lipid nanoparticle delivery formulations of SAM RNA vaccines. In specific embodiments, the LNPs comprise an ionizable cationic lipid (phosphatidylcholine:cholesterol/PEG-lipid (50:10:38.5:1.5 mol/mol). In certain embodiments, the RNA to total lipid ratio in the LNP is approximately 0.05 (wt/wt). In certain embodiments, the LNPs have a diameter of −80 nm

Method of Vaccination

Also provided herein is a method of treating, protecting against, and/or preventing disease associated with the one or more viral pathogens in a subject in need thereof by administering one or more vaccines to the subject. For example, a worker skilled in the art would readily appreciate that a SARS-CoV-2 vaccine may be used in treating, protecting against, and/or preventing disease associated with SARS-CoV-2 (i.e. COVID 19) and an influenza vaccine may be used in treating, protecting against and/or preventing disease associated with influenza. A combination vaccine targeting SARS-CoV-2 and influenza may be used in treating, protecting against and/or preventing COVID-19 and influenza. Administration of the vaccine to the subject can induce or elicit a specific immune response against the vaccine target in the subject.

The subject may be a human or other animals, including but not limited to other vertebrates including mammals, such as non-human primates (including but not limited to monkeys and apes), cats, dogs, equines (including but not limited to horses), sheep, goats; bovines (including but not limited to cows), pangolins and marsupials; birds; reptiles; amphibians and fish.

The induced immune response can be used to treat, prevent, and/or protect against disease related to the vaccine target. For example, a SARS-CoV-2 vaccine to the subject can induce or elicit a specific immune response against the SARS-CoV-2 in the subject. The induced immune response provides the subject administered the vaccine resistance to vaccine target, such as a SARS-CoV-2 vaccine provides resistance to SARS-CoV-2.

The induced immune response can include an induced humoral immune response and/or an induced cellular immune response. The induced humoral immune response can include IgG antibodies and/or neutralizing antibodies that are reactive to the antigen. The induced cellular immune response can include a CD8+ T cell response.

The number of vaccine doses for effective treatment can be 1, 2, 3, 4, 5, 6, 7, 8, 9, or 10.

In certain embodiments, a single type of vaccine is used. In other embodiments, multiple types of vaccines are used. For example, in certain embodiments, a prime and boost strategy of vaccination is used. In such embodiments, one vaccine where the contigs expressing immunogens are linked to CD74/HLA targeting sequences is used (this may promote DC cross priming of T cells). A second vaccine where the contigs expressing immunogens lack all signal sequences and transmembrane domains and (this may promote endogenous antigen presentation). These may be administered together or in 2 separate immunization of priming and boosting to promote optimum T cell response.

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject may be a human or other animals, including but not limited to other vertebrates including mammals, such as non-human primates (including but not limited to monkeys and apes), cats, dogs, equines (including but not limited to horses), sheep, goats; bovines (including but not limited to cows), pangolins and marsupials; birds; reptiles; amphibians and fish.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997). The nucleic acid of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccine can be delivered via a variety of routes. Typical delivery routes include parenteral administration, e.g., intradermal, intramuscular or subcutaneous delivery. Other routes include oral administration, intranasal, and intravaginal routes. The vaccine can be delivered to the interstitial spaces of tissues of an individual (Feigner et al., U.S. Pat. Nos. 5,580,859 and 5,703,055. The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be administered via electroporation, such as by a method described in U.S. Pat. No. 7,664,545. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359. The electroporation may be carried out via a minimally invasive device.

A method of inducing an antigen-specific immune response in a subject, the method comprising administering to the subject the vaccine comprising at least one nucleic acid sequence of SEQ ID NO. 1-51 or a mutated variant thereof capable of expressing a polypeptide in an amount effective to produce an antigen-specific immune response in the subject.

### EXAMPLES

### Example 1: Vaccine Containing CD74 Peptide Promotes Augmentation of Immune Response to Influenza M1 Protein

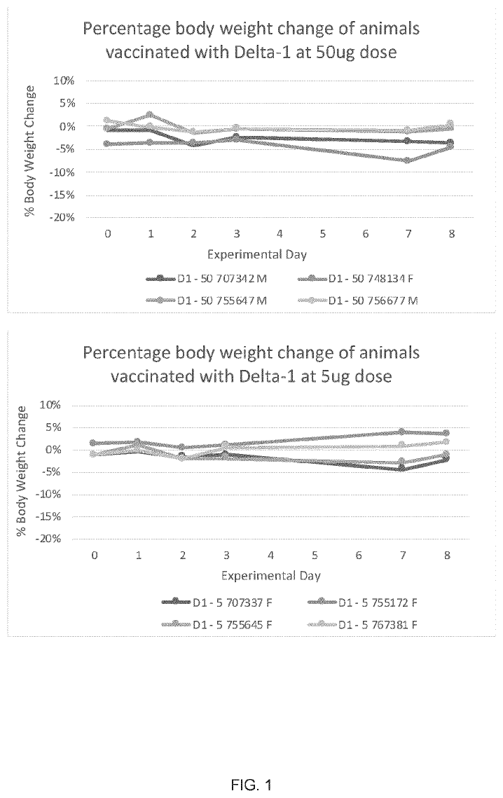

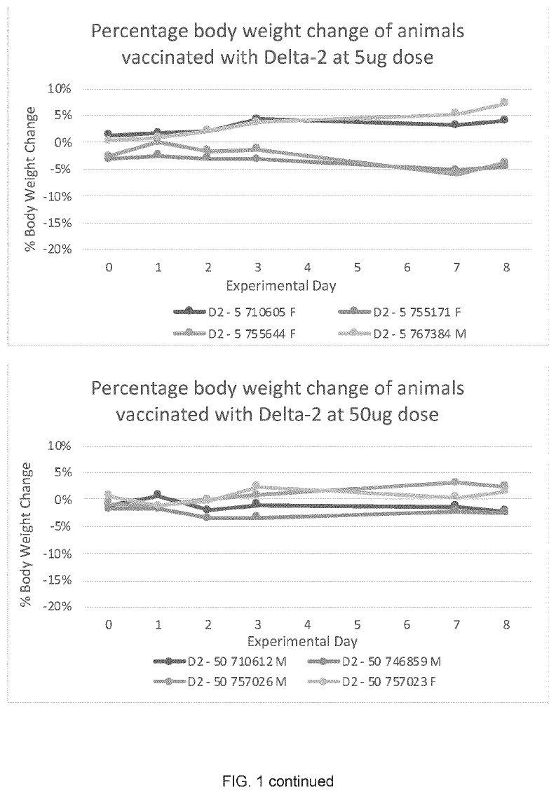

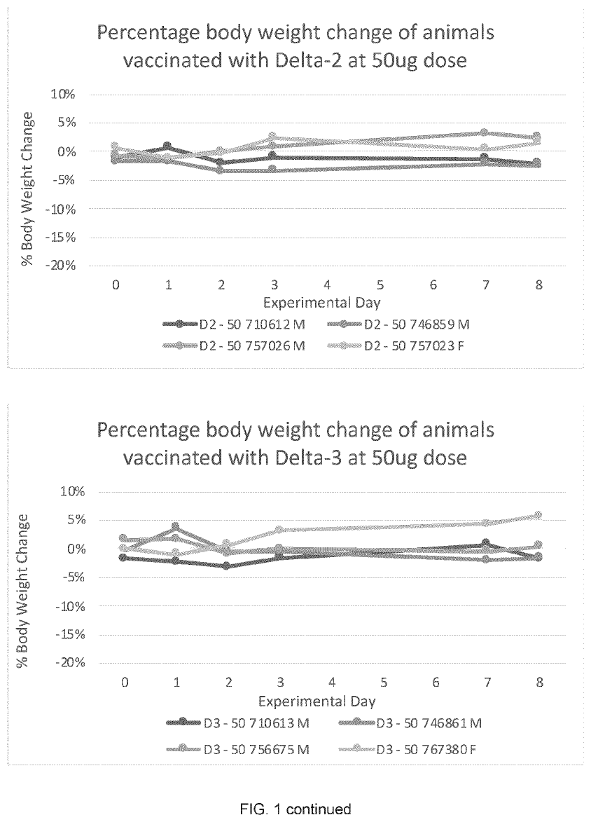

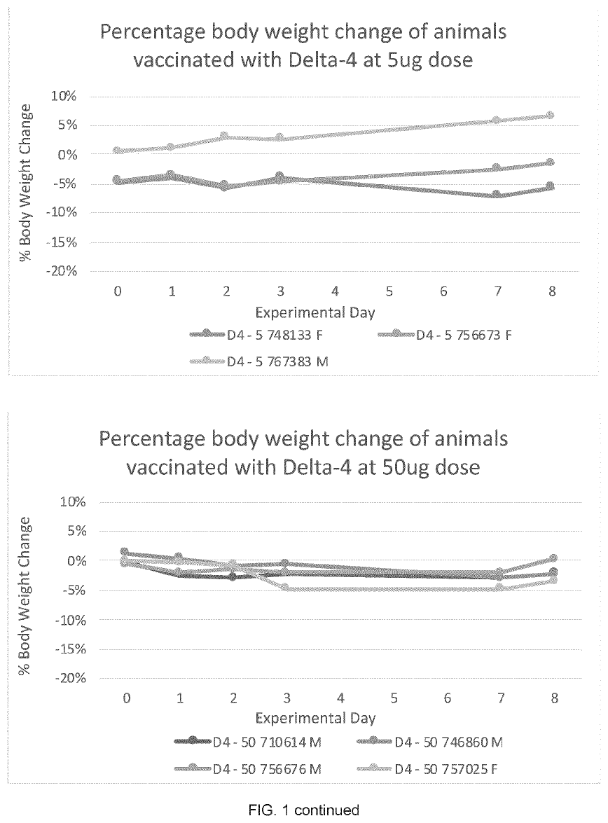

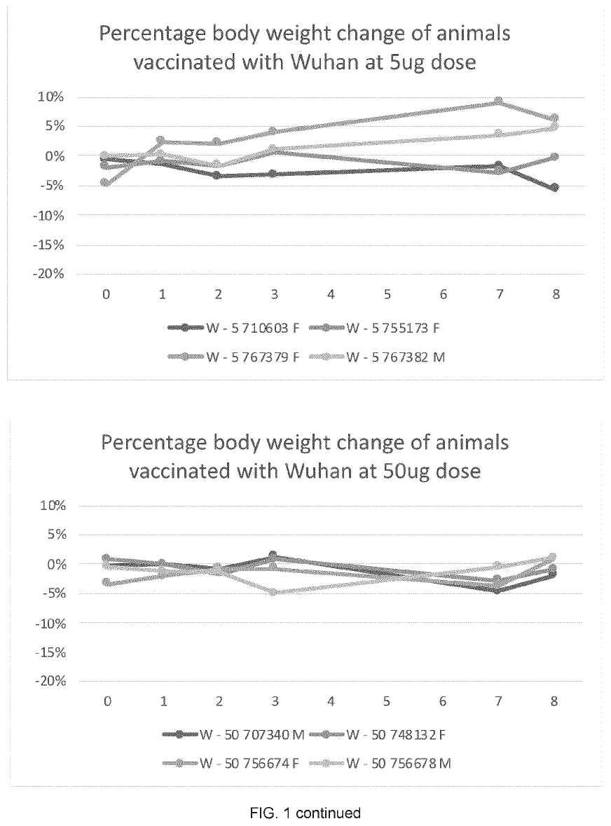

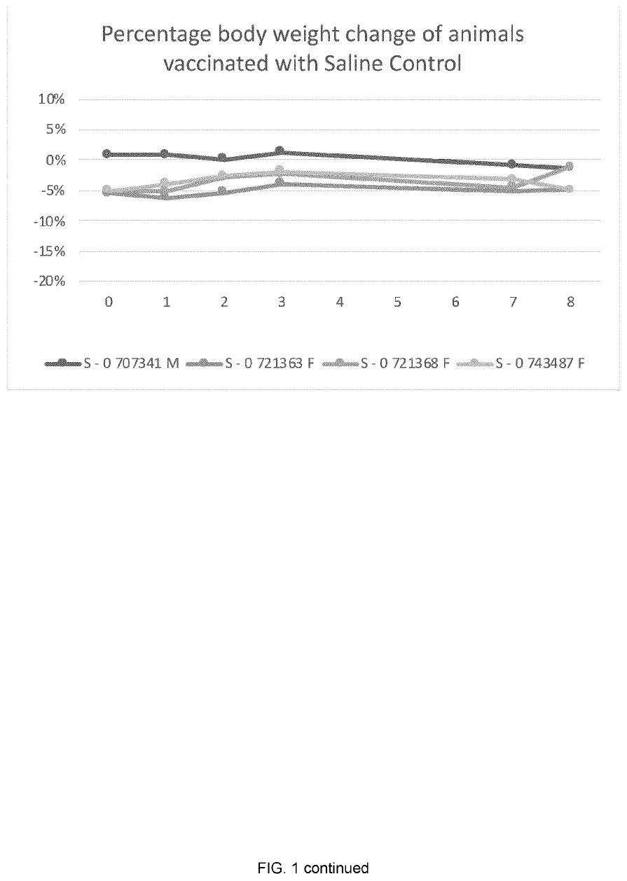

Mice vaccinated with CD74-M1 adenovirus vaccine display higher levels of HLA-A2 restricted M1 epitope in central memory T cells found in spleen (FIG. 1). Central memory T cells isolated from the spleen stained for CD4, CD8a, CD127, CD62L, CD25 and for the tetramer which recognized HLA-A2 with M1 peptide bound. Data shown in FIG. 1 represents a percentage of tetramer+/CD127+/CD62LhiTc cells within all splenic lymphocytes. At 10e7 pfu very few central memory T cells exist in either the Ad vector control or the Ad vector expressing wild-type M1; however, there is an elevated frequency of Central memory T cells in the Ad vector expressing M1 linked to the CD74 chaperone sequence. These results have been replicated greater than 5 times at identical and increased pfu. It was shown that the presentation of ovalbumin (OVA) or Influenza M1 protein by MHC I and MHC II molecules is increased when it is linked to the CD74 transmembrane and cytoplasmic domain that delivers the recombinant protein to the endolysosome for antigen processing and peptide loading on MHC I and II molecules for presentation to helper/inducer and cytolytic T Lymphocytes. Furthermore, the data showed enhancement of T cell memory immune responses and enhanced resistance to lethal Influenza A viral challenge and constructs using the CD74 targeting signal.

### Example 2

### Materials & Methods

A DNA plasmid expression vector containing a CMV promoter was utilized to express the various SARs-CoV2 immunogens.

The nomenclature of the DNA vaccine constructs is as follows:

Wuhan: Wuhan spike with Wuhan spike signal sequences and transmembrane domain and cytoplasmic domain.

D1: Delta spike variant (aka Indian) without signal sequences or transmembrane domain.

D2: Delta spike variant with CD74 cytoplasmic domain+HLA transmembrane fused at the N terminus.

D3: Delta spike variant with HLA signal sequences and transmembrane domain and cytoplasmic domain.

D4: Delta spike variant with Delta spike signal sequences or transmembrane domain and cytoplasmic domain.

Transfections: 1.7×10⁵ HEK293 cells were plated in 10% FBS+DMEM one day prior to the transfection in a 24 well plate. 1 ug of DNA for each vaccine construct was transfected with Lipofectamine3000 according to the manufacturer's instructions in healthy 70 to 90% confluent HEK293 cells. Transfected cells were incubated for 2 days before harvesting for protein expression.

Western Blotting: Samples of transfected HEK293 cells were separated on 15% sodium dodecyl sulfate-polyacrylamide precast gels (Bio-rad), transferred to nitrocellulose membranes (using 10% and not 20% methanol), blocked with 5% skim milk as the blocking buffer in TBST, probed with rabbit polyclonal alfa tag primary antibody (1:1000, Nanotag Technology) and goat anti rabbit secondary antibody conjugated with HRP (1:5000) (Thermofisher). 0.5% SDS was used in the transfer buffer to facilitate the transfer of large proteins. Bio-rad Bio-rad ECL XL was used for protein detection. Membranes were scanned with Bio-rad Chemidoc. Protein was quantified using ImageJ Software.

FACS Intracellular Staining: FACS intracellular staining was used to determine expression of constructs which express membrane bound proteins. Expression from the D1 construct cannot be measured with this method as the protein lacks a signal sequence and transmembrane domain. Briefly, Samples of transfected HEK293 cells were trypinised and washed three time in PBS, counted and transferred to 1.5 ml tubes to obtain 6×10⁵ cells per sample. Cells were fixed and permeabilised using the Invitrogen/eBioscience intracellular fixation and permeabilization kit. Briefly, for each sample, 200 μl fixation buffer was added and cells incubated for 30 minutes in the dark at room temp. Samples were washed 2 times in 500 μl permeabilization buffer, resuspended in 100 μl of the same buffer containing anti-ALFA antibody (NanoTag Bio, rabbit anti-ALFA at 1:500 dilution) and incubated for 30 minutes in the dark at room temperature. Samples were washed as before then 100 μl secondary antibody (Alex 488 goat anti-rabbit at 1:5,000 dilution) in permeabilization buffer was added and samples incubated for 30 in the dark at room temp. Samples were again washed as before then resuspended in 200 μl FACS buffer (PBS+2% FBS) and analysed by flow cytometry.

Mice: C57BL/6-Mcph1Tg(HLA-A2.1)1Enge/J mice, stock #003475, were purchased from The Jackson Laboratory (Bar Harbor, ME). Seven to thirty week old male and female mice were used and mixed for treatment groups. Homozygous progeny were bred for experiments and confirmed by in-house SNP analysis. Mice were maintained in the Centre for Disease Modelling (Life Sciences Building, Vancouver, British Columbia) and kept in a pathogen-free environment. All animal work was performed under strict accordance with the recommendations of the Canadian Council for Animal Care. The protocol was approved by the Animal Care Committee (ACC) of the University of British Columbia.

Immunizations: C57BL/6-Mcph1Tg(HLA-A2.1)1Enge/J and C57BL/6 mice were inoculated intramuscularly with either 0 ug, 5 ug, or 50 ug of unencapsulated DNA vaccine immersed in saline. Mice received one of five different DNA vaccines (four vaccines carrying modifications of the Delta variant Spike protein and one vaccine carrying the Wuhan Spike). Mice were monitored for weight loss (end point at 20% weight loss) and general health every day following immunization and blood collection. Mice that experienced 20% weight loss were euthanized for necropsy.

MIA: Saphenous blood was obtained from 4 mice per vaccine group at Days 0, 7, 14, and 21 post-immunization. Antigen-specific responses following vaccination were measured using an MIA. The MIA was developed and performed by MSD and is described in Folegatti et al. 17. Briefly, dried plates coated with SARS-CoV-2 spike protein and RBD were blocked, washed and incubated with samples, reference standards and controls. Internal quality controls (QCs) and reference standard reagents were developed from pooled human serum. Following incubation and washing steps, detection antibody was added (MSD SULFO-TAG anti-mouse IgG), incubated and plates washed again. MSD GOLD Read Buffer B was added and plates read using a MESO SECTOR S 600.

Proteomics: Cytokines pre- and post-immunization were measured by proteome array using the Proteome Profiler Mouse XL Cytokine Array according to the manufacturer's instructions (Cat #ARY028, Lot: P293326; bio-Techne).

Results

Protein Expression: Protein Expression was confirmed by Western Blotting (data not shown) and FACS Intracellular Staining. Briefly, with respect to FACS intracellular staining, expression (background control subtracted) in fluorescent units (FU) is as: follows: Wuhan: 448 FU; D2: 986 FU; D3: 852 FU; and D4: 864 FU.

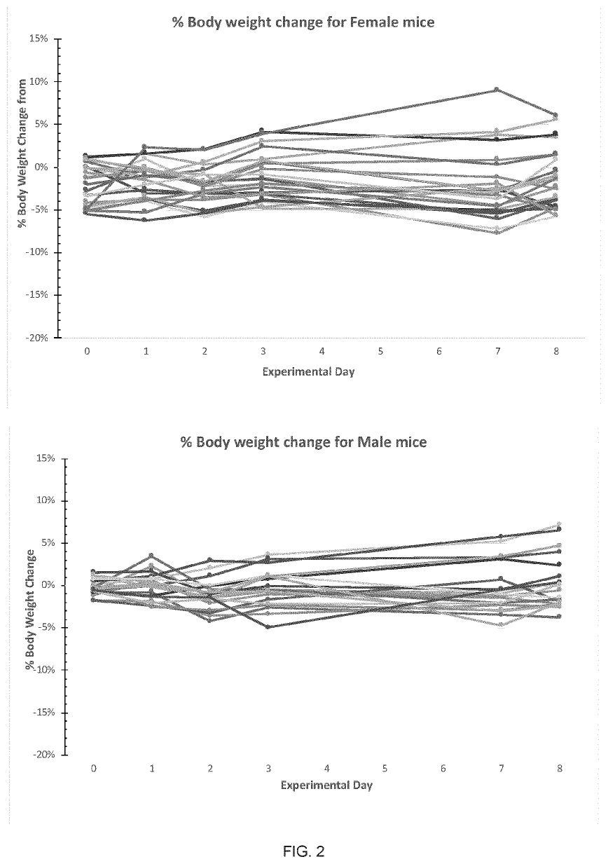

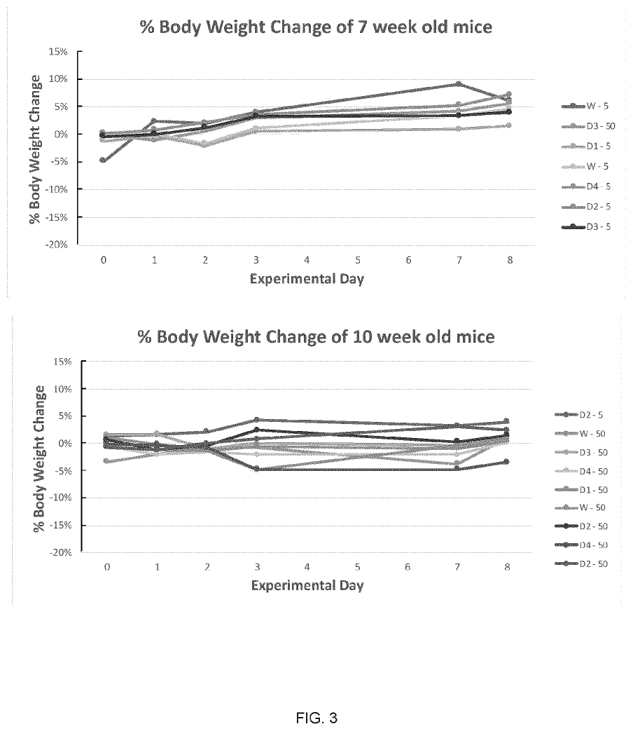

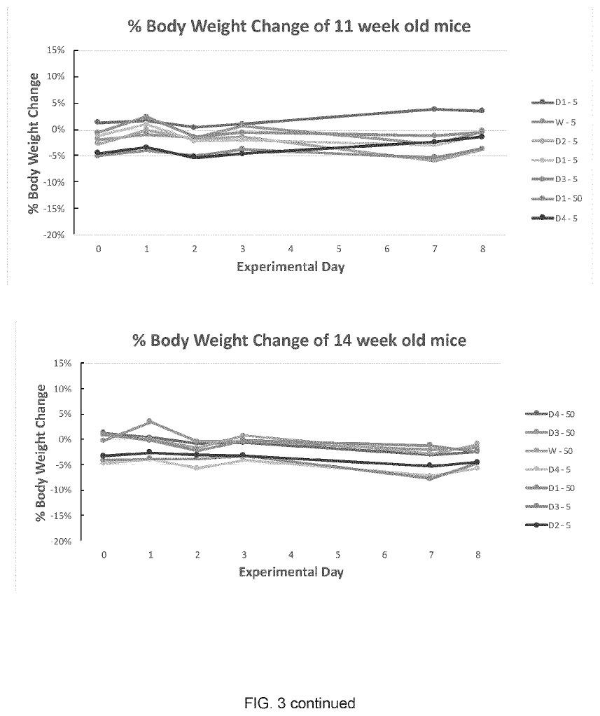

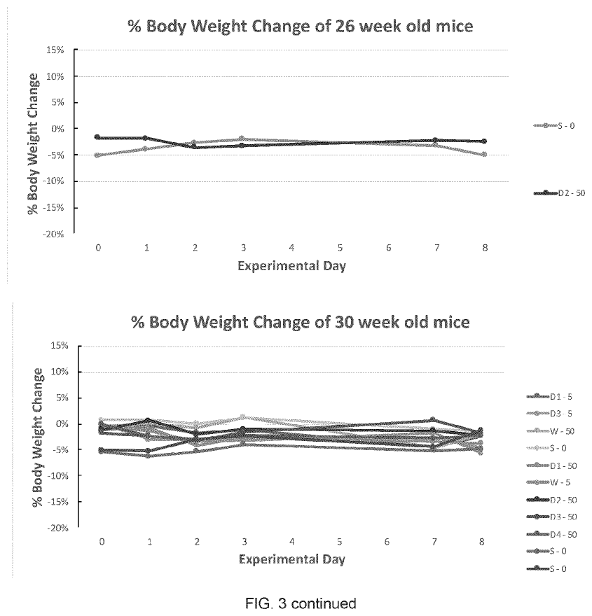

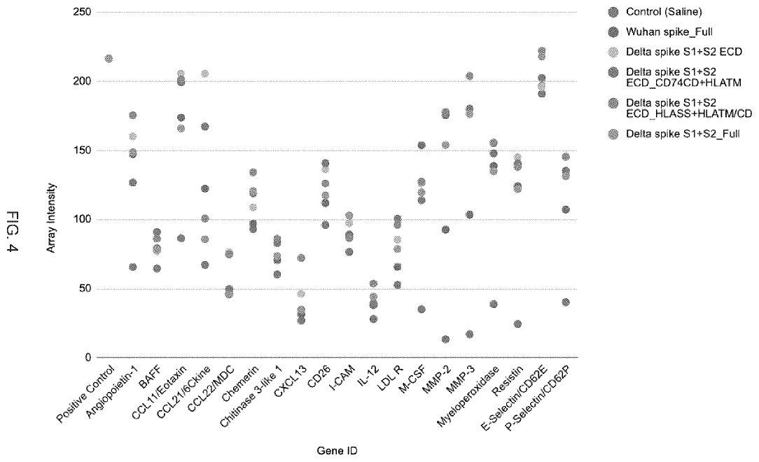

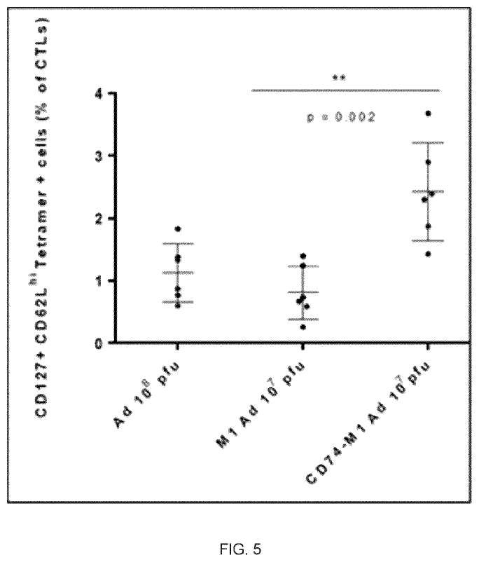

Toxicity: No toxicity events were observed for any vaccinated group and the mice retained their respective body weights irrespective of age or sex (see FIGS. 1 to 3).

Immune Biomarkers: Proteome arrays were used to assess vaccine performance by measuring immune responses after vaccination. Densiphotometry measurements were used to quantitate immune biomarker proteins that are elevated at day 7 and 14. There is no difference in vaccine biomarker expression at day 0 in the prebleeds of either the low dose (5 ug) or high dose (50 ug) doses of any vaccine versus the saline injected control group of animals. There is also no difference in vaccine biomarker Day 7 Low dose (5 ug) for any vaccine versus the controls at day 0 or the saline injected control group of animals. Several vaccine biomarker proteins were elevated in vaccine biomarker Day 7 High dose (50 ug) in all vaccinated animals versus the prebleed controls at day 0 or versus the saline injected control group of animals (see FIG. 4).

The biomarkers are specifically indicative of elevated humoral responses (B cell antibody) and elevated cellular responses (T cell) and elevated innate immune responses and indicate that all vaccines promote a robust immune response and occurred in in the High dose of all vaccines. These include: ANG-1—plasma levels of ANG-1 significantly correlated with the reconstitution of naive CD4+CD45RA+ and CD8+CD45RA+ T cell subsets, whereas plasma levels of VEGF displayed a positive correlation with CD4+CD45RO+ T cells https://pubmed.ncbi.nlm.nih.gov/27777141/; BAFF—BAFF is a B-cell-activating factor (BAFF)/B lymphocyte stimulator (BLyS), known to be essential for B lymphocyte homeostasis https://pubmed.ncbi.nlm.nih.gov/18155301/; Eotaxin—Eotaxin-1 regulates the chemiotaxis and activation of basophils, mast cells and T lymphocytes specificially on eosinophils, mast cells, Th2 type lymphocytes and even on keratinocytes https://pubmed.ncbi.nlm.nih.gov/14561178/; CCL21-CCL21 attract naive T cells as well as costimulate their proliferation and differentiation, and is a pivotal molecule for priming T cell. https://pubmed.ncbi.nlm.nih.gov/15919372/; CCL22-CCL22 is secreted by Dendritic cells as a major source of macrophage-derived chemokine/CCL22 to prime B and T cell responses https://pubmed.ncbi.nlm.nih.gov/11241286/; Chitinase 3-like-1-Chitinase 3-like-1, a master regulator of Th1/CTL responses, as a therapeutic target for increasing immunity https://pubmed.ncbi.nlm.nih.gov/29699605/; CD26-CD26 is preferentially restricted to the CD4+ helper/memory population, and CD26 delivers a potent co-stimulatory T-cell activation signal. https://pubmed.ncbi.nlm.nih.gov/9553764/; Interleukin 12 (IL-12)-Interleukin 12 (IL-12) is an interleukin that is naturally produced by dendritic cells,^{[1]}macrophaqes, neutrophils, and human B-lymphoblastoid cells (NC-37) in response to antigenic stimulation. https://www.ncbi.nlm.nih.gov/pmc/articles/PMC7825035/; LDL—LDL possesses both a Redox imbalance and immune functions https://pubmed.ncbi.nlm.nih.gov/11899429/; M-CSF—Macrophage colony stimulating factor (M-CSF) activates macrophages, and activates antigen-specific immune responses in vivo. https://pubmed.ncbi.nlm.nih.gov/9543701/; MMP-2-Matrix metalloproteinase-2 (MMP-2) plays an important roles in inflammation and immunity. https://pubmed.ncbi.nlm.nih.gov/19393189/; MMP-3-Matrix metalloproteinase-3 (MMP-3) is involved in Macrophage Activation where it up-regulates several matrix metalloproteinases through mitogen activated protein kinases and nuclear factor KB https://journals.plos.org/plosone/article?id=10.1371/journal.pone.0042507; Myeloperoxidase-Myeloperoxidase is a regulator of immune responses and a hereditary deficiency of the enzyme, predisposes to immune deficiency https://www.karger.com/Article/Abstract/41062; Resistin-Resistin, a Novel Host Defense Peptide of Innate Immunity https://www.ncbi.nlm.nih.gov/pmc/articles/PMC8253364/; E-selectin—The Adhesion to E-selectin primes macrophages for activation through AKT and mTOR https://pubmed.ncbi.nlm.nih.gov/33565143/; L-Selectin—L-Selectin enhanced T cells activation and function and improves the efficacy of immunotherapy: https://www.frontiersin.org/articles/10.3389/fimmu.2019.01321/full; CD62P—CD62P aids in T cells migrate and activates dendritic cells for T cell and memory T cell responses: https://pubmed.ncbi.nlm.nih.gov/18838544/; CD62L—L-selectin (CD62L) controls the capacity for naive and memory T cells to actively survey peripheral lymph nodes, whereas P- and E-selectin capture activated T cells on inflamed vascular endothelium to initiate extravasation into non-lymphoid tissues. https://www.frontiersin.org/articles/10.3389/fimmu.2017.00600/full; ICAM-1—ICAM-1 is master regulator of cellular immune responses: https://www.ncbi.nlm.nih.gov/pmc/articles/PMC7977775/

Quantitatively, the D2: Delta spike variant with CD74 cytoplasmic domain+HLA transmembrane fused at the N terminus and the D3: Delta spike variant with HLA signal sequences and transmembrane domain and cytoplasmic domain quantitatively outperformed the vaccine expressing the Wuhan spike with Wuhan spike signal sequences and transmembrane domain and cytoplasmic domain, the vaccine expressing the D1-Delta spike variant (aka Indian) without signal sequences or transmembrane domain and the vaccine expressing the D4: Delta spike variant with Delta spike signal sequences or transmembrane domain and cytoplasmic domain. Furthermore, the vaccine expressing the D3: Delta spike variant with HLA signal sequences and transmembrane domain and cytoplasmic domain outperformed the vaccine expressing the D2: Delta spike variant with CD74 cytoplasmic domain+HLA transmembrane fused at the N terminus.

Antibody Response: Day 14 specific antibody response against human coronavirus (HCoV-229E) spike protein (S1+S2 ECD) was determined. The following vaccines contain transmembrane regions and evoked a specific antibody response: Wuhan spike with Wuhan spike signal sequences and transmembrane domain and cytoplasmic domain; D2-Delta spike variant with CD74 cytoplasmic domain+HLA transmembrane fused at the N terminus; D3: Delta spike variant with HLA signal sequences and transmembrane domain and cytoplasmic domain; D4: Delta spike variant with spike signal sequences and transmembrane domain. While D1-Delta spike variant without signal sequences or transmembrane domain did not evoke an antibody response at day 14 likely due to its localization with the cytoplasm of cells expressing it.

### Example 3

### Testing Vaccines in a Hamster COVID-19 Disease Model

Golden Syrian hamsters are a current disease model for SARS-CoV-2 infection. Disease progression following SARS-CoV-2 infection in hamsters resembles that in human patients in multiple ways. SARS-CoV-2 replicates in their pulmonary and gastrointestinal epithelia, and viral antigens are expressed in airways and duodena up to 7 days post-inoculation. Community transmission to co-housed naïve contact hamsters occurs. Inoculated and naturally-infected hamsters display similar symptoms and disease progression, and neutralizing antibodies are detectable in survivors within 14 days of infection.

The additional advantage of the hamster model is that after SARS-CoV-2 infection with 10e5 of plaque forming units (pfu) of virus, the animals of both sexes lose over 20% of their body weight 7 days after infections with humane endpoint (20% loss is humane endpoint) and this is a useful parameter for assessing vaccine performance in viral challenge models. Weight loss does not appear in non-human primate models (Rhesus Macaques or African Green Monkeys), ferrets, or hACE2-mouse models of SARS-CoV-2 infection (Kobasa, member of the WHO Steering group on models of COVID-19). Therefore, hamsters provide one the best current models to validate vaccines for COVID-19 prior to entering clinical trials and act as a screen to reduce the risk of failure in clinical trials.

Vaccine constructs will be tested for their performance in evoking immune responses and to elicit protection in a SARS-CoV-2 lethal viral challenge model in Syrian hamsters.

Hamster Vaccination: SAM-LNPs vaccine candidates will be diluted in phosphate-buffered saline (PBS) and injected into animals intramuscularly (i.m.) using a 3/10 cc 29½G insulin syringe. Four sites of injection (30 μl each) over the lower back will be used. For a dose response curve, hamsters will be vaccinated (Primed) on day 0 and receive a booster injection on day 14. Groups will consist of a minimum of 5 animals per group for each vaccine tested, and a minimum of 3 dose ranges (e.g. 0.005 mg/kg-0.250 mg/kg) will be used for each vaccine 76. Equal numbers of males and females (5 males and 5 females) will be tested, as initial reports in humans have indicated there may be a sex difference in response to SAR-CoV-2 where males are twice as likely to develop pathological symptoms than females. A control group of unimmunized hamsters will be included. Body weights will be determined every day.

Measuring Cellular Immune Responses: Peripheral blood monocytic cells (PBMCs) will be obtained on day 0, 7, 14 and 28 post vaccination. We will harvest PBMCs to compare lymphocyte populations (T cell: CD4+, CD8+; B cell) using hamster-specific antibodies available from the Monoclonal Antibody Center at Washington State University. Inflammatory cytokines profiling will be undertaken using ELISA kits (MBL Intl) in samples collected at day 0, 4, 7, 14, 28 after challenge Quantitative reverse transcriptase polymerase chain reaction (qRT-PCR) will also be used to verify cytokine production from isolated cells and tissues, as previously described. Using blood obtained from the vaccinated hamsters above, we will screen for the production of antibodies which bind to recombinant SARS-CoV-2 proteins, using screening assays, e.g. Western blot; ELISA assay; gamma interferon EliSpot. Sera from vaccinated animals will further be tested for their ability to neutralize SARS-CoV-2 infection of HEK-293 cells by microneutralization assay as described 80. The microneutralization titer of test antisera will be determined by assessing the highest dilution of test antibody that shows inhibition in triplicate wells. Antibody subclasses concentration of IgG2a and IgG1 to examine TH1 skewing of the immune response.

Single Cell Sequencing for Measuring Immune Responses: Since reagents for hamsters are somewhat more limited than for mouse or human, another method to effective way to assess immune response will be to undertake single cell sequencing on peripheral blood monocytic cells using the 10X Genomics platform and 10X Genomics main this applicable to analysis in hamsters. This will allow the assessment of immune cells including: T cells (e.g. helper, cytotoxic, regulatory, memory, exhausted), B cells (e.g. plasma cell, memory, regulatory), in addition to innate cells (e.g. innate lymphocytes types 1, 2, and 3; monocytes/macrophages; eosinophils; mast cells).

Viral Challenge: The optimal dose of vaccine to use will be determined from the dose response trial. Hamsters will be vaccinated (Primed) on day 0 and receive a booster injection on day 14. Groups will consist of a minimum of 15 animals per group for each vaccine tested, (15 males and 15 females). At 28 days post vaccination, hamsters will be challenged with SARS-CoV-2 at a dose of 10e5 pfu in roughly 30 μl PBS via intranasal administration. Hamsters will be monitored every day for temperature, weight, and survival. Losing over 20% of their body weight will be considered a humane endpoint, and animals will be sacrificed. Tissues and cells will be harvested and examined, as described above, and bronchoalveolar lavage cell suspension will also be obtained at time of mortality.

Viral Load: Viral load and plaques will be compared by harvest lungs, and intestine and measure viral shedding by anal swabs and perform plaque assay of SARS-CoV-2 by RT-PCR and determine the pfu through incubation of serially diluted hamster samples with HEK-293 cells.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Titles, headings, or the like are provided to enhance the reader's comprehension of this document, and should not be read as limiting the scope of the present invention.

1. A vaccine comprising or encoding a first fusion protein comprising a first targeting domain and a coronavirus spike protein or fragment thereof.

2. The vaccine of claim 1, wherein said coronavirus is a SARs-CoV virus, optionally a SARs-CoV2 virus.

3. The vaccine according to claim 1, wherein the first targeting domain is a lysosomal targeting domain.

4. The vaccine according to claim 1, wherein the first targeting domain comprises (a) a HLA signal sequence and a HLA transmembrane domain, (b) a HLA signal sequence and a HLA transmembrane domain and a HLA Cytoplasmic domain, (c) CD74 Cytoplasmic domain and a HLA transmembrane domain, or (d) a HLA signal sequence.

5-7. (canceled)

8. The vaccine of claim 1, wherein the vaccine further comprises or encodes a second fusion protein comprising a second targeting domain and an influenza immunogen, optionally wherein the first targeting domain and the second targeting domain are the same or are different.

9. (canceled)

10. A vaccine comprising or encoding one or more polypeptides comprising the sequence as set forth in any one of SEQ ID NOs: 44, 46, 48, 50, 52, 54, 56, 58, 60, 62, 64 and 66 or comprising one or more sequences as set forth in any one of SEQ ID NOs: 43; 45, 47, 49, 51, 53, 55, 57, 59, 61, 63 and 65.

11. The vaccine of claim 1, wherein said vaccine is (a) a viral expression vector-based vaccine, optionally wherein the viral vector is an adenoviral vector, a vesicular stomatitis virus vector or a vaccinia vector, (b) a nucleic acid-based vaccine, or (c) a SAM RNA-based vaccine.

12-14. (canceled)

15. The vaccine of claim 11, wherein said SAM RNA-based vaccine is encapsulated in a lipid nanoparticle (LNP), optionally wherein the LNP comprises a cationic lipid, optionally wherein the LNP comprises phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA).

16-17. (canceled)

18. The vaccine of claim 1, wherein the vaccine further comprises an adjuvant.

19. A method of treating, protecting against, and/or preventing COVID-19 in a subject in need thereof, said method comprising administering one or more of vaccines of claim 1 to the subject.

20. A method of generating an immune response against one or more strains of SARS-CoV-2, said method comprising administering one or more of the vaccines of claim 1 to the subject.

21-24. (canceled)

25. A method of treating, protecting against, and/or preventing COVID-19 and influenza in a subject in need thereof, said method comprising administering one or more of the vaccines of claim 8 to the subject.

26. A method of generating an immune response against SARS-CoV-2 and influenza virus, said method comprising administering one or more of the vaccines of claim 8 to the subject.
